(19) United States
(12) Patent Application Publication
(10) Pub. No.: US 2026/0338794 A1
(43) Pub. Date: Oct. 29, 2026

(54) Methods of Assessing Tobacco Use Status

(71) Applicant: SomaLogic Operating Co., Inc., Boulder, CO (US)

(72) Inventors: Hannah Biegel, Boulder, CO (US); Yolanda Hagar, Boulder, CO (US); Kelsey M. Loupy, Boulder, CO (US); Clare Paterson, Boulder, CO (US); Rachel M. Ostroff, Boulder, CO (US); Laura Mae Sampson, Boulder, CO (US)

(73) Assignee: SomaLogic Operating Co., Inc., Boulder, CO (US)

(21) Appl. No.: 19/107,115

(22) PCT Filed: Sep. 22, 2023
(86) PCT No.: PCT/US2023/033449

§ 371 Date: Feb. 27, 2025

Related U.S. Application Data
(60) Provisional application No. 63/409,338, filed on Sep. 23, 2022.

(51) Int. Cl. G01N 33/68

(52) U.S. Cl. CPC G01N 33/6893

(57) ABSTRACT

The present disclosure includes biomarkers, methods, devices, reagents, systems, and kits for predicting an individual's tobacco use status. In one aspect, the disclosure provides biomarkers that can be used alone or in various combinations to predict an individual's tobacco use status. In another aspect, methods are provided for predicting an individual's tobacco use status, where the methods include detecting, in a biological sample from an individual, at least one biomarker value corresponding to at least one biomarker selected from the group of biomarkers provided in Table 4.

### CROSS-REFERENCE TO RELATED APPLICATIONS

This application claims the benefit of priority of U.S. Provisional Application No. 63/409,338, filed Sep. 23, 2022, which is incorporated by reference herein in its entirety for any purpose.

### FIELD

The present application relates generally to the detection of biomarkers and methods of predicting the tobacco use status of an individual and, more specifically, to one or more biomarkers, methods, devices, reagents, systems, and kits used to predict an individual's tobacco use status.

### BACKGROUND

The following description provides a summary of information that may be relevant to the present application and is not an admission that any of the information provided or publications referenced herein is prior art to the present application.

Smoking tobacco is the direct cause of over 480,000 deaths each year in the United States, according to the Centers for Disease Control and Prevention (CDC). Smoking is strongly associated with a number of chronic health conditions that decrease quality of life, including cardiovascular diseases, pulmonary diseases, and lung cancer, where the quantity and frequency of smoking are directly proportional to increased risk for disease. (Banks E, et al., BMC Med, 2019; 17 (1): 128.) Quitting smoking before the age of 40 can reduce the risk of dying from smoking-related disease by 90% thus, for nearly 60 years the U.S. Department of Health and Services has been encouraging mass cessation of cigarette smoking. (Brawley O W, et al. CA Cancer J Clin, 2014; 64 (1): 5-8.) “Former” smokers are also at a higher risk of developing comorbidities relative to “Never” smokers. (Kramarow E A. Health of former cigarette smokers aged 65 and over: United States, 2018. National Center for Health Statistics. July 2020. Available from: https://www.cdc.gov/nchs/data/nhsr/nhsr145-508.pdf.)

Current standard of care guidelines, as described by The Clinical Practice Guideline Treating Tobacco Use and Dependence 2008 Update Panel, Liaisons, and Staff, recommend the 5 A's for tobacco intervention by a primary care provider: “(1) ask the patient if he or she uses tobacco, (2) advise him or her to quit, (3) assess willingness to make a quit attempt, (4) assist those who are willing to make a quit attempt, and (5) arrange for follow-up contact to prevent relapse.” (The Clinical Practice Guideline Treating Tobacco Use and Dependence 2008 Update Panel, Liaisons, and Staff. A Clinical Practice Guideline for Treating Tobacco Use and Dependence: 2008 Update, A U.S. Public Health Service Report. Am J Prev Med, 2008; 35 (2): 158-176.) In practice, clinicians often rely on the patient's self-reported smoking habits to assess the patient's risk of smoking-related diseases and eligibility for screening. Secondarily, biochemical measures such as cotinine, carbon monoxide, or thiocyanate may be used to evaluate the accuracy of self-reported smoking habits; however, all biochemical measures of smoking are limited to the assessment of “Current” smoking habits. (Jarvis M J, et al. Am J Public Health, 1987; 77:1435-1438. Patrick D L, et al., Am J Public Health, 1994; 84 (7): 1086-1093.) The development of alternatives to assessing tobacco use status, for example by blood-based measurements, have the potential to identify clinically relevant populations that include “Former,” as well as “Current,” tobacco users.

Recently, the United States Preventative Services Task Force (USPSTF) concluded that behavioral and pharmacological interventions significantly increase smoking cessation in nonpregnant individuals. (Patnode C D, et al., JAMA, 2021; 325 (3): 280-298.) For patients who are willing to quit smoking, interventions include counseling or medication; for patients unwilling to quit, clinicians are encouraged to conduct interventions that would motivate the patient to consider quitting, such as providing information and counseling. For patients who have recently quit smoking, the clinician is advised to support the patient to prevent relapse; however, relapse prevention is not required in patients who have quit smoking for several years.

One of several first-line treatments may be prescribed to help a patient quit smoking: bupropion SR, nicotine gum, nicotine inhaler, nicotine lozenge, nicotine nasal spray, nicotine patch, or varenicline. Second-line treatments, including clonidine and nortriptyline, are not recommended unless contraindications (such as pregnancy) for first-line treatments are identified or the patient has not responded to first-line treatments. (The Clinical Practice Guideline Treating Tobacco Use and Dependence 2008 Update Panel, Liaisons, and Staff. A Clinical Practice Guideline for Treating Tobacco Use and Dependence: 2008 Update, A U.S. Public Health Service Report. Am J Prev Med, 2008; 35 (2): 158-176.)

Although cotinine, a nicotine metabolite, is often used to identify “Current” smoking habits, there are currently no biomarkers used in clinical practice that can identify “Former” smokers. (Centers for Disease Control and Prevention. Biomonitoring Summary: Cotinine. April 2017. Available from: www.cdc.gov/biomonitoring/Cotinine_BiomonitoringSummary.html). Epigenetic tests that measure DNA methylation at specific gene locations are available, such as the Smoke Signature® test by Behavioral Diagnostics, LLC; however, as marketed, this test only claims to stratify “Current” from “Never” smokers. (Philibert R, et al., Front Genet, 2018; 9:137; Dawes K, et al. Sci Rep, 2021; 11 (1): 21627.)

The development of a proteomic model for predicting an individual's probability of ever having been a tobacco user would be highly desirable. Such a proteomic model would provide knowledge of present or prior (i.e., “Ever”) tobacco use habits without reliance on subjective, misleading, or potentially falsified self-reports. A need exists for biomarkers, methods, devices, reagents, systems, and kits that enable the prediction of an individual's probability of ever having been a tobacco user.

### SUMMARY

The present application includes biomarkers, methods, reagents, devices, systems, and kits for predicting a probability of an individual ever having been a tobacco user. In some embodiments, methods of predicting a probability of an individual ever having been a tobacco user are provided. In some embodiments, methods of detecting levels of N biomarker proteins in a sample are provided.

In some embodiments, methods of predicting a probability of a subject ever having been a tobacco user are provided, comprising detecting a level of EPHA6 biomarker protein and a level of each of N biomarker proteins in a sample from the subject, wherein Nis at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments, methods of detecting levels of N biomarker proteins in a sample are provided, comprising obtaining the sample from a subject, and detecting the level of each of the N biomarker proteins in the sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments, methods of predicting a probability of a subject ever having been a tobacco user are provided, comprising detecting a level of Alkaline phosphatase-placental biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments, methods of predicting a probability of a subject ever having been a tobacco user are provided, comprising detecting a level of SCF biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments, methods of predicting a probability of a subject ever having been a tobacco user are provided, comprising detecting a level of MASP3: Heavy biomarker protein and a level of each of N biomarker proteins in a sample from the subject, wherein Nis at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments, methods of predicting a probability of a subject ever having been a tobacco user are provided, comprising detecting a level of DUS10 biomarker protein and a level of each of N biomarker proteins in a sample from the subject, wherein Nis at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments, methods of predicting a probability of a subject ever having been a tobacco user are provided, comprising detecting a level of IgG4-Kappa biomarker protein and a level of each of N biomarker proteins in a sample from the subject, wherein Nis at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, DUS10, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments, methods of predicting a probability of a subject ever having been a tobacco user are provided, comprising detecting a level of DCC biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, DUS10, IgG4-Kappa, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments, methods of predicting a probability of a subject ever having been a tobacco user are provided, comprising detecting a level of RCAN3 biomarker protein and a level of each of N biomarker proteins in a sample from the subject, wherein Nis at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, DUS10, IgG4-Kappa, DCC, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments, methods of predicting a probability of a subject ever having been a tobacco user are provided, comprising detecting a level of CD248 biomarker protein and a level of each of N biomarker proteins in a sample from the subject, wherein Nis at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, DUS10, IgG4-Kappa, DCC, RCAN3, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments, N is 2 to 39, or N is 3 to 39, or N is 4 to 39, or N is 5 to 39, or N is 6 to 39, or N is 7 to 39, or N is 8 to 39, or N is 9 to 39, or N is 10 to 39, or N is 11 to 39, or N is 12 to 39, or N is 13 to 39, or N is 14 to 39, N is 15 to 39, N is 16 to 39, N is 17 to 39, Nis 18 to 39, N is 19 to 39, N is 20 to 39, N is 21 to 39, N is 22 to 39, N is 23 to 39, N is 24 to 39, N is 25 to 39, N is 26 to 39, N is 27 to 39, N is 28 to 39, N is 29 to 39, N is 30 to 39, N is 31 to 39, N is 32 to 39, N is 33 to 39, N is 34 to 39, N is 35 to 39, N is 36 to 39, N is 37 to 39, or N is 38 to 39. In some embodiments, N is 2, N is 3, or N is 4, or N is 5, or N is 6, or N is 7, or Nis 8, or N is 9, or N is 10, or N is 11, or N is 12, or N is 13, or N is 14, or N is 15, or N is 16, or N is 17, or N is 18, or N is 19, or N is 20, or N is 21, or N is 22, or N is 23, or N is 24, N is 25, Nis 26, N is 27, N is 28, N is 29, N is 30, N is 31, N is 32, N is 33, N is 34, N is 35, N is 36, N is 37, Nis 38, or N is 39

In some embodiments, each of the N biomarker proteins is selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B. In some embodiments, at least one of the N biomarker proteins is selected from EPHA6, Alkaline phosphatase-placental, SCF, MASP3: Heavy, DUS10, IgG4-Kappa, DCC, RCAN3, and CD248. In some embodiments, at least 2, at least 3, at least 4, at least 5, at least 6, at least 7, at least 8, or at least 9 of the N protein biomarkers are selected from EPHA6, Alkaline phosphatase-placental, SCF, MASP3: Heavy, DUS10, IgG4-Kappa, DCC, RCAN3, and CD248.

In some embodiments, two of the N biomarker proteins are EPHA6 and Alkaline phosphatase-placental, or two of the N biomarker proteins are EPHA6 and SCF, or two of the N biomarker proteins are EPHA6 and MASP3: Heavy, or two of the N biomarker proteins are EPHA6 and DUS10, or two of the N biomarker proteins are EPHA6 and IgG4-Kappa, or two of the N biomarker proteins are EPHA6 and DCC, or two of the N biomarker proteins are EPHA6 and RCAN3, or two of the N biomarker proteins are EPHA6 and CD248. In some embodiments, wherein two of the N biomarker proteins are Alkaline phosphatase-placental and SCF, or two of the N biomarker proteins are Alkaline phosphatase-placental and MASP3: Heavy, or two of the N biomarker proteins are Alkaline phosphatase-placental and DUS10, or two of the N biomarker proteins are Alkaline phosphatase-placental and IgG4-Kappa, or two of the N biomarker proteins are Alkaline phosphatase-placental and DCC, or two of the N biomarker proteins are Alkaline phosphatase-placental and RCAN3, or two of the N biomarker proteins are Alkaline phosphatase-placental and CD248. In some embodiments, two of the N biomarker proteins are SCF and MASP3: Heavy, or two of the N biomarker proteins are SCF and DUS10, or two of the N biomarker proteins are SCF and IgG4-Kappa, or two of the N biomarker proteins are SCF and DCC, or two of the N biomarker proteins are SCF and RCAN3, or two of the N biomarker proteins are SCF and CD248. In some embodiments, two of the N biomarker proteins are MASP3: Heavy and DUS10, or two of the N biomarker proteins are MASP3: Heavy and IgG4-Kappa, or two of the N biomarker proteins are MASP3: Heavy and DCC, or two of the N biomarker proteins are MASP3: Heavy and RCAN3, or two of the N biomarker proteins are MASP3: Heavy and CD248. In some embodiments, two of the N biomarker proteins are DUS10 and IgG4-Kappa, or two of the N biomarker proteins are DUS10 and DCC, or two of the N biomarker proteins are DUS10 and RCAN3, or two of the N biomarker proteins are DUS10 and CD248. In some embodiments, two of the N biomarker proteins are IgG4-Kappa and DCC, or two of the N biomarker proteins are IgG4-Kappa and RCAN3, or two of the N biomarker proteins are IgG4-Kappa and CD248. In some embodiments, two of the N biomarker proteins are DCC and RCAN3, or two of the N biomarker proteins are DCC and CD248. In some embodiments, two of the N biomarker proteins are RCAN3 and CD248.

In some embodiments, a method of predicting a probability of a subject ever having been a tobacco user, comprising detecting a level of T11L1 biomarker protein is provided. In some embodiments a method of predicting a probability of a subject ever having been a tobacco user, comprising detecting a level of MXRA8 biomarker protein is provided. In some embodiments, a method of predicting a probability of a subject ever having been a tobacco user, comprising detecting a level of XTP3A biomarker protein is provided. In some embodiments, a method of predicting a probability of a subject ever having been a tobacco user, comprising detecting a level of CRAC1 biomarker protein is provided. In some embodiments, the method further comprises detecting at least one biomarker protein selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit.

In some embodiments, the sample is a blood sample, a plasma sample, or a serum sample. In some embodiments, detecting is performed using mass spectrometry, an aptamer based assay and/or an antibody based assay. In some embodiments, the method includes contacting biomarker proteins of the sample or samples with a set of biomarker capture reagents, wherein each biomarker capture reagent of the set of biomarker capture reagents specifically binds to a different biomarker protein being detected. In some embodiments, each biomarker capture reagent is an antibody or an aptamer. In some embodiments, each biomarker capture reagent is an aptamer. In some embodiments, at least one aptamer is a slow off-rate aptamer. In some embodiments, at least one slow off-rate aptamer includes at least one, at least two, at least three, at least four, at least five, at least six, at least seven, at least eight, at least nine, or at least 10 nucleotides with modifications. In some embodiments, each slow off-rate aptamer binds to its target protein with an off rate (t½) of ≥20 minutes, ≥30 minutes, ≥60 minutes, ≥90 minutes, ≥120 minutes, ≥150 minutes, ≥180 minutes, ≥210 minutes, or ≥240 minutes. In some embodiments, the level of each biomarker protein measured is determined from a relative florescence unit (RFU) or a protein concentration.

In some embodiments, predicting the probability of the subject ever having been a tobacco user is based on input of the levels of the N biomarker proteins measured in a statistical model. In some embodiments, the determining comprises analyzing the levels of the N biomarker protein using an elastic net logistic regression model. In some embodiments, the model has an area under the curve (AUC) selected from at least 0.65, at least 0.66, at least 0.67, at least 0.68, at least 0.69, at least 0.7, at least 0.75, at least 0.8, at least 0.85, at least 0.9, or at least 0.95. In some embodiments, the model provides an absolute risk probability of having ever been a tobacco user. In some embodiments, the model provides a value between 0 and 1, wherein a value >0.460 is predictive of an ever tobacco user. In some embodiments, the model provides the absolute probability of having been an ever tobacco user based on the level of each of the biomarker proteins selected from EPHA6, Alkaline phosphatase-placental, SCF, MASP3: Heavy, DUS10, IgG4-Kappa, DCC, RCAN3, and CD248. In some embodiments, the model provides the absolute probability of having been an ever tobacco user based on the level of each of the biomarker proteins selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments, methods are provided for predicting a probability of a subject ever having been a tobacco user for the purpose of determining a medical insurance premium or life insurance premium. In some embodiments, the methods further comprise determining coverage or premium for medical insurance or life insurance. In some embodiments, methods are provided that further comprise using information resulting from the method to predict and/or manage the utilization of medical resources. In some embodiments, the methods further comprise using information resulting from the method to enable a decision to acquire or purchase a medical practice, hospital, or company. In any of the embodiments, the tobacco user is a smoker.

In some embodiments, a kit is provided wherein the kit includes N biomarker protein capture reagents, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker protein capture reagents specifically binds to a biomarker protein selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

In some embodiments a kit including N biomarker protein capture reagents is provided, wherein the kit includes biomarker protein capture reagents for carrying out any of the methods described herein. In some embodiments, each of the N biomarker protein capture reagents is an antibody or an aptamer. In some embodiments, each biomarker protein capture reagent is an aptamer. In some embodiments, at least one aptamer is a slow off-rate aptamer. In some embodiments, at least one slow off-rate aptamer comprises at least one, at least two, at least three, at least four, at least five, at least six, at least seven, at least eight, at least nine, or at least 10 nucleotides with modifications. In some embodiments, each slow off-rate aptamer binds to its target protein with an off rate (t½) of ≥20 minutes, ≥30 minutes, ≥60 minutes, ≥90 minutes, ≥120 minutes, ≥150 minutes, ≥180 minutes, ≥210 minutes, or ≥240 minutes. In some embodiments, the kit is for use in detecting the N biomarker proteins in a sample from a subject. In some embodiments, the kit is for use in use in predicting a probability of a subject ever having been a tobacco user.

### BRIEF DESCRIPTION OF THE DRAWINGS

FIG. 1 shows a receiver operating characteristic (ROC) curve for the Tobacco use Status reference model on training, verification, and validation datasets.

FIG. 2 shows box plots of the predicted probabilities stratified by dataset (training, verification, or validation) and “Ever” versus “Never” tobacco use classification. The horizontal black line corresponds to the decision cutoff of 0.46.

FIG. 3 shows certain exemplary modifications in an aptamer that may be present at the 5-position of uridine. The chemical structure of the C-5 modification includes the exemplary amide linkage that links the modification to the 5-position of the uridine. The 5-position moieties shown include a benzyl moiety (e.g., Bn, PE and a PP), a naphthyl moiety (e.g., Nap, 2Nap, NE), a butyl moiety (e.g, iBu), a fluorobenzyl moiety (e.g., FBn), a tyrosyl moiety (e.g., a Tyr), a 3,4-methylenedioxy benzyl (e.g., MBn), a morpholino moiety (e.g., MOE), a benzofuranyl moiety (e.g., BF), an indole moiety (e.g, Trp) and a hydroxypropyl moiety (e.g., Thr).

FIG. 4 shows certain exemplary modifications in an aptamer that may be present at the 5-position of cytidine. The chemical structure of the C-5 modification includes the exemplary amide linkage that links the modification to the 5-position of the cytidine. The 5-position moieties shown include a benzyl moiety (e.g., Bn, PE and a PP), a naphthyl moiety (e.g., Nap, 2Nap, NE, and 2NE) and a tyrosyl moiety (e.g., a Tyr).

FIG. 5 illustrates an exemplary computer system for use with various computer-implemented methods described herein.

FIG. 6 is a flowchart for a method of evaluating probability of an individual having ever been a tobacco user in accordance with one or more embodiments.

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

Unless defined otherwise, technical and scientific terms used herein have the same meaning as commonly understood by one of ordinary skill in the art to which this invention belongs. Although any methods, devices, and materials similar or equivalent to those described herein can be used in the practice or testing of the invention, certain methods, devices and materials are now described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include plural references, unless the content clearly dictates otherwise, and are used interchangeably with “at least one” and “one or more.” Thus, reference to “a SOMAmer” includes mixtures of SOMAmers, reference to “a probe” includes mixtures of probes, and the like.

As used herein, the term “about” represents an insignificant modification or variation of the numerical value such that the basic function of the item to which the numerical value relates is unchanged.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

The present application includes biomarkers, methods, devices, reagents, systems, and kits for the determining the probability an individual ever having been a tobacco user.

“Tobacco use” as used herein includes smoking and chewing tobacco. “Tobacco use” as used herein does not include vaping.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), dried blood spots (e.g., obtained from infants), sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

For purposes of this specification, the phrase “data attributed to a biological sample from an individual” is intended to mean that the data in some form derived from, or were generated using, the biological sample of the individual. The data may have been reformatted, revised, or mathematically altered to some degree after having been generated, such as by conversion from units in one measurement system to units in another measurement system; but, the data are understood to have been derived from, or were generated using, the biological sample.

“Target”, “target molecule”, and “analyte” are used interchangeably herein to refer to any molecule of interest that may be present in a biological sample. A “molecule of interest” includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule”, “target”, or “analyte” is a set of copies of one type or species of molecule or multi-molecular structure. “Target molecules”, “targets”, and “analytes” refer to more than one such set of molecules. Exemplary target molecules include proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing. In some embodiments, a target molecule is a protein, in which case the target molecule may be referred to as a “target protein.”

As used herein, a “capture agent’ or “capture reagent” refers to a molecule that is capable of binding specifically to a biomarker. A “target protein capture reagent” refers to a molecule that is capable of binding specifically to a target protein. Nonlimiting exemplary capture reagents include aptamers, antibodies, adnectins, ankyrins, other antibody mimetics and other protein scaffolds, autoantibodies, chimeras, small molecules, nucleic acids, lectins, ligand-binding receptors, imprinted polymers, avimers, peptidomimetics, hormone receptors, cytokine receptors, synthetic receptors, and modifications and fragments of any of the aforementioned capture reagents. In some embodiments, a capture reagent is selected from an aptamer and an antibody.

As used herein, “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can be single chains or associated chains. Also included within the definition are preproteins and intact mature proteins; peptides or polypeptides derived from a mature protein; fragments of a protein; splice variants; recombinant forms of a protein; protein variants with amino acid modifications, deletions, or substitutions; digests; and post-translational modifications, such as glycosylation, acetylation, phosphorylation, and the like.

The term “antibody” refers to full-length antibodies of any species and fragments and derivatives of such antibodies, including Fab fragments, F(ab′) 2 fragments, single chain antibodies, Fv fragments, and single chain Fv fragments. The term “antibody” also refers to synthetically-derived antibodies, such as phage display-derived antibodies and fragments, affybodies, nanobodies, etc.

As used herein, “marker” and “biomarker” and “feature” are used interchangeably to refer to a target molecule that indicates or is a sign of a normal or abnormal process in an individual or of a disease or other condition in an individual. More specifically, a “marker” or “biomarker” or “feature” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. When a biomarker is a protein, it is also possible to use the expression of the corresponding gene as a surrogate measure of the amount or presence or absence of the corresponding protein biomarker in a biological sample or methylation state of the gene encoding the biomarker or proteins that control expression of the biomarker. In certain aspects, a feature is an analyte/SOMAmer reagent of other predictors in a statistical model.

As used herein, “biomarker value”, “value”, “biomarker level”, “feature level” and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “value” or “level” depends on the specific design and components of the particular analytical method employed to detect the biomarker.

When a biomarker indicates or is a sign of an abnormal process or a disease or other condition in an individual, that biomarker is generally described as being either over-expressed or under-expressed as compared to an expression level or value of the biomarker that indicates or is a sign of a normal process or an absence of a disease or other condition in an individual. “Up-regulation”, “up-regulated”, “over-expression”, “over-expressed”, and any variations thereof are used interchangeably to refer to a value or level of a biomarker in a biological sample that is greater than a value or level (or range of values or levels) of the biomarker that is typically detected in similar biological samples from healthy or normal individuals. The terms may also refer to a value or level of a biomarker in a biological sample that is greater than a value or level (or range of values or levels) of the biomarker that may be detected at a different stage of a particular disease.

“Down-regulation”, “down-regulated”, “under-expression”, “under-expressed”, and any variations thereof are used interchangeably to refer to a value or level of a biomarker in a biological sample that is less than a value or level (or range of values or levels) of the biomarker that is typically detected in similar biological samples from healthy or normal individuals. The terms may also refer to a value or level of a biomarker in a biological sample that is less than a value or level (or range of values or levels) of the biomarker that may be detected at a different stage of a particular disease.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

The term “differential gene expression” and “differential expression” are used interchangeably to refer to a gene (or its corresponding protein expression product) whose expression is activated to a higher or lower level in a subject suffering from a specific disease or condition, relative to its expression in a normal or control subject. The terms also include genes (or the corresponding protein expression products) whose expression is activated to a higher or lower level at different stages of the same disease or condition. It is also understood that a differentially expressed gene may be either activated or inhibited at the nucleic acid level or protein level, or may be subject to alternative splicing to result in a different polypeptide product. Such differences may be evidenced by a variety of changes including mRNA levels, surface expression, secretion or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

A “control level” of a target molecule refers to the level of the target molecule in a properly handled sample of the same sample type. Control level may refer to the average level of the target molecule in properly handled samples from a population of individuals.

As used herein, “individual” refers to a test subject or patient. The individual can be a mammal or a non-mammal. In various embodiments, the individual is a mammal. A mammalian individual can be a human or non-human. In various embodiments, the individual is a human.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual. The probability of ever having been a tobacco user includes distinguishing individuals who have ever been a tobacco user (former or current) from individuals who have never been a tobacco user.

“Prognose”, “prognosing”, “prognosis”, and variations thereof refer to the prediction of a future course of a disease or condition in an individual who has the disease or condition (e.g., predicting patient survival), and such terms encompass the evaluation of disease or condition response after the administration of a treatment or therapy to the individual.

“Evaluate”, “evaluating”, “evaluation”, and variations thereof encompass both “diagnose” and “prognose” and also encompass determinations or predictions about the future course of a disease or condition in an individual who does not have the disease as well as determinations or predictions regarding the risk that a disease or condition will recur in an individual who apparently has been cured of the disease or has had the condition resolved. The term “evaluate” also encompasses assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual.

As used herein, “additional biomedical information” refers to one or more evaluations of an individual, other than using any of the biomarkers described herein, that are associated with tobacco use. “Additional biomedical information” includes any of the following: physical descriptors of an individual, including the height and/or weight of an individual; the age of an individual; the gender of an individual; change in weight; the ethnicity of an individual; occupational history; family history of tobacco use; the presence of a genetic marker(s); clinical symptoms such as abdominal pain, weight gain or loss gene expression values; physical descriptors of an individual, including physical descriptors observed by radiologic imaging; tobacco use status; alcohol use history; occupational history; dietary habits-salt, saturated fat and cholesterol intake; caffeine consumption; and imaging information. Additional biomedical information can be obtained from an individual using routine techniques known in the art, such as from the individual themselves by use of a routine patient questionnaire or health history questionnaire, etc., or from a medical practitioner, etc.

As used herein, “detecting” or “determining” with respect to a biomarker value includes the use of both the instrument required to observe and record a signal corresponding to a biomarker value and the material/s required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

“Solid support” refers herein to any substrate having a surface to which molecules may be attached, directly or indirectly, through either covalent or non-covalent bonds. A “solid support” can have a variety of physical formats, which can include, for example, a membrane; a chip (e.g., a protein chip); a slide (e.g., a glass slide or coverslip); a column; a hollow, solid, semi-solid, pore- or cavity-containing particle, such as, for example, a bead; a gel; a fiber, including a fiber optic material; a matrix; and a sample receptacle. Exemplary sample receptacles include sample wells, tubes, capillaries, vials, and any other vessel, groove or indentation capable of holding a sample. A sample receptacle can be contained on a multi-sample platform, such as a microtiter plate, slide, microfluidics device, and the like. A support can be composed of a natural or synthetic material, an organic or inorganic material. The composition of the solid support on which capture reagents are attached generally depends on the method of attachment (e.g., covalent attachment). Other exemplary receptacles include microdroplets and microfluidic controlled or bulk oil/aqueous emulsions within which assays and related manipulations can occur. Suitable solid supports include, for example, plastics, resins, polysaccharides, silica or silica-based materials, functionalized glass, modified silicon, carbon, metals, inorganic glasses, membranes, nylon, natural fibers (such as, for example, silk, wool and cotton), polymers, and the like. The material composing the solid support can include reactive groups such as, for example, carboxy, amino, or hydroxyl groups, which are used for attachment of the capture reagents. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly) vinylidenefluoride, polycarbonate, and polymethylpentene. Suitable solid support particles that can be used include, e.g., encoded particles, such as Luminex®-type encoded particles, magnetic particles, and glass particles.

As used herein, “analyte” is the protein target of a capture reagent. In certain aspects, the capture reagent is an aptamer. In certain further aspects, the capture reagent is a SOMAmer.

As used herein, “Lin's CCC” means concordance correlation coefficient which measures the concordance between a new test and an existing test that is considered the gold standard.

As used herein, “study”, means a set of samples and clinical data that are analyzed to derive the test.

As used herein, “training dataset”, means a subset of data from a study used to fit a model.

As used herein, “validation dataset”, means a final subset of data used to assess the performance of a selected model developed on a verification dataset.

As used herein, “verification dataset”, means a separate subset of data used to provide an unbiased evaluation of a model fit on the training dataset while tuning model parameters.

As used herein, the term “need” or “needed” refers to a judgement made by a health care provider regarding treatment of a patient which is considered by the health care provider to be beneficial to the health status of the patient.

### Risk Analysis

In some aspects, an objective test for predicting an individual's probability of ever having been a tobacco user is disclosed herein.

The risk analysis profile may be described as in Table 1.

TABLE 1
Risk analysis

| | | Likelihood | | Need for |
|---|---|---|---|---|
| | | relative to | | Mitigating |
| Hazard | Hazardous Situation(s) | SoC | Harm(s) | Measures |
| False Positive Result | Doctor may need additional | [ ] Lower | HCP may be inclined to | [✓] Low |
| (erroneous up- | consulting with patient. | [✓] Equivalent | mistrust patient. | [ ] Med |
| classification of tobacco | Doctor may consider the | [ ] Higher | Doctor may recommend | [ ] High |
| use habits) | patient more at risk for | | or perform unnecessary | |
| | tobacco use-related diseases | | screening procedures for | |
| | than the patient actually is. | | tobacco use-related | |
| | | | disease. | |
| | Patient initiates lifestyle | [✓] Lower | Slight inconvenience for | |
| | changes to lower possible | [ ] Equivalent | patient but no physical | |
| | sources of tobacco use | [ ] Higher | harm, as these lifestyle | |
| | impact on proteome (i.e., | | choices are known to | |
| | reduce secondhand smoke | | improve overall health | |
| | exposure). | | and adhere to general | |
| | | | health guidelines. | |
| False Negative | Doctor may not obtain | [ ] Lower | HCP may alter | [ ] Low |
| Result (erroneous down- | correct information | [✓] Equivalent | recommendations (i.e., | [✓] Med |
| classification of tobacco | regarding disease risk due | [ ] Higher | not recommend quitting, | [ ] High |
| use status) | to tobacco tobacco use | | not recommend | |
| | habits. | | screening) for patient | |
| | | | based on this | |
| | | | misinformation. | |
| | Patient may believe that | | Patient may feel | |
| | their tobacco use habits are | | encouraged to continue | |
| | not affecting their | | or increase tobacco | |
| | physiology and thus are not | | smoke intake or | |
| | of negative consequence. | | exposure. | |
| Off Label Use | Physician uses this test to | [ ] Lower | Misguided information, | [ ] Low |
| | screen for tobacco use- | [✓] Equivalent | may cause undue stress | [✓] Med |
| | related disease. | [ ] Higher | to HCP and patient. | [ ] High |

The testing methods disclosed herein provide knowledge of present or prior (i.e., “Ever”) tobacco use habits without reliance on subjective, misleading, or falsified self-reports. The testing methods disclosed herein may influence positive behavior changes (i.e., cessation of tobacco use) that may prevent or delay tobacco use-related conditions. The testing methods disclosed herein may allow healthcare providers and patients to monitor tobacco use habits, compliance with tobacco use cessation, and tobacco use impact on the proteome across time. The testing methods disclosed herein may be taken into consideration for an individual's risk assessment for disease screening eligibility.

TABLE 2
Model Performance Target

| Validation Metric(s) | Performance Target |
|---|---|
| Area Under the | AUC greater than or |
| Curve (AUC) | equal to 0.717 |

The performance threshold was established based on the performance of the leading epigenetic predictor model available on the market, DNA methylation of the AHRR gene, to predict smoking habits. (Langdon R J, et al., Clin Epigenetics, 2021; 13 (1): 206; Philibert R, et al., Front Genet, 2018; 9:137.)

There is no available test on the market that predicts “Former”, as well as “Current”, tobacco use status. Cotinine, a nicotine metabolite, is commonly used in clinical settings to predict recent (i.e., less than 3 days) tobacco use. (Centers for Disease Control and Prevention. Biomonitoring Summary: Cotinine. April 2017. Available from: https://www.cdc.gov/biomonitoring/Cotinine_BiomonitoringSummary.html). However, cotinine is not a comparable reference tool because it does not indicate longer-term biological impact of tobacco smoke exposure. The testing methods disclosed herein are at least comparable to the performance of current epigenetic predictor models; for example, the Smoke Signature® test provided by Behavioral Diagnostics, LLC claims to stratify only “Current” smokers versus non-smokers using cutoffs for DNA methylation of the AHRR gene. (Philibert R, et al., Front Genet, 2018; 9:137; Dawes K, et al. Sci Rep, 2021; 11 (1): 21627.) Recently, AHIRR methylation was applied in a two-stage approach to assess its ability to classify ternary smoking status, performing with AUCs ranging from 0.717 to 0.902. (Langdon R J, et al., Clin Epigenetics, 2021; 13 (1): 206.) While epigenetic changes may be quantitative (Philibert R, et al., Front Psychiatry, 2016; 7:55), these changes require significant alterations to smoke exposure and take an extended period (i.e., years) to show a measurable difference in smoking status class; (McCartney D L, et al., EBioMedicine, 2018; 37:214-220) thus, the assessment of a dynamic proteome may be better positioned to predict more rapid changes in smoking habits. Embodiments of the methods disclosed herein perform with an AUC greater than or equal to 0.65, 0.66, 0.67. 0.68, 0.69, 0.70, 0.717, or greater, with 0.717 being the lower bounds of AHRR methylation performance reported for ternary classification.

In some embodiments, the number of biomarkers useful for a biomarker subset or panel is based on the sensitivity and specificity value for the particular combination of biomarker levels. The terms “sensitivity” and “specificity” are used herein with respect to the ability to correctly classify an individual, based on one or more biomarker levels detected in their biological sample, as having ever smoked or having never smoked. “Sensitivity” indicates the performance of the biomarker(s) with respect to correctly classifying individuals that have ever (current and former) smoked or never smoked. “Specificity” indicates the performance of the biomarker(s) with respect to correctly classifying individuals who have never smoked.

In some embodiments, overall performance of a panel of one or more biomarkers is represented by the area-under-the-curve (AUC) value. The AUC value is derived from a receiver operating characteristic (ROC) curve. The ROC curve is the plot of the true positive rate (sensitivity) of a test against the false positive rate (1-specificity) of the test. The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., ever tobacco users (former and current) and never tobacco users). ROC curves are useful for plotting the performance of a particular feature (e.g., any of the biomarkers described herein and/or any item of additional biomedical information) in distinguishing between two populations. Typically, the feature data across the entire population are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve.

### Exemplary Uses of Biomarkers

In various exemplary embodiments, methods are provided for predicting a probability of ever having been a tobacco user in an individual by detecting one or more biomarker values corresponding to one or more biomarkers that are present in the circulation of an individual, such as in blood, serum or plasma, by any number of analytical methods, including any of the analytical methods described herein. These biomarkers are, for example, differentially expressed in individuals who were ever (former and current) tobacco users as comparted to individual who never smoked. Detection of the differential expression of a biomarker in an individual can be used, for example, to permit the prediction of a probability of ever having been a tobacco user.

In addition to testing biomarker levels as a stand-alone diagnostic test, biomarker levels can also be done in conjunction with determination of SNPs or other genetic lesions or variability that are indicative of increased risk of susceptibility of disease or condition. (See, e.g., Amos et al., Nature Genetics 40, 616-622 (2009)).

Any of the described biomarkers may also be used in imaging tests. For example, an imaging agent can be coupled to any of the described biomarkers, which can be used to aid in prediction of an individual's probability of ever having been a tobacco user, to monitor response to therapeutic interventions, to select for target populations in a clinical trial among other uses.

### Detection and Determination of Biomarkers and Biomarker Levels

A biomarker level for the biomarkers described herein can be detected using any of a variety of known analytical methods. In one embodiment, a biomarker level is detected using a capture reagent. As used herein, a “capture agent” or “capture reagent” refers to a molecule that is capable of binding specifically to a biomarker. In various embodiments, the capture reagent can be exposed to the biomarker in solution or can be exposed to the biomarker while the capture reagent is immobilized on a solid support. In other embodiments, the capture reagent contains a feature that is reactive with a secondary feature on a solid support. In these embodiments, the capture reagent can be exposed to the biomarker in solution, and then the feature on the capture reagent can be used in conjunction with the secondary feature on the solid support to immobilize the biomarker on the solid support. The capture reagent is selected based on the type of analysis to be conducted. Capture reagents include but are not limited to SOMAmers, antibodies, adnectins, ankyrins, other antibody mimetics and other protein scaffolds, autoantibodies, chimeras, small molecules, a F(ab′)₂ fragment, a single chain antibody fragment, an Fv fragment, a single chain Fv fragment, a nucleic acid, a lectin, a ligand-binding receptor, affybodies, nanobodies, imprinted polymers, avimers, peptidomimetics, a hormone receptor, a cytokine receptor, and synthetic receptors, and modifications and fragments of these.

In some embodiments, a biomarker level is detected using a biomarker/capture reagent complex.

In other embodiments, the biomarker level is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, the biomarker level is detected directly from the biomarker in a biological sample.

In one embodiment, the biomarkers are detected using a multiplexed format that allows for the simultaneous detection of two or more biomarkers in a biological sample. In one embodiment of the multiplexed format, capture reagents are immobilized, directly or indirectly, covalently or non-covalently, in discrete locations on a solid support. In another embodiment, a multiplexed format uses discrete solid supports where each solid support has a unique capture reagent associated with that solid support, such as, for example quantum dots. In another embodiment, an individual device is used for the detection of each one of multiple biomarkers to be detected in a biological sample. Individual devices can be configured to permit each biomarker in the biological sample to be processed simultaneously. For example, a microtiter plate can be used such that each well in the plate is used to uniquely analyze one of multiple biomarkers to be detected in a biological sample.

In one or more of the foregoing embodiments, a fluorescent tag can be used to label a component of the biomarker/capture complex to enable the detection of the biomarker value. In various embodiments, the fluorescent label can be conjugated to a capture reagent specific to any of the biomarkers described herein using known techniques, and the fluorescent label can then be used to detect the corresponding biomarker value. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In one embodiment, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor molecule, such as, for example, AlexaFluor 488, AlexaFluor 532, AlexaFluor 647, AlexaFluor 680, or AlexaFluor 700. In other embodiments, the dye molecule includes a first type and a second type of dye molecule, such as, e.g., two different AlexaFluor molecules. In other embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

In one or more of the foregoing embodiments, a chemiluminescence tag can optionally be used to label a component of the biomarker/capture complex to enable the detection of a biomarker value. Suitable chemiluminescent materials include any of oxalyl chloride, Rodamin 6G, Ru (bipy) 32+, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

In yet other embodiments, the detection method includes an enzyme/substrate combination that generates a detectable signal that corresponds to the biomarker value. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In yet other embodiments, the detection method can be a combination of fluorescence, chemiluminescence, radionuclide or enzyme/substrate combinations that generate a measurable signal. Multimodal signaling could have unique and advantageous characteristics in biomarker assay formats.

More specifically, the biomarker levels for the biomarkers described herein can be detected using known analytical methods including, singleplex SOMAmer assays, multiplexed SOMAmer assays, singleplex or multiplexed immunoassays, mRNA expression profiling, miRNA expression profiling, mass spectrometric analysis, histological/cytological methods, etc. as detailed below.

### Determination of Biomarker Levels Using Aptamer-Based Assays

Assays directed to the detection and quantification of physiologically significant molecules in biological samples and other samples are important tools in scientific research and in the health care field. One class of such assays involves the use of a microarray that includes one or more aptamers immobilized on a solid support. The aptamers are each capable of binding to a target molecule in a highly specific manner and with very high affinity. See, e.g., U.S. Pat. No. 5,475,096 entitled “Nucleic Acid Ligands”; see also, e.g., U.S. Pat. Nos. 6,242,246, 6,458,543, and 6,503,715, each of which is entitled “Nucleic Acid Ligand Diagnostic Biochip”. Once the microarray is contacted with a sample, the aptamers bind to their respective target molecules present in the sample and thereby enable a determination of a biomarker value corresponding to a biomarker.

As used herein, an “aptamer” refers to a nucleic acid that has a specific binding affinity for a target molecule. It is recognized that affinity interactions are a matter of degree; however, in this context, the “specific binding affinity” of an aptamer for its target means that the aptamer binds to its target generally with a much higher degree of affinity than it binds to other components in a test sample. An “aptamer” is a set of copies of one type or species of nucleic acid molecule that has a particular nucleotide sequence. An aptamer can include any suitable number of nucleotides, including any number of chemically modified nucleotides. “Aptamers” refers to more than one such set of molecules. Different aptamers can have either the same or different numbers of nucleotides. Aptamers can be DNA or RNA or chemically modified nucleic acids and can be single stranded, double stranded, or contain double stranded regions, and can include higher ordered structures. An aptamer can also be a photoaptamer, where a photoreactive or chemically reactive functional group is included in the aptamer to allow it to be covalently linked to its corresponding target. Any of the aptamer methods disclosed herein can include the use of two or more aptamers that specifically bind the same target molecule. As further described below, an aptamer may include a tag. If an aptamer includes a tag, all copies of the aptamer need not have the same tag. Moreover, if different aptamers each include a tag, these different aptamers can have either the same tag or a different tag.

An aptamer can be identified using any known method, including the SELEX process. Once identified, an aptamer can be prepared or synthesized in accordance with any known method, including chemical synthetic methods and enzymatic synthetic methods.

As used herein, a “SOMAmer” or Slow Off-Rate Modified Aptamer refers to an aptamer having improved off-rate characteristics. SOMAmers can be generated using the improved SELEX methods described in U.S. Publication No. 2009/0004667, entitled “Method for Generating Aptamers with Improved Off-Rates.”

The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific target or biomarker.

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′—NH2), 2′-fluoro (2′-F), and/or 2′-O-methyl(2′-OMe). See also, U.S. Patent Application Publication 20090098549, entitled “SELEX and PHOTOSELEX”, which describes nucleic acid libraries having expanded physical and chemical properties and their use in SELEX and photoSELEX.

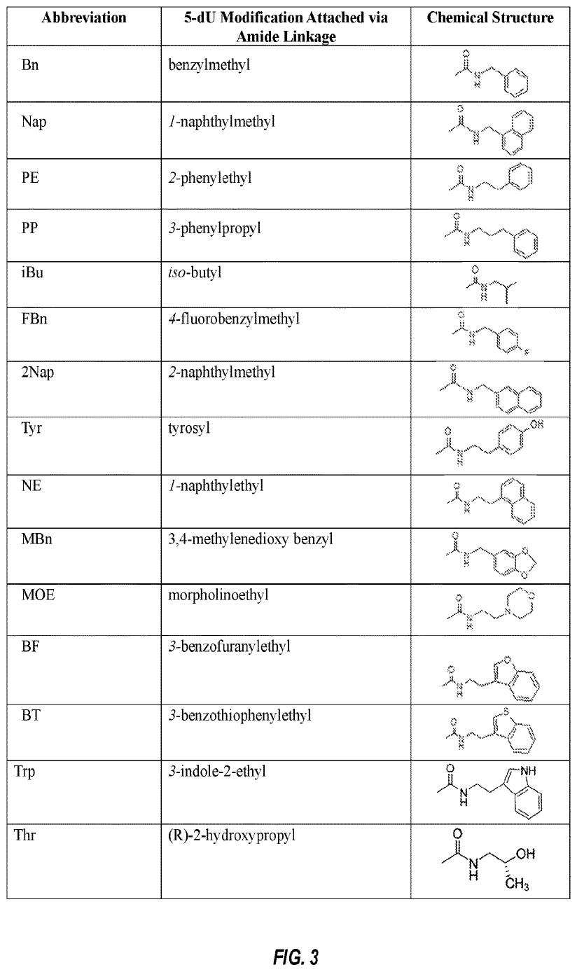

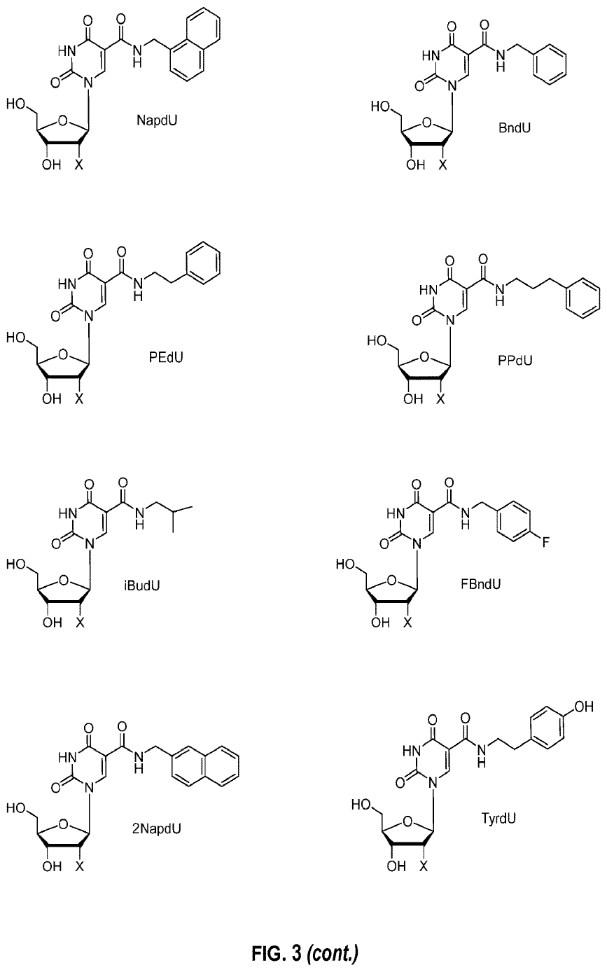

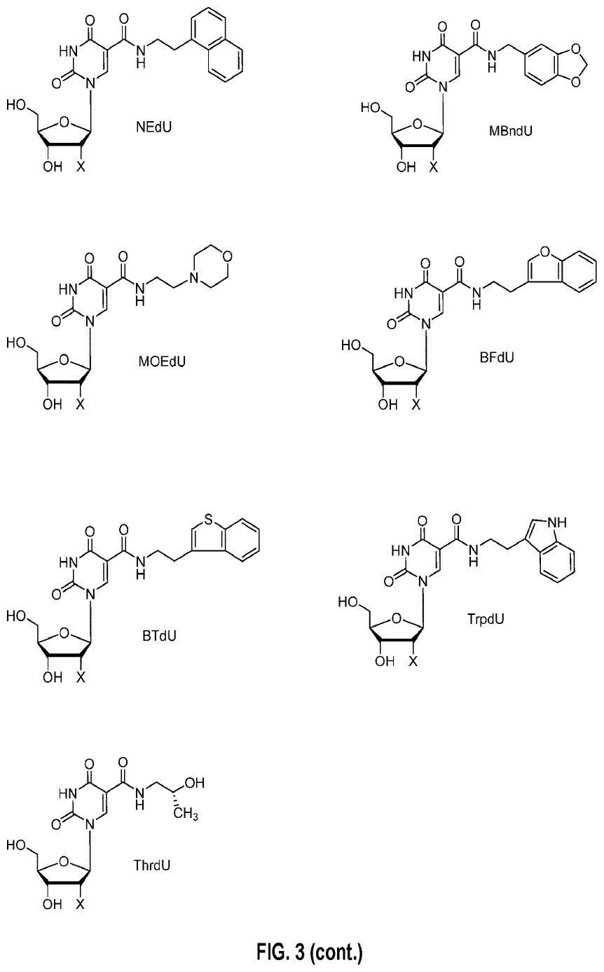

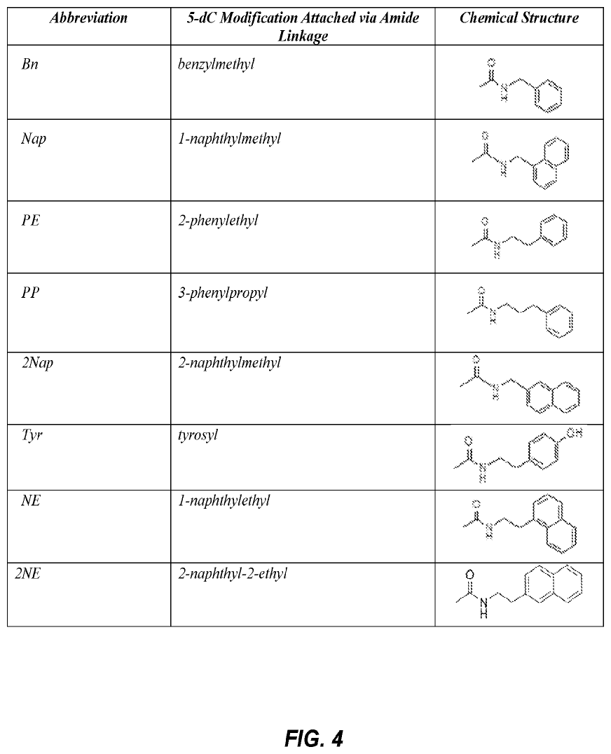

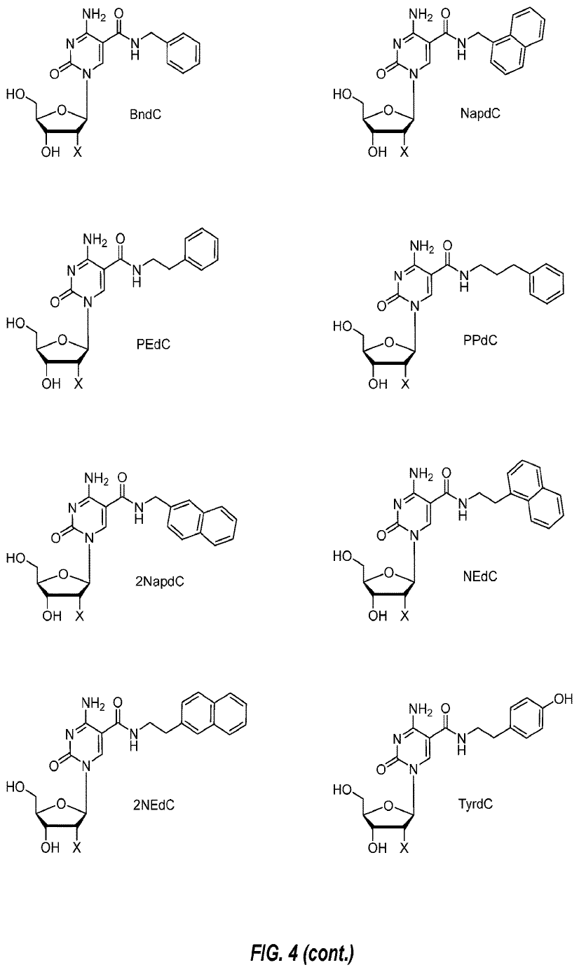

SELEX can also be used to identify aptamers that have desirable off-rate characteristics. See U.S. Patent Application Publication 20090004667, entitled “Method for Generating Aptamers with Improved Off-Rates”, which describes improved SELEX methods for generating aptamers that can bind to target molecules. As mentioned above, these slow off-rate aptamers are known as “SOMAmers.” Methods for producing aptamers or SOMAmers and photoaptamers or SOMAmers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers or SOMAmers with improved off-rate performance. Nonlimiting exemplary modified nucleotides include, for example, the modified pyrimidines shown in FIGS. 3 and 4.

A variation of this assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. Nos. 5,763,177, 6,001,577, and 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers. In this manner, the assay enables the detection of a biomarker value corresponding to a biomarker in the test sample.

In both of these assay formats, the aptamers or SOMAmers are immobilized on the solid support prior to being contacted with the sample. Under certain circumstances, however, immobilization of the aptamers or SOMAmers prior to contact with the sample may not provide an optimal assay. For example, pre-immobilization of the aptamers or SOMAmers may result in inefficient mixing of the aptamers or SOMAmers with the target molecules on the surface of the solid support, perhaps leading to lengthy reaction times and, therefore, extended incubation periods to permit efficient binding of the aptamers or SOMAmers to their target molecules. Further, when photoaptamers or photoSOMAmers are employed in the assay and depending upon the material utilized as a solid support, the solid support may tend to scatter or absorb the light used to effect the formation of covalent bonds between the photoaptamers or photoSOMAmers and their target molecules. Moreover, depending upon the method employed, detection of target molecules bound to their aptamers or photoSOMAmers can be subject to imprecision, since the surface of the solid support may also be exposed to and affected by any labeling agents that are used. Finally, immobilization of the aptamers or SOMAmers on the solid support generally involves an aptamer or SOMAmer-preparation step (i.e., the immobilization) prior to exposure of the aptamers or SOMAmers to the sample, and this preparation step may affect the activity or functionality of the aptamers or SOMAmers.

SOMAmer assays that permit a SOMAmer to capture its target in solution and then employ separation steps that are designed to remove specific components of the SOMAmer-target mixture prior to detection have also been described (see U.S. Patent Application Publication 20090042206, entitled “Multiplexed Analyses of Test Samples”). The described SOMAmer assay methods enable the detection and quantification of a non-nucleic acid target (e.g., a protein target) in a test sample by detecting and quantifying a nucleic acid (i.e., a SOMAmer). The described methods create a nucleic acid surrogate (i.e, the SOMAmer) for detecting and quantifying a non-nucleic acid target, thus allowing the wide variety of nucleic acid technologies, including amplification, to be applied to a broader range of desired targets, including protein targets.

SOMAmers can be constructed to facilitate the separation of the assay components from a SOMAmer biomarker complex (or photoSOMAmer biomarker covalent complex) and permit isolation of the SOMAmer for detection and/or quantification. In some embodiments, these constructs can include a cleavable or releasable element within the SOMAmer sequence. In other embodiments, additional functionality can be introduced into the SOMAmer, for example, a labeled or detectable component, a spacer component, or a specific binding tag or immobilization element. For example, the SOMAmer can include a tag connected to the SOMAmer via a cleavable moiety, a label, a spacer component separating the label, and the cleavable moiety. In one embodiment, a cleavable element is a photocleavable linker. The photocleavable linker can be attached to a biotin moiety and a spacer section, can include an NHS group for derivatization of amines, and can be used to introduce a biotin group to an aptamer, thereby allowing for the release of the aptamer later in an assay method.

Homogenous assays, done with all assay components in solution, do not require separation of sample and reagents prior to the detection of signal. These methods are rapid and easy to use. These methods generate signal based on a molecular capture or binding reagent that reacts with its specific target. For prediction of tobacco use status, the molecular capture reagents would be an aptamer or an antibody or the like and the specific target would be a tobacco use biomarker as in Table 4.

In some embodiments, a method for signal generation takes advantage of anisotropy signal change due to the interaction of a fluorophore-labeled capture reagent with its specific biomarker target. When the labeled capture reagent reacts with its target, the increased molecular weight causes the rotational motion of the fluorophore attached to the complex to become much slower changing the anisotropy value. By monitoring the anisotropy change, binding events may be used to quantitatively measure the biomarkers in solutions. Other methods include fluorescence polarization assays, molecular beacon methods, time resolved fluorescence quenching, chemiluminescence, fluorescence resonance energy transfer, and the like.

An exemplary solution-based aptamer assay that can be used to detect a biomarker value corresponding to a biomarker in a biological sample includes the following: (a) preparing a mixture by contacting the biological sample with an aptamer that includes a first tag and has a specific affinity for the biomarker, wherein an aptamer affinity complex is formed when the biomarker is present in the sample; (b) exposing the mixture to a first solid support including a first capture element, and allowing the first tag to associate with the first capture element; (c) removing any components of the mixture not associated with the first solid support; (d) attaching a second tag to the biomarker component of the aptamer affinity complex; (e) releasing the aptamer affinity complex from the first solid support; (f) exposing the released aptamer affinity complex to a second solid support that includes a second capture element and allowing the second tag to associate with the second capture element; (g) removing any non-complexed aptamer from the mixture by partitioning the non-complexed aptamer from the aptamer affinity complex; (h) eluting the aptamer from the solid support; and (i) detecting the biomarker by detecting the aptamer component of the aptamer affinity complex.

Any means known in the art can be used to detect a biomarker value by detecting the aptamer component of an aptamer affinity complex. A number of different detection methods can be used to detect the aptamer component of an affinity complex, such as, for example, hybridization assays, mass spectroscopy, or QPCR. In some embodiments, nucleic acid sequencing methods can be used to detect the aptamer component of an aptamer affinity complex and thereby detect a biomarker value. Briefly, a test sample can be subjected to any kind of nucleic acid sequencing method to identify and quantify the sequence or sequences of one or more aptamers present in the test sample. In some embodiments, the sequence includes the entire aptamer molecule or any portion of the molecule that may be used to uniquely identify the molecule. In other embodiments, the identifying sequencing is a specific sequence added to the aptamer; such sequences are often referred to as “tags,” “barcodes,” or “zipcodes.” In some embodiments, the sequencing method includes enzymatic steps to amplify the aptamer sequence or to convert any kind of nucleic acid, including RNA and DNA that contain chemical modifications to any position, to any other kind of nucleic acid appropriate for sequencing.

In some embodiments, the sequencing method includes one or more cloning steps. In other embodiments the sequencing method includes a direct sequencing method without cloning.

In some embodiments, the sequencing method includes a directed approach with specific primers that target one or more aptamer in the test sample. In other embodiments, the sequencing method includes a shotgun approach that targets all aptamer in the test sample.

In some embodiments, the sequencing method includes enzymatic steps to amplify the molecule targeted for sequencing. In other embodiments, the sequencing method directly sequences single molecules. An exemplary nucleic acid sequencing-based method that can be used to detect a biomarker value corresponding to a biomarker in a biological sample includes the following: (a) converting a mixture of aptamers that contain chemically modified nucleotides to unmodified nucleic acids with an enzymatic step; (b) shotgun sequencing the resulting unmodified nucleic acids with a massively parallel sequencing platform such as, for example, the 454 Sequencing System (454 Life Sciences/Roche), the Illumina Sequencing System (Illumina), the ABI SOLID Sequencing System (Applied Biosystems), the HeliScope Single Molecule Sequencer (Helicos Biosciences), or the Pacific Biosciences Real Time Single-Molecule Sequencing System (Pacific BioSciences) or the Polonator G Sequencing System (Dover Systems); and (c) identifying and quantifying the SOMAmers present in the mixture by specific sequence and sequence count.

### Determination of Biomarker Values Using Immunoassays

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I125) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Determination of Biomarker Values Using Gene Expression Profiling

Measuring mRNA in a biological sample may be used as a surrogate for detection of the level of the corresponding protein in the biological sample. Thus, any of the biomarkers or biomarker panels described herein can also be detected by detecting the appropriate RNA.

mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

miRNA molecules are small RNAs that are non-coding but may regulate gene expression. Any of the methods suited to the measurement of mRNA expression levels can also be used for the corresponding miRNA. Recently many laboratories have investigated the use of miRNAs as biomarkers for disease. Many diseases involve wide-spread transcriptional regulation, and it is not surprising that miRNAs might find a role as biomarkers. The connection between miRNA concentrations and disease is often even less clear than the connections between protein levels and disease, yet the value of miRNA biomarkers might be substantial. Of course, as with any RNA expressed differentially during disease, the problems facing the development of an in vitro diagnostic product will include the requirement that the miRNAs survive in the diseased cell and are easily extracted for analysis, or that the miRNAs are released into blood or other matrices where they must survive long enough to be measured. Protein biomarkers have similar requirements, although many potential protein biomarkers are secreted intentionally at the site of pathology and function, during disease, in a paracrine fashion. Many potential protein biomarkers are designed to function outside the cells within which those proteins are synthesized.

### Detection of Biomarkers Using In Vivo Molecular Imaging Technologies

Any of the described biomarkers (see, e.g., Table 4) may also be used in molecular imaging tests. For example, an imaging agent can be coupled to any of the described biomarkers, which can be used to aid in assessing tobacco use status, to monitor response to therapeutic interventions, to select a population for clinical trials among other uses.

In vivo imaging technologies provide non-invasive methods for determining the state of a particular disease or condition in the body of an individual. For example, entire portions of the body, or even the entire body, may be viewed as a three dimensional image, thereby providing valuable information concerning morphology and structures in the body. Such technologies may be combined with the detection of the biomarkers described herein to provide information concerning the tobacco use status of an individual.

The use of in vivo molecular imaging technologies is expanding due to various advances in technology. These advances include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, such as an aptamer or an antibody, for example, and/or with a peptide or protein, or an oligonucleotide (for example, for the detection of gene expression), or a complex containing any of these with one or more macromolecules and/or other particulate forms.

The contrast agent may also feature a radioactive atom that is useful in imaging. Suitable radioactive atoms include technetium-99m or iodine-123 for scintigraphic studies. Other readily detectable moieties include, for example, spin labels for magnetic resonance imaging (MRI) such as, for example, iodine-123 again, iodine-131, indium-111, fluorine-19, carbon-13, nitrogen-15, oxygen-17, gadolinium, manganese or iron. Such labels are well known in the art and could easily be selected by one of ordinary skill in the art.

Standard imaging techniques include but are not limited to magnetic resonance imaging, computed tomography scanning (coronary calcium score), positron emission tomography (PET), single photon emission computed tomography (SPECT), computed tomography angiography, and the like. For diagnostic in vivo imaging, the type of detection instrument available is a major factor in selecting a given contrast agent, such as a given radionuclide and the particular biomarker that it is used to target (protein, mRNA, and the like). The radionuclide chosen typically has a type of decay that is detectable by a given type of instrument. Also, when selecting a radionuclide for in vivo diagnosis, its half-life should be long enough to enable detection at the time of maximum uptake by the target tissue but short enough that deleterious radiation of the host is minimized.

Exemplary imaging techniques include but are not limited to PET and SPECT, which are imaging techniques in which a radionuclide is synthetically or locally administered to an individual. The subsequent uptake of the radiotracer is measured over time and used to obtain information about the targeted tissue and the biomarker. Because of the high-energy (gamma-ray) emissions of the specific isotopes employed and the sensitivity and sophistication of the instruments used to detect them, the two-dimensional distribution of radioactivity may be inferred from outside of the body.

Commonly used positron-emitting nuclides in PET include, for example, carbon-11, nitrogen-13, oxygen-15, and fluorine-18. Isotopes that decay by electron capture and/or gamma-emission are used in SPECT and include, for example iodine-123 and technetium-99m. An exemplary method for labeling amino acids with technetium-99m is the reduction of pertechnetate ion in the presence of a chelating precursor to form the labile technetium-99m-precursor complex, which, in turn, reacts with the metal binding group of a bifunctionally modified chemotactic peptide to form a technetium-99m-chemotactic peptide conjugate.

Antibodies are frequently used for such in vivo imaging diagnostic methods. The preparation and use of antibodies for in vivo diagnosis is well known in the art. Labeled antibodies which specifically bind any of the biomarkers in Table 4 can be injected into an individual, detectable according to the particular biomarker used, for the purpose of diagnosing or evaluating the disease status or condition of the individual. The label used will be selected in accordance with the imaging modality to be used, as previously described. Localization of the label permits determination of the tissue damage or other indications related to the individual's probability of ever having been a tobacco user. The amount of label within an organ or tissue also allows determination of the involvement of the biomarkers due to being an “ever’ tobacco user in that organ or tissue.

Similarly, aptamers may be used for such in vivo imaging diagnostic methods. For example, an aptamer that was used to identify a particular biomarker described in Table 4 (and therefore binds specifically to that particular biomarker) may be appropriately labeled and injected into an individual being evaluated for determination of tobacco use status, detectable according to the particular biomarker, for the purpose of diagnosing or evaluating the levels of tissue damage, atherosclerotic plaques, components of inflammatory response and other factors associated with the tobacco use status in the individual. The label used will be selected in accordance with the imaging modality to be used, as previously described. Localization of the label permits determination of the site of the processes leading to increased risk. The amount of label within an organ or tissue also allows determination of the infiltration of the pathological process in that organ or tissue. Aptamer-directed imaging agents could have unique and advantageous characteristics relating to tissue penetration, tissue distribution, kinetics, elimination, potency, and selectivity as compared to other imaging agents.

Such techniques may also optionally be performed with labeled oligonucleotides, for example, for detection of gene expression through imaging with antisense oligonucleotides. These methods are used for in situ hybridization, for example, with fluorescent molecules or radionuclides as the label. Other methods for detection of gene expression include, for example, detection of the activity of a reporter gene.

Another general type of imaging technology is optical imaging, in which fluorescent signals within the subject are detected by an optical device that is external to the subject. These signals may be due to actual fluorescence and/or to bioluminescence. Improvements in the sensitivity of optical detection devices have increased the usefulness of optical imaging for in vivo diagnostic assays.

The use of in vivo molecular biomarker imaging is increasing, including for clinical trials, for example, to more rapidly measure clinical efficacy in trials for new disease or condition therapies and/or to avoid prolonged treatment with a placebo for those diseases, such as multiple sclerosis, in which such prolonged treatment may be considered to be ethically questionable.

For a review of other techniques, see N. Blow, Nature Methods, 6, 465-469, 2009.

### Determination of Biomarker Values Using Mass Spectrometry Methods

A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS) n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS) N, atmospheric pressure photoionization mass spectrometry (APPI-MS), APPI-MS/MS, and APPI-(MS) N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′) 2 fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Determination of Biomarker Values Using a Proximity Ligation Assay

A proximity ligation assay can be used to determine biomarker values. Briefly, a test sample is contacted with a pair of affinity probes that may be a pair of antibodies or a pair of aptamers, with each member of the pair extended with an oligonucleotide. The targets for the pair of affinity probes may be two distinct determinates on one protein or one determinate on each of two different proteins, which may exist as homo- or hetero-multimeric complexes. When probes bind to the target determinates, the free ends of the oligonucleotide extensions are brought into sufficiently close proximity to hybridize together. The hybridization of the oligonucleotide extensions is facilitated by a common connector oligonucleotide which serves to bridge together the oligonucleotide extensions when they are positioned in sufficient proximity. Once the oligonucleotide extensions of the probes are hybridized, the ends of the extensions are joined together by enzymatic DNA ligation.

Each oligonucleotide extension comprises a primer site for PCR amplification. Once the oligonucleotide extensions are ligated together, the oligonucleotides form a continuous DNA sequence which, through PCR amplification, reveals information regarding the identity and amount of the target protein, as well as information regarding protein-protein interactions where the target determinates are on two different proteins. Proximity ligation can provide a highly sensitive and specific assay for real-time protein concentration and interaction information through use of real-time PCR. Probes that do not bind the determinates of interest do not have the corresponding oligonucleotide extensions brought into proximity and no ligation or PCR amplification can proceed, resulting in no signal being produced.

The foregoing assays enable the detection of biomarker levels that are useful in methods for predicting a probability of ever having been a tobacco user, where the methods comprise detecting, in a biological sample from an individual, biomarker levels that each correspond to a biomarker selected from the group consisting of the biomarkers provided in Table 4, wherein a classification, as described in detail below, using the biomarker levels indicates whether the individual has a probability of ever having been a tobacco user. While certain of the described biomarkers are useful alone for determining a probability of an individual ever having been a tobacco user, methods are also described herein for the grouping of multiple subsets of the biomarkers that are each useful as a panel of two or more biomarkers. In accordance with any of the methods described herein, biomarker levels can be detected and classified individually or they can be detected and classified collectively, as for example in a multiplex assay format.

### Classification of Biomarkers and Calculation of Disease Scores

In some embodiments, biomarker “signature” for a given diagnostic or predictive test contains a set of markers, each marker having different levels in the populations of interest. Different levels, in this context, may refer to different means of the marker levels for the individuals in two or more groups, or different variances in the two or more groups, or a combination of both. For the simplest form of a diagnostic test, these markers can be used to assign an unknown sample from an individual into one of two groups, such as having or not having a history of tobacco use. The assignment of a sample into one of two or more groups is known as classification, and the procedure used to accomplish this assignment is known as a classifier or a classification method. Classification methods may also be referred to as scoring methods. There are many classification methods that can be used to construct a diagnostic classifier from a set of biomarker values. In general, classification methods are most easily performed using supervised learning techniques where a data set is collected using samples obtained from individuals within two (or more, for multiple classification states) distinct groups one wishes to distinguish. Since the class (group or population) to which each sample belongs is known in advance for each sample, the classification method can be trained to give the desired classification response. It is also possible to use unsupervised learning techniques to produce a diagnostic classifier.

Common approaches for developing diagnostic classifiers include decision trees; bagging, boosting, forests and random forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning; and classifiers may be combined either simply or in ways which minimize particular objective functions. For a review, see, e.g., Pattern Classification, R. O. Duda, et al., editors, John Wiley & Sons, 2nd edition, 2001; see also, The Elements of Statistical Learning-Data Mining, Inference, and Prediction, T. Hastie, et al., editors, Springer Science+Business Media, LLC, 2nd edition, 2009; each of which is incorporated by reference in its entirety.

To produce a classifier using supervised learning techniques, a set of samples called training data are obtained. In the context of diagnostic tests, training data includes samples from the distinct groups (classes) to which unknown samples will later be assigned. For example, samples collected from individuals in a control population and individuals in a particular disease, condition or event population, such as “ever” tobacco users (former or current tobacco users), can constitute training data to develop a classifier that can classify unknown samples (or, more particularly, the individuals from whom the samples were obtained) as either being an “ever” tobacco user or being a “never” tobacco user. The development of the classifier from the training data is known as training the classifier. Specific details on classifier training depend on the nature of the supervised learning technique (see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009).

Since typically there are many more potential biomarker values than samples in a training set, care must be used to avoid over-fitting. Over-fitting occurs when a statistical model describes random error or noise instead of the underlying relationship. Over-fitting can be avoided in a variety of ways, including, for example, by limiting the number of markers used in developing the classifier, by assuming that the marker responses are independent of one another, by limiting the complexity of the underlying statistical model employed, and by ensuring that the underlying statistical model conforms to the data.

In order to identify a set of biomarkers associated with occurrence of events, the combined set of control and early event samples were analyzed using Principal Component Analysis (PCA). PCA displays the samples with respect to the axes defined by the strongest variations between all the samples, without regard to the case or control outcome, thus mitigating the risk of overfitting the distinction between case and control. Since the occurrence of serious thrombotic events has a strong component of chance involved, requiring unstable plaque to rupture in vital vessels to be reported, one would not expect to see a clear separation between the control and event sample sets. While the observed separation between case and control is not large, it occurs on the second principal component, corresponding to around 10% of the total variation in this set of samples, which indicates that the underlying biological variation is relatively simple to quantify.

In the next set of analyses, biomarkers can be analyzed for those components of difference between samples which were specific to the separation between the control samples and early event samples. One method that may be employed is the use of DSGA (Bair, E. and Tibshirani, R. (2004) Semi-supervised methods to predict patient survival from gene expression data. PLOS Biol., 2, 511-522) to remove (deflate) the first three principal component directions of variation between the samples in the control set. Although the dimensionality reduction is performed on the control set to discover, both the samples in the control and the samples from the early event samples are run through the PCA. Separation of cases from early events can be observed along the horizontal axis.

### Cross Validated Selection of Proteins Relevant to Tobacco Use Status

In order to avoid over-fitting of protein predictive power to idiosyncratic features of a particular selection of samples, a cross-validation and dimensional reduction approach can be taken. Cross-validation involves the multiple selection of sets of samples to determine the association of risk by protein combined with the use of the unselected samples to monitor the ability of the method to apply to samples which were not used in producing the model of risk (The Elements of Statistical Learning-Data Mining, Inference, and Prediction, T. Hastie, et al., editors, Springer Science+Business Media, LLC, 2nd edition, 2009). We applied the supervised PCA method of Tibshirani et al (Bair, E. and Tibshirani, R. (2004) Semi-supervised methods to predict patient survival from gene expression data. PLOS Biol., 2, 511-522.) which is applicable to high dimensional datasets in the modeling of tobacco use status. The supervised PCA (SPCA) method involves the univariate selection of a set of proteins statistically associated with the observed event hazard in the data and the determination of the correlated component which combines information from all of these proteins. This determination of the correlated component is a dimensionality reduction step which not only combines information across proteins, but also mitigates the likelihood of overfitting by reducing the number of independent variables from the full protein menu of over 1000 proteins down to a few principal components (in this work, we only examined the first principal component).

### Univariate Analysis and Multivariate Analysis of the Relationship of Individual Proteins to Time to Event

The Cox proportional hazard model (Cox, David R (1972). “Regression Models and Life-Tables”. Journal of the Royal Statistical Society. Series B (Methodological) 34 (2): 187-220.)) is widely used in medical statistics. Cox regression avoids fitting a specific function of time to the cumulative survival, and instead employs a model of relative risk referred to a baseline hazard function (which may vary with time). The baseline hazard function describes the common shape of the survival time distribution for all individuals, while the relative risk gives the level of the hazard for a set of covariate values (such as a single individual or group), as a multiple of the baseline hazard. The relative risk is constant with time in the Cox model.

Accelerated failure time (AFT) models are a sub-class of survival models. Survival models predict time-to-event data under partial information. Because survival models account for censoring, they can still use the data from those censored subjects, where other longitudinal models trying to predict when an event occurs can only use the information from subjects with determined outcomes. And because survival models take into account time-to-event, they can produce predicted probabilities of the event occurring within any time frame, which is different from most classification models (logistic regression, random forest).

AFT survival models in particular are a regression model which specifies/assumes a linear relationship between the model's covariates and log (time-to-event). So, a subject with 2× higher covariates (protein RFU counts) than baseline may be predicted to “survive” a diagnosis 2× longer than baseline.

The two most common survival models are AFT models and proportional hazards models, and an AFT Weibull model is both. The definition of a proportional hazards model is a little more complicated than that of an AFT model—in a proportional hazards model, a subject with 2× higher covariates than baseline may have a 2× higher hazard at any time point, where hazard is the negative derivative of the survival curve over time.

Other common proportional hazards models are exponential and Cox models. Exponential models are a sub-type of Weibull model. Cox models are more limited in use-predicted probabilities of time-to-event are not available from Cox models, only relative risk. AFT models can give both absolute and relative risk.

### Kits

Any combination of the biomarkers of Table 4 can be detected using a suitable kit, such as for use in performing the methods disclosed herein. Furthermore, any kit can contain one or more detectable labels as described herein, such as a fluorescent moiety, etc.

In one embodiment, a kit includes (a) one or more capture reagents (such as, for example, at least one aptamer or antibody) for detecting one or more biomarkers in a biological sample, wherein the biomarkers include any of the biomarkers set forth in Table 4 and optionally (b) one or more software or computer program products for classifying the individual from whom the biological sample was obtained as either having or not having a probability of being an “Ever” tobacco user, as further described herein. Alternatively, rather than one or more computer program products, one or more instructions for manually performing the above steps by a human can be provided.

The combination of a solid support with a corresponding capture reagent having a signal generating material is referred to herein as a “detection device” or “kit”. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, blocking agents, mass spectrometry matrix materials, antibody capture agents, positive control samples, negative control samples, software and information such as protocols, guidance and reference data.

In one aspect, the invention provides kits for the assessment of tobacco use status. The kits include PCR primers for one or more aptamers specific to biomarkers selected from Table 4. The kit may further include instructions for use and correlation of the biomarkers with prediction of probability of being an “Ever” tobacco user. The kit may also include a DNA array containing the complement of one or more of the aptamers specific for the biomarkers selected from Table 4, reagents, and/or enzymes for amplifying or isolating sample DNA. The kits may include reagents for real-time PCR, for example, TaqMan probes and/or primers, and enzymes.

For example, a kit can comprise (a) reagents comprising at least capture reagent for quantifying one or more biomarkers in a test sample, wherein said biomarkers comprise the biomarkers set forth in Table 4, or any other biomarkers or biomarkers panels described herein, and optionally (b) one or more algorithms or computer programs for performing the steps of comparing the amount of each biomarker quantified in the test sample to one or more predetermined cutoffs and assigning a score for each biomarker quantified based on said comparison, combining the assigned scores for each biomarker quantified to obtain a total score, comparing the total score with a predetermined score, and using said comparison to determine the probability of an individual being an “Ever” tobacco user. Alternatively, rather than one or more algorithms or computer programs, one or more instructions for manually performing the above steps by a human can be provided.

### Computer Methods and Software

Once a biomarker or biomarker panel is selected, a method for diagnosing an individual can comprise the following: 1) collect or otherwise obtain a biological sample; 2) perform an analytical method to detect and measure the biomarker or biomarkers in the panel in the biological sample; 3) perform any data normalization or standardization required for the method used to collect biomarker levels; 4) calculate the marker score; 5) combine the marker scores to obtain a total diagnostic or predictive score; and 6) report the individual's diagnostic or predictive score. In this approach, the diagnostic or predictive score may be a single number determined from the sum of all the marker calculations that is compared to a preset threshold value that is an indication of the presence or absence of disease or being an “Ever” tobacco user or a “Never” tobacco user. Or the diagnostic or predictive score may be a series of bars that each represent a biomarker level and the pattern of the responses may be compared to a pre-set pattern for determination of the presence or absence of disease, condition or the increased risk (or not) of an event.

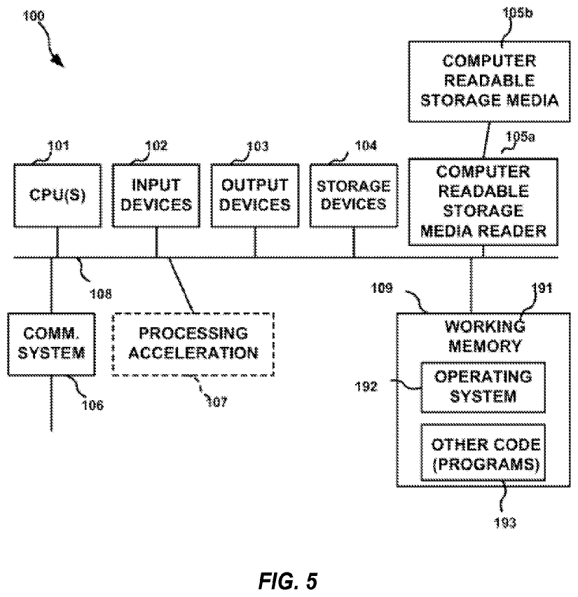

At least some embodiments of the methods described herein can be implemented with the use of a computer. An example of a computer system 100 is shown in FIG. 5. With reference to FIG. 5, system 100 is shown comprised of hardware elements that are electrically coupled via bus 108, including a processor 101, input device 102, output device 103, storage device 104, computer-readable storage media reader 105a, communications system 106, processing acceleration (e.g., DSP or special-purpose processors) 107 and memory 109. Computer-readable storage media reader 105a is further coupled to computer-readable storage media 105b, the combination comprehensively representing remote, local, fixed and/or removable storage devices plus storage media, memory, etc. for temporarily and/or more permanently containing computer-readable information, which can include storage device 104, memory 109 and/or any other such accessible system 100 resource. System 100 also comprises software elements (shown as being currently located within working memory 191) including an operating system 192 and other code 193, such as programs, data and the like.

With respect to FIG. 5, system 100 has extensive flexibility and configurability. Thus, for example, a single architecture might be utilized to implement one or more servers that can be further configured in accordance with currently desirable protocols, protocol variations, extensions, etc. However, it will be apparent to those skilled in the art that embodiments may well be utilized in accordance with more specific application requirements. For example, one or more system elements might be implemented as sub-elements within a system 100 component (e.g., within communications system 106). Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system can comprise a database containing features of biomarkers characteristic of prediction of the probability of an individual being an “Ever” tobacco user. The biomarker data (or biomarker information) can be utilized as an input to the computer for use as part of a computer implemented method. The biomarker data can include the data as described herein.

In one aspect, the system further comprises one or more devices for providing input data to the one or more processors.

The system further comprises a memory for storing a data set of ranked data elements.

In another aspect, the device for providing input data comprises a detector for detecting the characteristic of the data element, e.g., such as a mass spectrometer or gene chip reader.

The system additionally may comprise a database management system. User requests or queries can be formatted in an appropriate language understood by the database management system that processes the query to extract the relevant information from the database of training sets.

The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests.

The system may include an operating system (e.g., UNIX or Linux) for executing instructions from a database management system. In one aspect, the operating system can operate on a global communications network, such as the internet, and utilize a global communications network server to connect to such a network.

The system may include one or more devices that comprise a graphical display interface comprising interface elements such as buttons, pull down menus, scroll bars, fields for entering text, and the like as are routinely found in graphical user interfaces known in the art. Requests entered on a user interface can be transmitted to an application program in the system for formatting to search for relevant information in one or more of the system databases. Requests or queries entered by a user may be constructed in any suitable database language.

The graphical user interface may be generated by a graphical user interface code as part of the operating system and can be used to input data and/or to display inputted data. The result of processed data can be displayed in the interface, printed on a printer in communication with the system, saved in a memory device, and/or transmitted over the network or can be provided in the form of the computer readable medium.

The system can be in communication with an input device for providing data regarding data elements to the system (e.g., expression values). In one aspect, the input device can include a gene expression profiling system including, e.g., a mass spectrometer, gene chip or array reader, and the like.

The methods and apparatus for analyzing tobacco use status biomarker information according to various embodiments may be implemented in any suitable manner, for example, using a computer program operating on a computer system. A conventional computer system comprising a processor and a random access memory, such as a remotely-accessible application server, network server, personal computer or workstation may be used. Additional computer system components may include memory devices or information storage systems, such as a mass storage system and a user interface, for example a conventional monitor, keyboard and tracking device. The computer system may be a stand-alone system or part of a network of computers including a server and one or more databases.

The tobacco use assessment biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the tobacco use assessment prediction biomarkers. The computer program may comprise multiple modules performing various functions or operations, such as a processing module for processing raw data and generating supplemental data and an analysis module for analyzing raw data and supplemental data to generate a tobacco use prediction status. Determination of the probability of an individual being an “Ever” tobacco user may optionally comprise generating or collecting any other information, including additional biomedical information, regarding the condition of the individual relative to the disease, condition or event, identifying whether further tests may be desirable, or otherwise evaluating the health status of the individual.

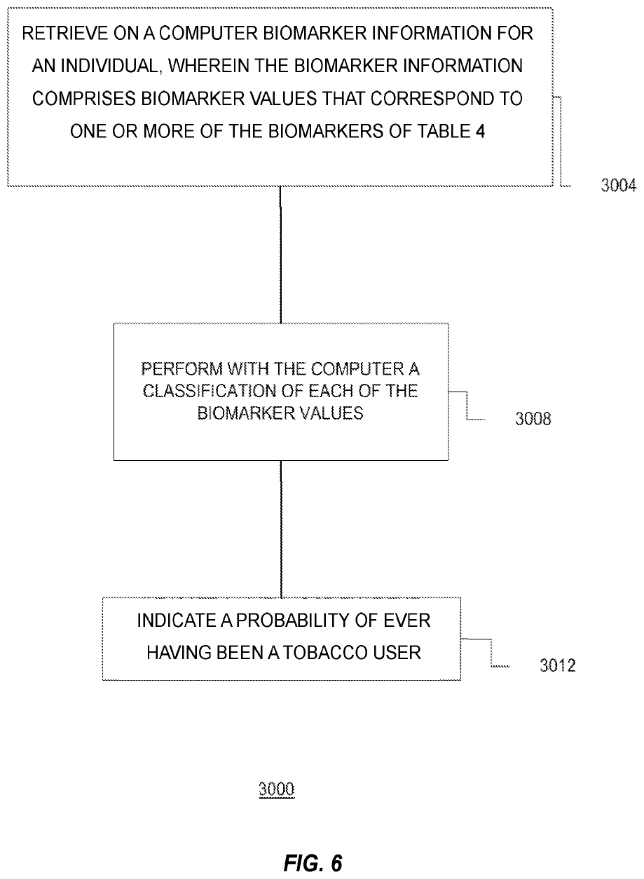

Referring now to FIG. 6, an example of a method of utilizing a computer in accordance with principles of a disclosed embodiment can be seen. In FIG. 6, a flowchart 3000 is shown. In block 3004, biomarker information can be retrieved for an individual. The biomarker information can be retrieved from a computer database, for example, after testing of the individual's biological sample is performed. The biomarker information can comprise biomarker levels that each correspond to one or more of the biomarkers of Table 4. In block 3008, a computer can be utilized to classify each of the biomarker levels. And, in block 3012, a determination can be made as to the probability that an individual is an “Ever” tobacco user upon a plurality of classifications. The indication can be output to a display or other indicating device so that it is viewable by a person. Thus, for example, it can be displayed on a display screen of a computer or other output device.

Some embodiments described herein can be implemented so as to include a computer program product. A computer program product may include a computer readable medium having computer readable program code embodied in the medium for causing an application program to execute on a computer with a database.

As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents.

In one aspect, a computer program product is provided for assessment of tobacco use status. The computer program product includes a computer readable medium embodying program code executable by a processor of a computing device or system, the program code comprising: code that retrieves data attributed to a biological sample from an individual, wherein the data comprises biomarker values that each correspond to one or more of the biomarkers of Table 4; and code that executes a classification method that indicates a probability of an “Ever” tobacco user status of the individual as a function of the biomarker values.

While various embodiments have been described as methods or apparatuses, it should be understood that embodiments can be implemented through code coupled with a computer, e.g., code resident on a computer or accessible by the computer. For example, software and databases could be utilized to implement many of the methods discussed above. Thus, in addition to embodiments accomplished by hardware, it is also noted that these embodiments can be accomplished through the use of an article of manufacture comprised of a computer usable medium having a computer readable program code embodied therein, which causes the enablement of the functions disclosed in this description. Therefore, it is desired that embodiments also be considered protected by this patent in their program code means as well. Furthermore, the embodiments may be embodied as code stored in a computer-readable memory of virtually any kind including, without limitation, RAM, ROM, magnetic media, optical media, or magneto-optical media. Even more generally, the embodiments could be implemented in software, or in hardware, or any combination thereof including, but not limited to, software running on a general purpose processor, microcode, PLAs, or ASICs.

It is also envisioned that embodiments could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

It is also noted that many of the structures, materials, and acts recited herein can be recited as means for performing a function or step for performing a function. Therefore, it should be understood that such language is entitled to cover all such structures, materials, or acts disclosed within this specification and their equivalents, including the matter incorporated by reference.

The biomarker identification process, the utilization of the biomarkers disclosed herein, and the various methods for determining biomarker values are described in detail above with respect to evaluation of a probability of an individual being an “Ever” tobacco user. However, the application of the process, the use of identified biomarkers, and the methods for determining biomarker values are fully applicable to other specific types of diseases or medical conditions, or to the identification of individuals who may or may not be benefited by an ancillary medical treatment.

### Other Methods

In some embodiments, the biomarkers and methods described herein are used to determine a medical insurance premium or coverage decision and/or a life insurance premium or coverage decision. In some embodiments, the results of the methods described herein are used to determine a medical insurance premium and/or a life insurance premium. In some such instances, an organization that provides medical insurance or life insurance requests or otherwise obtains information concerning a subject's tobacco use status and uses that information to determine an appropriate medical insurance or life insurance premium for the subject. In some embodiments, the test is requested by, and paid for by, the organization that provides medical insurance or life insurance. In some embodiments, the test is used by the potential acquirer of a practice or health system or company to predict future liabilities or costs should the acquisition go ahead.

In some embodiments, the biomarkers and methods described herein are used to predict and/or manage the utilization of medical resources. In some such embodiments, the methods are not carried out for the purpose of such prediction, but the information obtained from the method is used in such a prediction and/or management of the utilization of medical resources. For example, a testing facility or hospital may assemble information from the present methods for many subjects in order to predict and/or manage the utilization of medical resources at a particular facility or in a particular geographic area.

### EXAMPLES

The following examples are provided for illustrative purposes only and are not intended to limit the scope of the application as defined by the appended claims. All examples described herein were carried out using standard techniques, which are well known and routine to those of skill in the art. Routine molecular biology techniques described in the following examples can be carried out as described in standard laboratory manuals, such as Sambrook et al., Molecular Cloning: A Laboratory Manual, 3rd. ed., Cold Spring Harbor Laboratory Press, Cold Spring Harbor, N.Y., (2001).

### Example 1. Multiplex Aptamer Assay and Statistical Approaches for Biomarker Identification

A multiplex aptamer assay was used to analyze test samples and control samples to identify biomarkers predictive of a probability of an individual being an “Ever” tobacco user. The multiplexed analysis used in this experiment included aptamers to detect approximately 5,000 proteins in blood from small sample volumes (~65 μl of serum or plasma), with low limits of detection (1 μM median), ~7 logs of dynamic range, and ~5% median coefficient of variation. The multiplex aptamer assay is described, generally, e.g., in Gold et al. (2010) Aptamer-Based Multiplexed Proteomic Technology for Biomarker Discovery. PLoS ONE 5 (12): e15004; and U.S. Publication Nos: 2012/0101002 and 2012/0077695.

### Example 2. Model Specification

The primary endpoint was tobacco use status, a categorical endpoint of self-reported tobacco use habits with three levels: “Never,” “Past,” or “Current” tobacco user. “Past” (e.g., “Former”) tobacco users were combined with “Current” tobacco users and were labeled “Ever” and were compared to the “Never” group for model development.

The selected model is a 39-feature, protein-only (see Table 4) elastic net logistic regression model. This model was trained on a binary classification endpoint of “Ever” (“Current” and “Former”) or “Never” tobacco users.

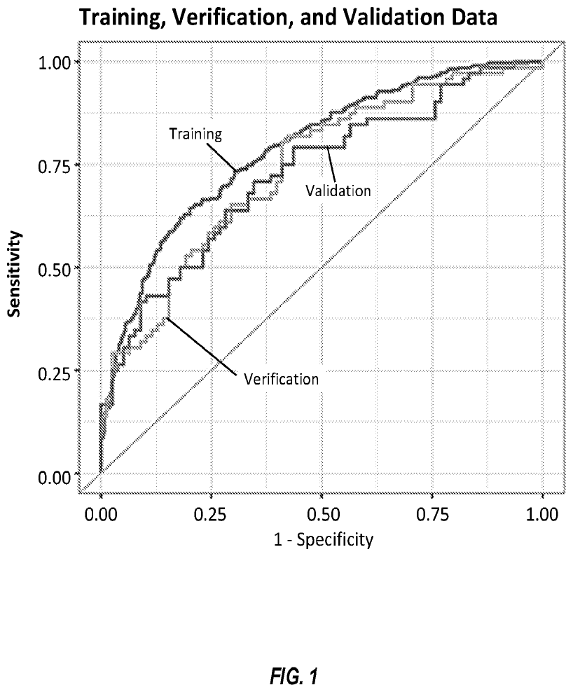

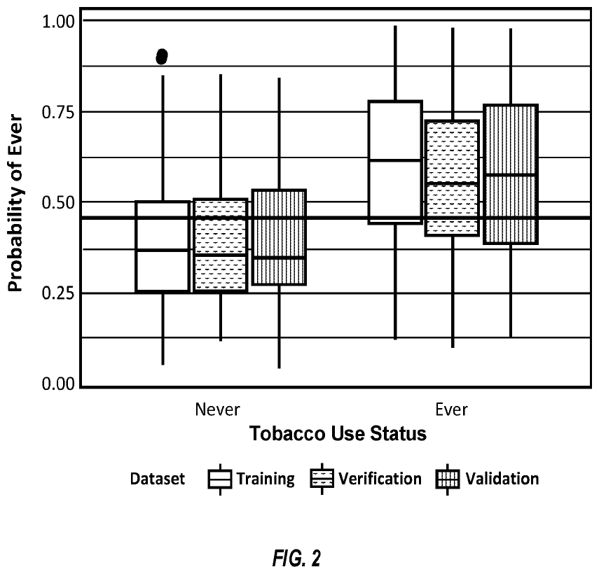

The model provides a single prediction output, predicted probability: This output is the absolute probability (i.e., absolute risk) of being an “Ever” tobacco user, a value between 0 and 1, at time of blood draw. Based on absolute probability and rounded to 3 places, the predictions are classified into two classes: “Never” tobacco user and “Ever” tobacco user. Table 3 below gives the absolute probability ranges and their associated categorical labels. The receiver operating characteristic (ROC) curve for the final model is shown in FIG. 1, and the box plots of the predicted probabilities for “Never” and “Ever” tobacco user classifications can be found in FIG. 2.

TABLE 3
Scoring rules for predicted probability

| Test Outcome AR | Predicted | |
|---|---|---|
| (absolute risk) | Class | Plain Language |
| AR ≤0.460 | “Never” | Absolute risk predictions |
| | | less than or equal to |
| | | 0.460 are classified as |
| | | “Never” tobacco users. |
| AR >0.460 | “Ever” | Absolute risk predictions |
| | | strictly greater than |
| | | 0.460 are classified as |
| | | “Ever” tobacco users. |

TABLE 4
Features included in the selected model

| | | | Entrez Gene |
|---|---|---|---|
| Uniprot | Target | Target Full Name | Symbol |
| Q9UF33 | EPHA6 | Ephrin type-A receptor 6 | EPHA6 |
| P05187 | Alkaline phosphatase, | Alkaline phosphatase, placental type | ALPP |
| | placental | | |
| P21583 | SCF | Kit ligand | KITLG |
| O43854 | EDIL3 | EGF-like repeat and discoidin I-like | EDIL3 |
| | | domain-containing protein 3 | |
| P48740 | MASP3: Heavy | Mannan-binding lectin serine protease | MASP1 |
| | | 1: Mannan-binding lectin serine | |
| | | protease 1 heavy chain | |
| P00797 | Renin | Renin | REN |
| P08118 | PSP-94 | Beta-microseminoprotein | MSMB |
| Q9Y6W6 | DUS10 | Dual specificity protein phosphatase | DUSP10 |
| | | 10 | |
| P01833 | PIGR | Polymeric immunoglobulin receptor | PIGR |
| P01861 | IgG4, Kappa | Ig gamma-4, Kappa | IGHG4 |
| Q76M96 | URB | Coiled-coil domain-containing protein | CCDC80 |
| | | 80 | |
| P43146 | DCC | DCC | DCC |
| Q9UKA8 | RCAN3 | Calcipressin-3 | RCAN3 |
| O00468 | Agrin | Agrin | AGRN |
| Q96GP6 | Secretoglobin family | Secretoglobin family 3A member 1 | SCGB3A1 |
| | 3A member 1 | | |
| Q96QR1 | SOX2 | Transcription factor SOX-2 | SOX2 |
| P48431 | SREC-II | Scavenger receptor class F member 2 | SCARF2 |
| P35858 | IGFALS | Insulin-like growth factor-binding | IGFALS |
| | | protein complex acid labile subunit | |
| P03973 | SLPI | Antileukoproteinase | SLPI |
| Q9BYZ8 | WFDC1 | WAP four-disulfide core domain | WFDC1 |
| | | protein 1 | |
| Q9HC57 | REG4 | Regenerating islet-derived protein 4 | REG4 |
| Q8TDL5 | MMP-8 | Neutrophil collagenase | MMP8 |
| P22894 | LPLC1 | BPI fold-containing family B member | BPIFB1 |
| | | 1 | |
| Q9HB63 | NET4 | Netrin-4 | NTN4 |
| P05451 | PSP | Lithostathine-1-alpha | REG1A |
| P09622 | DLDH | Dihydrolipoyl dehydrogenase, | DLD |
| | | mitochondrial | |
| Q8TDR4 | TCP10 | T-complex protein 10A homolog | TCP10L3 |
| Q9HCU0 | CD248 | Endosialin | CD248 |
| P43121 | MUC18 | Melanoma-associated antigen MUC18 | MCAM |
| Q01995 | TAGL | Transgelin | TAGLN |
| Q99727 | TIMP-4 | Metalloproteinase inhibitor 4 | TIMP4 |
| P58499 | FAM3B | Protein FAM3B | FAM3B |
| P23142 | fibulin 1 | Fibulin-1 | FBLN1 |
| Q9NQ79 | CRAC1 | Cartilage acidic protein 1 | CRTAC1 |
| P10696 | PPBN | Alkaline phosphatase, placental-like | ALPG |
| O00585 | 6Ckine | C-C motif chemokine 21 | CCL21 |
| O60911 | Cathepsin V | Cathepsin L2 | CTSV |
| Q14508 | HE4 | WAP four-disulfide core domain | WFDC2 |
| | | protein 2 | |
| Q06190 | PP2A, subunit B | Serine/threonine-protein phosphatase | PPP2R3A |
| | | 2A regulatory subunit B″ subunit alpha | |

Table 5 shows the performance metrics for the model.

TABLE 5
Performance of the selected tobacco use status model
on training, verification, and validation data.

| | N per | AUC | Sensitivity | Specificity |
|---|---|---|---|---|
| Dataset | group | (95% CI) | (95% CI) | (95% CI) |
| Training | 697 | 0.790 | 0.734 | 0.689 |
| | | (0.757, 0.823) | (0.679, 0.788) | (0.634, 0.743) |
| Verification | 150 | 0.740 | 0.653 | 0.679 |
| | | (0.661, 0.818) | (0.527, 0.778) | (0.561, 0.798) |
| Validation | 150 | 0.729 | 0.639 | 0.667 |
| | | (0.648, 0.809) | (0.512, 0.765) | (0.547, 0.786) |

Training and verification results exceed performance criteria of an AUC greater than or equal to 0.717. Sensitivity and specificity values were calculated with a decision cutoff of 0.46 for “Ever” versus “Never” tobacco users. Abbreviations: AUC, Area Under the Curve; CI, Confidence Intervals.

The model output is a binary classification of “Ever” or “Never” tobacco user at time of blood draw. Predicted probabilities below 0.460 will be labeled as “Never” tobacco users and “Ever” tobacco users otherwise. For RUO purposes, the probability of ever having been a tobacco user will be reported as a categorical class (“Ever” versus “Never”) alongside the predicted probability score. Because the output of this model is based on a probability, values outside of the range [0,1] are failures and will not be reported.

TABLE 6

| Usage | Rule | Rationale |
|---|---|---|
| LDT | Tobacco use status will be reported as a categorical label of | Providing categorical labels of |
| | “Ever” or “Never” based on our reference population and | tobacco use status will allow a |
| | decision cutoff of 0.460 (FIG. 2), where predicted | simple interpretation of clinically |
| | probabilities greater than 0.460 are classified as “Ever” and | meaningful tobacco use habits or |
| | predicted probabilities less than or equal to 0.460 are | exposure. |
| | classified as “Never.” | |
| RUO | Predicted probabilities (possible range 0-1) at three decimal | Reporting the absolute probability |
| | places will be reported along with the associated tobacco | along with categorical labels is |
| | use class label. | necessary for research use. |

### Example 3. Datasets for Test Development and Validation

Development and validation cohort(s). The Covance study, also called the Collection of Blood Samples and Baseline Information From Healthy Individuals for Biomarker Studies, is an observational study conducted across six sites in the United States: Austin, TX; Boise, ID; Dallas, TX; Honolulu, HI; Portland, OR; San Diego, CA. Between years 2008 and 2009, the Covance study enrolled 1029 individuals aged 19-90, matching ethnic/racial and sex demographics of the U.S. Self-reported medical history and demographics, as well as blood samples, were taken at a single time point. For the present test development, there were plasma samples available for 1,020 individuals aged 19-89.

Exclusion criteria for this study included:
1. Uncontrolled hypertension (ie, 2 measures >160/95, 10 minutes apart);
2. Self-reported treatment for a malignancy other than squamous cell or basal cell carcinoma of the skin in the last 2 years;
3. Self-reported pregnancy;
4. Self-reported chronic infectious (eg, hepatitis B, hepatitis C, HIV), autoimmune, or other inflammatory condition(s) such as SLE, scleroderma, MS, Crohn's Disease, or ulcerative colitis;
5. Self-reported chronic kidney or liver disease,
6. Self-reported chronic heart failure or diagnosed with myocardial infarction in the last 3 months;
7. Self-reported uncontrolled diabetes (HbA1c>8 if known);
8. Self-reported acute viral or bacterial infection or a temperature >38 °C within 24 hours of enrollment;
9. Self-reported participation in any therapeutic study in the 14 days prior to blood sampling;
10. Taking more than 20 mg/day of prednisone or related drug (self-reported).

The selected model has been developed based on self-report tobacco use status at the time of blood draw. The Covance dataset is representative of U.S. tobacco use rates; overall, the prevalence of “Current” tobacco users is 14.6% (13-15% between model development and validation datasets). A 2020 report from the Surgeon General had found that the prevalence of “Current” smokers in the United States dropped to an all-time low in 2018 (since assessments began in 1965) at 13.8% of the population. The overall prevalence of “Former” smokers in the Covance dataset is 33.3% (33-35% between model development and validation datasets), higher than the 2018 prevalence of 20.9%, which may be due to sampling location or biases in demographics such as race/ethnicity. (United States Public Health Service Office of the Surgeon General; National Center for Chronic Disease Prevention and Health Promotion (US) Office on Smoking and Health. Smoking Cessation: A Report of the Surgeon General. Washington (DC): US Department of Health and Human Services; 2020.)

Dataset Stratification. For this test, the data were split independently into three sets (training 70%/verification 15%/validation 15%) stratified by ever versus never tobacco users, allowing identification of a robust model while mitigating overfitting issues. The validation data set was not used in the POC or refinement stages.

### Model Development Data

TABLE 7
Demographic information for the Covance training dataset used for the Tobacco use Status model development stratified by tobacco use status.

| | | | Tobacco use Status | | | |
|---|---|---|---|---|---|---|
| | | | | | “Ever” | |
| Covariate | Measure | Total | “Never” | “Ever” | “Former” | “Current” |
| Sample Size | N | 697 | 363 | 334 | 229 | 105 |
| Subject | F | 389 (55.811%) | 219 (60.331%) | 170 (50.898%) | 129 (56.332%) | 41 (39.048%) |
| Gender | M | 308 (44.189%) | 144 (39.669%) | 164 (49.102%) | 100 (43.668%) | 64 (60.952%) |
| Subject Site | Austin | 62 (8.895%) | 36 (9.917%) | 26 (7.784%) | 22 (9.607%) | 4 (3.810%) |
| | Boise | 146 (20.947%) | 64 (17.631%) | 82 (24.551%) | 42 (18.341%) | 40 (38.095%) |
| | Dallas | 36 (5.165%) | 19 (5.234%) | 17 (5.090%) | 12 (5.240%) | 5 (4.762%) |
| | Honolulu | 111 (15.925%) | 58 (15.978%) | 53 (15.868%) | 46 (20.087%) | 7 (6.667%) |
| | Portland | 165 (23.673%) | 96 (26.446%) | 69 (20.659%) | 41 (17.904%) | 28 (26.667%) |
| | San Diego | 177 (25.395%) | 90 (24.793%) | 87 (26.048%) | 66 (28.821%) | 21 (20.000%) |
| Subject | Missing | 1 (0.143%) | 0 (0.000%) | 1 (0.299%) | 1 (0.437%) | 0 (0.000%) |
| Ethnicity | African | 77 (11.047%) | 39 (10.744%) | 38 (11.377%) | 19 (8.297%) | 19 (18.095%) |
| | American | | | | | |
| | Asian | 46 (6.600%) | 24 (6.612%) | 22 (6.587%) | 18 (7.860%) | 4 (3.810%) |
| | Caucasian | 472 (67.719%) | 245 (67.493%) | 227 (67.9464%) | 165 (72.052%) | 62 (59.048%) |
| | Hispanic | 81 (11.621%) | 48 (13.223%) | 33 (9.880%) | 20 (8.734%) | 13 (12.381%) |
| | Native | 12 (1.722%) | 3 (0.826%) | 9 (2.695%) | 4 (1.747%) | 5 (4.762%) |
| | American | | | | | |
| | Pacific | 8 (1.148%) | 4 (1.101%) | 4 (1.198%) | 2 (0.873%) | 2 (1.905%) |
| | Islander | | | | | |
| Subject Age | Mean (SD) | 50.918 (17.210) | 50.050 (17.642) | 51.862 (16.704) | 55.384 (16.243) | 44.181 (15.104) |
| | Median | 52 | 51 | 53 | 58 | 47 |
| | Range | 20-89 | 20-89 | 20-88 | 22-88 | 20-78 |

### Model Verification Data

TABLE 8
Demographic information for the Covance verification dataset used for the Tobacco use Status model development stratified by tobacco use status.

| | | | Tobacco use Status | | | |
|---|---|---|---|---|---|---|
| | | | | | “Ever” | |
| Covariate | Measure | Total | “Never” | “Ever” | “Former” | “Current” |
| Sample Size | N | 150 | 78 | 72 | 52 | 20 |
| Subject | F | 80 (53.333%) | 50 (64.103%) | 30 (41.667%) | 23 (44.231%) | 7 (35.000%) |
| Gender | M | 70 (46.667%) | 28 (35.897%) | 42 (58.333%) | 29 (55.769%) | 13 (65.000%) |
| Subject Site | Austin | 14 (9.333%) | 10 (12.821%) | 4 (5.556%) | 4 (7.692%) | 0 (0.000%) |
| | Boise | 28 (18.667%) | 16 (20.513%) | 12 (16.667%) | 6 (11.538%) | 6 (30.000%) |
| | Dallas | 6 (4.000%) | 1 (1.282%) | 5 (6.944%) | 5 (9.615%) | 0 (0.000%) |
| | Honolulu | 32 (21.333%) | 10 (12.821%) | 22 (30.556%) | 19 (36.538%) | 3 (15.000%) |
| | Portland | 36 (24.000%) | 22 (28.205%) | 14 (19.444%) | 10 (19.231%) | 4 (20.000%) |
| | San Diego | 34 (22.667%) | 19 (24.359%) | 15 (20.833%) | 8 (15.385%) | 7 (35.000%) |
| Subject | African | 14 (9.333%) | 5 (6.410%) | 9 (12.500%) | 6 (11.538%) | 3 (15.000%) |
| Ethnicity | American | | | | | |
| | Asian | 10 (6.667%) | 3 (3.846%) | 7 (9.722%) | 4 (7.692%) | 3 (15.000%) |
| | Caucasian | 92 (61.333%) | 52 (66.667%) | 40 (55.556%) | 28 (53.846%) | 12 (60.000%) |
| | Hispanic | 27 (18.000%) | 15 (19.231%) | 12 (16.667%) | 11 (21.154%) | 1 (5.000%) |
| | Native | 2 (1.333%) | 1 (1.282%) | 1 (1.389%) | 0 (0.000%) | 1 (5.000%) |
| | American | | | | | |
| | Pacific | 5 (3.333%) | 2 (2.564%) | 3 (4.167%) | 3 (5.769%) | 0 (0.000%) |
| | Islander | | | | | |
| Subject Age | Mean (SD) | 50.413 (18.067) | 49.5326 (18.312) | 51.375 (17.877) | 53.346 (17.916) | 46.250 (17.158) |
| | Median | 50 | 48 | 53 | 55 | 46 |
| | Range | 20-87 | 21-87 | 20-82 | 21-82 | 20-77 |

### Model Validation Data

TABLE 9
Demographic information for the Covance validation dataset used for the Tobacco use Status model validation stratified by tobacco use status.

| | | | Tobacco use Status | | | |
|---|---|---|---|---|---|---|
| | | | | | “Ever” | |
| Covariate | Measure | Total | “Never” | “Ever” | “Former” | “Current” |
| Sample Size | N | 150 | 78 | 72 | 51 | 21 |
| Subject | F | 87 (58.000%) | 49 (62.821%) | 38 (52.778%) | 27 (52.941%) | 11 (52.381%) |
| Gender | M | 63 (42.000%) | 29 (37.179%) | 34 (47.222%) | 24 (47.059%) | 10 (47.619%) |
| Subject Site | Austin | 7 (4.667%) | 6 (7.692%) | 1 (1.389%) | 1 (1.961%) | 0 (0.000%) |
| | Boise | 35 (23.3333%) | 7 (8.974%) | 28 (38.889%) | 14 (27.451%) | 14 (66.667%) |
| | Dallas | 9 (6.000%) | 4 (5.128%) | 5 (6.944%) | 5 (9.804%) | 0 (0.000%) |
| | Honolulu | 21 (14.000%) | 19 (24.359%) | 2 (2.778%) | 2 (3.922%) | 0 (0.000%) |
| | Portland | 44 (29.333%) | 22 (28.205%) | 22 (30.556%) | 17 (33.333%) | 5 (23.810%) |
| | San Diego | 34 (22.667%) | 20 (25.641%) | 14 (19.444%) | 12 (23.529%) | 2 (9.524%) |
| Subject | African | 18 (12.000%) | 10 (12.821%) | 8 (11.111%) | 6 (11.765%) | 2 (9.524%) |
| Ethnicity | American | | | | | |
| | Asian | 11 (7.333%) | 10 (12.821%) | 1 (1.389%) | 1 (1.961%) | 0 (0.000%) |
| | Caucasian | 101 (67.333%) | 44 (56.410%) | 57 (79.167%) | 40 (78.431%) | 17 (80.952%) |
| | Hispanic | 12 (8.000%) | 9 (11.538%) | 3 (4.167%) | 2 (3.922%) | 1 (4.762%) |
| | Native | 7 (4.667%) | 4 (5.128%) | 3 (4.167%) | 2 (3.922%) | 1 (4.762%) |
| | American | | | | | |
| | Pacific | 1 (0.667%) | 1 (1.282%) | 0 (0.000%) | 0 (0.000%) | 0 (0.000%) |
| | Islander | | | | | |
| Subject Age | Mean (SD) | 50.753 (16.523) | 48.269 (17.234) | 53.444 (15.385) | 57.373 (14.190) | 43.905 (14.212) |
| | Median | 51 | 49 | 54 | 59 | 44 |
| | Range | 19-83 | 19-83 | 21-82 | 25-82 | 21-74 |

### Example 4. Results from Development

Data QC and Pre-Analytics Results. The original dataset included 1,020 samples, although some samples were removed based on data QC analysis results. The removals are detailed in Table 10.

TABLE 10
Number of samples removed at each data cleaning step
and corresponding number of samples remaining.

| Step | N (removed) | N (remaining) |
|---|---|---|
| Baseline | | 1,020 |
| Outside range normalization | 19 | 1,001 |
| scale factors | | |
| Outliers | 0 | 1,001 |
| Missing tobacco use | 4 | 997 |

Note: Outliers are samples with >5% of SOMAmer measurements exceeding 6 median absolute deviations from the median. RowCheck failures are samples with at least one scale factor outside the acceptable range of 0.4 to 2.5.

Additionally, 363 analytes were removed before analyses began as they did not pass target confirmation specificity testing leaving 7,233 analytes available for analyses. No other issues were identified during Data QC or Pre-Analytics.

POC Approach and Results. The model performance requirement was met with an AUC≥0.717.

Refinement Approach and Results. The final model for the Tobacco use Status Test is a 39-analyte, protein-only elastic net logistic regression model. The model was trained on 70% of the Covance dataset. Verification metrics were calculated on a separated 15% dataset, and an additional 15% dataset was held-out for use in validation. The primary model output is predicted probability of ever having been a tobacco user at the time of blood draw.

Feature selection was based on the significant features for the univariate analyses during POC. In particular, the top univariate features from the “Ever” versus “Never,” “Former” versus “Current,” “Never” versus “Current,” and “Never” versus “Former” comparisons were considered for inclusion in the “Ever” versus “Never” model.

Candidate models were fit using cross validation with 10 folds and 5 repeats. Up-sampling was utilized to mitigate the class imbalance. The cross-validated AUC for the “Ever” versus “Never” endpoint was the main metric used to evaluate model performance. Models with a cross-validated AUC less than 90% of the AUC calculated on the full training set were removed from consideration due to being overfit.

Logistic regression models built on 27 different feature sets were compared. Of these, 14 models met the AUC threshold and did not show signs of overfitting. The top three models were chosen based on AUC on the verification dataset.

The selected model was chosen based on robustness assessed through various model hardening measures. In particular, the final model had the smallest tolerance intervals, passed QC replicates testing.

The final model is an elastic net linear regression model and has 39 non-zero features and 4 zero-coefficient features with α=0.100 and λ=0.0254. In development, the decision cutoff (for labeling “Ever” versus “Never”) was set as 0.46 which was the average of the optimal decision cutoffs to two decimal places. Here “optimal” was chosen to be the cutoff that maximizes Youden's J21 for the “Ever” versus “Never” test computed on both the training and verification datasets. The final model will report predicted probabilities at three decimal places, using a decision cutoff of 0.460.

Performance metrics for the training data and verification data are presented in Table 11. The training and verification AUC values meet the requirement of AUC greater than or equal to 0.717.

TABLE 11
Performance metrics and 95% CI for the training and verification
data from the selected Tobacco use Status model.

| | N per | AUC | Sensitivity | Specificity |
|---|---|---|---|---|
| Dataset | group | (95% CI) | (95% CI) | (95% CI) |
| Training | 697 | 0.790 | 0.734 | 0.689 |
| | | (0.757, 0.823) | (0.679, 0.788) | (0.634, 0.743) |
| Verification | 150 | 0.740 | 0.653 | 0.679 |
| | | (0.661, 0.818) | (0.527, 0.778) | (0.561, 0.798) |

Performance of stratifying “Current” and “Former” tobacco users within “Ever” tobacco user class. Predicted probabilities were stratified across the three tobacco use classes of “Current,” “Former,” and “Never.”

### Example 5. Model Validation Plan and Results

Clinical Validation Plan. Validation will be assessed on the 15% hold out dataset of the Covance data that have not yet been used. The final model from refinement is a 39-feature elastic net logistic regression model. Predicted probabilities for being an “Ever” tobacco user will be calculated using this final model and these predictions will be compared to the true “Ever” versus “Never” truth standard. The model will need to have an AUC of at least 0.65, 0.66, 0.67, 0.68, 0.69, 0.70, 0.717 or greater on the validation dataset.

Clinical Results on Validation Data. The required passing criteria for validation was that the Tobacco use Status model have an AUC of at least 0.65, 0.66, 0.67, 0.68, 0.69, 0.70, 0.717 or greater on the validation dataset. The model passed validation with an AUC equal to 0.729 as shown in Table 12.

TABLE 12
Performance metrics and 95% confidence intervals for the selected
Tobacco use Status model over the validation dataset.

| Evaluation | | AUC | Sensitivity | Specificity |
|---|---|---|---|---|
| Dataset | N | (95% CI) | (95% CI) | (95% CI) |
| Class label at | 150 | 0.729 | 0.639 | 0.667 |
| time of blood | | (0.648- | (0.512- | (0.547- |
| draw Validation | | 0.809) | 0.765) | 0.786) |

Model performance metrics are slightly lower over the validation data than in the training or verification data sets. However, the AUC is sufficiently high to pass the performance criterion.

Results from simulations, in Table 13 below, show that the best approach for handling out-of-range aptamers is analyte removal.

TABLE 13
The impact of winsorization and analyte removal on the validation
data. The table presents the accuracy of the model predictions in
the original data, winsorized data, and data after analyte removal.

| Imputation Method | Accuracy |
|---|---|
| Original validation data | 0.680 |
| Winsorized validation data | 0.661 |
| Analyte removal in validation data | 0.676 |

Results from tolerance bounds simulations describe the variance in the model resulting from assay noise. The 95% and 99% upper and lower bounds in linear space and the maximal bounds in probability space are described in Table 14.

TABLE 14
95% and 99% upper and lower bounds for variation
in linear and probability space.

| Prediction | | | |
|---|---|---|---|
| Space | Bound | 95% | 99% |
| Probability* | Lower | −0.148 | −0.194 |
| | Upper | 0.148 | 0.194 |
| Linear | Lower | −0.591 | −0.777 |
| | Upper | 0.591 | 0.777 |

*These bounds are not constant across probability space. The reported bounds are the maximal bounds which occur at a predicted probability of 0.5. These bounds are for interpretation purposes only and will not be used during regulatory activities as it relates to proficiency testing.

In some embodiments, one or more biomarkers may be included in the analysis and/or in determining univariate performances. By way of non-limiting example, biomarkers may include dCTP pyrophosphatase 1 (target name XTP3A, Entrez gene symbol DCTPP1, Uniprot ID Q9H773); Matrix-remodeling-associated protein 8 (target name MXRA8, Entrez gene symbol MXRA8, Uniprot ID Q9BRK3); T-complex protein 11-like protein 1 (target name T11L1, Entrez gene symbol TCP11L1, Uniprot ID Q9NUJ3) and/or Cartilage acidic protein 1 (target name CRAC1, Entrez gene symbol CRTAC1, Uniprot ID Q9NQ79). The univariate performance is shown in Table 25.

### Example 6: Analysis of Tobacco Use Model Biomarker Panels

Model biomarker panels comprising various combinations of the biomarkers listed in Table 4 were analyzed to determine the Area Under the Curve (AUC) value for the various combinations. The model biomarker panels may be based on a panel of N biomarker proteins having an AUC value of at least 0.65, at least 0.66, at least 0.67, at least 0.68, at least 0.69, at least 0.7, at least 0.75, at least 0.8, at least 0.85, at least 0.9, or at least 0.95, where N is 1, 2, 3, 4, 5, 6, 7, 8, 9, 10, 11, 12, 13, 14, 15, 16, 17, 18, 19, 20, 21, 22, 23, 24, 26, 27, 28, 29, 30, 31, 32, 33, 34, 35, 36, 37, 38, and/or 39 of the biomarker proteins listed in Table 4. The Tables below show exemplary model results when various combinations comprising 1 to 39 biomarker proteins were measured.

TABLE 15
EPHA6 Biomarker Panels

| EPHA6 | Alkaline phosphatase, placental | SCF | MASP3 | DUS10 | IgG4, Kappa | DCC | RCAN3 | CD248 | AUC |
|---|---|---|---|---|---|---|---|---|---|
| EPHA6 | Alkaline phosphatase, placental | SCF | MASP3 | DUS10 | IgG4, Kappa | DCC | RCAN3 | CD248 | 0.698 |
| EPHA6 | Alkaline phosphatase, placental | SCF | MASP3 | DUS10 | IgG4, Kappa | DCC | | CD248 | 0.697 |
| EPHA6 | Alkaline phosphatase, placental | SCF | MASP3 | DUS10 | IgG4, Kappa | | | CD248 | 0.696 |
| EPHA6 | Alkaline phosphatase, placental | SCF | MASP3 | DUS10 | IgG4, Kappa | | RCAN3 | CD248 | 0.696 |
| EPHA6 | Alkaline phosphatase, placental | SCF | MASP3 | DUS10 | IgG4, Kappa | DCC | RCAN3 | | 0.696 |
| EPHA6 | Alkaline phosphatase, placental | SCF | MASP3 | DUS10 | IgG4, Kappa | | RCAN3 | | 0.695 |
| EPHA6 | Alkaline phosphatase, placental | SCF | MASP3 | DUS10 | IgG4, Kappa | DCC | | | 0.695 |
| EPHA6 | Alkaline phosphatase, placental | SCF | MASP3 | | IgG4, Kappa | DCC | RCAN3 | CD248 | 0.694 |
| EPHA6 | Alkaline phosphatase, placental | SCF | MASP3 | DUS10 | IgG4, Kappa | | | | 0.694 |
| EPHA6 | Alkaline phosphatase, placental | SCF | MASP3 | | IgG4, Kappa | | RCAN3 | CD248 | 0.693 |
| EPHA6 | Alkaline phosphatase, placental | SCF | MASP3 | | IgG4, Kappa | DCC | | CD248 | 0.693 |
| EPHA6 | Alkaline phosphatase, placental | SCF | MASP3 | | IgG4, Kappa | | | CD248 | 0.692 |
| EPHA6 | Alkaline phosphatase, placental | SCF | MASP3 | | IgG4, Kappa | | RCAN3 | | 0.692 |
| EPHA6 | Alkaline phosphatase, placental | SCF | MASP3 | | IgG4, Kappa | DCC | RCAN3 | | 0.692 |
| EPHA6 | Alkaline phosphatase, placental | SCF | MASP3 | | IgG4, Kappa | | | | 0.691 |
| EPHA6 | Alkaline phosphatase, placental | SCF | MASP3 | | IgG4, Kappa | DCC | | | 0.69 |
| EPHA6 | Alkaline phosphatase, placental | SCF | MASP3 | DUS10 | | DCC | RCAN3 | CD248 | 0.689 |
| EPHA6 | Alkaline phosphatase, placental | SCF | | DUS10 | IgG4, Kappa | DCC | RCAN3 | CD248 | 0.687 |
| EPHA6 | Alkaline phosphatase, placental | SCF | MASP3 | DUS10 | | DCC | | CD248 | 0.687 |
| EPHA6 | Alkaline phosphatase, placental | SCF | | DUS10 | IgG4, Kappa | DCC | | CD248 | 0.686 |
| EPHA6 | Alkaline phosphatase, placental | SCF | MASP3 | DUS10 | | DCC | RCAN3 | | 0.686 |
| EPHA6 | Alkaline phosphatase, placental | SCF | MASP3 | DUS10 | | | RCAN3 | CD248 | 0.685 |
| EPHA6 | Alkaline phosphatase, placental | SCF | | DUS10 | IgG4, Kappa | DCC | RCAN3 | | 0.684 |
| EPHA6 | Alkaline phosphatase, placental | SCF | MASP3 | | | DCC | RCAN3 | CD248 | 0.684 |
| EPHA6 | Alkaline phosphatase, placental | SCF | MASP3 | DUS10 | | | | CD248 | 0.684 |
| EPHA6 | Alkaline phosphatase, placental | SCF | MASP3 | DUS10 | | DCC | | | 0.684 |
| EPHA6 | Alkaline phosphatase, placental | | MASP3 | DUS10 | IgG4, Kappa | DCC | | CD248 | 0.683 |
| EPHA6 | Alkaline phosphatase, placental | SCF | | DUS10 | IgG4, Kappa | DCC | | | 0.683 |
| EPHA6 | Alkaline phosphatase, placental | SCF | MASP3 | DUS10 | | | RCAN3 | | 0.683 |
| EPHA6 | Alkaline phosphatase, placental | | MASP3 | DUS10 | IgG4, Kappa | DCC | RCAN3 | CD248 | 0.682 |
| EPHA6 | Alkaline phosphatase, placental | SCF | | | IgG4, Kappa | DCC | RCAN3 | CD248 | 0.682 |
| EPHA6 | Alkaline phosphatase, placental | SCF | MASP3 | | | DCC | | CD248 | 0.682 |
| EPHA6 | Alkaline phosphatase, placental | SCF | MASP3 | DUS10 | | | | | 0.682 |
| EPHA6 | Alkaline phosphatase, placental | | MASP3 | DUS10 | IgG4, Kappa | | | CD248 | 0.681 |
| EPHA6 | Alkaline phosphatase, placental | | MASP3 | DUS10 | IgG4, Kappa | | RCAN3 | CD248 | 0.681 |
| EPHA6 | Alkaline phosphatase, placental | SCF | | DUS10 | IgG4, Kappa | | RCAN3 | CD248 | 0.681 |
| EPHA6 | Alkaline phosphatase, placental | SCF | MASP3 | | | | RCAN3 | CD248 | 0.681 |
| EPHA6 | Alkaline phosphatase, placental | SCF | MASP3 | | | DCC | RCAN3 | | 0.681 |
| EPHA6 | Alkaline phosphatase, placental | | MASP3 | DUS10 | IgG4, Kappa | | | | 0.68 |
| EPHA6 | Alkaline phosphatase, placental | | MASP3 | DUS10 | IgG4, Kappa | | RCAN3 | | 0.68 |
| EPHA6 | Alkaline phosphatase, placental | | MASP3 | DUS10 | IgG4, Kappa | DCC | | | 0.68 |
| EPHA6 | Alkaline phosphatase, placental | | MASP3 | DUS10 | IgG4, Kappa | DCC | RCAN3 | | 0.68 |
| EPHA6 | Alkaline phosphatase, placental | SCF | | | IgG4, Kappa | DCC | | CD248 | 0.68 |
| EPHA6 | Alkaline phosphatase, placental | SCF | | | IgG4, Kappa | DCC | RCAN3 | | 0.68 |
| EPHA6 | Alkaline phosphatase, placental | SCF | | DUS10 | IgG4, Kappa | | | CD248 | 0.68 |
| EPHA6 | Alkaline phosphatase, placental | SCF | | DUS10 | IgG4, Kappa | | RCAN3 | | 0.68 |
| EPHA6 | Alkaline phosphatase, placental | SCF | MASP3 | | | | | CD248 | 0.68 |
| EPHA6 | Alkaline phosphatase, placental | SCF | MASP3 | | | | RCAN3 | | 0.68 |
| EPHA6 | Alkaline phosphatase, placental | SCF | MASP3 | | | DCC | | | 0.68 |
| EPHA6 | Alkaline phosphatase, placental | | MASP3 | | IgG4, Kappa | DCC | | CD248 | 0.678 |
| EPHA6 | Alkaline phosphatase, placental | | MASP3 | | IgG4, Kappa | DCC | RCAN3 | CD248 | 0.678 |
| EPHA6 | Alkaline phosphatase, placental | SCF | | | IgG4, Kappa | DCC | | | 0.678 |
| EPHA6 | Alkaline phosphatase, placental | SCF | MASP3 | | | | | | 0.678 |
| EPHA6 | Alkaline phosphatase, placental | | MASP3 | | IgG4, Kappa | DCC | | | 0.677 |
| EPHA6 | Alkaline phosphatase, placental | | MASP3 | | IgG4, Kappa | DCC | RCAN3 | | 0.677 |
| EPHA6 | Alkaline phosphatase, placental | SCF | | DUS10 | IgG4, Kappa | | | | 0.677 |
| EPHA6 | Alkaline phosphatase, placental | | MASP3 | | IgG4, Kappa | | | CD248 | 0.676 |
| EPHA6 | Alkaline phosphatase, placental | SCF | | | IgG4, Kappa | | RCAN3 | CD248 | 0.676 |
| EPHA6 | Alkaline phosphatase, placental | | MASP3 | | IgG4, Kappa | | | | 0.675 |
| EPHA6 | Alkaline phosphatase, placental | | MASP3 | | IgG4, Kappa | | RCAN3 | | 0.675 |
| EPHA6 | Alkaline phosphatase, placental | | MASP3 | | IgG4, Kappa | | RCAN3 | CD248 | 0.675 |
| EPHA6 | Alkaline phosphatase, placental | | MASP3 | DUS10 | | DCC | | CD248 | 0.674 |
| EPHA6 | Alkaline phosphatase, placental | | MASP3 | DUS10 | | DCC | RCAN3 | CD248 | 0.674 |
| EPHA6 | Alkaline phosphatase, placental | SCF | | | IgG4, Kappa | | | CD248 | 0.674 |
| EPHA6 | Alkaline phosphatase, placental | SCF | | | IgG4, Kappa | | RCAN3 | | 0.674 |
| EPHA6 | Alkaline phosphatase, placental | SCF | | DUS10 | | DCC | RCAN3 | CD248 | 0.674 |
| EPHA6 | Alkaline phosphatase, placental | | MASP3 | DUS10 | | DCC | | | 0.673 |
| EPHA6 | Alkaline phosphatase, placental | | MASP3 | DUS10 | | DCC | RCAN3 | | 0.673 |
| EPHA6 | Alkaline phosphatase, placental | SCF | | | IgG4, Kappa | | | | 0.673 |
| EPHA6 | Alkaline phosphatase, placental | SCF | | DUS10 | | DCC | | CD248 | 0.672 |
| EPHA6 | Alkaline phosphatase, placental | SCF | | DUS10 | | DCC | RCAN3 | | 0.672 |
| EPHA6 | Alkaline phosphatase, placental | | MASP3 | DUS10 | | | RCAN3 | CD248 | 0.671 |
| EPHA6 | Alkaline phosphatase, placental | | MASP3 | DUS10 | | | | | 0.67 |
| EPHA6 | Alkaline phosphatase, placental | | MASP3 | DUS10 | | | | CD248 | 0.67 |
| EPHA6 | Alkaline phosphatase, placental | | MASP3 | DUS10 | | | RCAN3 | | 0.67 |
| EPHA6 | Alkaline phosphatase, placental | SCF | | DUS10 | | DCC | | | 0.669 |
| EPHA6 | Alkaline phosphatase, placental | | MASP3 | | | DCC | | CD248 | 0.668 |
| EPHA6 | Alkaline phosphatase, placental | | MASP3 | | | DCC | RCAN3 | CD248 | 0.668 |
| EPHA6 | Alkaline phosphatase, placental | | | DUS10 | IgG4, Kappa | DCC | | CD248 | 0.667 |
| EPHA6 | Alkaline phosphatase, placental | | | DUS10 | IgG4, Kappa | DCC | RCAN3 | CD248 | 0.667 |
| EPHA6 | Alkaline phosphatase, placental | SCF | | | | DCC | RCAN3 | CD248 | 0.667 |
| EPHA6 | Alkaline phosphatase, placental | | | DUS10 | IgG4, Kappa | DCC | | | 0.666 |
| EPHA6 | Alkaline phosphatase, placental | | | DUS10 | IgG4, Kappa | DCC | RCAN3 | | 0.666 |
| EPHA6 | Alkaline phosphatase, placental | | MASP3 | | | DCC | | | 0.666 |
| EPHA6 | Alkaline phosphatase, placental | | MASP3 | | | DCC | RCAN3 | | 0.666 |
| EPHA6 | Alkaline phosphatase, placental | SCF | | | | DCC | RCAN3 | | 0.665 |
| EPHA6 | Alkaline phosphatase, placental | SCF | | DUS10 | | | RCAN3 | CD248 | 0.665 |
| EPHA6 | Alkaline phosphatase, placental | | MASP3 | | | | | | 0.664 |
| EPHA6 | Alkaline phosphatase, placental | | MASP3 | | | | | CD248 | 0.664 |
| EPHA6 | Alkaline phosphatase, placental | | MASP3 | | | | RCAN3 | | 0.664 |
| EPHA6 | Alkaline phosphatase, placental | | MASP3 | | | | RCAN3 | CD248 | 0.664 |
| EPHA6 | Alkaline phosphatase, placental | SCF | | | | DCC | | CD248 | 0.664 |
| EPHA6 | Alkaline phosphatase, placental | SCF | | DUS10 | | | RCAN3 | | 0.664 |
| EPHA6 | Alkaline phosphatase, placental | SCF | | | | DCC | | | 0.662 |
| EPHA6 | Alkaline phosphatase, placental | SCF | | DUS10 | | | | CD248 | 0.662 |
| EPHA6 | Alkaline phosphatase, placental | | | | IgG4, Kappa | DCC | | CD248 | 0.661 |
| EPHA6 | Alkaline phosphatase, placental | | | | lgG4, Kappa | DCC | RCAN3 | CD248 | 0.661 |
| EPHA6 | Alkaline phosphatase, placental | SCF | | DUS10 | | | | | 0.661 |
| EPHA6 | Alkaline phosphatase, placental | | | DUS10 | IgG4, Kappa | | | | 0.66 |
| EPHA6 | Alkaline phosphatase, placental | | | DUS10 | IgG4, Kappa | | | CD248 | 0.66 |
| EPHA6 | Alkaline phosphatase, placental | | | DUS10 | IgG4, Kappa | | RCAN3 | | 0.66 |
| EPHA6 | Alkaline phosphatase, placental | | | DUS10 | IgG4, Kappa | | RCAN3 | CD248 | 0.66 |
| EPHA6 | Alkaline phosphatase, placental | | | | IgG4, Kappa | DCC | | | 0.659 |
| EPHA6 | Alkaline phosphatase, placental | | | | IgG4, Kappa | DCC | RCAN3 | | 0.659 |
| EPHA6 | Alkaline phosphatase, placental | SCF | | | | | RCAN3 | | 0.658 |
| EPHA6 | Alkaline phosphatase, placental | SCF | | | | | RCAN3 | CD248 | 0.658 |
| EPHA6 | Alkaline phosphatase, placental | | | DUS10 | | DCC | | CD248 | 0.656 |
| EPHA6 | Alkaline phosphatase, placental | | | DUS10 | | DCC | RCAN3 | CD248 | 0.656 |
| EPHA6 | Alkaline phosphatase, placental | | | DUS10 | | DCC | | | 0.655 |
| EPHA6 | Alkaline phosphatase, placental | | | DUS10 | | DCC | RCAN3 | | 0.655 |
| EPHA6 | Alkaline phosphatase, placental | SCF | | | | | | CD248 | 0.654 |
| EPHA6 | Alkaline phosphatase, placental | SCF | | | | | | | 0.653 |
| EPHA6 | Alkaline phosphatase, placental | | | | IgG4, Kappa | | | | 0.652 |
| EPHA6 | Alkaline phosphatase, placental | | | | IgG4, Kappa | | RCAN3 | | 0.652 |
| EPHA6 | Alkaline phosphatase, placental | | | | IgG4, Kappa | | RCAN3 | CD248 | 0.652 |
| EPHA6 | Alkaline phosphatase, placental | | | | IgG4, Kappa | | | CD248 | 0.651 |
| EPHA6 | Alkaline phosphatase, placental | | | | | DCC | RCAN3 | CD248 | 0.648 |
| EPHA6 | Alkaline phosphatase, placental | | | | | DCC | | CD248 | 0.647 |
| EPHA6 | Alkaline phosphatase, placental | | | | | DCC | RCAN3 | | 0.647 |
| EPHA6 | Alkaline phosphatase, placental | | | | | DCC | | | 0.646 |
| EPHA6 | Alkaline phosphatase, placental | | | DUS10 | | | RCAN3 | | 0.646 |
| EPHA6 | Alkaline phosphatase, placental | | | DUS10 | | | | | 0.645 |
| EPHA6 | Alkaline phosphatase, placental | | | DUS10 | | | RCAN3 | CD248 | 0.645 |
| EPHA6 | Alkaline phosphatase, placental | | | DUS10 | | | | CD248 | 0.644 |
| EPHA6 | | SCF | MASP3 | | IgG4, Kappa | DCC | | CD248 | 0.635 |
| EPHA6 | | SCF | MASP3 | | IgG4, Kappa | DCC | RCAN3 | CD248 | 0.635 |
| EPHA6 | | SCF | MASP3 | DUS10 | IgG4, Kappa | DCC | | | 0.634 |
| EPHA6 | | SCF | MASP3 | DUS10 | IgG4, Kappa | DCC | | CD248 | 0.634 |
| EPHA6 | | SCF | MASP3 | DUS10 | IgG4, Kappa | DCC | RCAN3 | | 0.634 |
| EPHA6 | | SCF | MASP3 | DUS10 | IgG4, Kappa | DCC | RCAN3 | CD248 | 0.634 |
| EPHA6 | | SCF | MASP3 | | IgG4, Kappa | DCC | | | 0.633 |
| EPHA6 | | SCF | MASP3 | | IgG4, Kappa | DCC | RCAN3 | | 0.633 |
| EPHA6 | Alkaline phosphatase, placental | | | | | | RCAN3 | | 0.631 |
| EPHA6 | Alkaline phosphatase, placental | | | | | | | | 0.63 |
| EPHA6 | Alkaline phosphatase, placental | | | | | | RCAN3 | CD248 | 0.63 |
| EPHA6 | | SCF | | | IgG4, Kappa | DCC | | CD248 | 0.629 |
| EPHA6 | | SCF | | | IgG4, Kappa | DCC | RCAN3 | | 0.629 |
| EPHA6 | | SCF | | | IgG4, Kappa | DCC | RCAN3 | CD248 | 0.629 |
| EPHA6 | | SCF | | DUS10 | IgG4, Kappa | DCC | | | 0.629 |
| EPHA6 | | SCF | | DUS10 | IgG4, Kappa | DCC | | CD248 | 0.629 |
| EPHA6 | | SCF | | DUS10 | IgG4, Kappa | DCC | RCAN3 | | 0.629 |
| EPHA6 | | SCF | | DUS10 | IgG4, Kappa | DCC | RCAN3 | CD248 | 0.629 |
| EPHA6 | Alkaline phosphatase, placental | | | | | | | CD248 | 0.629 |
| EPHA6 | | SCF | | | IgG4, Kappa | DCC | | | 0.628 |
| EPHA6 | | SCF | MASP3 | DUS10 | IgG4, Kappa | | | CD248 | 0.627 |
| EPHA6 | | SCF | MASP3 | DUS10 | IgG4, Kappa | | RCAN3 | | 0.627 |
| EPHA6 | | SCF | MASP3 | DUS10 | IgG4, Kappa | | RCAN3 | CD248 | 0.627 |
| EPHA6 | | SCF | MASP3 | | IgG4, Kappa | | | | 0.626 |
| EPHA6 | | SCF | MASP3 | | IgG4, Kappa | | | CD248 | 0.626 |
| EPHA6 | | SCF | MASP3 | | IgG4, Kappa | | RCAN3 | CD248 | 0.626 |
| EPHA6 | | SCF | MASP3 | DUS10 | IgG4, Kappa | | | | 0.626 |
| EPHA6 | | SCF | MASP3 | | IgG4, Kappa | | RCAN3 | | 0.625 |
| EPHA6 | | SCF | | DUS10 | IgG4, Kappa | | | | 0.618 |
| EPHA6 | | SCF | | DUS10 | IgG4, Kappa | | | CD248 | 0.618 |
| EPHA6 | | SCF | | DUS10 | IgG4, Kappa | | RCAN3 | | 0.618 |
| EPHA6 | | SCF | | DUS10 | IgG4, Kappa | | RCAN3 | CD248 | 0.618 |
| EPHA6 | | SCF | | | IgG4, Kappa | | RCAN3 | | 0.616 |
| EPHA6 | | SCF | | | IgG4, Kappa | | RCAN3 | CD248 | 0.616 |
| EPHA6 | | SCF | | | IgG4, Kappa | | | | 0.615 |
| EPHA6 | | SCF | | | IgG4, Kappa | | | CD248 | 0.615 |
| EPHA6 | | SCF | MASP3 | | | DCC | | CD248 | 0.615 |
| EPHA6 | | SCF | MASP3 | | | DCC | RCAN3 | CD248 | 0.615 |
| EPHA6 | | SCF | MASP3 | DUS10 | | DCC | | | 0.615 |
| EPHA6 | | SCF | MASP3 | DUS10 | | DCC | | CD248 | 0.615 |
| EPHA6 | | SCF | MASP3 | DUS10 | | DCC | RCAN3 | | 0.615 |
| EPHA6 | | SCF | MASP3 | DUS10 | | DCC | RCAN3 | CD248 | 0.615 |
| EPHA6 | | SCF | MASP3 | | | DCC | | | 0.614 |
| EPHA6 | | SCF | MASP3 | | | DCC | RCAN3 | | 0.614 |
| EPHA6 | | SCF | | DUS10 | | DCC | RCAN3 | CD248 | 0.613 |
| EPHA6 | | SCF | | DUS10 | | DCC | | | 0.612 |
| EPHA6 | | SCF | | DUS10 | | DCC | | CD248 | 0.612 |
| EPHA6 | | SCF | | DUS10 | | DCC | RCAN3 | | 0.612 |
| EPHA6 | | SCF | | | | DCC | RCAN3 | CD248 | 0.611 |
| EPHA6 | | SCF | | | | DCC | | CD248 | 0.61 |
| EPHA6 | | SCF | | | | DCC | | | 0.609 |
| EPHA6 | | SCF | | | | DCC | RCAN3 | | 0.609 |
| EPHA6 | | SCF | MASP3 | DUS10 | | | | CD248 | 0.605 |
| EPHA6 | | SCF | MASP3 | DUS10 | | | RCAN3 | | 0.605 |
| EPHA6 | | SCF | MASP3 | DUS10 | | | RCAN3 | CD248 | 0.605 |
| EPHA6 | | SCF | MASP3 | | | | | CD248 | 0.604 |
| EPHA6 | | SCF | MASP3 | | | | RCAN3 | | 0.604 |
| EPHA6 | | SCF | MASP3 | | | | RCAN3 | CD248 | 0.604 |
| EPHA6 | | SCF | MASP3 | DUS10 | | | | | 0.604 |
| EPHA6 | | SCF | MASP3 | | | | | | 0.603 |
| EPHA6 | | | MASP3 | DUS10 | IgG4, Kappa | DCC | | | 0.602 |
| EPHA6 | | | MASP3 | DUS10 | IgG4, Kappa | DCC | | CD248 | 0.602 |
| EPHA6 | | | MASP3 | DUS10 | IgG4, Kappa | DCC | RCAN3 | CD248 | 0.602 |
| EPHA6 | | | MASP3 | DUS10 | IgG4, Kappa | | RCAN3 | | 0.601 |
| EPHA6 | | | MASP3 | DUS10 | IgG4, Kappa | | RCAN3 | CD248 | 0.601 |
| EPHA6 | | | MASP3 | DUS10 | IgG4, Kappa | DCC | RCAN3 | | 0.601 |
| EPHA6 | | | MASP3 | | IgG4, Kappa | DCC | | CD248 | 0.6 |
| EPHA6 | | | MASP3 | | IgG4, Kappa | DCC | RCAN3 | CD248 | 0.6 |

TABLE 16
Alkaline phosphatase, placental Biomarker Panels

| Alkaline phosphatase, placental | EPHA6 | SCF | MASP3 | DUS10 | IgG4, Kappa | DCC | RCAN3 | CD248 | AUC |
|---|---|---|---|---|---|---|---|---|---|
| Alkaline phosphatase, placental | EPHA6 | SCF | MASP3 | DUS10 | IgG4, Kappa | DCC | RCAN3 | CD248 | 0.698 |
| Alkaline phosphatase, placental | | SCF | MASP3 | DUS10 | IgG4, Kappa | DCC | RCAN3 | CD248 | 0.697 |
| Alkaline phosphatase, placental | EPHA6 | SCF | MASP3 | DUS10 | IgG4, Kappa | DCC | | CD248 | 0.697 |
| Alkaline phosphatase, placental | | SCF | MASP3 | DUS10 | IgG4, Kappa | | | CD248 | 0.696 |
| Alkaline phosphatase, placental | | SCF | MASP3 | DUS10 | IgG4, Kappa | | RCAN3 | CD248 | 0.696 |
| Alkaline phosphatase, placental | | SCF | MASP3 | DUS10 | IgG4, Kappa | DCC | | CD248 | 0.696 |
| Alkaline phosphatase, placental | EPHA6 | SCF | MASP3 | DUS10 | IgG4, Kappa | | | CD248 | 0.696 |
| Alkaline phosphatase, placental | EPHA6 | SCF | MASP3 | DUS10 | IgG4, Kappa | | RCAN3 | CD248 | 0.696 |
| Alkaline phosphatase, placental | EPHA6 | SCF | MASP3 | DUS10 | IgG4, Kappa | DCC | RCAN3 | | 0.696 |
| Alkaline phosphatase, placental | EPHA6 | SCF | MASP3 | DUS10 | IgG4, Kappa | | RCAN3 | | 0.695 |
| Alkaline phosphatase, placental | EPHA6 | SCF | MASP3 | DUS10 | IgG4, Kappa | DCC | | | 0.695 |
| Alkaline phosphatase, placental | | SCF | MASP3 | | IgG4, Kappa | DCC | RCAN3 | CD248 | 0.694 |
| Alkaline phosphatase, placental | | SCF | MASP3 | DUS10 | IgG4, Kappa | | RCAN3 | | 0.694 |
| Alkaline phosphatase, placental | | SCF | MASP3 | DUS10 | IgG4, Kappa | DCC | RCAN3 | | 0.694 |
| Alkaline phosphatase, placental | EPHA6 | SCF | MASP3 | | IgG4, Kappa | DCC | RCAN3 | CD248 | 0.694 |
| Alkaline phosphatase, placental | EPHA6 | SCF | MASP3 | DUS10 | IgG4, Kappa | | | | 0.694 |
| Alkaline phosphatase, placental | | SCF | MASP3 | | IgG4, Kappa | | | CD248 | 0.693 |
| Alkaline phosphatase, placental | | SCF | MASP3 | | IgG4, Kappa | | RCAN3 | CD248 | 0.693 |
| Alkaline phosphatase, placental | | SCF | MASP3 | | IgG4, Kappa | DCC | | CD248 | 0.693 |
| Alkaline phosphatase, placental | | SCF | MASP3 | DUS10 | IgG4, Kappa | | | | 0.693 |
| Alkaline phosphatase, placental | | SCF | MASP3 | DUS10 | IgG4, Kappa | DCC | | | 0.693 |
| Alkaline phosphatase, placental | EPHA6 | SCF | MASP3 | | IgG4, Kappa | | RCAN3 | CD248 | 0.693 |
| Alkaline phosphatase, placental | EPHA6 | SCF | MASP3 | | IgG4, Kappa | DCC | | CD248 | 0.693 |
| Alkaline phosphatase, placental | EPHA6 | SCF | MASP3 | | IgG4, Kappa | | | CD248 | 0.692 |
| Alkaline phosphatase, placental | EPHA6 | SCF | MASP3 | | IgG4, Kappa | | RCAN3 | | 0.692 |
| Alkaline phosphatase, placental | EPHA6 | SCF | MASP3 | | IgG4, Kappa | DCC | RCAN3 | | 0.692 |
| Alkaline phosphatase, placental | | SCF | MASP3 | | IgG4, Kappa | | RCAN3 | | 0.691 |
| Alkaline phosphatase, placental | | SCF | MASP3 | | IgG4, Kappa | DCC | RCAN3 | | 0.691 |
| Alkaline phosphatase, placental | EPHA6 | SCF | MASP3 | | IgG4, Kappa | | | | 0.691 |
| Alkaline phosphatase, placental | | SCF | MASP3 | | IgG4, Kappa | | | | 0.69 |
| Alkaline phosphatase, placental | | SCF | MASP3 | | IgG4, Kappa | DCC | | | 0.69 |
| Alkaline phosphatase, placental | EPHA6 | SCF | MASP3 | | IgG4, Kappa | DCC | | | 0.69 |
| Alkaline phosphatase, placental | EPHA6 | SCF | MASP3 | DUS10 | | DCC | RCAN3 | CD248 | 0.689 |
| Alkaline phosphatase, placental | | SCF | | DUS10 | IgG4, Kappa | DCC | RCAN3 | CD248 | 0.687 |
| Alkaline phosphatase, placental | | SCF | MASP3 | DUS10 | | DCC | RCAN3 | CD248 | 0.687 |
| Alkaline phosphatase, placental | EPHA6 | SCF | | DUS10 | IgG4, Kappa | DCC | RCAN3 | CD248 | 0.687 |
| Alkaline phosphatase, placental | EPHA6 | SCF | MASP3 | DUS10 | | DCC | | CD248 | 0.687 |
| Alkaline phosphatase, placental | | SCF | MASP3 | DUS10 | | DCC | | CD248 | 0.686 |
| Alkaline phosphatase, placental | EPHA6 | SCF | | DUS10 | IgG4, Kappa | DCC | | CD248 | 0.686 |
| Alkaline phosphatase, placental | EPHA6 | SCF | MASP3 | DUS10 | | DCC | RCAN3 | | 0.686 |
| Alkaline phosphatase, placental | | SCF | | DUS10 | IgG4, Kappa | DCC | | CD248 | 0.685 |
| Alkaline phosphatase, placental | | SCF | MASP3 | DUS10 | | | RCAN3 | CD248 | 0.685 |
| Alkaline phosphatase, placental | EPHA6 | SCF | MASP3 | DUS10 | | | RCAN3 | CD248 | 0.685 |
| Alkaline phosphatase, placental | | SCF | MASP3 | DUS10 | | | | CD248 | 0.684 |
| Alkaline phosphatase, placental | EPHA6 | SCF | | DUS10 | IgG4, Kappa | DCC | RCAN3 | | 0.684 |
| Alkaline phosphatase, placental | EPHA6 | SCF | MASP3 | | | DCC | RCAN3 | CD248 | 0.684 |
| Alkaline phosphatase, placental | EPHA6 | SCF | MASP3 | DUS10 | | | | CD248 | 0.684 |
| Alkaline phosphatase, placental | EPHA6 | SCF | MASP3 | DUS10 | | DCC | | | 0.684 |
| Alkaline phosphatase, placental | | | MASP3 | DUS10 | IgG4, Kappa | DCC | | CD248 | 0.683 |
| Alkaline phosphatase, placental | | SCF | MASP3 | | | DCC | RCAN3 | CD248 | 0.683 |
| Alkaline phosphatase, placental | | SCF | MASP3 | DUS10 | | | RCAN3 | | 0.683 |
| Alkaline phosphatase, placental | | SCF | MASP3 | DUS10 | | DCC | RCAN3 | | 0.683 |
| Alkaline phosphatase, placental | EPHA6 | | MASP3 | DUS10 | IgG4, Kappa | DCC | | CD248 | 0.683 |
| Alkaline phosphatase, placental | EPHA6 | SCF | | DUS10 | IgG4, Kappa | DCC | | | 0.683 |
| Alkaline phosphatase, placental | EPHA6 | SCF | MASP3 | DUS10 | | | RCAN3 | | 0.683 |
| Alkaline phosphatase, placental | | | MASP3 | DUS10 | IgG4, Kappa | DCC | RCAN3 | CD248 | 0.682 |
| Alkaline phosphatase, placental | | SCF | MASP3 | DUS10 | | DCC | | | 0.682 |
| Alkaline phosphatase, placental | EPHA6 | | MASP3 | DUS10 | IgG4, Kappa | DCC | RCAN3 | CD248 | 0.682 |
| Alkaline phosphatase, placental | EPHA6 | SCF | | | IgG4, Kappa | DCC | RCAN3 | CD248 | 0.682 |
| Alkaline phosphatase, placental | EPHA6 | SCF | MASP3 | | | DCC | | CD248 | 0.682 |
| Alkaline phosphatase, placental | EPHA6 | SCF | MASP3 | DUS10 | | | | | 0.682 |
| Alkaline phosphatase, placental | | SCF | | | IgG4, Kappa | DCC | RCAN3 | CD248 | 0.681 |
| Alkaline phosphatase, placental | | SCF | | DUS10 | IgG4, Kappa | | RCAN3 | CD248 | 0.681 |
| Alkaline phosphatase, placental | | SCF | | DUS10 | IgG4, Kappa | DCC | RCAN3 | | 0.681 |
| Alkaline phosphatase, placental | | SCF | MASP3 | | | | RCAN3 | CD248 | 0.681 |
| Alkaline phosphatase, placental | | SCF | MASP3 | | | DCC | | CD248 | 0.681 |
| Alkaline phosphatase, placental | | SCF | MASP3 | DUS10 | | | | | 0.681 |
| Alkaline phosphatase, placental | EPHA6 | | MASP3 | DUS10 | IgG4, Kappa | | | CD248 | 0.681 |
| Alkaline phosphatase, placental | EPHA6 | | MASP3 | DUS10 | IgG4, Kappa | | RCAN3 | CD248 | 0.681 |
| Alkaline phosphatase, placental | EPHA6 | SCF | | DUS10 | IgG4, Kappa | | RCAN3 | CD248 | 0.681 |
| Alkaline phosphatase, placental | EPHA6 | SCF | MASP3 | | | | RCAN3 | CD248 | 0.681 |
| Alkaline phosphatase, placental | EPHA6 | SCF | MASP3 | | | DCC | RCAN3 | | 0.681 |
| Alkaline phosphatase, placental | | | MASP3 | DUS10 | IgG4, Kappa | | | | 0.68 |
| Alkaline phosphatase, placental | | | MASP3 | DUS10 | IgG4, Kappa | | | CD248 | 0.68 |
| Alkaline phosphatase, placental | | | MASP3 | DUS10 | IgG4, Kappa | | RCAN3 | | 0.68 |
| Alkaline phosphatase, placental | | | MASP3 | DUS10 | IgG4, Kappa | | RCAN3 | CD248 | 0.68 |
| Alkaline phosphatase, placental | | | MASP3 | DUS10 | IgG4, Kappa | DCC | | | 0.68 |
| Alkaline phosphatase, placental | | | MASP3 | DUS10 | IgG4, Kappa | DCC | RCAN3 | | 0.68 |
| Alkaline phosphatase, placental | | SCF | | DUS10 | IgG4, Kappa | | | CD248 | 0.68 |
| Alkaline phosphatase, placental | | SCF | MASP3 | | | | | CD248 | 0.68 |
| Alkaline phosphatase, placental | EPHA6 | | MASP3 | DUS10 | IgG4, Kappa | | | | 0.68 |
| Alkaline phosphatase, placental | EPHA6 | | MASP3 | DUS10 | IgG4, Kappa | | RCAN3 | | 0.68 |
| Alkaline phosphatase, placental | EPHA6 | | MASP3 | DUS10 | IgG4, Kappa | DCC | | | 0.68 |
| Alkaline phosphatase, placental | EPHA6 | | MASP3 | DUS10 | IgG4, Kappa | DCC | RCAN3 | | 0.68 |
| Alkaline phosphatase, placental | EPHA6 | SCF | | | IgG4, Kappa | DCC | | CD248 | 0.68 |
| Alkaline phosphatase, placental | EPHA6 | SCF | | | IgG4, Kappa | DCC | RCAN3 | | 0.68 |
| Alkaline phosphatase, placental | EPHA6 | SCF | | DUS10 | IgG4, Kappa | | | CD248 | 0.68 |
| Alkaline phosphatase, placental | EPHA6 | SCF | | DUS10 | IgG4, Kappa | | RCAN3 | | 0.68 |
| Alkaline phosphatase, placental | EPHA6 | SCF | MASP3 | | | | | CD248 | 0.68 |
| Alkaline phosphatase, placental | EPHA6 | SCF | MASP3 | | | | RCAN3 | | 0.68 |
| Alkaline phosphatase, placental | EPHA6 | SCF | MASP3 | | | DCC | | | 0.68 |
| Alkaline phosphatase, placental | | SCF | | | IgG4, Kappa | DCC | | CD248 | 0.679 |
| Alkaline phosphatase, placental | | SCF | | DUS10 | IgG4, Kappa | | RCAN3 | | 0.679 |
| Alkaline phosphatase, placental | | SCF | | DUS10 | IgG4, Kappa | DCC | | | 0.679 |
| Alkaline phosphatase, placental | | SCF | MASP3 | | | | RCAN3 | | 0.679 |
| Alkaline phosphatase, placental | | | MASP3 | | IgG4, Kappa | DCC | | CD248 | 0.678 |
| Alkaline phosphatase, placental | | | MASP3 | | IgG4, Kappa | DCC | RCAN3 | CD248 | 0.678 |
| Alkaline phosphatase, placental | | SCF | MASP3 | | | DCC | RCAN3 | | 0.678 |
| Alkaline phosphatase, placental | EPHA6 | | MASP3 | | IgG4, Kappa | DCC | | CD248 | 0.678 |
| Alkaline phosphatase, placental | EPHA6 | | MASP3 | | IgG4, Kappa | DCC | RCAN3 | CD248 | 0.678 |
| Alkaline phosphatase, placental | EPHA6 | SCF | | | IgG4, Kappa | DCC | | | 0.678 |
| Alkaline phosphatase, placental | EPHA6 | SCF | MASP3 | | | | | | 0.678 |
| Alkaline phosphatase, placental | | SCF | | DUS10 | IgG4, Kappa | | | | 0.677 |
| Alkaline phosphatase, placental | | SCF | MASP3 | | | | | | 0.677 |
| Alkaline phosphatase, placental | | SCF | MASP3 | | | DCC | | | 0.677 |
| Alkaline phosphatase, placental | EPHA6 | | MASP3 | | IgG4, Kappa | DCC | | | 0.677 |
| Alkaline phosphatase, placental | EPHA6 | | MASP3 | | IgG4, Kappa | DCC | RCAN3 | | 0.677 |
| Alkaline phosphatase, placental | EPHA6 | SCF | | DUS10 | IgG4, Kappa | | | | 0.677 |
| Alkaline phosphatase, placental | | | MASP3 | | IgG4, Kappa | | | | 0.676 |
| Alkaline phosphatase, placental | | | MASP3 | | IgG4, Kappa | | RCAN3 | | 0.676 |
| Alkaline phosphatase, placental | | | MASP3 | | IgG4, Kappa | DCC | RCAN3 | | 0.676 |
| Alkaline phosphatase, placental | | SCF | | | IgG4, Kappa | | RCAN3 | CD248 | 0.676 |
| Alkaline phosphatase, placental | | SCF | | | IgG4, Kappa | DCC | RCAN3 | | 0.676 |
| Alkaline phosphatase, placental | EPHA6 | | MASP3 | | IgG4, Kappa | | | CD248 | 0.676 |
| Alkaline phosphatase, placental | EPHA6 | SCF | | | IgG4, Kappa | | RCAN3 | CD248 | 0.676 |
| Alkaline phosphatase, placental | | | MASP3 | | IgG4, Kappa | | | CD248 | 0.675 |
| Alkaline phosphatase, placental | | | MASP3 | | IgG4, Kappa | | RCAN3 | CD248 | 0.675 |
| Alkaline phosphatase, placental | | | MASP3 | | IgG4, Kappa | DCC | | | 0.675 |
| Alkaline phosphatase, placental | | | MASP3 | DUS10 | | DCC | | CD248 | 0.675 |
| Alkaline phosphatase, placental | | | MASP3 | DUS10 | | DCC | RCAN3 | CD248 | 0.675 |
| Alkaline phosphatase, placental | | SCF | | | IgG4, Kappa | | | CD248 | 0.675 |
| Alkaline phosphatase, placental | EPHA6 | | MASP3 | | IgG4, Kappa | | | | 0.675 |
| Alkaline phosphatase, placental | EPHA6 | | MASP3 | | IgG4, Kappa | | RCAN3 | | 0.675 |
| Alkaline phosphatase, placental | EPHA6 | | MASP3 | | IgG4, Kappa | | RCAN3 | CD248 | 0.675 |
| Alkaline phosphatase, placental | | SCF | | | IgG4, Kappa | | RCAN3 | | 0.674 |
| Alkaline phosphatase, placental | EPHA6 | | MASP3 | DUS10 | | DCC | | CD248 | 0.674 |
| Alkaline phosphatase, placental | EPHA6 | | MASP3 | DUS10 | | DCC | RCAN3 | CD248 | 0.674 |
| Alkaline phosphatase, placental | EPHA6 | SCF | | | IgG4, Kappa | | | CD248 | 0.674 |
| Alkaline phosphatase, placental | EPHA6 | SCF | | | IgG4, Kappa | | RCAN3 | | 0.674 |
| Alkaline phosphatase, placental | EPHA6 | SCF | | DUS10 | | DCC | RCAN3 | CD248 | 0.674 |
| Alkaline phosphatase, placental | | SCF | | | IgG4, Kappa | DCC | | | 0.673 |
| Alkaline phosphatase, placental | EPHA6 | | MASP3 | DUS10 | | DCC | | | 0.673 |
| Alkaline phosphatase, placental | EPHA6 | | MASP3 | DUS10 | | DCC | RCAN3 | | 0.673 |
| Alkaline phosphatase, placental | EPHA6 | SCF | | | IgG4, Kappa | | | | 0.673 |
| Alkaline phosphatase, placental | | | MASP3 | DUS10 | | DCC | | | 0.672 |
| Alkaline phosphatase, placental | | | MASP3 | DUS10 | | DCC | RCAN3 | | 0.672 |
| Alkaline phosphatase, placental | | SCF | | DUS10 | | DCC | RCAN3 | CD248 | 0.672 |
| Alkaline phosphatase, placental | EPHA6 | SCF | | DUS10 | | DCC | | CD248 | 0.672 |
| Alkaline phosphatase, placental | EPHA6 | SCF | | DUS10 | | DCC | RCAN3 | | 0.672 |
| Alkaline phosphatase, placental | | | MASP3 | DUS10 | | | | | 0.671 |
| Alkaline phosphatase, placental | | | MASP3 | DUS10 | | | | CD248 | 0.671 |
| Alkaline phosphatase, placental | | | MASP3 | DUS10 | | | RCAN3 | | 0.671 |
| Alkaline phosphatase, placental | | | MASP3 | DUS10 | | | RCAN3 | CD248 | 0.671 |
| Alkaline phosphatase, placental | | SCF | | | IgG4, Kappa | | | | 0.671 |
| Alkaline phosphatase, placental | EPHA6 | | MASP3 | DUS10 | | | RCAN3 | CD248 | 0.671 |
| Alkaline phosphatase, placental | | SCF | | DUS10 | | DCC | | CD248 | 0.67 |
| Alkaline phosphatase, placental | EPHA6 | | MASP3 | DUS10 | | | | | 0.67 |
| Alkaline phosphatase, placental | EPHA6 | | MASP3 | DUS10 | | | | CD248 | 0.67 |
| Alkaline phosphatase, placental | EPHA6 | | MASP3 | DUS10 | | | RCAN3 | | 0.67 |
| Alkaline phosphatase, placental | EPHA6 | SCF | | DUS10 | | DCC | | | 0.669 |
| Alkaline phosphatase, placental | | | MASP3 | | | DCC | | CD248 | 0.668 |
| Alkaline phosphatase, placental | | | MASP3 | | | DCC | RCAN3 | CD248 | 0.668 |
| Alkaline phosphatase, placental | EPHA6 | | MASP3 | | | DCC | | CD248 | 0.668 |
| Alkaline phosphatase, placental | EPHA6 | | MASP3 | | | DCC | RCAN3 | CD248 | 0.668 |
| Alkaline phosphatase, placental | | | | DUS10 | IgG4, Kappa | DCC | | CD248 | 0.667 |
| Alkaline phosphatase, placental | | | | DUS10 | IgG4, Kappa | DCC | RCAN3 | CD248 | 0.667 |
| Alkaline phosphatase, placental | | SCF | | DUS10 | | DCC | RCAN3 | | 0.667 |
| Alkaline phosphatase, placental | EPHA6 | | | DUS10 | IgG4, Kappa | DCC | | CD248 | 0.667 |
| Alkaline phosphatase, placental | EPHA6 | | | DUS10 | IgG4, Kappa | DCC | RCAN3 | CD248 | 0.667 |
| Alkaline phosphatase, placental | EPHA6 | SCF | | | | DCC | RCAN3 | CD248 | 0.667 |
| Alkaline phosphatase, placental | | | MASP3 | | | DCC | | | 0.666 |
| Alkaline phosphatase, placental | | | MASP3 | | | DCC | RCAN3 | | 0.666 |
| Alkaline phosphatase, placental | EPHA6 | | | DUS10 | IgG4, Kappa | DCC | | | 0.666 |
| Alkaline phosphatase, placental | EPHA6 | | | DUS10 | IgG4, Kappa | DCC | RCAN3 | | 0.666 |
| Alkaline phosphatase, placental | EPHA6 | | MASP3 | | | DCC | | | 0.666 |
| Alkaline phosphatase, placental | EPHA6 | | MASP3 | | | DCC | RCAN3 | | 0.666 |
| Alkaline phosphatase, placental | | | | DUS10 | IgG4, Kappa | DCC | | | 0.665 |
| Alkaline phosphatase, placental | | | | DUS10 | IgG4, Kappa | DCC | RCAN3 | | 0.665 |
| Alkaline phosphatase, placental | | | MASP3 | | | | | | 0.665 |
| Alkaline phosphatase, placental | | SCF | | | | DCC | RCAN3 | CD248 | 0.665 |
| Alkaline phosphatase, placental | | SCF | | DUS10 | | | RCAN3 | CD248 | 0.665 |
| Alkaline phosphatase, placental | EPHA6 | SCF | | | | DCC | RCAN3 | | 0.665 |
| Alkaline phosphatase, placental | EPHA6 | SCF | | DUS10 | | | RCAN3 | CD248 | 0.665 |
| Alkaline phosphatase, placental | | | MASP3 | | | | | CD248 | 0.664 |
| Alkaline phosphatase, placental | | | MASP3 | | | | RCAN3 | | 0.664 |
| Alkaline phosphatase, placental | | | MASP3 | | | | RCAN3 | CD248 | 0.664 |
| Alkaline phosphatase, placental | | SCF | | DUS10 | | DCC | | | 0.664 |
| Alkaline phosphatase, placental | EPHA6 | | MASP3 | | | | | | 0.664 |
| Alkaline phosphatase, placental | EPHA6 | | MASP3 | | | | | CD248 | 0.664 |
| Alkaline phosphatase, placental | EPHA6 | | MASP3 | | | | RCAN3 | | 0.664 |
| Alkaline phosphatase, placental | EPHA6 | | MASP3 | | | | RCAN3 | CD248 | 0.664 |
| Alkaline phosphatase, placental | EPHA6 | SCF | | | | DCC | | CD248 | 0.664 |
| Alkaline phosphatase, placental | EPHA6 | SCF | | DUS10 | | | RCAN3 | | 0.664 |
| Alkaline phosphatase, placental | | SCF | | DUS10 | | | RCAN3 | | 0.663 |
| Alkaline phosphatase, placental | | SCF | | | | DCC | | CD248 | 0.662 |
| Alkaline phosphatase, placental | | SCF | | DUS10 | | | | CD248 | 0.662 |
| Alkaline phosphatase, placental | EPHA6 | SCF | | | | DCC | | | 0.662 |
| Alkaline phosphatase, placental | EPHA6 | SCF | | DUS10 | | | | CD248 | 0.662 |
| Alkaline phosphatase, placental | | | | DUS10 | IgG4, Kappa | | | | 0.661 |
| Alkaline phosphatase, placental | | | | DUS10 | IgG4, Kappa | | RCAN3 | | 0.661 |
| Alkaline phosphatase, placental | EPHA6 | | | | IgG4, Kappa | DCC | | CD248 | 0.661 |
| Alkaline phosphatase, placental | EPHA6 | | | | IgG4, Kappa | DCC | RCAN3 | CD248 | 0.661 |
| Alkaline phosphatase, placental | EPHA6 | SCF | | DUS10 | | | | | 0.661 |
| Alkaline phosphatase, placental | | | | | IgG4, Kappa | DCC | | CD248 | 0.66 |
| Alkaline phosphatase, placental | | | | | IgG4, Kappa | DCC | RCAN3 | CD248 | 0.66 |
| Alkaline phosphatase, placental | | | | DUS10 | IgG4, Kappa | | | CD248 | 0.66 |
| Alkaline phosphatase, placental | | | | DUS10 | IgG4, Kappa | | RCAN3 | CD248 | 0.66 |
| Alkaline phosphatase, placental | | SCF | | | | DCC | RCAN3 | | 0.66 |
| Alkaline phosphatase, placental | EPHA6 | | | DUS10 | IgG4, Kappa | | | | 0.66 |
| Alkaline phosphatase, placental | EPHA6 | | | DUS10 | IgG4, Kappa | | | CD248 | 0.66 |
| Alkaline phosphatase, placental | EPHA6 | | | DUS10 | IgG4, Kappa | | RCAN3 | | 0.66 |
| Alkaline phosphatase, placental | EPHA6 | | | DUS10 | IgG4, Kappa | | RCAN3 | CD248 | 0.66 |
| Alkaline phosphatase, placental | | SCF | | DUS10 | | | | | 0.659 |
| Alkaline phosphatase, placental | EPHA6 | | | | IgG4, Kappa | DCC | | | 0.659 |
| Alkaline phosphatase, placental | EPHA6 | | | | IgG4, Kappa | DCC | RCAN3 | | 0.659 |
| Alkaline phosphatase, placental | | SCF | | | | | RCAN3 | CD248 | 0.658 |
| Alkaline phosphatase, placental | EPHA6 | SCF | | | | | RCAN3 | | 0.658 |
| Alkaline phosphatase, placental | EPHA6 | SCF | | | | | RCAN3 | CD248 | 0.658 |
| Alkaline phosphatase, placental | | | | | IgG4, Kappa | DCC | | | 0.657 |
| Alkaline phosphatase, placental | | | | | IgG4, Kappa | DCC | RCAN3 | | 0.657 |
| Alkaline phosphatase, placental | | SCF | | | | | RCAN3 | | 0.656 |
| Alkaline phosphatase, placental | EPHA6 | | | DUS10 | | DCC | | CD248 | 0.656 |
| Alkaline phosphatase, placental | EPHA6 | | | DUS10 | | DCC | RCAN3 | CD248 | 0.656 |
| Alkaline phosphatase, placental | | | | DUS10 | | DCC | | CD248 | 0.655 |
| Alkaline phosphatase, placental | | | | DUS10 | | DCC | RCAN3 | CD248 | 0.655 |
| Alkaline phosphatase, placental | | SCF | | | | | | CD248 | 0.655 |
| Alkaline phosphatase, placental | | SCF | | | | DCC | | | 0.655 |
| Alkaline phosphatase, placental | EPHA6 | | | DUS10 | | DCC | | | 0.655 |
| Alkaline phosphatase, placental | EPHA6 | | | DUS10 | | DCC | RCAN3 | | 0.655 |
| Alkaline phosphatase, placental | | | | | IgG4, Kappa | | | | 0.654 |
| Alkaline phosphatase, placental | | | | | IgG4, Kappa | | RCAN3 | | 0.654 |
| Alkaline phosphatase, placental | EPHA6 | SCF | | | | | | CD248 | 0.654 |
| Alkaline phosphatase, placental | | | | DUS10 | | DCC | RCAN3 | | 0.653 |
| Alkaline phosphatase, placental | EPHA6 | SCF | | | | | | | 0.653 |
| Alkaline phosphatase, placental | | | | DUS10 | | DCC | | | 0.652 |
| Alkaline phosphatase, placental | | SCF | | | | | | | 0.652 |
| Alkaline phosphatase, placental | EPHA6 | | | | IgG4, Kappa | | | | 0.652 |
| Alkaline phosphatase, placental | EPHA6 | | | | IgG4, Kappa | | RCAN3 | | 0.652 |
| Alkaline phosphatase, placental | EPHA6 | | | | IgG4, Kappa | | RCAN3 | CD248 | 0.652 |
| Alkaline phosphatase, placental | | | | | IgG4, Kappa | | | CD248 | 0.651 |
| Alkaline phosphatase, placental | | | | | IgG4, Kappa | | RCAN3 | CD248 | 0.651 |
| Alkaline phosphatase, placental | EPHA6 | | | | IgG4, Kappa | | | CD248 | 0.651 |
| Alkaline phosphatase, placental | | | | DUS10 | | | RCAN3 | | 0.648 |
| Alkaline phosphatase, placental | EPHA6 | | | | | DCC | RCAN3 | CD248 | 0.648 |
| Alkaline phosphatase, placental | | | | | | DCC | RCAN3 | CD248 | 0.647 |
| Alkaline phosphatase, placental | | | | DUS10 | | | | | 0.647 |
| Alkaline phosphatase, placental | EPHA6 | | | | | DCC | | CD248 | 0.647 |
| Alkaline phosphatase, placental | EPHA6 | | | | | DCC | RCAN3 | | 0.647 |
| Alkaline phosphatase, placental | | | | DUS10 | | | RCAN3 | CD248 | 0.646 |
| Alkaline phosphatase, placental | EPHA6 | | | | | DCC | | | 0.646 |
| Alkaline phosphatase, placental | EPHA6 | | | DUS10 | | | RCAN3 | | 0.646 |
| Alkaline phosphatase, placental | | | | | | DCC | | CD248 | 0.645 |
| Alkaline phosphatase, placental | | | | DUS10 | | | | CD248 | 0.645 |
| Alkaline phosphatase, placental | EPHA6 | | | DUS10 | | | | | 0.645 |
| Alkaline phosphatase, placental | EPHA6 | | | DUS10 | | | RCAN3 | CD248 | 0.645 |
| Alkaline phosphatase, placental | | | | | | DCC | RCAN3 | | 0.644 |
| Alkaline phosphatase, placental | EPHA6 | | | DUS10 | | | | CD248 | 0.644 |
| Alkaline phosphatase, placental | | | | | | DCC | | | 0.643 |
| Alkaline phosphatase, placental | | | | | | | RCAN3 | | 0.633 |
| Alkaline phosphatase, placental | | | | | | | | | 0.632 |
| Alkaline phosphatase, placental | | | | | | | RCAN3 | CD248 | 0.631 |
| Alkaline phosphatase, placental | EPHA6 | | | | | | RCAN3 | | 0.631 |
| Alkaline phosphatase, placental | | | | | | | | CD248 | 0.63 |
| Alkaline phosphatase, placental | EPHA6 | | | | | | | | 0.63 |
| Alkaline phosphatase, placental | EPHA6 | | | | | | RCAN3 | CD248 | 0.63 |
| Alkaline phosphatase, placental | EPHA6 | | | | | | | CD248 | 0.629 |

TABLE 17
SCF Biomarker Panels

| SCF | EPHA6 | Alkaline phosphatase, placental | MASP3 | DUS10 | IgG4, Kappa | DCC | RCAN3 | CD248 | AUC |
|---|---|---|---|---|---|---|---|---|---|
| SCF | EPHA6 | Alkaline phosphatase, placental | MASP3 | DUS10 | IgG4, Kappa | DCC | RCAN3 | CD248 | 0.698 |
| SCF | | Alkaline phosphatase, placental | MASP3 | DUS10 | IgG4, Kappa | DCC | RCAN3 | CD248 | 0.697 |
| SCF | EPHA6 | Alkaline phosphatase, placental | MASP3 | DUS10 | IgG4, Kappa | DCC | | CD248 | 0.697 |
| SCF | | Alkaline phosphatase, placental | MASP3 | DUS10 | IgG4, Kappa | | | CD248 | 0.696 |
| SCF | | Alkaline phosphatase, placental | MASP3 | DUS10 | IgG4, Kappa | | RCAN3 | CD248 | 0.696 |
| SCF | | Alkaline phosphatase, placental | MASP3 | DUS10 | IgG4, Kappa | DCC | | CD248 | 0.696 |
| SCF | EPHA6 | Alkaline phosphatase, placental | MASP3 | DUS10 | IgG4, Kappa | | | CD248 | 0.696 |
| SCF | EPHA6 | Alkaline phosphatase, placental | MASP3 | DUS10 | IgG4, Kappa | | RCAN3 | CD248 | 0.696 |
| SCF | EPHA6 | Alkaline phosphatase, placental | MASP3 | DUS10 | IgG4, Kappa | DCC | RCAN3 | | 0.696 |
| SCF | EPHA6 | Alkaline phosphatase, placental | MASP3 | DUS10 | IgG4, Kappa | | RCAN3 | | 0.695 |
| SCF | EPHA6 | Alkaline phosphatase, placental | MASP3 | DUS10 | IgG4, Kappa | DCC | | | 0.695 |
| SCF | | Alkaline phosphatase, placental | MASP3 | | IgG4, Kappa | DCC | RCAN3 | CD248 | 0.694 |
| SCF | | Alkaline phosphatase, placental | MASP3 | DUS10 | IgG4, Kappa | | RCAN3 | | 0.694 |
| SCF | | Alkaline phosphatase, placental | MASP3 | DUS10 | IgG4, Kappa | DCC | RCAN3 | | 0.694 |
| SCF | EPHA6 | Alkaline phosphatase, placental | MASP3 | | IgG4, Kappa | DCC | RCAN3 | CD248 | 0.694 |
| SCF | EPHA6 | Alkaline phosphatase, placental | MASP3 | DUS10 | IgG4, Kappa | | | | 0.694 |
| SCF | | Alkaline phosphatase, placental | MASP3 | | IgG4, Kappa | | | CD248 | 0.693 |
| SCF | | Alkaline phosphatase, placental | MASP3 | | IgG4, Kappa | | RCAN3 | CD248 | 0.693 |
| SCF | | Alkaline phosphatase, placental | MASP3 | | IgG4, Kappa | DCC | | CD248 | 0.693 |
| SCF | | Alkaline phosphatase, placental | MASP3 | DUS10 | IgG4, Kappa | | | | 0.693 |
| SCF | | Alkaline phosphatase, placental | MASP3 | DUS10 | IgG4, Kappa | DCC | | | 0.693 |
| SCF | EPHA6 | Alkaline phosphatase, placental | MASP3 | | IgG4, Kappa | | RCAN3 | CD248 | 0.693 |
| SCF | EPHA6 | Alkaline phosphatase, placental | MASP3 | | IgG4, Kappa | DCC | | CD248 | 0.693 |
| SCF | EPHA6 | Alkaline phosphatase, placental | MASP3 | | IgG4, Kappa | | | CD248 | 0.692 |
| SCF | EPHA6 | Alkaline phosphatase, placental | MASP3 | | IgG4, Kappa | | RCAN3 | | 0.692 |
| SCF | EPHA6 | Alkaline phosphatase, placental | MASP3 | | IgG4, Kappa | DCC | RCAN3 | | 0.692 |
| SCF | | Alkaline phosphatase, placental | MASP3 | | IgG4, Kappa | | RCAN3 | | 0.691 |
| SCF | | Alkaline phosphatase, placental | MASP3 | | IgG4, Kappa | DCC | RCAN3 | | 0.691 |
| SCF | EPHA6 | Alkaline phosphatase, placental | MASP3 | | IgG4, Kappa | | | | 0.691 |
| SCF | | Alkaline phosphatase, placental | MASP3 | | IgG4, Kappa | | | | 0.69 |
| SCF | | Alkaline phosphatase, placental | MASP3 | | IgG4, Kappa | DCC | | | 0.69 |
| SCF | EPHA6 | Alkaline phosphatase, placental | MASP3 | | IgG4, Kappa | DCC | | | 0.69 |
| SCF | EPHA6 | Alkaline phosphatase, placental | MASP3 | DUS10 | | DCC | RCAN3 | CD248 | 0.689 |
| SCF | | Alkaline phosphatase, placental | | DUS10 | IgG4, Kappa | DCC | RCAN3 | CD248 | 0.687 |
| SCF | | Alkaline phosphatase, placental | MASP3 | DUS10 | | DCC | RCAN3 | CD248 | 0.687 |
| SCF | EPHA6 | Alkaline phosphatase, placental | | DUS10 | IgG4, Kappa | DCC | RCAN3 | CD248 | 0.687 |
| SCF | EPHA6 | Alkaline phosphatase, placental | MASP3 | DUS10 | | DCC | | CD248 | 0.687 |
| SCF | | Alkaline phosphatase, placental | MASP3 | DUS10 | | DCC | | CD248 | 0.686 |
| SCF | EPHA6 | Alkaline phosphatase, placental | | DUS10 | IgG4, Kappa | DCC | | CD248 | 0.686 |
| SCF | EPHA6 | Alkaline phosphatase, placental | MASP3 | DUS10 | | DCC | RCAN3 | | 0.686 |
| SCF | | Alkaline phosphatase, placental | | DUS10 | IgG4, Kappa | DCC | | CD248 | 0.685 |
| SCF | | Alkaline phosphatase, placental | MASP3 | DUS10 | | | RCAN3 | CD248 | 0.685 |
| SCF | EPHA6 | Alkaline phosphatase, placental | MASP3 | DUS10 | | | RCAN3 | CD248 | 0.685 |
| SCF | | Alkaline phosphatase, placental | MASP3 | DUS10 | | | | CD248 | 0.684 |
| SCF | EPHA6 | Alkaline phosphatase, placental | | DUS10 | IgG4, Kappa | DCC | RCAN3 | | 0.684 |
| SCF | EPHA6 | Alkaline phosphatase, placental | MASP3 | | | DCC | RCAN3 | CD248 | 0.684 |
| SCF | EPHA6 | Alkaline phosphatase, placental | MASP3 | DUS10 | | | | CD248 | 0.684 |
| SCF | EPHA6 | Alkaline phosphatase, placental | MASP3 | DUS10 | | DCC | | | 0.684 |
| SCF | | Alkaline phosphatase, placental | MASP3 | | | DCC | RCAN3 | CD248 | 0.683 |
| SCF | | Alkaline phosphatase, placental | MASP3 | DUS10 | | | RCAN3 | | 0.683 |
| SCF | | Alkaline phosphatase, placental | MASP3 | DUS10 | | DCC | RCAN3 | | 0.683 |
| SCF | EPHA6 | Alkaline phosphatase, placental | | DUS10 | IgG4, Kappa | DCC | | | 0.683 |
| SCF | EPHA6 | Alkaline phosphatase, placental | MASP3 | DUS10 | | | RCAN3 | | 0.683 |
| SCF | | Alkaline phosphatase, placental | MASP3 | DUS10 | | DCC | | | 0.682 |
| SCF | EPHA6 | Alkaline phosphatase, placental | | | IgG4, Kappa | DCC | RCAN3 | CD248 | 0.682 |
| SCF | EPHA6 | Alkaline phosphatase, placental | MASP3 | | | DCC | | CD248 | 0.682 |
| SCF | EPHA6 | Alkaline phosphatase, placental | MASP3 | DUS10 | | | | | 0.682 |
| SCF | | Alkaline phosphatase, placental | | | IgG4, Kappa | DCC | RCAN3 | CD248 | 0.681 |
| SCF | | Alkaline phosphatase, placental | | DUS10 | IgG4, Kappa | | RCAN3 | CD248 | 0.681 |
| SCF | | Alkaline phosphatase, placental | | DUS10 | IgG4, Kappa | DCC | RCAN3 | | 0.681 |
| SCF | | Alkaline phosphatase, placental | MASP3 | | | | RCAN3 | CD248 | 0.681 |
| SCF | | Alkaline phosphatase, placental | MASP3 | | | DCC | | CD248 | 0.681 |
| SCF | | Alkaline phosphatase, placental | MASP3 | DUS10 | | | | | 0.681 |
| SCF | EPHA6 | Alkaline phosphatase, placental | | DUS10 | IgG4, Kappa | | RCAN3 | CD248 | 0.681 |
| SCF | EPHA6 | Alkaline phosphatase, placental | MASP3 | | | | RCAN3 | CD248 | 0.681 |
| SCF | EPHA6 | Alkaline phosphatase, placental | MASP3 | | | DCC | RCAN3 | | 0.681 |
| SCF | | Alkaline phosphatase, placental | | DUS10 | IgG4, Kappa | | | CD248 | 0.68 |
| SCF | | Alkaline phosphatase, placental | MASP3 | | | | | CD248 | 0.68 |
| SCF | EPHA6 | Alkaline phosphatase, placental | | | IgG4, Kappa | DCC | | CD248 | 0.68 |
| SCF | EPHA6 | Alkaline phosphatase, placental | | | IgG4, Kappa | DCC | RCAN3 | | 0.68 |
| SCF | EPHA6 | Alkaline phosphatase, placental | | DUS10 | IgG4, Kappa | | | CD248 | 0.68 |
| SCF | EPHA6 | Alkaline phosphatase, placental | | DUS10 | IgG4, Kappa | | RCAN3 | | 0.68 |
| SCF | EPHA6 | Alkaline phosphatase, placental | MASP3 | | | | | CD248 | 0.68 |
| SCF | EPHA6 | Alkaline phosphatase, placental | MASP3 | | | | RCAN3 | | 0.68 |
| SCF | EPHA6 | Alkaline phosphatase, placental | MASP3 | | | DCC | | | 0.68 |
| SCF | | Alkaline phosphatase, placental | | | IgG4, Kappa | DCC | | CD248 | 0.679 |
| SCF | | Alkaline phosphatase, placental | | DUS10 | IgG4, Kappa | | RCAN3 | | 0.679 |
| SCF | | Alkaline phosphatase, placental | | DUS10 | IgG4, Kappa | DCC | | | 0.679 |
| SCF | | Alkaline phosphatase, placental | MASP3 | | | | RCAN3 | | 0.679 |
| SCF | | Alkaline phosphatase, placental | MASP3 | | | DCC | RCAN3 | | 0.678 |
| SCF | EPHA6 | Alkaline phosphatase, placental | | | IgG4, Kappa | DCC | | | 0.678 |
| SCF | EPHA6 | Alkaline phosphatase, placental | MASP3 | | | | | | 0.678 |
| SCF | | Alkaline phosphatase, placental | | DUS10 | IgG4, Kappa | | | | 0.677 |
| SCF | | Alkaline phosphatase, placental | MASP3 | | | | | | 0.677 |
| SCF | | Alkaline phosphatase, placental | MASP3 | | | DCC | | | 0.677 |
| SCF | EPHA6 | Alkaline phosphatase, placental | | DUS10 | IgG4, Kappa | | | | 0.677 |
| SCF | | Alkaline phosphatase, placental | | | IgG4, Kappa | | RCAN3 | CD248 | 0.676 |
| SCF | | Alkaline phosphatase, placental | | | IgG4, Kappa | DCC | RCAN3 | | 0.676 |
| SCF | EPHA6 | Alkaline phosphatase, placental | | | IgG4, Kappa | | RCAN3 | CD248 | 0.676 |
| SCF | | Alkaline phosphatase, placental | | | IgG4, Kappa | | | CD248 | 0.675 |
| SCF | | Alkaline phosphatase, placental | | | IgG4, Kappa | | RCAN3 | | 0.674 |
| SCF | EPHA6 | Alkaline phosphatase, placental | | | IgG4, Kappa | | | CD248 | 0.674 |
| SCF | EPHA6 | Alkaline phosphatase, placental | | | IgG4, Kappa | | RCAN3 | | 0.674 |
| SCF | EPHA6 | Alkaline phosphatase, placental | | DUS10 | | DCC | RCAN3 | CD248 | 0.674 |
| SCF | | Alkaline phosphatase, placental | | | IgG4, Kappa | DCC | | | 0.673 |
| SCF | EPHA6 | Alkaline phosphatase, placental | | | IgG4, Kappa | | | | 0.673 |
| SCF | | Alkaline phosphatase, placental | | DUS10 | | DCC | RCAN3 | CD248 | 0.672 |
| SCF | EPHA6 | Alkaline phosphatase, placental | | DUS10 | | DCC | | CD248 | 0.672 |
| SCF | EPHA6 | Alkaline phosphatase, placental | | DUS10 | | DCC | RCAN3 | | 0.672 |
| SCF | | Alkaline phosphatase, placental | | | IgG4, Kappa | | | | 0.671 |
| SCF | | Alkaline phosphatase, placental | | DUS10 | | DCC | | CD248 | 0.67 |
| SCF | EPHA6 | Alkaline phosphatase, placental | | DUS10 | | DCC | | | 0.669 |
| SCF | | Alkaline phosphatase, placental | | DUS10 | | DCC | RCAN3 | | 0.667 |
| SCF | EPHA6 | Alkaline phosphatase, placental | | | | DCC | RCAN3 | CD248 | 0.667 |
| SCF | | Alkaline phosphatase, placental | | | | DCC | RCAN3 | CD248 | 0.665 |
| SCF | | Alkaline phosphatase, placental | | DUS10 | | | RCAN3 | CD248 | 0.665 |
| SCF | EPHA6 | Alkaline phosphatase, placental | | | | DCC | RCAN3 | | 0.665 |
| SCF | EPHA6 | Alkaline phosphatase, placental | | DUS10 | | | RCAN3 | CD248 | 0.665 |
| SCF | | Alkaline phosphatase, placental | | DUS10 | | DCC | | | 0.664 |
| SCF | EPHA6 | Alkaline phosphatase, placental | | | | DCC | | CD248 | 0.664 |
| SCF | EPHA6 | Alkaline phosphatase, placental | | DUS10 | | | RCAN3 | | 0.664 |
| SCF | | Alkaline phosphatase, placental | | DUS10 | | | RCAN3 | | 0.663 |
| SCF | | Alkaline phosphatase, placental | | | | DCC | | CD248 | 0.662 |
| SCF | | Alkaline phosphatase, placental | | DUS10 | | | | CD248 | 0.662 |
| SCF | EPHA6 | Alkaline phosphatase, placental | | | | DCC | | | 0.662 |
| SCF | EPHA6 | Alkaline phosphatase, placental | | DUS10 | | | | CD248 | 0.662 |
| SCF | EPHA6 | Alkaline phosphatase, placental | | DUS10 | | | | | 0.661 |
| SCF | | Alkaline phosphatase, placental | | | | DCC | RCAN3 | | 0.66 |
| SCF | | Alkaline phosphatase, placental | | DUS10 | | | | | 0.659 |
| SCF | | Alkaline phosphatase, placental | | | | | RCAN3 | CD248 | 0.658 |
| SCF | EPHA6 | Alkaline phosphatase, placental | | | | | RCAN3 | | 0.658 |
| SCF | EPHA6 | Alkaline phosphatase, placental | | | | | RCAN3 | CD248 | 0.658 |
| SCF | | Alkaline phosphatase, placental | | | | | RCAN3 | | 0.656 |
| SCF | | Alkaline phosphatase, placental | | | | | | CD248 | 0.655 |
| SCF | | Alkaline phosphatase, placental | | | | DCC | | | 0.655 |
| SCF | EPHA6 | Alkaline phosphatase, placental | | | | | | CD248 | 0.654 |
| SCF | EPHA6 | Alkaline phosphatase, placental | | | | | | | 0.653 |
| SCF | | Alkaline phosphatase, placental | | | | | | | 0.652 |
| SCF | EPHA6 | | MASP3 | | IgG4, Kappa | DCC | | CD248 | 0.635 |
| SCF | EPHA6 | | MASP3 | | IgG4, Kappa | DCC | RCAN3 | CD248 | 0.635 |
| SCF | EPHA6 | | MASP3 | DUS10 | IgG4, Kappa | DCC | | | 0.634 |
| SCF | EPHA6 | | MASP3 | DUS10 | IgG4, Kappa | DCC | | CD248 | 0.634 |
| SCF | EPHA6 | | MASP3 | DUS10 | IgG4, Kappa | DCC | RCAN3 | | 0.634 |
| SCF | EPHA6 | | MASP3 | DUS10 | IgG4, Kappa | DCC | RCAN3 | CD248 | 0.634 |
| SCF | EPHA6 | | MASP3 | | IgG4, Kappa | DCC | | | 0.633 |
| SCF | EPHA6 | | MASP3 | | IgG4, Kappa | DCC | RCAN3 | | 0.633 |
| SCF | | | MASP3 | | IgG4, Kappa | DCC | | CD248 | 0.631 |
| SCF | | | MASP3 | | IgG4, Kappa | DCC | RCAN3 | CD248 | 0.631 |
| SCF | | | MASP3 | DUS10 | IgG4, Kappa | DCC | | CD248 | 0.631 |
| SCF | | | MASP3 | DUS10 | IgG4, Kappa | DCC | RCAN3 | CD248 | 0.63 |
| SCF | | | MASP3 | | IgG4, Kappa | DCC | | | 0.629 |
| SCF | | | MASP3 | | IgG4, Kappa | DCC | RCAN3 | | 0.629 |
| SCF | | | MASP3 | DUS10 | IgG4, Kappa | DCC | | | 0.629 |
| SCF | | | MASP3 | DUS10 | IgG4, Kappa | DCC | RCAN3 | | 0.629 |
| SCF | EPHA6 | | | | IgG4, Kappa | DCC | | CD248 | 0.629 |
| SCF | EPHA6 | | | | IgG4, Kappa | DCC | RCAN3 | | 0.629 |
| SCF | EPHA6 | | | | IgG4, Kappa | DCC | RCAN3 | CD248 | 0.629 |
| SCF | EPHA6 | | | DUS10 | IgG4, Kappa | DCC | | | 0.629 |
| SCF | EPHA6 | | | DUS10 | IgG4, Kappa | DCC | | CD248 | 0.629 |
| SCF | EPHA6 | | | DUS10 | IgG4, Kappa | DCC | RCAN3 | | 0.629 |
| SCF | EPHA6 | | | DUS10 | IgG4, Kappa | DCC | RCAN3 | CD248 | 0.629 |
| SCF | EPHA6 | | | | IgG4, Kappa | DCC | | | 0.628 |
| SCF | | | | DUS10 | IgG4, Kappa | DCC | | CD248 | 0.627 |
| SCF | | | | DUS10 | IgG4, Kappa | DCC | RCAN3 | CD248 | 0.627 |
| SCF | | | MASP3 | DUS10 | IgG4, Kappa | | | CD248 | 0.627 |
| SCF | | | MASP3 | DUS10 | IgG4, Kappa | | RCAN3 | CD248 | 0.627 |
| SCF | EPHA6 | | MASP3 | DUS10 | IgG4, Kappa | | | CD248 | 0.627 |
| SCF | EPHA6 | | MASP3 | DUS10 | IgG4, Kappa | | RCAN3 | | 0.627 |
| SCF | EPHA6 | | MASP3 | DUS10 | IgG4, Kappa | | RCAN3 | CD248 | 0.627 |
| SCF | | | | | IgG4, Kappa | DCC | | CD248 | 0.626 |
| SCF | | | | | IgG4, Kappa | DCC | RCAN3 | CD248 | 0.626 |
| SCF | | | MASP3 | | IgG4, Kappa | | | CD248 | 0.626 |
| SCF | | | MASP3 | | IgG4, Kappa | | RCAN3 | CD248 | 0.626 |
| SCF | | | MASP3 | DUS10 | IgG4, Kappa | | | | 0.626 |
| SCF | | | MASP3 | DUS10 | IgG4, Kappa | | RCAN3 | | 0.626 |
| SCF | EPHA6 | | MASP3 | | IgG4, Kappa | | | | 0.626 |
| SCF | EPHA6 | | MASP3 | | IgG4, Kappa | | | CD248 | 0.626 |
| SCF | EPHA6 | | MASP3 | | IgG4, Kappa | | RCAN3 | CD248 | 0.626 |
| SCF | EPHA6 | | MASP3 | DUS10 | IgG4, Kappa | | | | 0.626 |
| SCF | | | MASP3 | | IgG4, Kappa | | | | 0.625 |
| SCF | | | MASP3 | | IgG4, Kappa | | RCAN3 | | 0.625 |
| SCF | EPHA6 | | MASP3 | | IgG4, Kappa | | RCAN3 | | 0.625 |
| SCF | | | | DUS10 | IgG4, Kappa | DCC | | | 0.623 |
| SCF | | | | DUS10 | IgG4, Kappa | DCC | RCAN3 | | 0.623 |
| SCF | | | | | IgG4, Kappa | DCC | RCAN3 | | 0.622 |
| SCF | | | | | IgG4, Kappa | DCC | | | 0.621 |
| SCF | | | | DUS10 | IgG4, Kappa | | | CD248 | 0.618 |
| SCF | | | | DUS10 | IgG4, Kappa | | RCAN3 | CD248 | 0.618 |
| SCF | EPHA6 | | | DUS10 | IgG4, Kappa | | | | 0.618 |
| SCF | EPHA6 | | | DUS10 | IgG4, Kappa | | | CD248 | 0.618 |
| SCF | EPHA6 | | | DUS10 | IgG4, Kappa | | RCAN3 | | 0.618 |
| SCF | EPHA6 | | | DUS10 | IgG4, Kappa | | RCAN3 | CD248 | 0.618 |
| SCF | | | | | IgG4, Kappa | | | CD248 | 0.616 |
| SCF | | | | | IgG4, Kappa | | RCAN3 | CD248 | 0.616 |
| SCF | | | | DUS10 | IgG4, Kappa | | | | 0.616 |
| SCF | | | | DUS10 | IgG4, Kappa | | RCAN3 | | 0.616 |
| SCF | EPHA6 | | | | IgG4, Kappa | | RCAN3 | | 0.616 |
| SCF | EPHA6 | | | | IgG4, Kappa | | RCAN3 | CD248 | 0.616 |
| SCF | | | | | IgG4, Kappa | | RCAN3 | | 0.615 |
| SCF | EPHA6 | | | | IgG4, Kappa | | | | 0.615 |
| SCF | EPHA6 | | | | IgG4, Kappa | | | CD248 | 0.615 |
| SCF | EPHA6 | | MASP3 | | | DCC | | CD248 | 0.615 |
| SCF | EPHA6 | | MASP3 | | | DCC | RCAN3 | CD248 | 0.615 |
| SCF | EPHA6 | | MASP3 | DUS10 | | DCC | | | 0.615 |
| SCF | EPHA6 | | MASP3 | DUS10 | | DCC | | CD248 | 0.615 |
| SCF | EPHA6 | | MASP3 | DUS10 | | DCC | RCAN3 | | 0.615 |
| SCF | EPHA6 | | MASP3 | DUS10 | | DCC | RCAN3 | CD248 | 0.615 |
| SCF | | | | | IgG4, Kappa | | | | 0.614 |
| SCF | EPHA6 | | MASP3 | | | DCC | | | 0.614 |
| SCF | EPHA6 | | MASP3 | | | DCC | RCAN3 | | 0.614 |
| SCF | EPHA6 | | | DUS10 | | DCC | RCAN3 | CD248 | 0.613 |
| SCF | EPHA6 | | | DUS10 | | DCC | | | 0.612 |
| SCF | EPHA6 | | | DUS10 | | DCC | | CD248 | 0.612 |
| SCF | EPHA6 | | | DUS10 | | DCC | RCAN3 | | 0.612 |
| SCF | | | MASP3 | DUS10 | | DCC | | CD248 | 0.611 |
| SCF | | | MASP3 | DUS10 | | DCC | RCAN3 | CD248 | 0.611 |
| SCF | EPHA6 | | | | | DCC | RCAN3 | CD248 | 0.611 |
| SCF | | | MASP3 | | | DCC | | CD248 | 0.61 |
| SCF | | | MASP3 | | | DCC | RCAN3 | CD248 | 0.61 |
| SCF | EPHA6 | | | | | DCC | | CD248 | 0.61 |
| SCF | EPHA6 | | | | | DCC | | | 0.609 |
| SCF | EPHA6 | | | | | DCC | RCAN3 | | 0.609 |
| SCF | | | MASP3 | DUS10 | | DCC | | | 0.607 |
| SCF | | | MASP3 | DUS10 | | DCC | RCAN3 | | 0.607 |
| SCF | | | MASP3 | | | DCC | | | 0.606 |
| SCF | | | MASP3 | | | DCC | RCAN3 | | 0.606 |
| SCF | | | | DUS10 | | DCC | | CD248 | 0.605 |
| SCF | | | | DUS10 | | DCC | RCAN3 | CD248 | 0.605 |
| SCF | EPHA6 | | MASP3 | DUS10 | | | | CD248 | 0.605 |
| SCF | EPHA6 | | MASP3 | DUS10 | | | RCAN3 | | 0.605 |
| SCF | EPHA6 | | MASP3 | DUS10 | | | RCAN3 | CD248 | 0.605 |
| SCF | | | MASP3 | DUS10 | | | | CD248 | 0.604 |
| SCF | | | MASP3 | DUS10 | | | RCAN3 | CD248 | 0.604 |
| SCF | EPHA6 | | MASP3 | | | | | CD248 | 0.604 |
| SCF | EPHA6 | | MASP3 | | | | RCAN3 | | 0.604 |
| SCF | EPHA6 | | MASP3 | | | | RCAN3 | CD248 | 0.604 |
| SCF | EPHA6 | | MASP3 | DUS10 | | | | | 0.604 |
| SCF | | | | | | DCC | RCAN3 | CD248 | 0.603 |
| SCF | | | MASP3 | | | | | CD248 | 0.603 |
| SCF | | | MASP3 | | | | RCAN3 | CD248 | 0.603 |
| SCF | | | MASP3 | DUS10 | | | | | 0.603 |
| SCF | | | MASP3 | DUS10 | | | RCAN3 | | 0.603 |
| SCF | EPHA6 | | MASP3 | | | | | | 0.603 |
| SCF | | | | | | DCC | | CD248 | 0.602 |
| SCF | | | | DUS10 | | DCC | | | 0.602 |
| SCF | | | | DUS10 | | DCC | RCAN3 | | 0.602 |
| SCF | | | MASP3 | | | | | | 0.602 |
| SCF | | | MASP3 | | | | RCAN3 | | 0.602 |

TABLE 18
MASP3: Heavy Biomarker Panels

| | | Alkaline | | | | | | | |
|---|---|---|---|---|---|---|---|---|---|
| | | phosphatase, | | | IgG4, | | | | |
| MASP3 | EPHA6 | placental | SCF | DUS10 | Kappa | DCC | RCAN3 | CD248 | AUC |
| MASP3 | EPHA6 | Alkaline | SCF | DUS10 | IgG4, | DCC | RCAN3 | CD248 | 0.698 |
| | | phosphatase, | | | Kappa | | | | |
| | | placental | | | | | | | |
| MASP3 | | Alkaline | SCF | DUS10 | IgG4, | DCC | RCAN3 | CD248 | 0.697 |
| | | phosphatase, | | | Kappa | | | | |
| | | placental | | | | | | | |
| MASP3 | EPHA6 | Alkaline | SCF | DUS10 | IgG4, | DCC | | CD248 | 0.697 |
| | | phosphatase, | | | Kappa | | | | |
| | | placental | | | | | | | |
| MASP3 | | Alkaline | SCF | DUS10 | IgG4, | | | CD248 | 0.696 |
| | | phosphatase, | | | Kappa | | | | |
| | | placental | | | | | | | |
| MASP3 | | Alkaline | SCF | DUS10 | IgG4, | | RCAN3 | CD248 | 0.696 |
| | | phosphatase, | | | Kappa | | | | |
| | | placental | | | | | | | |
| MASP3 | | Alkaline | SCF | DUS10 | IgG4, | DCC | | CD248 | 0.696 |
| | | phosphatase, | | | Kappa | | | | |
| | | placental | | | | | | | |
| MASP3 | EPHA6 | Alkaline | SCF | DUS10 | IgG4, | | | CD248 | 0.696 |
| | | phosphatase, | | | Kappa | | | | |
| | | placental | | | | | | | |
| MASP3 | EPHA6 | Alkaline | SCF | DUS10 | IgG4, | | RCAN3 | CD248 | 0.696 |
| | | phosphatase, | | | Kappa | | | | |
| | | placental | | | | | | | |
| MASP3 | EPHA6 | Alkaline | SCF | DUS10 | IgG4, | DCC | RCAN3 | | 0.696 |
| | | phosphatase, | | | Kappa | | | | |
| | | placental | | | | | | | |
| MASP3 | EPHA6 | Alkaline | SCF | DUS10 | IgG4, | | RCAN3 | | 0.695 |
| | | phosphatase, | | | Kappa | | | | |
| | | placental | | | | | | | |
| MASP3 | EPHA6 | Alkaline | SCF | DUS10 | IgG4, | DCC | | | 0.695 |
| | | phosphatase, | | | Kappa | | | | |
| | | placental | | | | | | | |
| MASP3 | | Alkaline | SCF | | IgG4, | DCC | RCAN3 | CD248 | 0.694 |
| | | phosphatase, | | | Kappa | | | | |
| | | placental | | | | | | | |
| MASP3 | | Alkaline | SCF | DUS10 | IgG4, | | RCAN3 | | 0.694 |
| | | phosphatase, | | | Kappa | | | | |
| | | placental | | | | | | | |
| MASP3 | | Alkaline | SCF | DUS10 | IgG4, | DCC | RCAN3 | | 0.694 |
| | | phosphatase, | | | Kappa | | | | |
| | | placental | | | | | | | |
| MASP3 | EPHA6 | Alkaline | SCF | | IgG4, | DCC | RCAN3 | CD248 | 0.694 |
| | | phosphatase, | | | Kappa | | | | |
| | | placental | | | | | | | |
| MASP3 | EPHA6 | Alkaline | SCF | DUS10 | IgG4, | | | | 0.694 |
| | | phosphatase, | | | Kappa | | | | |
| | | placental | | | | | | | |
| MASP3 | | Alkaline | SCF | | IgG4, | | | CD248 | 0.693 |
| | | phosphatase, | | | Kappa | | | | |
| | | placental | | | | | | | |
| MASP3 | | Alkaline | SCF | | IgG4, | | RCAN3 | CD248 | 0.693 |
| | | phosphatase, | | | Kappa | | | | |
| | | placental | | | | | | | |
| MASP3 | | Alkaline | SCF | | IgG4, | DCC | | CD248 | 0.693 |
| | | phosphatase, | | | Kappa | | | | |
| | | placental | | | | | | | |
| MASP3 | | Alkaline | SCF | DUS10 | IgG4, | | | | 0.693 |
| | | phosphatase, | | | Kappa | | | | |
| | | placental | | | | | | | |
| MASP3 | | Alkaline | SCF | DUS10 | IgG4, | DCC | | | 0.693 |
| | | phosphatase, | | | Kappa | | | | |
| | | placental | | | | | | | |
| MASP3 | EPHA6 | Alkaline | SCF | | IgG4, | | RCAN3 | CD248 | 0.693 |
| | | phosphatase, | | | Kappa | | | | |
| | | placental | | | | | | | |
| MASP3 | EPHA6 | Alkaline | SCF | | IgG4, | DCC | | CD248 | 0.693 |
| | | phosphatase, | | | Kappa | | | | |
| | | placental | | | | | | | |
| MASP3 | EPHA6 | Alkaline | SCF | | IgG4, | | | CD248 | 0.692 |
| | | phosphatase, | | | Kappa | | | | |
| | | placental | | | | | | | |
| MASP3 | EPHA6 | Alkaline | SCF | | IgG4, | | RCAN3 | | 0.692 |
| | | phosphatase, | | | Kappa | | | | |
| | | placental | | | | | | | |
| MASP3 | EPHA6 | Alkaline | SCF | | IgG4, | DCC | RCAN3 | | 0.692 |
| | | phosphatase, | | | Kappa | | | | |
| | | placental | | | | | | | |
| MASP3 | | Alkaline | SCF | | IgG4, | | RCAN3 | | 0.691 |
| | | phosphatase, | | | Kappa | | | | |
| | | placental | | | | | | | |
| MASP3 | | Alkaline | SCF | | IgG4, | DCC | RCAN3 | | 0.691 |
| | | phosphatase, | | | Kappa | | | | |
| | | placental | | | | | | | |
| MASP3 | EPHA6 | Alkaline | SCF | | IgG4, | | | | 0.691 |
| | | phosphatase, | | | Kappa | | | | |
| | | placental | | | | | | | |
| MASP3 | | Alkaline | SCF | | IgG4, | | | | 0.69 |
| | | phosphatase, | | | Kappa | | | | |
| | | placental | | | | | | | |
| MASP3 | | Alkaline | SCF | | IgG4, | DCC | | | 0.69 |
| | | phosphatase, | | | Kappa | | | | |
| | | placental | | | | | | | |
| MASP3 | EPHA6 | Alkaline | SCF | | IgG4, | DCC | | | 0.69 |
| | | phosphatase, | | | Kappa | | | | |
| | | placental | | | | | | | |
| MASP3 | EPHA6 | Alkaline | SCF | DUS10 | | DCC | RCAN3 | CD248 | 0.689 |
| | | phosphatase, | | | | | | | |
| | | placental | | | | | | | |
| MASP3 | | Alkaline | SCF | DUS10 | | DCC | RCAN3 | CD248 | 0.687 |
| | | phosphatase, | | | | | | | |
| | | placental | | | | | | | |
| MASP3 | EPHA6 | Alkaline | SCF | DUS10 | | DCC | | CD248 | 0.687 |
| | | phosphatase, | | | | | | | |
| | | placental | | | | | | | |
| MASP3 | | Alkaline | SCF | DUS10 | | DCC | | CD248 | 0.686 |
| | | phosphatase, | | | | | | | |
| | | placental | | | | | | | |
| MASP3 | EPHA6 | Alkaline | SCF | DUS10 | | DCC | RCAN3 | | 0.686 |
| | | phosphatase, | | | | | | | |
| | | placental | | | | | | | |
| MASP3 | | Alkaline | SCF | DUS10 | | | RCAN3 | CD248 | 0.685 |
| | | phosphatase, | | | | | | | |
| | | placental | | | | | | | |
| MASP3 | EPHA6 | Alkaline | SCF | DUS10 | | | RCAN3 | CD248 | 0.685 |
| | | phosphatase, | | | | | | | |
| | | placental | | | | | | | |
| MASP3 | | Alkaline | SCF | DUS10 | | | | CD248 | 0.684 |
| | | phosphatase, | | | | | | | |
| | | placental | | | | | | | |
| MASP3 | EPHA6 | Alkaline | SCF | | | DCC | RCAN3 | CD248 | 0.684 |
| | | phosphatase, | | | | | | | |
| | | placental | | | | | | | |
| MASP3 | EPHA6 | Alkaline | SCF | DUS10 | | | | CD248 | 0.684 |
| | | phosphatase, | | | | | | | |
| | | placental | | | | | | | |
| MASP3 | EPHA6 | Alkaline | SCF | DUS10 | | DCC | | | 0.684 |
| | | phosphatase, | | | | | | | |
| | | placental | | | | | | | |
| MASP3 | | Alkaline | | DUS10 | IgG4, | DCC | | CD248 | 0.683 |
| | | phosphatase, | | | Kappa | | | | |
| | | placental | | | | | | | |
| MASP3 | | Alkaline | SCF | | | DCC | RCAN3 | CD248 | 0.683 |
| | | phosphatase, | | | | | | | |
| | | placental | | | | | | | |
| MASP3 | | Alkaline | SCF | DUS10 | | | RCAN3 | | 0.683 |
| | | phosphatase, | | | | | | | |
| | | placental | | | | | | | |
| MASP3 | | Alkaline | SCF | DUS10 | | DCC | RCAN3 | | 0.683 |
| | | phosphatase, | | | | | | | |
| | | placental | | | | | | | |
| MASP3 | EPHA6 | Alkaline | | DUS10 | IgG4, | DCC | | CD248 | 0.683 |
| | | phosphatase, | | | Kappa | | | | |
| | | placental | | | | | | | |
| MASP3 | EPHA6 | Alkaline | SCF | DUS10 | | | RCAN3 | | 0.683 |
| | | phosphatase, | | | | | | | |
| | | placental | | | | | | | |
| MASP3 | | Alkaline | | DUS10 | IgG4, | DCC | RCAN3 | CD248 | 0.682 |
| | | phosphatase, | | | Kappa | | | | |
| | | placental | | | | | | | |
| MASP3 | | Alkaline | SCF | DUS10 | | DCC | | | 0.682 |
| | | phosphatase, | | | | | | | |
| | | placental | | | | | | | |
| MASP3 | EPHA6 | Alkaline | | DUS10 | IgG4, | DCC | RCAN3 | CD248 | 0.682 |
| | | phosphatase, | | | Kappa | | | | |
| | | placental | | | | | | | |
| MASP3 | EPHA6 | Alkaline | SCF | | | DCC | | CD248 | 0.682 |
| | | phosphatase, | | | | | | | |
| | | placental | | | | | | | |
| MASP3 | EPHA6 | Alkaline | SCF | DUS10 | | | | | 0.682 |
| | | phosphatase, | | | | | | | |
| | | placental | | | | | | | |
| MASP3 | | Alkaline | SCF | | | | RCAN3 | CD248 | 0.681 |
| | | phosphatase, | | | | | | | |
| | | placental | | | | | | | |
| MASP3 | | Alkaline | SCF | | | DCC | | CD248 | 0.681 |
| | | phosphatase, | | | | | | | |
| | | placental | | | | | | | |
| MASP3 | | Alkaline | SCF | DUS10 | | | | | 0.681 |
| | | phosphatase, | | | | | | | |
| | | placental | | | | | | | |
| MASP3 | EPHA6 | Alkaline | | DUS10 | IgG4, | | | CD248 | 0.681 |
| | | phosphatase, | | | Kappa | | | | |
| | | placental | | | | | | | |
| MASP3 | EPHA6 | Alkaline | | DUS10 | IgG4, | | RCAN3 | CD248 | 0.681 |
| | | phosphatase, | | | Kappa | | | | |
| | | placental | | | | | | | |
| MASP3 | EPHA6 | Alkaline | SCF | | | | RCAN3 | CD248 | 0.681 |
| | | phosphatase, | | | | | | | |
| | | placental | | | | | | | |
| MASP3 | EPHA6 | Alkaline | SCF | | | DCC | RCAN3 | | 0.681 |
| | | phosphatase, | | | | | | | |
| | | placental | | | | | | | |
| MASP3 | | Alkaline | | DUS10 | IgG4, | | | | 0.68 |
| | | phosphatase, | | | Kappa | | | | |
| | | placental | | | | | | | |
| MASP3 | | Alkaline | | DUS10 | IgG4, | | | CD248 | 0.68 |
| | | phosphatase, | | | Kappa | | | | |
| | | placental | | | | | | | |
| MASP3 | | Alkaline | | DUS10 | IgG4, | | RCAN3 | | 0.68 |
| | | phosphatase, | | | Kappa | | | | |
| | | placental | | | | | | | |
| MASP3 | | Alkaline | | DUS10 | IgG4, | | RCAN3 | CD248 | 0.68 |
| | | phosphatase, | | | Kappa | | | | |
| | | placental | | | | | | | |
| MASP3 | | Alkaline | | DUS10 | IgG4, | DCC | | | 0.68 |
| | | phosphatase, | | | Kappa | | | | |
| | | placental | | | | | | | |
| MASP3 | | Alkaline | | DUS10 | IgG4, | DCC | RCAN3 | | 0.68 |
| | | phosphatase, | | | Kappa | | | | |
| | | placental | | | | | | | |
| MASP3 | | Alkaline | SCF | | | | | CD248 | 0.68 |
| | | phosphatase, | | | | | | | |
| | | placental | | | | | | | |
| MASP3 | EPHA6 | Alkaline | | DUS10 | IgG4, | | | | 0.68 |
| | | phosphatase, | | | Kappa | | | | |
| | | placental | | | | | | | |
| MASP3 | EPHA6 | Alkaline | | DUS10 | IgG4, | | RCAN3 | | 0.68 |
| | | phosphatase, | | | Kappa | | | | |
| | | placental | | | | | | | |
| MASP3 | EPHA6 | Alkaline | | DUS10 | IgG4, | DCC | | | 0.68 |
| | | phosphatase, | | | Kappa | | | | |
| | | placental | | | | | | | |
| MASP3 | EPHA6 | Alkaline | | DUS10 | IgG4, | DCC | RCAN3 | | 0.68 |
| | | phosphatase, | | | Kappa | | | | |
| | | placental | | | | | | | |
| MASP3 | EPHA6 | Alkaline | SCF | | | | | CD248 | 0.68 |
| | | phosphatase, | | | | | | | |
| | | placental | | | | | | | |
| MASP3 | EPHA6 | Alkaline | SCF | | | | RCAN3 | | 0.68 |
| | | phosphatase, | | | | | | | |
| | | placental | | | | | | | |
| MASP3 | EPHA6 | Alkaline | SCF | | | DCC | | | 0.68 |
| | | phosphatase, | | | | | | | |
| | | placental | | | | | | | |
| MASP3 | | Alkaline | SCF | | | | RCAN3 | | 0.679 |
| | | phosphatase, | | | | | | | |
| | | placental | | | | | | | |
| MASP3 | | Alkaline | | | IgG4, | DCC | | CD248 | 0.678 |
| | | phosphatase, | | | Kappa | | | | |
| | | placental | | | | | | | |
| MASP3 | | Alkaline | | | IgG4, | DCC | RCAN3 | CD248 | 0.678 |
| | | phosphatase, | | | Kappa | | | | |
| | | placental | | | | | | | |
| MASP3 | | Alkaline | SCF | | | DCC | RCAN3 | | 0.678 |
| | | phosphatase, | | | | | | | |
| | | placental | | | | | | | |
| MASP3 | EPHA6 | Alkaline | | | IgG4, | DCC | | CD248 | 0.678 |
| | | phosphatase, | | | Kappa | | | | |
| | | placental | | | | | | | |
| MASP3 | EPHA6 | Alkaline | | | IgG4, | DCC | RCAN3 | CD248 | 0.678 |
| | | phosphatase, | | | Kappa | | | | |
| | | placental | | | | | | | |
| MASP3 | EPHA6 | Alkaline | SCF | | | | | | 0.678 |
| | | phosphatase, | | | | | | | |
| | | placental | | | | | | | |
| MASP3 | | Alkaline | SCF | | | | | | 0.677 |
| | | phosphatase, | | | | | | | |
| | | placental | | | | | | | |
| MASP3 | | Alkaline | SCF | | | DCC | | | 0.677 |
| | | phosphatase, | | | | | | | |
| | | placental | | | | | | | |
| MASP3 | EPHA6 | Alkaline | | | IgG4, | DCC | | | 0.677 |
| | | phosphatase, | | | Kappa | | | | |
| | | placental | | | | | | | |
| MASP3 | EPHA6 | Alkaline | | | IgG4, | DCC | RCAN3 | | 0.677 |
| | | phosphatase, | | | Kappa | | | | |
| | | placental | | | | | | | |
| MASP3 | | Alkaline | | | IgG4, | | | | 0.676 |
| | | phosphatase, | | | Kappa | | | | |
| | | placental | | | | | | | |
| MASP3 | | Alkaline | | | IgG4, | | RCAN3 | | 0.676 |
| | | phosphatase, | | | Kappa | | | | |
| | | placental | | | | | | | |
| MASP3 | | Alkaline | | | IgG4, | DCC | RCAN3 | | 0.676 |
| | | phosphatase, | | | Kappa | | | | |
| | | placental | | | | | | | |
| MASP3 | EPHA6 | Alkaline | | | IgG4, | | | CD248 | 0.676 |
| | | phosphatase, | | | Kappa | | | | |
| | | placental | | | | | | | |
| MASP3 | | Alkaline | | | IgG4, | | | CD248 | 0.675 |
| | | phosphatase, | | | Kappa | | | | |
| | | placental | | | | | | | |
| MASP3 | | Alkaline | | | IgG4, | | RCAN3 | CD248 | 0.675 |
| | | phosphatase, | | | Kappa | | | | |
| | | placental | | | | | | | |
| MASP3 | | Alkaline | | | IgG4, | DCC | | | 0.675 |
| | | phosphatase, | | | Kappa | | | | |
| | | placental | | | | | | | |
| MASP3 | | Alkaline | | DUS10 | | DCC | | CD248 | 0.675 |
| | | phosphatase, | | | | | | | |
| | | placental | | | | | | | |
| MASP3 | | Alkaline | | DUS10 | | DCC | RCAN3 | CD248 | 0.675 |
| | | phosphatase, | | | | | | | |
| | | placental | | | | | | | |
| MASP3 | EPHA6 | Alkaline | | | IgG4, | | | | 0.675 |
| | | phosphatase, | | | Kappa | | | | |
| | | placental | | | | | | | |
| MASP3 | EPHA6 | Alkaline | | | IgG4, | | RCAN3 | | 0.675 |
| | | phosphatase, | | | Kappa | | | | |
| | | placental | | | | | | | |
| MASP3 | EPHA6 | Alkaline | | | IgG4, | | RCAN3 | CD248 | 0.675 |
| | | phosphatase, | | | Kappa | | | | |
| | | placental | | | | | | | |
| MASP3 | EPHA6 | Alkaline | | DUS10 | | DCC | | CD248 | 0.674 |
| | | phosphatase, | | | | | | | |
| | | placental | | | | | | | |
| MASP3 | EPHA6 | Alkaline | | DUS10 | | DCC | RCAN3 | CD248 | 0.674 |
| | | phosphatase, | | | | | | | |
| | | placental | | | | | | | |
| MASP3 | EPHA6 | Alkaline | | DUS10 | | DCC | | | 0.673 |
| | | phosphatase, | | | | | | | |
| | | placental | | | | | | | |
| MASP3 | EPHA6 | Alkaline | | DUS10 | | DCC | RCAN3 | | 0.673 |
| | | phosphatase, | | | | | | | |
| | | placental | | | | | | | |
| MASP3 | | Alkaline | | DUS10 | | DCC | | | 0.672 |
| | | phosphatase, | | | | | | | |
| | | placental | | | | | | | |
| MASP3 | | Alkaline | | DUS10 | | DCC | RCAN3 | | 0.672 |
| | | phosphatase, | | | | | | | |
| | | placental | | | | | | | |
| MASP3 | | Alkaline | | DUS10 | | | | | 0.671 |
| | | phosphatase, | | | | | | | |
| | | placental | | | | | | | |
| MASP3 | | Alkaline | | DUS10 | | | | CD248 | 0.671 |
| | | phosphatase, | | | | | | | |
| | | placental | | | | | | | |
| MASP3 | | Alkaline | | DUS10 | | | RCAN3 | | 0.671 |
| | | phosphatase, | | | | | | | |
| | | placental | | | | | | | |
| MASP3 | | Alkaline | | DUS10 | | | RCAN3 | CD248 | 0.671 |
| | | phosphatase, | | | | | | | |
| | | placental | | | | | | | |
| MASP3 | EPHA6 | Alkaline | | DUS10 | | | RCAN3 | CD248 | 0.671 |
| | | phosphatase, | | | | | | | |
| | | placental | | | | | | | |
| MASP3 | EPHA6 | Alkaline | | DUS10 | | | | | 0.67 |
| | | phosphatase, | | | | | | | |
| | | placental | | | | | | | |
| MASP3 | EPHA6 | Alkaline | | DUS10 | | | | CD248 | 0.67 |
| | | phosphatase, | | | | | | | |
| | | placental | | | | | | | |
| MASP3 | EPHA6 | Alkaline | | DUS10 | | | RCAN3 | | 0.67 |
| | | phosphatase, | | | | | | | |
| | | placental | | | | | | | |
| MASP3 | | Alkaline | | | | DCC | | CD248 | 0.668 |
| | | phosphatase, | | | | | | | |
| | | placental | | | | | | | |
| MASP3 | | Alkaline | | | | DCC | RCAN3 | CD248 | 0.668 |
| | | phosphatase, | | | | | | | |
| | | placental | | | | | | | |
| MASP3 | EPHA6 | Alkaline | | | | DCC | | CD248 | 0.668 |
| | | phosphatase, | | | | | | | |
| | | placental | | | | | | | |
| MASP3 | EPHA6 | Alkaline | | | | DCC | RCAN3 | CD248 | 0.668 |
| | | phosphatase, | | | | | | | |
| | | placental | | | | | | | |
| MASP3 | | Alkaline | | | | DCC | | | 0.666 |
| | | phosphatase, | | | | | | | |
| | | placental | | | | | | | |
| MASP3 | | Alkaline | | | | DCC | RCAN3 | | 0.666 |
| | | phosphatase, | | | | | | | |
| | | placental | | | | | | | |
| MASP3 | EPHA6 | Alkaline | | | | DCC | | | 0.666 |
| | | phosphatase, | | | | | | | |
| | | placental | | | | | | | |
| MASP3 | EPHA6 | Alkaline | | | | DCC | RCAN3 | | 0.666 |
| | | phosphatase, | | | | | | | |
| | | placental | | | | | | | |
| MASP3 | | Alkaline | | | | | | | 0.665 |
| | | phosphatase, | | | | | | | |
| | | placental | | | | | | | |
| MASP3 | | Alkaline | | | | | | CD248 | 0.664 |
| | | phosphatase, | | | | | | | |
| | | placental | | | | | | | |
| MASP3 | | Alkaline | | | | | RCAN3 | | 0.664 |
| | | phosphatase, | | | | | | | |
| | | placental | | | | | | | |
| MASP3 | | Alkaline | | | | | RCAN3 | CD248 | 0.664 |
| | | phosphatase, | | | | | | | |
| | | placental | | | | | | | |
| MASP3 | EPHA6 | Alkaline | | | | | | | 0.664 |
| | | phosphatase, | | | | | | | |
| | | placental | | | | | | | |
| MASP3 | EPHA6 | Alkaline | | | | | | CD 248 | 0.664 |
| | | phosphatase, | | | | | | | |
| | | placental | | | | | | | |
| MASP3 | EPHA6 | Alkaline | | | | | RCAN3 | | 0.664 |
| | | phosphatase, | | | | | | | |
| | | placental | | | | | | | |
| MASP3 | EPHA6 | Alkaline | | | | | RCAN3 | CD248 | 0.664 |
| | | phosphatase, | | | | | | | |
| | | placental | | | | | | | |
| MASP3 | EPHA6 | | SCF | | IgG4, | DCC | | CD248 | 0.635 |
| | | | | | Kappa | | | | |
| MASP3 | EPHA6 | | SCF | | IgG4, | DCC | RCAN3 | CD248 | 0.635 |
| | | | | | Kappa | | | | |
| MASP3 | EPHA6 | | SCF | DUS10 | IgG4, | DCC | | | 0.634 |
| | | | | | Kappa | | | | |
| MASP3 | EPHA6 | | SCF | DUS10 | IgG4, | DCC | | CD248 | 0.634 |
| | | | | | Kappa | | | | |
| MASP3 | EPHA6 | | SCF | DUS10 | IgG4, | DCC | RCAN3 | | 0.634 |
| | | | | | Kappa | | | | |
| MASP3 | EPHA6 | | SCF | DUS10 | IgG4, | DCC | RCAN3 | CD248 | 0.634 |
| | | | | | Kappa | | | | |
| MASP3 | EPHA6 | | SCF | | IgG4, | DCC | | | 0.633 |
| | | | | | Kappa | | | | |
| MASP3 | EPHA6 | | SCF | | IgG4, | DCC | RCAN3 | | 0.633 |
| | | | | | Kappa | | | | |
| MASP3 | | | SCF | | IgG4, | DCC | | CD248 | 0.631 |
| | | | | | Kappa | | | | |
| MASP3 | | | SCF | | IgG4, | DCC | RCAN3 | CD248 | 0.631 |
| | | | | | Kappa | | | | |
| MASP3 | | | SCF | DUS10 | IgG4, | DCC | | CD248 | 0.631 |
| | | | | | Kappa | | | | |
| MASP3 | | | SCF | DUS10 | IgG4, | DCC | RCAN3 | CD248 | 0.63 |
| | | | | | Kappa | | | | |
| MASP3 | | | SCF | | IgG4, | DCC | | | 0.629 |
| | | | | | Kappa | | | | |
| MASP3 | | | SCF | | IgG4, | DCC | RCAN3 | | 0.629 |
| | | | | | Kappa | | | | |
| MASP3 | | | SCF | DUS10 | IgG4, | DCC | | | 0.629 |
| | | | | | Kappa | | | | |
| MASP3 | | | SCF | DUS10 | IgG4, | DCC | RCAN3 | | 0.629 |
| | | | | | Kappa | | | | |
| MASP3 | | | SCF | DUS10 | IgG4, | | | CD248 | 0.627 |
| | | | | | Kappa | | | | |
| MASP3 | | | SCF | DUS10 | IgG4, | | RCAN3 | CD248 | 0.627 |
| | | | | | Kappa | | | | |
| MASP3 | EPHA6 | | SCF | DUS10 | IgG4, | | | CD248 | 0.627 |
| | | | | | Kappa | | | | |
| MASP3 | EPHA6 | | SCF | DUS10 | IgG4, | | RCAN3 | | 0.627 |
| | | | | | Kappa | | | | |
| MASP3 | EPHA6 | | SCF | DUS10 | IgG4, | | RCAN3 | CD248 | 0.627 |
| | | | | | Kappa | | | | |
| MASP3 | | | SCF | | IgG4, | | | CD248 | 0.626 |
| | | | | | Kappa | | | | |
| MASP3 | | | SCF | | IgG4, | | RCAN3 | CD248 | 0.626 |
| | | | | | Kappa | | | | |
| MASP3 | | | SCF | DUS10 | IgG4, | | | | 0.626 |
| | | | | | Kappa | | | | |
| MASP3 | | | SCF | DUS10 | IgG4, | | RCAN3 | | 0.626 |
| | | | | | Kappa | | | | |
| MASP3 | EPHA6 | | SCF | | IgG4, | | | | 0.626 |
| | | | | | Kappa | | | | |
| MASP3 | EPHA6 | | SCF | | IgG4, | | | CD248 | 0.626 |
| | | | | | Kappa | | | | |
| MASP3 | EPHA6 | | SCF | | IgG4, | | RCAN3 | CD248 | 0.626 |
| | | | | | Kappa | | | | |
| MASP3 | EPHA6 | | SCF | DUS10 | IgG4, | | | | 0.626 |
| | | | | | Kappa | | | | |
| MASP3 | | | SCF | | IgG4, | | | | 0.625 |
| | | | | | Kappa | | | | |
| MASP3 | | | SCF | | IgG4, | | RCAN3 | | 0.625 |
| | | | | | Kappa | | | | |
| MASP3 | EPHA6 | | SCF | | IgG4, | | RCAN3 | | 0.625 |
| | | | | | Kappa | | | | |
| MASP3 | EPHA6 | | SCF | | | DCC | | CD248 | 0.615 |
| MASP3 | EPHA6 | | SCF | | | DCC | RCAN3 | CD248 | 0.615 |
| MASP3 | EPHA6 | | SCF | DUS10 | | DCC | | | 0.615 |
| MASP3 | EPHA6 | | SCF | DUS10 | | DCC | | CD248 | 0.615 |
| MASP3 | EPHA6 | | SCF | DUS10 | | DCC | RCAN3 | | 0.615 |
| MASP3 | EPHA6 | | SCF | DUS10 | | DCC | RCAN3 | CD248 | 0.615 |
| MASP3 | EPHA6 | | SCF | | | DCC | | | 0.614 |
| MASP3 | EPHA6 | | SCF | | | DCC | RCAN3 | | 0.614 |
| MASP3 | | | SCF | DUS10 | | DCC | | CD248 | 0.611 |
| MASP3 | | | SCF | DUS10 | | DCC | RCAN3 | CD248 | 0.611 |
| MASP3 | | | SCF | | | DCC | | CD248 | 0.61 |
| MASP3 | | | SCF | | | DCC | RCAN3 | CD248 | 0.61 |
| MASP3 | | | SCF | DUS10 | | DCC | | | 0.607 |
| MASP3 | | | SCF | DUS10 | | DCC | RCAN3 | | 0.607 |
| MASP3 | | | SCF | | | DCC | | | 0.606 |
| MASP3 | | | SCF | | | DCC | RCAN3 | | 0.606 |
| MASP3 | EPHA6 | | SCF | DUS10 | | | | CD248 | 0.605 |
| MASP3 | EPHA6 | | SCF | DUS10 | | | RCAN3 | | 0.605 |
| MASP3 | EPHA6 | | SCF | DUS10 | | | RCAN3 | CD248 | 0.605 |
| MASP3 | | | SCF | DUS10 | | | | CD248 | 0.604 |
| MASP3 | | | SCF | DUS10 | | | RCAN3 | CD248 | 0.604 |
| MASP3 | EPHA6 | | SCF | | | | | CD248 | 0.604 |
| MASP3 | EPHA6 | | SCF | | | | RCAN3 | | 0.604 |
| MASP3 | EPHA6 | | SCF | | | | RCAN3 | CD248 | 0.604 |
| MASP3 | EPHA6 | | SCF | DUS10 | | | | | 0.604 |
| MASP3 | | | SCF | | | | | CD248 | 0.603 |
| MASP3 | | | SCF | | | | RCAN3 | CD248 | 0.603 |
| MASP3 | | | SCF | DUS10 | | | | | 0.603 |
| MASP3 | | | SCF | DUS10 | | | RCAN3 | | 0.603 |
| MASP3 | EPHA6 | | SCF | | | | | | 0.603 |
| MASP3 | | | SCF | | | | | | 0.602 |
| MASP3 | | | SCF | | | | RCAN3 | | 0.602 |
| MASP3 | EPHA6 | | | DUS10 | IgG4, | DCC | | | 0.602 |
| | | | | | Kappa | | | | |
| MASP3 | EPHA6 | | | DUS10 | IgG4, | DCC | | CD248 | 0.602 |
| | | | | | Kappa | | | | |
| MASP3 | EPHA6 | | | DUS10 | IgG4, | DCC | RCAN3 | CD248 | 0.602 |
| | | | | | Kappa | | | | |
| MASP3 | | | | DUS10 | IgG4, | | RCAN3 | | 0.601 |
| | | | | | Kappa | | | | |
| MASP3 | | | | DUS10 | IgG4, | DCC | | | 0.601 |
| | | | | | Kappa | | | | |
| MASP3 | | | | DUS10 | IgG4, | DCC | | CD248 | 0.601 |
| | | | | | Kappa | | | | |
| MASP3 | | | | DUS10 | IgG4, | DCC | RCAN3 | CD248 | 0.601 |
| | | | | | Kappa | | | | |
| MASP3 | EPHA6 | | | DUS10 | IgG4, | | RCAN3 | | 0.601 |
| | | | | | Kappa | | | | |
| MASP3 | EPHA6 | | | DUS10 | IgG4, | | RCAN3 | CD248 | 0.601 |
| | | | | | Kappa | | | | |
| MASP3 | EPHA6 | | | DUS10 | IgG4, | DCC | RCAN3 | | 0.601 |
| | | | | | Kappa | | | | |
| MASP3 | | | | DUS10 | IgG4, | | RCAN3 | CD248 | 0.6 |
| | | | | | Kappa | | | | |
| MASP3 | | | | DUS10 | IgG4, | DCC | RCAN3 | | 0.6 |
| | | | | | Kappa | | | | |
| MASP3 | EPHA6 | | | | IgG4, | DCC | | CD248 | 0.6 |
| | | | | | Kappa | | | | |
| MASP3 | EPHA6 | | | | IgG4, | DCC | RCAN3 | CD248 | 0.6 |
| | | | | | Kappa | | | | |

TABLE 19
DUS10 Biomarker Panels

| | | Alkaline | | | | | | | |
|---|---|---|---|---|---|---|---|---|---|
| | | phosphatase, | | | IgG4, | | | | |
| DUS10 | EPHA6 | placental | SCF | MASP3 | Kappa | DCC | RCAN3 | CD248 | AUC |
| DUS10 | EPHA6 | Alkaline | SCF | MASP3 | IgG4, | DCC | RCAN3 | CD248 | 0.698 |
| | | phosphatase, | | | Kappa | | | | |
| | | placental | | | | | | | |
| DUS10 | | Alkaline | SCF | MASP3 | IgG4, | DCC | RCAN3 | CD248 | 0.697 |
| | | phosphatase, | | | Kappa | | | | |
| | | placental | | | | | | | |
| DUS10 | EPHA6 | Alkaline | SCF | MASP3 | IgG4, | DCC | | CD248 | 0.697 |
| | | phosphatase, | | | Kappa | | | | |
| | | placental | | | | | | | |
| DUS10 | | Alkaline | SCF | MASP3 | IgG4, | | | CD248 | 0.696 |
| | | phosphatase, | | | Kappa | | | | |
| | | placental | | | | | | | |
| DUS10 | | Alkaline | SCF | MASP3 | IgG4, | | RCAN3 | CD248 | 0.696 |
| | | phosphatase, | | | Kappa | | | | |
| | | placental | | | | | | | |
| DUS10 | | Alkaline | SCF | MASP3 | IgG4, | DCC | | CD248 | 0.696 |
| | | phosphatase, | | | Kappa | | | | |
| | | placental | | | | | | | |
| DUS10 | EPHA6 | Alkaline | SCF | MASP3 | IgG4, | | | CD248 | 0.696 |
| | | phosphatase, | | | Kappa | | | | |
| | | placental | | | | | | | |
| DUS10 | EPHA6 | Alkaline | SCF | MASP3 | IgG4, | | RCAN3 | CD248 | 0.696 |
| | | phosphatase, | | | Kappa | | | | |
| | | placental | | | | | | | |
| DUS10 | EPHA6 | Alkaline | SCF | MASP3 | IgG4, | DCC | RCAN3 | | 0.696 |
| | | phosphatase, | | | Kappa | | | | |
| | | placental | | | | | | | |
| DUS10 | EPHA6 | Alkaline | SCF | MASP3 | IgG4, | | RCAN3 | | 0.695 |
| | | phosphatase, | | | Kappa | | | | |
| | | placental | | | | | | | |
| DUS10 | EPHA6 | Alkaline | SCF | MASP3 | IgG4, | DCC | | | 0.695 |
| | | phosphatase, | | | Kappa | | | | |
| | | placental | | | | | | | |
| DUS10 | | Alkaline | SCF | MASP3 | IgG4, | | RCAN3 | | 0.694 |
| | | phosphatase, | | | Kappa | | | | |
| | | placental | | | | | | | |
| DUS10 | | Alkaline | SCF | MASP3 | IgG4, | DCC | RCAN3 | | 0.694 |
| | | phosphatase, | | | Kappa | | | | |
| | | placental | | | | | | | |
| DUS10 | EPHA6 | Alkaline | SCF | MASP3 | IgG4, | | | | 0.694 |
| | | phosphatase, | | | Kappa | | | | |
| | | placental | | | | | | | |
| DUS10 | | Alkaline | SCF | MASP3 | IgG4, | | | | 0.693 |
| | | phosphatase, | | | Kappa | | | | |
| | | placental | | | | | | | |
| DUS10 | | Alkaline | SCF | MASP3 | IgG4, | DCC | | | 0.693 |
| | | phosphatase, | | | Kappa | | | | |
| | | placental | | | | | | | |
| DUS10 | EPHA6 | Alkaline | SCF | MASP3 | | DCC | RCAN3 | CD248 | 0.689 |
| | | phosphatase, | | | | | | | |
| | | placental | | | | | | | |
| DUS10 | | Alkaline | SCF | | IgG4, | DCC | RCAN3 | CD248 | 0.687 |
| | | phosphatase, | | | Kappa | | | | |
| | | placental | | | | | | | |
| DUS10 | | Alkaline | SCF | MASP3 | | DCC | RCAN3 | CD248 | 0.687 |
| | | phosphatase, | | | | | | | |
| | | placental | | | | | | | |
| DUS10 | EPHA6 | Alkaline | SCF | | IgG4, | DCC | RCAN3 | CD248 | 0.687 |
| | | phosphatase, | | | Kappa | | | | |
| | | placental | | | | | | | |
| DUS10 | EPHA6 | Alkaline | SCF | MASP3 | | DCC | | CD248 | 0.687 |
| | | phosphatase, | | | | | | | |
| | | placental | | | | | | | |
| DUS10 | | Alkaline | SCF | MASP3 | | DCC | | CD248 | 0.686 |
| | | phosphatase, | | | | | | | |
| | | placental | | | | | | | |
| DUS10 | EPHA6 | Alkaline | SCF | | IgG4, | DCC | | CD248 | 0.686 |
| | | phosphatase, | | | Kappa | | | | |
| | | placental | | | | | | | |
| DUS10 | EPHA6 | Alkaline | SCF | MASP3 | | DCC | RCAN3 | | 0.686 |
| | | phosphatase, | | | | | | | |
| | | placental | | | | | | | |
| DUS10 | | Alkaline | SCF | | IgG4, | DCC | | CD248 | 0.685 |
| | | phosphatase, | | | Kappa | | | | |
| | | placental | | | | | | | |
| DUS10 | | Alkaline | SCF | MASP3 | | | RCAN3 | CD248 | 0.685 |
| | | phosphatase, | | | | | | | |
| | | placental | | | | | | | |
| DUS10 | EPHA6 | Alkaline | SCF | MASP3 | | | RCAN3 | CD248 | 0.685 |
| | | phosphatase, | | | | | | | |
| | | placental | | | | | | | |
| DUS10 | | Alkaline | SCF | MASP3 | | | | CD248 | 0.684 |
| | | phosphatase, | | | | | | | |
| | | placental | | | | | | | |
| DUS10 | EPHA6 | Alkaline | SCF | | IgG4, | DCC | RCAN3 | | 0.684 |
| | | phosphatase, | | | Kappa | | | | |
| | | placental | | | | | | | |
| DUS10 | EPHA6 | Alkaline | SCF | MASP3 | | | | CD248 | 0.684 |
| | | phosphatase, | | | | | | | |
| | | placental | | | | | | | |
| DUS10 | EPHA6 | Alkaline | SCF | MASP3 | | DCC | | | 0.684 |
| | | phosphatase, | | | | | | | |
| | | placental | | | | | | | |
| DUS10 | | Alkaline | | MASP3 | IgG4, | DCC | | CD248 | 0.683 |
| | | phosphatase, | | | Kappa | | | | |
| | | placental | | | | | | | |
| DUS10 | | Alkaline | SCF | MASP3 | | | RCAN3 | | 0.683 |
| | | phosphatase, | | | | | | | |
| | | placental | | | | | | | |
| DUS10 | | Alkaline | SCF | MASP3 | | DCC | RCAN3 | | 0.683 |
| | | phosphatase, | | | | | | | |
| | | placental | | | | | | | |
| DUS10 | EPHA6 | Alkaline | | MASP3 | IgG4, | DCC | | CD248 | 0.683 |
| | | phosphatase, | | | Kappa | | | | |
| | | placental | | | | | | | |
| DUS10 | EPHA6 | Alkaline | SCF | | IgG4, | DCC | | | 0.683 |
| | | phosphatase, | | | Kappa | | | | |
| | | placental | | | | | | | |
| DUS10 | EPHA6 | Alkaline | SCF | MASP3 | | | RCAN3 | | 0.683 |
| | | phosphatase, | | | | | | | |
| | | placental | | | | | | | |
| DUS10 | | Alkaline | | MASP3 | IgG4, | DCC | RCAN3 | CD248 | 0.682 |
| | | phosphatase, | | | Kappa | | | | |
| | | placental | | | | | | | |
| DUS10 | | Alkaline | SCF | MASP3 | | DCC | | | 0.682 |
| | | phosphatase, | | | | | | | |
| | | placental | | | | | | | |
| DUS10 | EPHA6 | Alkaline | | MASP3 | IgG4, | DCC | RCAN3 | CD248 | 0.682 |
| | | phosphatase, | | | Kappa | | | | |
| | | placental | | | | | | | |
| DUS10 | EPHA6 | Alkaline | SCF | MASP3 | | | | | 0.682 |
| | | phosphatase, | | | | | | | |
| | | placental | | | | | | | |
| DUS10 | | Alkaline | SCF | | IgG4, | | RCAN3 | CD248 | 0.681 |
| | | phosphatase, | | | Kappa | | | | |
| | | placental | | | | | | | |
| DUS10 | | Alkaline | SCF | | IgG4, | DCC | RCAN3 | | 0.681 |
| | | phosphatase, | | | Kappa | | | | |
| | | placental | | | | | | | |
| DUS10 | | Alkaline | SCF | MASP3 | | | | | 0.681 |
| | | phosphatase, | | | | | | | |
| | | placental | | | | | | | |
| DUS10 | EPHA6 | Alkaline | | MASP3 | IgG4, | | | CD248 | 0.681 |
| | | phosphatase, | | | Kappa | | | | |
| | | placental | | | | | | | |
| DUS10 | EPHA6 | Alkaline | | MASP3 | IgG4, | | RCAN3 | CD248 | 0.681 |
| | | phosphatase, | | | Kappa | | | | |
| | | placental | | | | | | | |
| DUS10 | EPHA6 | Alkaline | SCF | | IgG4, | | RCAN3 | CD248 | 0.681 |
| | | phosphatase, | | | Kappa | | | | |
| | | placental | | | | | | | |
| DUS10 | | Alkaline | | MASP3 | IgG4, | | | | 0.68 |
| | | phosphatase, | | | Kappa | | | | |
| | | placental | | | | | | | |
| DUS10 | | Alkaline | | MASP3 | IgG4, | | | CD248 | 0.68 |
| | | phosphatase, | | | Kappa | | | | |
| | | placental | | | | | | | |
| DUS10 | | Alkaline | | MASP3 | IgG4, | | RCAN3 | | 0.68 |
| | | phosphatase, | | | Kappa | | | | |
| | | placental | | | | | | | |
| DUS10 | | Alkaline | | MASP3 | IgG4, | | RCAN3 | CD248 | 0.68 |
| | | phosphatase, | | | Kappa | | | | |
| | | placental | | | | | | | |
| DUS10 | | Alkaline | | MASP3 | IgG4, | DCC | | | 0.68 |
| | | phosphatase, | | | Kappa | | | | |
| | | placental | | | | | | | |
| DUS10 | | Alkaline | | MASP3 | IgG4, | DCC | RCAN3 | | 0.68 |
| | | phosphatase, | | | Kappa | | | | |
| | | placental | | | | | | | |
| DUS10 | | Alkaline | SCF | | IgG4, | | | CD248 | 0.68 |
| | | phosphatase, | | | Kappa | | | | |
| | | placental | | | | | | | |
| DUS10 | EPHA6 | Alkaline | | MASP3 | IgG4, | | | | 0.68 |
| | | phosphatase, | | | Kappa | | | | |
| | | placental | | | | | | | |
| DUS10 | EPHA6 | Alkaline | | MASP3 | IgG4, | | RCAN3 | | 0.68 |
| | | phosphatase, | | | Kappa | | | | |
| | | placental | | | | | | | |
| DUS10 | EPHA6 | Alkaline | | MASP3 | IgG4, | DCC | | | 0.68 |
| | | phosphatase, | | | Kappa | | | | |
| | | placental | | | | | | | |
| DUS10 | EPHA6 | Alkaline | | MASP3 | IgG4, | DCC | RCAN3 | | 0.68 |
| | | phosphatase, | | | Kappa | | | | |
| | | placental | | | | | | | |
| DUS10 | EPHA6 | Alkaline | SCF | | IgG4, | | | CD248 | 0.68 |
| | | phosphatase, | | | Kappa | | | | |
| | | placental | | | | | | | |
| DUS10 | EPHA6 | Alkaline | SCF | | IgG4, | | RCAN3 | | 0.68 |
| | | phosphatase, | | | Kappa | | | | |
| | | placental | | | | | | | |
| DUS10 | | Alkaline | SCF | | IgG4, | | RCAN3 | | 0.679 |
| | | phosphatase, | | | Kappa | | | | |
| | | placental | | | | | | | |
| DUS10 | | Alkaline | SCF | | IgG4, | DCC | | | 0.679 |
| | | phosphatase, | | | Kappa | | | | |
| | | placental | | | | | | | |
| DUS10 | | Alkaline | SCF | | IgG4, | | | | 0.677 |
| | | phosphatase, | | | Kappa | | | | |
| | | placental | | | | | | | |
| DUS10 | EPHA6 | Alkaline | SCF | | IgG4, | | | | 0.677 |
| | | phosphatase, | | | Kappa | | | | |
| | | placental | | | | | | | |
| DUS10 | | Alkaline | | MASP3 | | DCC | | CD248 | 0.675 |
| | | phosphatase, | | | | | | | |
| | | placental | | | | | | | |
| DUS10 | | Alkaline | | MASP3 | | DCC | RCAN3 | CD248 | 0.675 |
| | | phosphatase, | | | | | | | |
| | | placental | | | | | | | |
| DUS10 | EPHA6 | Alkaline | | MASP3 | | DCC | | CD248 | 0.674 |
| | | phosphatase, | | | | | | | |
| | | placental | | | | | | | |
| DUS10 | EPHA6 | Alkaline | | MASP3 | | DCC | RCAN3 | CD248 | 0.674 |
| | | phosphatase, | | | | | | | |
| | | placental | | | | | | | |
| DUS10 | EPHA6 | Alkaline | SCF | | | DCC | RCAN3 | CD248 | 0.674 |
| | | phosphatase, | | | | | | | |
| | | placental | | | | | | | |
| DUS10 | EPHA6 | Alkaline | | MASP3 | | DCC | | | 0.673 |
| | | phosphatase, | | | | | | | |
| | | placental | | | | | | | |
| DUS10 | EPHA6 | Alkaline | | MASP3 | | DCC | RCAN3 | | 0.673 |
| | | phosphatase, | | | | | | | |
| | | placental | | | | | | | |
| DUS10 | | Alkaline | | MASP3 | | DCC | | | 0.672 |
| | | phosphatase, | | | | | | | |
| | | placental | | | | | | | |
| DUS10 | | Alkaline | | MASP3 | | DCC | RCAN3 | | 0.672 |
| | | phosphatase, | | | | | | | |
| | | placental | | | | | | | |
| DUS10 | | Alkaline | SCF | | | DCC | RCAN3 | CD248 | 0.672 |
| | | phosphatase, | | | | | | | |
| | | placental | | | | | | | |
| DUS10 | EPHA6 | Alkaline | SCF | | | DCC | | CD248 | 0.672 |
| | | phosphatase, | | | | | | | |
| | | placental | | | | | | | |
| DUS10 | EPHA6 | Alkaline | SCF | | | DCC | RCAN3 | | 0.672 |
| | | phosphatase, | | | | | | | |
| | | placental | | | | | | | |
| DUS10 | | Alkaline | | MASP3 | | | | | 0.671 |
| | | phosphatase, | | | | | | | |
| | | placental | | | | | | | |
| DUS10 | | Alkaline | | MASP3 | | | | CD248 | 0.671 |
| | | phosphatase, | | | | | | | |
| | | placental | | | | | | | |
| DUS10 | | Alkaline | | MASP3 | | | RCAN3 | | 0.671 |
| | | phosphatase, | | | | | | | |
| | | placental | | | | | | | |
| DUS10 | | Alkaline | | MASP3 | | | RCAN3 | CD248 | 0.671 |
| | | phosphatase, | | | | | | | |
| | | placental | | | | | | | |
| DUS10 | EPHA6 | Alkaline | | MASP3 | | | RCAN3 | CD248 | 0.671 |
| | | phosphatase, | | | | | | | |
| | | placental | | | | | | | |
| DUS10 | | Alkaline | SCF | | | DCC | | CD248 | 0.67 |
| | | phosphatase, | | | | | | | |
| | | placental | | | | | | | |
| DUS10 | EPHA6 | Alkaline | | MASP3 | | | | | 0.67 |
| | | phosphatase, | | | | | | | |
| | | placental | | | | | | | |
| DUS10 | EPHA6 | Alkaline | | MASP3 | | | | CD248 | 0.67 |
| | | phosphatase, | | | | | | | |
| | | placental | | | | | | | |
| DUS10 | EPHA6 | Alkaline | | MASP3 | | | RCAN3 | | 0.67 |
| | | phosphatase, | | | | | | | |
| | | placental | | | | | | | |
| DUS10 | EPHA6 | Alkaline | SCF | | | DCC | | | 0.669 |
| | | phosphatase, | | | | | | | |
| | | placental | | | | | | | |
| DUS10 | | Alkaline | | | IgG4, | DCC | | CD248 | 0.667 |
| | | phosphatase, | | | Kappa | | | | |
| | | placental | | | | | | | |
| DUS10 | | Alkaline | | | IgG4, | DCC | RCAN3 | CD248 | 0.667 |
| | | phosphatase, | | | Kappa | | | | |
| | | placental | | | | | | | |
| DUS10 | | Alkaline | SCF | | | DCC | RCAN3 | | 0.667 |
| | | phosphatase, | | | | | | | |
| | | placental | | | | | | | |
| DUS10 | EPHA6 | Alkaline | | | IgG4, | DCC | | CD248 | 0.667 |
| | | phosphatase, | | | Kappa | | | | |
| | | placental | | | | | | | |
| DUS10 | EPHA6 | Alkaline | | | IgG4, | DCC | RCAN3 | CD248 | 0.667 |
| | | phosphatase, | | | Kappa | | | | |
| | | placental | | | | | | | |
| DUS10 | EPHA6 | Alkaline | | | IgG4, | DCC | | | 0.666 |
| | | phosphatase, | | | Kappa | | | | |
| | | placental | | | | | | | |
| DUS10 | EPHA6 | Alkaline | | | IgG4, | DCC | RCAN3 | | 0.666 |
| | | phosphatase, | | | Kappa | | | | |
| | | placental | | | | | | | |
| DUS10 | | Alkaline | | | IgG4, | DCC | | | 0.665 |
| | | phosphatase, | | | Kappa | | | | |
| | | placental | | | | | | | |
| DUS10 | | Alkaline | | | IgG4, | DCC | RCAN3 | | 0.665 |
| | | phosphatase, | | | Kappa | | | | |
| | | placental | | | | | | | |
| DUS10 | | Alkaline | SCF | | | | RCAN3 | CD248 | 0.665 |
| | | phosphatase, | | | | | | | |
| | | placental | | | | | | | |
| DUS10 | EPHA6 | Alkaline | SCF | | | | RCAN3 | CD248 | 0.665 |
| | | phosphatase, | | | | | | | |
| | | placental | | | | | | | |
| DUS10 | | Alkaline | SCF | | | DCC | | | 0.664 |
| | | phosphatase, | | | | | | | |
| | | placental | | | | | | | |
| DUS10 | EPHA6 | Alkaline | SCF | | | | RCAN3 | | 0.664 |
| | | phosphatase, | | | | | | | |
| | | placental | | | | | | | |
| DUS10 | | Alkaline | SCF | | | | RCAN3 | | 0.663 |
| | | phosphatase, | | | | | | | |
| | | placental | | | | | | | |
| DUS10 | | Alkaline | SCF | | | | | CD248 | 0.662 |
| | | phosphatase, | | | | | | | |
| | | placental | | | | | | | |
| DUS10 | EPHA6 | Alkaline | SCF | | | | | CD248 | 0.662 |
| | | phosphatase, | | | | | | | |
| | | placental | | | | | | | |
| DUS10 | | Alkaline | | | IgG4, | | | | 0.661 |
| | | phosphatase, | | | Kappa | | | | |
| | | placental | | | | | | | |
| DUS10 | | Alkaline | | | IgG4, | | RCAN3 | | 0.661 |
| | | phosphatase, | | | Kappa | | | | |
| | | placental | | | | | | | |
| DUS10 | EPHA6 | Alkaline | SCF | | | | | | 0.661 |
| | | phosphatase, | | | | | | | |
| | | placental | | | | | | | |
| DUS1C | | Alkaline | | | IgG4, | | | CD248 | 0.66 |
| | | phosphatase, | | | Kappa | | | | |
| | | placental | | | | | | | |
| DUS10 | | Alkaline | | | IgG4, | | RCAN3 | CD248 | 0.66 |
| | | phosphatase, | | | Kappa | | | | |
| | | placental | | | | | | | |
| DUS10 | EPHA6 | Alkaline | | | IgG4, | | | | 0.66 |
| | | phosphatase, | | | Kappa | | | | |
| | | placental | | | | | | | |
| DUS10 | EPHA6 | Alkaline | | | IgG4, | | | CD248 | 0.66 |
| | | phosphatase, | | | Kappa | | | | |
| | | placental | | | | | | | |
| DUS10 | EPHA6 | Alkaline | | | IgG4, | | RCAN3 | | 0.66 |
| | | phosphatase, | | | Kappa | | | | |
| | | placental | | | | | | | |
| DUS10 | EPHA6 | Alkaline | | | IgG4, | | RCAN3 | CD248 | 0.66 |
| | | phosphatase, | | | Kappa | | | | |
| | | placental | | | | | | | |
| DUS10 | | Alkaline | SCF | | | | | | 0.659 |
| | | phosphatase, | | | | | | | |
| | | placental | | | | | | | |
| DUS10 | EPHA6 | Alkaline | | | | DCC | | CD248 | 0.656 |
| | | phosphatase, | | | | | | | |
| | | placental | | | | | | | |
| DUS10 | EPHA6 | Alkaline | | | | DCC | RCAN3 | CD248 | 0.656 |
| | | phosphatase, | | | | | | | |
| | | placental | | | | | | | |
| DUS10 | | Alkaline | | | | DCC | | CD248 | 0.655 |
| | | phosphatase, | | | | | | | |
| | | placental | | | | | | | |
| DUS10 | | Alkaline | | | | DCC | RCAN3 | CD248 | 0.655 |
| | | phosphatase, | | | | | | | |
| | | placental | | | | | | | |
| DUS10 | EPHA6 | Alkaline | | | | DCC | | | 0.655 |
| | | phosphatase, | | | | | | | |
| | | placental | | | | | | | |
| DUS10 | EPHA6 | Alkaline | | | | DCC | RCAN3 | | 0.655 |
| | | phosphatase, | | | | | | | |
| | | placental | | | | | | | |
| DUS10 | | Alkaline | | | | DCC | RCAN3 | | 0.653 |
| | | phosphatase, | | | | | | | |
| | | placental | | | | | | | |
| DUS10 | | Alkaline | | | | DCC | | | 0.652 |
| | | phosphatase, | | | | | | | |
| | | placental | | | | | | | |
| DUS10 | | Alkaline | | | | | RCAN3 | | 0.648 |
| | | phosphatase, | | | | | | | |
| | | placental | | | | | | | |
| DUS10 | | Alkaline | | | | | | | 0.647 |
| | | phosphatase, | | | | | | | |
| | | placental | | | | | | | |
| DUS10 | | Alkaline | | | | | RCAN3 | CD248 | 0.646 |
| | | phosphatase, | | | | | | | |
| | | placental | | | | | | | |
| DUS10 | EPHA6 | Alkaline | | | | | RCAN3 | | 0.646 |
| | | phosphatase, | | | | | | | |
| | | placental | | | | | | | |
| DUS10 | | Alkaline | | | | | | CD248 | 0.645 |
| | | phosphatase, | | | | | | | |
| | | placental | | | | | | | |
| DUS10 | EPHA6 | Alkaline | | | | | | | 0.645 |
| | | phosphatase, | | | | | | | |
| | | placental | | | | | | | |
| DUS10 | EPHA6 | Alkaline | | | | | RCAN3 | CD248 | 0.645 |
| | | phosphatase, | | | | | | | |
| | | placental | | | | | | | |
| DUS10 | EPHA6 | Alkaline | | | | | | CD248 | 0.644 |
| | | phosphatase, | | | | | | | |
| | | placental | | | | | | | |
| DUS10 | EPHA6 | | SCF | MASP3 | IgG4, | DCC | | | 0.634 |
| | | | | | Kappa | | | | |
| DUS10 | EPHA6 | | SCF | MASP3 | IgG4, | DCC | | CD248 | 0.634 |
| | | | | | Kappa | | | | |
| DUS10 | EPHA6 | | SCF | MASP3 | IgG4, | DCC | RCAN3 | | 0.634 |
| | | | | | Kappa | | | | |
| DUS10 | EPHA6 | | SCF | MASP3 | IgG4, | DCC | RCAN3 | CD248 | 0.634 |
| | | | | | Kappa | | | | |
| DUS10 | | | SCF | MASP3 | IgG4, | DCC | | CD248 | 0.631 |
| | | | | | Kappa | | | | |
| DUS10 | | | SCF | MASP3 | IgG4, | DCC | RCAN3 | CD248 | 0.63 |
| | | | | | Kappa | | | | |
| DUS10 | | | SCF | MASP3 | IgG4, | DCC | | | 0.629 |
| | | | | | Kappa | | | | |
| DUS10 | | | SCF | MASP3 | IgG4, | DCC | RCAN3 | | 0.629 |
| | | | | | Kappa | | | | |
| DUS10 | EPHA6 | | SCF | | IgG4, | DCC | | | 0.629 |
| | | | | | Kappa | | | | |
| DUS10 | EPHA6 | | SCF | | IgG4, | DCC | | CD248 | 0.629 |
| | | | | | Kappa | | | | |
| DUS10 | EPHA6 | | SCF | | IgG4, | DCC | RCAN3 | | 0.629 |
| | | | | | Kappa | | | | |
| DUS10 | EPHA6 | | SCF | | IgG4, | DCC | RCAN3 | CD248 | 0.629 |
| | | | | | Kappa | | | | |
| DUS10 | | | SCF | | IgG4, | DCC | | CD248 | 0.627 |
| | | | | | Kappa | | | | |
| DUS10 | | | SCF | | IgG4, | DCC | RCAN3 | CD248 | 0.627 |
| | | | | | Kappa | | | | |
| DUS10 | | | SCF | MASP3 | IgG4, | | | CD248 | 0.627 |
| | | | | | Kappa | | | | |
| DUS10 | | | SCF | MASP3 | IgG4, | | RCAN3 | CD248 | 0.627 |
| | | | | | Kappa | | | | |
| DUS10 | EPHA6 | | SCF | MASP3 | IgG4, | | | CD248 | 0.627 |
| | | | | | Kappa | | | | |
| DUS10 | EPHA6 | | SCF | MASP3 | IgG4, | | RCAN3 | | 0.627 |
| | | | | | Kappa | | | | |
| DUS10 | EPHA6 | | SCF | MASP3 | IgG4, | | RCAN3 | CD248 | 0.627 |
| | | | | | Kappa | | | | |
| DUS10 | | | SCF | MASP3 | IgG4, | | | | 0.626 |
| | | | | | Kappa | | | | |
| DUS10 | | | SCF | MASP3 | IgG4, | | RCAN3 | | 0.626 |
| | | | | | Kappa | | | | |
| DUS10 | EPHA6 | | SCF | MASP3 | IgG4, | | | | 0.626 |
| | | | | | Kappa | | | | |
| DUS10 | | | SCF | | IgG4, | DCC | | | 0.623 |
| | | | | | Kappa | | | | |
| DUS10 | | | SCF | | IgG4, | DCC | RCAN3 | | 0.623 |
| | | | | | Kappa | | | | |
| DUS10 | | | SCF | | IgG4, | | | CD248 | 0.618 |
| | | | | | Kappa | | | | |
| DUS10 | | | SCF | | IgG4, | | RCAN3 | CD248 | 0.618 |
| | | | | | Kappa | | | | |
| DUS10 | EPHA6 | | SCF | | IgG4, | | | | 0.618 |
| | | | | | Kappa | | | | |
| DUS10 | EPHA6 | | SCF | | IgG4, | | | CD248 | 0.618 |
| | | | | | Kappa | | | | |
| DUS10 | EPHA6 | | SCF | | IgG4, | | RCAN3 | | 0.618 |
| | | | | | Kappa | | | | |
| DUS10 | EPHA6 | | SCF | | IgG4, | | RCAN3 | CD248 | 0.618 |
| | | | | | Kappa | | | | |
| DUS10 | | | SCF | | IgG4, | | | | 0.616 |
| | | | | | Kappa | | | | |
| DUS10 | | | SCF | | IgG4, | | RCAN3 | | 0.616 |
| | | | | | Kappa | | | | |
| DUS10 | EPHA6 | | SCF | MASP3 | | DCC | | | 0.615 |
| DUS10 | EPHA6 | | SCF | MASP3 | | DCC | | CD248 | 0.615 |
| DUS10 | EPHA6 | | SCF | MASP3 | | DCC | RCAN3 | | 0.615 |
| DUS10 | EPHA6 | | SCF | MASP3 | | DCC | RCAN3 | CD248 | 0.615 |
| DUS10 | EPHA6 | | SCF | | | DCC | RCAN3 | CD248 | 0.613 |
| DUS10 | EPHA6 | | SCF | | | DCC | | | 0.612 |
| DUS10 | EPHA6 | | SCF | | | DCC | | CD248 | 0.612 |
| DUS10 | EPHA6 | | SCF | | | DCC | RCAN3 | | 0.612 |
| DUS10 | | | SCF | MASP3 | | DCC | | CD248 | 0.611 |
| DUS10 | | | SCF | MASP3 | | DCC | RCAN3 | CD248 | 0.611 |
| DUS10 | | | SCF | MASP3 | | DCC | | | 0.607 |
| DUS10 | | | SCF | MASP3 | | DCC | RCAN3 | | 0.607 |
| DUS10 | | | SCF | | | DCC | | CD248 | 0.605 |
| DUS10 | | | SCF | | | DCC | RCAN3 | CD248 | 0.605 |
| DUS10 | EPHA6 | | SCF | MASP3 | | | | CD248 | 0.605 |
| DUS10 | EPHA6 | | SCF | MASP3 | | | RCAN3 | | 0.605 |
| DUS10 | EPHA6 | | SCF | MASP3 | | | RCAN3 | CD248 | 0.605 |
| DUS10 | | | SCF | MASP3 | | | | CD248 | 0.604 |
| DUS10 | | | SCF | MASP3 | | | RCAN3 | CD248 | 0.604 |
| DUS10 | EPHA6 | | SCF | MASP3 | | | | | 0.604 |
| DUS10 | | | SCF | MASP3 | | | | | 0.603 |
| DUS10 | | | SCF | MASP3 | | | RCAN3 | | 0.603 |
| DUS10 | | | SCF | | | DCC | | | 0.602 |
| DUS10 | | | SCF | | | DCC | RCAN3 | | 0.602 |
| DUS10 | EPHA6 | | | MASP3 | IgG4, | DCC | | | 0.602 |
| | | | | | Kappa | | | | |
| DUS10 | EPHA6 | | | MASP3 | IgG4, | DCC | | CD248 | 0.602 |
| | | | | | Kappa | | | | |
| DUS10 | EPHA6 | | | MASP3 | IgG4, | DCC | RCAN3 | CD248 | 0.602 |
| | | | | | Kappa | | | | |
| DUS10 | | | | MASP3 | IgG4, | | RCAN3 | | 0.601 |
| | | | | | Kappa | | | | |
| DUS10 | | | | MASP3 | IgG4, | DCC | | | 0.601 |
| | | | | | Kappa | | | | |
| DUS10 | | | | MASP3 | IgG4, | DCC | | CD248 | 0.601 |
| | | | | | Kappa | | | | |
| DUS10 | | | | MASP3 | IgG4, | DCC | RCAN3 | CD248 | 0.601 |
| | | | | | Kappa | | | | |
| DUS10 | EPHA6 | | | MASP3 | IgG4, | | RCAN3 | | 0.601 |
| | | | | | Kappa | | | | |
| DUS10 | EPHA6 | | | MASP3 | IgG4, | | RCAN3 | CD248 | 0.601 |
| | | | | | Kappa | | | | |
| DUS10 | EPHA6 | | | MASP3 | IgG4, | DCC | RCAN3 | | 0.601 |
| | | | | | Kappa | | | | |
| DUS10 | | | | MASP3 | IgG4, | | RCAN3 | CD248 | 0.6 |
| | | | | | Kappa | | | | |
| DUS10 | | | | MASP3 | IgG4, | DCC | RCAN3 | | 0.6 |
| | | | | | Kappa | | | | |

TABLE 20
IgG4, Kappa Biomarker Panels

| | | Alkaline | | | | | | | |
|---|---|---|---|---|---|---|---|---|---|
| IgG4, | | phosphatase, | | | | | | | |
| Kappa | EPHA6 | placental | SCF | MASP3 | DUS10 | DCC | RCAN3 | CD248 | AUC |
| IgG4, | EPHA6 | Alkaline | SCF | MASP3 | DUS10 | DCC | RCAN3 | CD248 | 0.698 |
| Kappa | | phosphatase, | | | | | | | |
| | | placental | | | | | | | |
| IgG4, | | Alkaline | SCF | MASP3 | DUS10 | DCC | RCAN3 | CD248 | 0.697 |
| Kappa | | phosphatase, | | | | | | | |
| | | placental | | | | | | | |
| IgG4, | EPHA6 | Alkaline | SCF | MASP3 | DUS10 | DCC | | CD248 | 0.697 |
| Kappa | | phosphatase, | | | | | | | |
| | | placental | | | | | | | |
| IgG4, | | Alkaline | SCF | MASP3 | DUS10 | | | CD248 | 0.696 |
| Kappa | | phosphatase, | | | | | | | |
| | | placental | | | | | | | |
| IgG4, | | Alkaline | SCF | MASP3 | DUS10 | | RCAN3 | CD248 | 0.696 |
| Kappa | | phosphatase, | | | | | | | |
| | | placental | | | | | | | |
| IgG4, | | Alkaline | SCF | MASP3 | DUS10 | DCC | | CD248 | 0.696 |
| Kappa | | phosphatase, | | | | | | | |
| | | placental | | | | | | | |
| IgG4, | EPHA6 | Alkaline | SCF | MASP3 | DUS10 | | | CD248 | 0.696 |
| Kappa | | phosphatase, | | | | | | | |
| | | placental | | | | | | | |
| IgG4, | EPHA6 | Alkaline | SCF | MASP3 | DUS10 | | RCAN3 | CD248 | 0.696 |
| Kappa | | phosphatase, | | | | | | | |
| | | placental | | | | | | | |
| IgG4, | EPHA6 | Alkaline | SCF | MASP3 | DUS10 | DCC | RCAN3 | | 0.696 |
| Kappa | | phosphatase, | | | | | | | |
| | | placental | | | | | | | |
| IgG4, | EPHA6 | Alkaline | SCF | MASP3 | DUS10 | | RCAN3 | | 0.695 |
| Kappa | | phosphatase, | | | | | | | |
| | | placental | | | | | | | |
| IgG4, | EPHA6 | Alkaline | SCF | MASP3 | DUS10 | DCC | | | 0.695 |
| Kappa | | phosphatase, | | | | | | | |
| | | placental | | | | | | | |
| IgG4, | | Alkaline | SCF | MASP3 | | DCC | RCAN3 | CD248 | 0.694 |
| Kappa | | phosphatase, | | | | | | | |
| | | placental | | | | | | | |
| IgG4, | | Alkaline | SCF | MASP3 | DUS10 | | RCAN3 | | 0.694 |
| Kappa | | phosphatase, | | | | | | | |
| | | placental | | | | | | | |
| IgG4, | | Alkaline | SCF | MASP3 | DUS10 | DCC | RCAN3 | | 0.694 |
| Kappa | | phosphatase, | | | | | | | |
| | | placental | | | | | | | |
| IgG4, | EPHA6 | Alkaline | SCF | MASP3 | | DCC | RCAN3 | CD248 | 0.694 |
| Kappa | | phosphatase, | | | | | | | |
| | | placental | | | | | | | |
| IgG4, | EPHA6 | Alkaline | SCF | MASP3 | DUS10 | | | | 0.694 |
| Kappa | | phosphatase, | | | | | | | |
| | | placental | | | | | | | |
| IgG4, | | Alkaline | SCF | MASP3 | | | | CD248 | 0.693 |
| Kappa | | phosphatase, | | | | | | | |
| | | placental | | | | | | | |
| IgG4, | | Alkaline | SCF | MASP3 | | | RCAN3 | CD248 | 0.693 |
| Kappa | | phosphatase, | | | | | | | |
| | | placental | | | | | | | |
| IgG4, | | Alkaline | SCF | MASP3 | | DCC | | CD248 | 0.693 |
| Kappa | | phosphatase, | | | | | | | |
| | | placental | | | | | | | |
| IgG4, | | Alkaline | SCF | MASP3 | DUS10 | | | | 0.693 |
| Kappa | | phosphatase, | | | | | | | |
| | | placental | | | | | | | |
| IgG4, | | Alkaline | SCF | MASP3 | DUS10 | DCC | | | 0.693 |
| Kappa | | phosphatase, | | | | | | | |
| | | placental | | | | | | | |
| IgG4, | EPHA6 | Alkaline | SCF | MASP3 | | | RCAN3 | CD248 | 0.693 |
| Kappa | | phosphatase, | | | | | | | |
| | | placental | | | | | | | |
| IgG4, | EPHA6 | Alkaline | SCF | MASP3 | | DCC | | CD248 | 0.693 |
| Kappa | | phosphatase, | | | | | | | |
| | | placental | | | | | | | |
| IgG4, | EPHA6 | Alkaline | SCF | MASP3 | | | | CD248 | 0.692 |
| Kappa | | phosphatase, | | | | | | | |
| | | placental | | | | | | | |
| IgG4, | EPHA6 | Alkaline | SCF | MASP3 | | | RCAN3 | | 0.692 |
| Kappa | | phosphatase, | | | | | | | |
| | | placental | | | | | | | |
| IgG4, | EPHA6 | Alkaline | SCF | MASP3 | | DCC | RCAN3 | | 0.692 |
| Kappa | | phosphatase, | | | | | | | |
| | | placental | | | | | | | |
| IgG4, | | Alkaline | SCF | MASP3 | | | RCAN3 | | 0.691 |
| Kappa | | phosphatase, | | | | | | | |
| | | placental | | | | | | | |
| IgG4, | | Alkaline | SCF | MASP3 | | DCC | RCAN3 | | 0.691 |
| Kappa | | phosphatase, | | | | | | | |
| | | placental | | | | | | | |
| IgG4, | EPHA6 | Alkaline | SCF | MASP3 | | | | | 0.691 |
| Kappa | | phosphatase, | | | | | | | |
| | | placental | | | | | | | |
| IgG4, | | Alkaline | SCF | MASP3 | | | | | 0.69 |
| Kappa | | phosphatase, | | | | | | | |
| | | placental | | | | | | | |
| IgG4, | | Alkaline | SCF | MASP3 | | DCC | | | 0.69 |
| Kappa | | phosphatase, | | | | | | | |
| | | placental | | | | | | | |
| IgG4, | EPHA6 | Alkaline | SCF | MASP3 | | DCC | | | 0.69 |
| Kappa | | phosphatase, | | | | | | | |
| | | placental | | | | | | | |
| IgG4, | | Alkaline | SCF | | DUS10 | DCC | RCAN3 | CD248 | 0.687 |
| Kappa | | phosphatase, | | | | | | | |
| | | placental | | | | | | | |
| IgG4, | EPHA6 | Alkaline | SCF | | DUS10 | DCC | RCAN3 | CD248 | 0.687 |
| Kappa | | phosphatase, | | | | | | | |
| | | placental | | | | | | | |
| IgG4, | EPHA6 | Alkaline | SCF | | DUS10 | DCC | | CD248 | 0.686 |
| Kappa | | phosphatase, | | | | | | | |
| | | placental | | | | | | | |
| IgG4, | | Alkaline | SCF | | DUS10 | DCC | | CD248 | 0.685 |
| Kappa | | phosphatase, | | | | | | | |
| | | placental | | | | | | | |
| IgG4, | EPHA6 | Alkaline | SCF | | DUS10 | DCC | RCAN3 | | 0.684 |
| Kappa | | phosphatase, | | | | | | | |
| | | placental | | | | | | | |
| IgG4, | | Alkaline | | MASP3 | DUS10 | DCC | | CD248 | 0.683 |
| Kappa | | phosphatase, | | | | | | | |
| | | placental | | | | | | | |
| IgG4, | EPHA6 | Alkaline | | MASP3 | DUS10 | DCC | | CD248 | 0.683 |
| Kappa | | phosphatase, | | | | | | | |
| | | placental | | | | | | | |
| IgG4, | EPHA6 | Alkaline | SCF | | DUS10 | DCC | | | 0.683 |
| Kappa | | phosphatase, | | | | | | | |
| | | placental | | | | | | | |
| IgG4, | | Alkaline | | MASP3 | DUS10 | DCC | RCAN3 | CD248 | 0.682 |
| Kappa | | phosphatase, | | | | | | | |
| | | placental | | | | | | | |
| IgG4, | EPHA6 | Alkaline | | MASP3 | DUS10 | DCC | RCAN3 | CD248 | 0.682 |
| Kappa | | phosphatase, | | | | | | | |
| | | placental | | | | | | | |
| IgG4, | EPHA6 | Alkaline | SCF | | | DCC | RCAN3 | CD248 | 0.682 |
| Kappa | | phosphatase, | | | | | | | |
| | | placental | | | | | | | |
| IgG4, | | Alkaline | SCF | | | DCC | RCAN3 | CD248 | 0.681 |
| Kappa | | phosphatase, | | | | | | | |
| | | placental | | | | | | | |
| IgG4, | | Alkaline | SCF | | DUS10 | | RCAN3 | CD248 | 0.681 |
| Kappa | | phosphatase, | | | | | | | |
| | | placental | | | | | | | |
| IgG4, | | Alkaline | SCF | | DUS10 | DCC | RCAN3 | | 0.681 |
| Kappa | | phosphatase, | | | | | | | |
| | | placental | | | | | | | |
| IgG4, | EPHA6 | Alkaline | | MASP3 | DUS10 | | | CD248 | 0.681 |
| Kappa | | phosphatase, | | | | | | | |
| | | placental | | | | | | | |
| IgG4, | EPHA6 | Alkaline | | MASP3 | DUS10 | | RCAN3 | CD248 | 0.681 |
| Kappa | | phosphatase, | | | | | | | |
| | | placental | | | | | | | |
| IgG4, | EPHA6 | Alkaline | SCF | | DUS10 | | RCAN3 | CD248 | 0.681 |
| Kappa | | phosphatase, | | | | | | | |
| | | placental | | | | | | | |
| IgG4, | | Alkaline | | MASP3 | DUS10 | | | | 0.68 |
| Kappa | | phosphatase, | | | | | | | |
| | | placental | | | | | | | |
| IgG4, | | Alkaline | | MASP3 | DUS10 | | | CD248 | 0.68 |
| Kappa | | phosphatase, | | | | | | | |
| | | placental | | | | | | | |
| IgG4, | | Alkaline | | MASP3 | DUS10 | | RCAN3 | | 0.68 |
| Kappa | | phosphatase, | | | | | | | |
| | | placental | | | | | | | |
| IgG4, | | Alkaline | | MASP3 | DUS10 | | RCAN3 | CD248 | 0.68 |
| Kappa | | phosphatase, | | | | | | | |
| | | placental | | | | | | | |
| IgG4, | | Alkaline | | MASP3 | DUS10 | DCC | | | 0.68 |
| Kappa | | phosphatase, | | | | | | | |
| | | placental | | | | | | | |
| IgG4, | | Alkaline | | MASP3 | DUS10 | DCC | RCAN3 | | 0.68 |
| Kappa | | phosphatase, | | | | | | | |
| | | placental | | | | | | | |
| IgG4, | | Alkaline | SCF | | DUS10 | | | CD248 | 0.68 |
| Kappa | | phosphatase, | | | | | | | |
| | | placental | | | | | | | |
| IgG4, | EPHA6 | Alkaline | | MASP3 | DUS10 | | | | 0.68 |
| Kappa | | phosphatase, | | | | | | | |
| | | placental | | | | | | | |
| IgG4, | EPHA6 | Alkaline | | MASP3 | DUS10 | | RCAN3 | | 0.68 |
| Kappa | | phosphatase, | | | | | | | |
| | | placental | | | | | | | |
| IgG4, | EPHA6 | Alkaline | | MASP3 | DUS10 | DCC | | | 0.68 |
| Kappa | | phosphatase, | | | | | | | |
| | | placental | | | | | | | |
| IgG4, | EPHA6 | Alkaline | | MASP3 | DUS10 | DCC | RCAN3 | | 0.68 |
| Kappa | | phosphatase, | | | | | | | |
| | | placental | | | | | | | |
| IgG4, | EPHA6 | Alkaline | SCF | | | DCC | | CD248 | 0.68 |
| Kappa | | phosphatase, | | | | | | | |
| | | placental | | | | | | | |
| IgG4, | EPHA6 | Alkaline | SCF | | | DCC | RCAN3 | | 0.68 |
| Kappa | | phosphatase, | | | | | | | |
| | | placental | | | | | | | |
| IgG4, | EPHA6 | Alkaline | SCF | | DUS10 | | | CD248 | 0.68 |
| Kappa | | phosphatase, | | | | | | | |
| | | placental | | | | | | | |
| IgG4, | EPHA6 | Alkaline | SCF | | DUS10 | | RCAN3 | | 0.68 |
| Kappa | | phosphatase, | | | | | | | |
| | | placental | | | | | | | |
| IgG4, | | Alkaline | SCF | | | DCC | | CD248 | 0.679 |
| Kappa | | phosphatase, | | | | | | | |
| | | placental | | | | | | | |
| IgG4, | | Alkaline | SCF | | DUS10 | | RCAN3 | | 0.679 |
| Kappa | | phosphatase, | | | | | | | |
| | | placental | | | | | | | |
| IgG4, | | Alkaline | SCF | | DUS10 | DCC | | | 0.679 |
| Kappa | | phosphatase, | | | | | | | |
| | | placental | | | | | | | |
| IgG4, | | Alkaline | | MASP3 | | DCC | | CD248 | 0.678 |
| Kappa | | phosphatase, | | | | | | | |
| | | placental | | | | | | | |
| IgG4, | | Alkaline | | MASP3 | | DCC | RCAN3 | CD248 | 0.678 |
| Kappa | | phosphatase, | | | | | | | |
| | | placental | | | | | | | |
| IgG4, | EPHA6 | Alkaline | | MASP3 | | DCC | | CD248 | 0.678 |
| Kappa | | phosphatase, | | | | | | | |
| | | placental | | | | | | | |
| IgG4, | EPHA6 | Alkaline | | MASP3 | | DCC | RCAN3 | CD248 | 0.678 |
| Kappa | | phosphatase, | | | | | | | |
| | | placental | | | | | | | |
| IgG4, | EPHA6 | Alkaline | SCF | | | DCC | | | 0.678 |
| Kappa | | phosphatase, | | | | | | | |
| | | placental | | | | | | | |
| IgG4, | | Alkaline | SCF | | DUS10 | | | | 0.677 |
| Kappa | | phosphatase, | | | | | | | |
| | | placental | | | | | | | |
| IgG4, | EPHA6 | Alkaline | | MASP3 | | DCC | | | 0.677 |
| Kappa | | phosphatase, | | | | | | | |
| | | placental | | | | | | | |
| IgG4, | EPHA6 | Alkaline | | MASP3 | | DCC | RCAN3 | | 0.677 |
| Kappa | | phosphatase, | | | | | | | |
| | | placental | | | | | | | |
| IgG4, | EPHA6 | Alkaline | SCF | | DUS10 | | | | 0.677 |
| Kappa | | phosphatase, | | | | | | | |
| | | placental | | | | | | | |
| IgG4, | | Alkaline | | MASP3 | | | | | 0.676 |
| Kappa | | phosphatase, | | | | | | | |
| | | placental | | | | | | | |
| IgG4, | | Alkaline | | MASP3 | | | RCAN3 | | 0.676 |
| Kappa | | phosphatase, | | | | | | | |
| | | placental | | | | | | | |
| IgG4, | | Alkaline | | MASP3 | | DCC | RCAN3 | | 0.676 |
| Kappa | | phosphatase, | | | | | | | |
| | | placental | | | | | | | |
| IgG4, | | Alkaline | SCF | | | | RCAN3 | CD248 | 0.676 |
| Kappa | | phosphatase, | | | | | | | |
| | | placental | | | | | | | |
| IgG4, | | Alkaline | SCF | | | DCC | RCAN3 | | 0.676 |
| Kappa | | phosphatase, | | | | | | | |
| | | placental | | | | | | | |
| IgG4, | EPHA6 | Alkaline | | MASP3 | | | | CD248 | 0.676 |
| Kappa | | phosphatase, | | | | | | | |
| | | placental | | | | | | | |
| IgG4, | EPHA6 | Alkaline | SCF | | | | RCAN3 | CD248 | 0.676 |
| Kappa | | phosphatase, | | | | | | | |
| | | placental | | | | | | | |
| IgG4, | | Alkaline | | MASP3 | | | | CD248 | 0.675 |
| Kappa | | phosphatase, | | | | | | | |
| | | placental | | | | | | | |
| IgG4, | | Alkaline | | MASP3 | | | RCAN3 | CD248 | 0.675 |
| Kappa | | phosphatase, | | | | | | | |
| | | placental | | | | | | | |
| IgG4, | | Alkaline | | MASP3 | | DCC | | | 0.675 |
| Kappa | | phosphatase, | | | | | | | |
| | | placental | | | | | | | |
| IgG4, | | Alkaline | SCF | | | | | CD248 | 0.675 |
| Kappa | | phosphatase, | | | | | | | |
| | | placental | | | | | | | |
| IgG4, | EPHA6 | Alkaline | | MASP3 | | | | | 0.675 |
| Kappa | | phosphatase, | | | | | | | |
| | | placental | | | | | | | |
| IgG4, | EPHA6 | Alkaline | | MASP3 | | | RCAN3 | | 0.675 |
| Kappa | | phosphatase, | | | | | | | |
| | | placental | | | | | | | |
| IgG4, | EPHA6 | Alkaline | | MASP3 | | | RCAN3 | CD248 | 0.675 |
| Kappa | | phosphatase, | | | | | | | |
| | | placental | | | | | | | |
| IgG4, | | Alkaline | SCF | | | | RCAN3 | | 0.674 |
| Kappa | | phosphatase, | | | | | | | |
| | | placental | | | | | | | |
| IgG4, | EPHA6 | Alkaline | SCF | | | | | CD248 | 0.674 |
| Kappa | | phosphatase, | | | | | | | |
| | | placental | | | | | | | |
| IgG4, | EPHA6 | Alkaline | SCF | | | | RCAN3 | | 0.674 |
| Kappa | | phosphatase, | | | | | | | |
| | | placental | | | | | | | |
| IgG4, | | Alkaline | SCF | | | DCC | | | 0.673 |
| Kappa | | phosphatase, | | | | | | | |
| | | placental | | | | | | | |
| IgG4, | EPHA6 | Alkaline | SCF | | | | | | 0.673 |
| Kappa | | phosphatase, | | | | | | | |
| | | placental | | | | | | | |
| IgG4, | | Alkaline | SCF | | | | | | 0.671 |
| Kappa | | phosphatase, | | | | | | | |
| | | placental | | | | | | | |
| IgG4, | | Alkaline | | | DUS10 | DCC | | CD248 | 0.667 |
| Kappa | | phosphatase, | | | | | | | |
| | | placental | | | | | | | |
| IgG4, | | Alkaline | | | DUS10 | DCC | RCAN3 | CD248 | 0.667 |
| Kappa | | phosphatase, | | | | | | | |
| | | placental | | | | | | | |
| IgG4, | EPHA6 | Alkaline | | | DUS10 | DCC | | CD248 | 0.667 |
| Kappa | | phosphatase, | | | | | | | |
| | | placental | | | | | | | |
| IgG4, | EPHA6 | Alkaline | | | DUS10 | DCC | RCAN3 | CD248 | 0.667 |
| Kappa | | phosphatase, | | | | | | | |
| | | placental | | | | | | | |
| IgG4, | EPHA6 | Alkaline | | | DUS10 | DCC | | | 0.666 |
| Kappa | | phosphatase, | | | | | | | |
| | | placental | | | | | | | |
| IgG4, | EPHA6 | Alkaline | | | DUS10 | DCC | RCAN3 | | 0.666 |
| Kappa | | phosphatase, | | | | | | | |
| | | placental | | | | | | | |
| IgG4, | | Alkaline | | | DUS10 | DCC | | | 0.665 |
| Kappa | | phosphatase, | | | | | | | |
| | | placental | | | | | | | |
| IgG4, | | Alkaline | | | DUS10 | DCC | RCAN3 | | 0.665 |
| Kappa | | phosphatase, | | | | | | | |
| | | placental | | | | | | | |
| IgG4, | | Alkaline | | | DUS10 | | | | 0.661 |
| Kappa | | phosphatase, | | | | | | | |
| | | placental | | | | | | | |
| IgG4, | | Alkaline | | | DUS10 | | RCAN3 | | 0.661 |
| Kappa | | phosphatase, | | | | | | | |
| | | placental | | | | | | | |
| IgG4, | EPHA6 | Alkaline | | | | DCC | | CD248 | 0.661 |
| Kappa | | phosphatase, | | | | | | | |
| | | placental | | | | | | | |
| IgG4, | EPHA6 | Alkaline | | | | DCC | RCAN3 | CD248 | 0.661 |
| Kappa | | phosphatase, | | | | | | | |
| | | placental | | | | | | | |
| IgG4, | | Alkaline | | | | DCC | | CD248 | 0.66 |
| Kappa | | phosphatase, | | | | | | | |
| | | placental | | | | | | | |
| IgG4, | | Alkaline | | | | DCC | RCAN3 | CD248 | 0.66 |
| Kappa | | phosphatase, | | | | | | | |
| | | placental | | | | | | | |
| IgG4, | | Alkaline | | | DUS10 | | | CD248 | 0.66 |
| Kappa | | phosphatase, | | | | | | | |
| | | placental | | | | | | | |
| IgG4, | | Alkaline | | | DUS10 | | RCAN3 | CD248 | 0.66 |
| Kappa | | phosphatase, | | | | | | | |
| | | placental | | | | | | | |
| IgG4, | EPHA6 | Alkaline | | | DUS10 | | | | 0.66 |
| Kappa | | phosphatase, | | | | | | | |
| | | placental | | | | | | | |
| IgG4, | EPHA6 | Alkaline | | | DUS10 | | | CD248 | 0.66 |
| Kappa | | phosphatase, | | | | | | | |
| | | placental | | | | | | | |
| IgG4, | EPHA6 | Alkaline | | | DUS10 | | RCAN3 | | 0.66 |
| Kappa | | phosphatase, | | | | | | | |
| | | placental | | | | | | | |
| IgG4, | EPHA6 | Alkaline | | | DUS10 | | RCAN3 | CD248 | 0.66 |
| Kappa | | phosphatase, | | | | | | | |
| | | placental | | | | | | | |
| IgG4, | EPHA6 | Alkaline | | | | DCC | | | 0.659 |
| Kappa | | phosphatase, | | | | | | | |
| | | placental | | | | | | | |
| IgG4, | EPHA6 | Alkaline | | | | DCC | RCAN3 | | 0.659 |
| Kappa | | phosphatase, | | | | | | | |
| | | placental | | | | | | | |
| IgG4, | | Alkaline | | | | DCC | | | 0.657 |
| Kappa | | phosphatase, | | | | | | | |
| | | placental | | | | | | | |
| IgG4, | | Alkaline | | | | DCC | RCAN3 | | 0.657 |
| Kappa | | phosphatase, | | | | | | | |
| | | placental | | | | | | | |
| IgG4, | | Alkaline | | | | | | | 0.654 |
| Kappa | | phosphatase, | | | | | | | |
| | | placental | | | | | | | |
| IgG4, | | Alkaline | | | | | RCAN3 | | 0.654 |
| Kappa | | phosphatase, | | | | | | | |
| | | placental | | | | | | | |
| IgG4, | EPHA6 | Alkaline | | | | | | | 0.652 |
| Kappa | | phosphatase, | | | | | | | |
| | | placental | | | | | | | |
| IgG4, | EPHA6 | Alkaline | | | | | RCAN3 | | 0.652 |
| Kappa | | phosphatase, | | | | | | | |
| | | placental | | | | | | | |
| IgG4, | EPHA6 | Alkaline | | | | | RCAN3 | CD248 | 0.652 |
| Kappa | | phosphatase, | | | | | | | |
| | | placental | | | | | | | |
| IgG4, | | Alkaline | | | | | | CD248 | 0.651 |
| Kappa | | phosphatase, | | | | | | | |
| | | placental | | | | | | | |
| IgG4, | | Alkaline | | | | | RCAN3 | CD248 | 0.651 |
| Kappa | | phosphatase, | | | | | | | |
| | | placental | | | | | | | |
| IgG4, | EPHA6 | Alkaline | | | | | | CD248 | 0.651 |
| Kappa | | phosphatase, | | | | | | | |
| | | placental | | | | | | | |
| IgG4, | EPHA6 | | SCF | MASP3 | | DCC | | CD248 | 0.635 |
| Kappa | | | | | | | | | |
| IgG4, | EPHA6 | | SCF | MASP3 | | DCC | RCAN3 | CD248 | 0.635 |
| Kappa | | | | | | | | | |
| IgG4, | EPHA6 | | SCF | MASP3 | DUS10 | DCC | | | 0.634 |
| Kappa | | | | | | | | | |
| IgG4, | EPHA6 | | SCF | MASP3 | DUS10 | DCC | | CD248 | 0.634 |
| Kappa | | | | | | | | | |
| IgG4, | EPHA6 | | SCF | MASP3 | DUS10 | DCC | RCAN3 | | 0.634 |
| Kappa | | | | | | | | | |
| IgG4, | EPHA6 | | SCF | MASP3 | DUS10 | DCC | RCAN3 | CD248 | 0.634 |
| Kappa | | | | | | | | | |
| IgG4, | EPHA6 | | SCF | MASP3 | | DCC | | | 0.633 |
| Kappa | | | | | | | | | |
| IgG4, | EPHA6 | | SCF | MASP3 | | DCC | RCAN3 | | 0.633 |
| Kappa | | | | | | | | | |
| IgG4, | | | SCF | MASP3 | | DCC | | CD248 | 0.631 |
| Kappa | | | | | | | | | |
| IgG4, | | | SCF | MASP3 | | DCC | RCAN3 | CD248 | 0.631 |
| Kappa | | | | | | | | | |
| IgG4, | | | SCF | MASP3 | DUS10 | DCC | | CD248 | 0.631 |
| Kappa | | | | | | | | | |
| IgG4, | | | SCF | MASP3 | DUS10 | DCC | RCAN3 | CD248 | 0.63 |
| Kappa | | | | | | | | | |
| IgG4, | | | SCF | MASP3 | | DCC | | | 0.629 |
| Kappa | | | | | | | | | |
| IgG4, | | | SCF | MASP3 | | DCC | RCAN3 | | 0.629 |
| Kappa | | | | | | | | | |
| IgG4, | | | SCF | MASP3 | DUS10 | DCC | | | 0.629 |
| Kappa | | | | | | | | | |
| IgG4, | | | SCF | MASP3 | DUS10 | DCC | RCAN3 | | 0.629 |
| Kappa | | | | | | | | | |
| IgG4, | EPHA6 | | SCF | | | DCC | | CD248 | 0.629 |
| Kappa | | | | | | | | | |
| IgG4, | EPHA6 | | SCF | | | DCC | RCAN3 | | 0.629 |
| Kappa | | | | | | | | | |
| IgG4, | EPHA6 | | SCF | | | DCC | RCAN3 | CD248 | 0.629 |
| Kappa | | | | | | | | | |
| IgG4, | EPHA6 | | SCF | | DUS10 | DCC | | | 0.629 |
| Kappa | | | | | | | | | |
| IgG4, | EPHA6 | | SCF | | DUS10 | DCC | | CD248 | 0.629 |
| Kappa | | | | | | | | | |
| IgG4, | EPHA6 | | SCF | | DUS10 | DCC | RCAN3 | | 0.629 |
| Kappa | | | | | | | | | |
| IgG4, | EPHA6 | | SCF | | DUS10 | DCC | RCAN3 | CD248 | 0.629 |
| Kappa | | | | | | | | | |
| IgG4, | EPHA6 | | SCF | | | DCC | | | 0.628 |
| Kappa | | | | | | | | | |
| IgG4, | | | SCF | | DUS10 | DCC | | CD248 | 0.627 |
| Kappa | | | | | | | | | |
| IgG4, | | | SCF | | DUS10 | DCC | RCAN3 | CD248 | 0.627 |
| Kappa | | | | | | | | | |
| IgG4, | | | SCF | MASP3 | DUS10 | | | CD248 | 0.627 |
| Kappa | | | | | | | | | |
| IgG4, | | | SCF | MASP3 | DUS10 | | RCAN3 | CD248 | 0.627 |
| Kappa | | | | | | | | | |
| IgG4, | EPHA6 | | SCF | MASP3 | DUS10 | | | CD248 | 0.627 |
| Kappa | | | | | | | | | |
| IgG4, | EPHA6 | | SCF | MASP3 | DUS10 | | RCAN3 | | 0.627 |
| Kappa | | | | | | | | | |
| IgG4, | EPHA6 | | SCF | MASP3 | DUS10 | | RCAN3 | CD248 | 0.627 |
| Kappa | | | | | | | | | |
| IgG4, | | | SCF | | | DCC | | CD248 | 0.626 |
| Kappa | | | | | | | | | |
| IgG4, | | | SCF | | | DCC | RCAN3 | CD248 | 0.626 |
| Kappa | | | | | | | | | |
| IgG4, | | | SCF | MASP3 | | | | CD248 | 0.626 |
| Kappa | | | | | | | | | |
| IgG4, | | | SCF | MASP3 | | | RCAN3 | CD248 | 0.626 |
| Kappa | | | | | | | | | |
| IgG4, | | | SCF | MASP3 | DUS10 | | | | 0.626 |
| Kappa | | | | | | | | | |
| IgG4, | | | SCF | MASP3 | DUS10 | | RCAN3 | | 0.626 |
| Kappa | | | | | | | | | |
| IgG4, | EPHA6 | | SCF | MASP3 | | | | | 0.626 |
| Kappa | | | | | | | | | |
| IgG4, | EPHA6 | | SCF | MASP3 | | | | CD248 | 0.626 |
| Kappa | | | | | | | | | |
| IgG4, | EPHA6 | | SCF | MASP3 | | | RCAN3 | CD248 | 0.626 |
| Kappa | | | | | | | | | |
| IgG4, | EPHA6 | | SCF | MASP3 | DUS10 | | | | 0.626 |
| Kappa | | | | | | | | | |
| IgG4, | | | SCF | MASP3 | | | | | 0.625 |
| Kappa | | | | | | | | | |
| IgG4, | | | SCF | MASP3 | | | RCAN3 | | 0.625 |
| Kappa | | | | | | | | | |
| IgG4, | EPHA6 | | SCF | MASP3 | | | RCAN3 | | 0.625 |
| Kappa | | | | | | | | | |
| IgG4, | | | SCF | | DUS10 | DCC | | | 0.623 |
| Kappa | | | | | | | | | |
| IgG4, | | | SCF | | DUS10 | DCC | RCAN3 | | 0.623 |
| Kappa | | | | | | | | | |
| IgG4, | | | SCF | | | DCC | RCAN3 | | 0.622 |
| Kappa | | | | | | | | | |
| IgG4, | | | SCF | | | DCC | | | 0.621 |
| Kappa | | | | | | | | | |
| IgG4, | | | SCF | | DUS10 | | | CD248 | 0.618 |
| Kappa | | | | | | | | | |
| IgG4, | | | SCF | | DUS10 | | RCAN3 | CD248 | 0.618 |
| Kappa | | | | | | | | | |
| IgG4, | EPHA6 | | SCF | | DUS10 | | | | 0.618 |
| Kappa | | | | | | | | | |
| IgG4, | EPHA6 | | SCF | | DUS10 | | | CD248 | 0.618 |
| Kappa | | | | | | | | | |
| IgG4, | EPHA6 | | SCF | | DUS10 | | RCAN3 | | 0.618 |
| Kappa | | | | | | | | | |
| IgG4, | EPHA6 | | SCF | | DUS10 | | RCAN3 | CD248 | 0.618 |
| Kappa | | | | | | | | | |
| IgG4, | | | SCF | | | | | CD248 | 0.616 |
| Kappa | | | | | | | | | |
| IgG4, | | | SCF | | | | RCAN3 | CD248 | 0.616 |
| Kappa | | | | | | | | | |
| IgG4, | | | SCF | | DUS10 | | | | 0.616 |
| Kappa | | | | | | | | | |
| IgG4, | | | SCF | | DUS10 | | RCAN3 | | 0.616 |
| Kappa | | | | | | | | | |
| IgG4, | EPHA6 | | SCF | | | | RCAN3 | | 0.616 |
| Kappa | | | | | | | | | |
| IgG4, | EPHA6 | | SCF | | | | RCAN3 | CD248 | 0.616 |
| Kappa | | | | | | | | | |
| IgG4, | | | SCF | | | | RCAN3 | | 0.615 |
| Kappa | | | | | | | | | |
| IgG4, | EPHA6 | | SCF | | | | | | 0.615 |
| Kappa | | | | | | | | | |
| IgG4, | EPHA6 | | SCF | | | | | CD248 | 0.615 |
| Kappa | | | | | | | | | |
| IgG4, | | | SCF | | | | | | 0.614 |
| Kappa | | | | | | | | | |
| IgG4, | EPHA6 | | | MASP3 | DUS10 | DCC | | | 0.602 |
| Kappa | | | | | | | | | |
| IgG4, | EPHA6 | | | MASP3 | DUS10 | DCC | | CD248 | 0.602 |
| Kappa | | | | | | | | | |
| IgG4, | EPHA6 | | | MASP3 | DUS10 | DCC | RCAN3 | CD248 | 0.602 |
| Kappa | | | | | | | | | |
| IgG4, | | | | MASP3 | DUS10 | | RCAN3 | | 0.601 |
| Kappa | | | | | | | | | |
| IgG4, | | | | MASP3 | DUS10 | DCC | | | 0.601 |
| Kappa | | | | | | | | | |
| IgG4, | | | | MASP3 | DUS10 | DCC | | CD248 | 0.601 |
| Kappa | | | | | | | | | |
| IgG4, | | | | MASP3 | DUS10 | DCC | RCAN3 | CD248 | 0.601 |
| Kappa | | | | | | | | | |
| IgG4, | EPHA6 | | | MASP3 | DUS10 | | RCAN3 | | 0.601 |
| Kappa | | | | | | | | | |
| IgG4, | EPHA6 | | | MASP3 | DUS10 | | RCAN3 | CD248 | 0.601 |
| Kappa | | | | | | | | | |
| IgG4, | EPHA6 | | | MASP3 | DUS10 | DCC | RCAN3 | | 0.601 |
| Kappa | | | | | | | | | |
| IgG4, | | | | MASP3 | DUS10 | | RCAN3 | CD248 | 0.6 |
| Kappa | | | | | | | | | |
| IgG4, | | | | MASP3 | DUS10 | DCC | RCAN3 | | 0.6 |
| Kappa | | | | | | | | | |
| IgG4, | EPHA6 | | | MASP3 | | DCC | | CD248 | 0.6 |
| Kappa | | | | | | | | | |
| IgG4, | EPHA6 | | | MASP3 | | DCC | RCAN3 | CD248 | 0.6 |
| Kappa | | | | | | | | | |

TABLE 21
DCC Biomarker Panels

| | | Alkaline | | | | | | | |
|---|---|---|---|---|---|---|---|---|---|
| | | phosphatase, | | | | IgG4, | | | |
| DCC | EPHA6 | placental | SCF | MASP3 | DUS10 | Kappa | RCAN3 | CD248 | AUC |
| DCC | EPHA6 | Alkaline | SCF | MASP3 | DUS10 | IgG4, | RCAN3 | CD248 | 0.698 |
| | | phosphatase, | | | | Kappa | | | |
| | | placental | | | | | | | |
| DCC | | Alkaline | SCF | MASP3 | DUS10 | IgG4, | RCAN3 | CD248 | 0.697 |
| | | phosphatase, | | | | Kappa | | | |
| | | placental | | | | | | | |
| DCC | EPHA6 | Alkaline | SCF | MASP3 | DUS10 | IgG4, | | CD248 | 0.697 |
| | | phosphatase, | | | | Kappa | | | |
| | | placental | | | | | | | |
| DCC | | Alkaline | SCF | MASP3 | DUS10 | IgG4, | | CD248 | 0.696 |
| | | phosphatase, | | | | Kappa | | | |
| | | placental | | | | | | | |
| DCC | EPHA6 | Alkaline | SCF | MASP3 | DUS10 | IgG4, | RCAN3 | | 0.696 |
| | | phosphatase, | | | | Kappa | | | |
| | | placental | | | | | | | |
| DCC | EPHA6 | Alkaline | SCF | MASP3 | DUS10 | IgG4, | | | 0.695 |
| | | phosphatase, | | | | Kappa | | | |
| | | placental | | | | | | | |
| DCC | | Alkaline | SCF | MASP3 | | IgG4, | RCAN3 | CD248 | 0.694 |
| | | phosphatase, | | | | Kappa | | | |
| | | placental | | | | | | | |
| DCC | | Alkaline | SCF | MASP3 | DUS10 | IgG4, | RCAN3 | | 0.694 |
| | | phosphatase, | | | | Kappa | | | |
| | | placental | | | | | | | |
| DCC | EPHA6 | Alkaline | SCF | MASP3 | | IgG4, | RCAN3 | CD248 | 0.694 |
| | | phosphatase, | | | | Kappa | | | |
| | | placental | | | | | | | |
| DCC | | Alkaline | SCF | MASP3 | | IgG4, | | CD248 | 0.693 |
| | | phosphatase, | | | | Kappa | | | |
| | | placental | | | | | | | |
| DCC | | Alkaline | SCF | MASP3 | DUS10 | IgG4, | | | 0.693 |
| | | phosphatase, | | | | Kappa | | | |
| | | placental | | | | | | | |
| DCC | EPHA6 | Alkaline | SCF | MASP3 | | IgG4, | | CD248 | 0.693 |
| | | phosphatase, | | | | Kappa | | | |
| | | placental | | | | | | | |
| DCC | EPHA6 | Alkaline | SCF | MASP3 | | IgG4, | RCAN3 | | 0.692 |
| | | phosphatase, | | | | Kappa | | | |
| | | placental | | | | | | | |
| DCC | | Alkaline | SCF | MASP3 | | IgG4, | RCAN3 | | 0.691 |
| | | phosphatase, | | | | Kappa | | | |
| | | placental | | | | | | | |
| DCC | | Alkaline | SCF | MASP3 | | IgG4, | | | 0.69 |
| | | phosphatase, | | | | Kappa | | | |
| | | placental | | | | | | | |
| DCC | EPHA6 | Alkaline | SCF | MASP3 | | IgG4, | | | 0.69 |
| | | phosphatase, | | | | Kappa | | | |
| | | placental | | | | | | | |
| DCC | EPHA6 | Alkaline | SCF | MASP3 | DUS10 | | RCAN3 | CD248 | 0.689 |
| | | phosphatase, | | | | | | | |
| | | placental | | | | | | | |
| DCC | | Alkaline | SCF | | DUS10 | IgG4, | RCAN3 | CD248 | 0.687 |
| | | phosphatase, | | | | Kappa | | | |
| | | placental | | | | | | | |
| DCC | | Alkaline | SCF | MASP3 | DUS10 | | RCAN3 | CD248 | 0.687 |
| | | phosphatase, | | | | | | | |
| | | placental | | | | | | | |
| DCC | EPHA6 | Alkaline | SCF | | DUS10 | IgG4, | RCAN3 | CD248 | 0.687 |
| | | phosphatase, | | | | Kappa | | | |
| | | placental | | | | | | | |
| DCC | EPHA6 | Alkaline | SCF | MASP3 | DUS10 | | | CD248 | 0.687 |
| | | phosphatase, | | | | | | | |
| | | placental | | | | | | | |
| DCC | | Alkaline | SCF | MASP3 | DUS10 | | | CD248 | 0.686 |
| | | phosphatase, | | | | | | | |
| | | placental | | | | | | | |
| DCC | EPHA6 | Alkaline | SCF | | DUS10 | IgG4, | | CD248 | 0.686 |
| | | phosphatase, | | | | Kappa | | | |
| | | placental | | | | | | | |
| DCC | EPHA6 | Alkaline | SCF | MASP3 | DUS10 | | RCAN3 | | 0.686 |
| | | phosphatase, | | | | | | | |
| | | placental | | | | | | | |
| DCC | | Alkaline | SCF | | DUS10 | IgG4, | | CD248 | 0.685 |
| | | phosphatase, | | | | Kappa | | | |
| | | placental | | | | | | | |
| DCC | EPHA6 | Alkaline | SCF | | DUS10 | IgG4, | RCAN3 | | 0.684 |
| | | phosphatase, | | | | Kappa | | | |
| | | placental | | | | | | | |
| DCC | EPHA6 | Alkaline | SCF | MASP3 | | | RCAN3 | CD248 | 0.684 |
| | | phosphatase, | | | | | | | |
| | | placental | | | | | | | |
| DCC | EPHA6 | Alkaline | SCF | MASP3 | DUS10 | | | | 0.684 |
| | | phosphatase, | | | | | | | |
| | | placental | | | | | | | |
| DCC | | Alkaline | | MASP3 | DUS10 | IgG4, | | CD248 | 0.683 |
| | | phosphatase, | | | | Kappa | | | |
| | | placental | | | | | | | |
| DCC | | Alkaline | SCF | MASP3 | | | RCAN3 | CD248 | 0.683 |
| | | phosphatase, | | | | | | | |
| | | placental | | | | | | | |
| DCC | | Alkaline | SCF | MASP3 | DUS10 | | RCAN3 | | 0.683 |
| | | phosphatase, | | | | | | | |
| | | placental | | | | | | | |
| DCC | EPHA6 | Alkaline | | MASP3 | DUS10 | IgG4, | | CD248 | 0.683 |
| | | phosphatase, | | | | Kappa | | | |
| | | placental | | | | | | | |
| DCC | EPHA6 | Alkaline | SCF | | DUS10 | IgG4, | | | 0.683 |
| | | phosphatase, | | | | Kappa | | | |
| | | placental | | | | | | | |
| DCC | | Alkaline | | MASP3 | DUS10 | IgG4, | RCAN3 | CD248 | 0.682 |
| | | phosphatase, | | | | Kappa | | | |
| | | placental | | | | | | | |
| DCC | | Alkaline | SCF | MASP3 | DUS10 | | | | 0.682 |
| | | phosphatase, | | | | | | | |
| | | placental | | | | | | | |
| DCC | EPHA6 | Alkaline | | MASP3 | DUS10 | IgG4, | RCAN3 | CD248 | 0.682 |
| | | phosphatase, | | | | Kappa | | | |
| | | placental | | | | | | | |
| DCC | EPHA6 | Alkaline | SCF | | | IgG4, | RCAN3 | CD248 | 0.682 |
| | | phosphatase, | | | | Kappa | | | |
| | | placental | | | | | | | |
| DCC | EPHA6 | Alkaline | SCF | MASP3 | | | | CD248 | 0.682 |
| | | phosphatase, | | | | | | | |
| | | placental | | | | | | | |
| DCC | | Alkaline | SCF | | | IgG4, | RCAN3 | CD248 | 0.681 |
| | | phosphatase, | | | | Kappa | | | |
| | | placental | | | | | | | |
| DCC | | Alkaline | SCF | | DUS10 | IgG4, | RCAN3 | | 0.681 |
| | | phosphatase, | | | | Kappa | | | |
| | | placental | | | | | | | |
| DCC | | Alkaline | SCF | MASP3 | | | | CD248 | 0.681 |
| | | phosphatase, | | | | | | | |
| | | placental | | | | | | | |
| DCC | EPHA6 | Alkaline | SCF | MASP3 | | | RCAN3 | | 0.681 |
| | | phosphatase, | | | | | | | |
| | | placental | | | | | | | |
| DCC | | Alkaline | | MASP3 | DUS10 | IgG4, | | | 0.68 |
| | | phosphatase, | | | | Kappa | | | |
| | | placental | | | | | | | |
| DCC | | Alkaline | | MASP3 | DUS10 | IgG4, | RCAN3 | | 0.68 |
| | | phosphatase, | | | | Kappa | | | |
| | | placental | | | | | | | |
| DCC | EPHA6 | Alkaline | | MASP3 | DUS10 | IgG4, | | | 0.68 |
| | | phosphatase, | | | | Kappa | | | |
| | | placental | | | | | | | |
| DCC | EPHA6 | Alkaline | | MASP3 | DUS10 | IgG4, | RCAN3 | | 0.68 |
| | | phosphatase, | | | | Kappa | | | |
| | | placental | | | | | | | |
| DCC | EPHA6 | Alkaline | SCF | | | IgG4, | | CD248 | 0.68 |
| | | phosphatase, | | | | Kappa | | | |
| | | placental | | | | | | | |
| DCC | EPHA6 | Alkaline | SCF | | | IgG4, | RCAN3 | | 0.68 |
| | | phosphatase, | | | | Kappa | | | |
| | | placental | | | | | | | |
| DCC | EPHA6 | Alkaline | SCF | MASP3 | | | | | 0.68 |
| | | phosphatase, | | | | | | | |
| | | placental | | | | | | | |
| DCC | | Alkaline | SCF | | | IgG4, | | CD248 | 0.679 |
| | | phosphatase, | | | | Kappa | | | |
| | | placental | | | | | | | |
| DCC | | Alkaline | SCF | | DUS10 | IgG4, | | | 0.679 |
| | | phosphatase, | | | | Kappa | | | |
| | | placental | | | | | | | |
| DCC | | Alkaline | | MASP3 | | IgG4, | | CD248 | 0.678 |
| | | phosphatase, | | | | Kappa | | | |
| | | placental | | | | | | | |
| DCC | | Alkaline | | MASP3 | | IgG4, | RCAN3 | CD248 | 0.678 |
| | | phosphatase, | | | | Kappa | | | |
| | | placental | | | | | | | |
| DCC | | Alkaline | SCF | MASP3 | | | RCAN3 | | 0.678 |
| | | phosphatase, | | | | | | | |
| | | placental | | | | | | | |
| DCC | EPHA6 | Alkaline | | MASP3 | | IgG4, | | CD248 | 0.678 |
| | | phosphatase, | | | | Kappa | | | |
| | | placental | | | | | | | |
| DCC | EPHA6 | Alkaline | | MASP3 | | IgG4, | RCAN3 | CD248 | 0.678 |
| | | phosphatase, | | | | Kappa | | | |
| | | placental | | | | | | | |
| DCC | EPHA6 | Alkaline | SCF | | | IgG4, | | | 0.678 |
| | | phosphatase, | | | | Kappa | | | |
| | | placental | | | | | | | |
| DCC | | Alkaline | SCF | MASP3 | | | | | 0.677 |
| | | phosphatase, | | | | | | | |
| | | placental | | | | | | | |
| DCC | EPHA6 | Alkaline | | MASP3 | | IgG4, | | | 0.677 |
| | | phosphatase, | | | | Kappa | | | |
| | | placental | | | | | | | |
| DCC | EPHA6 | Alkaline | | MASP3 | | IgG4, | RCAN3 | | 0.677 |
| | | phosphatase, | | | | Kappa | | | |
| | | placental | | | | | | | |
| DCC | | Alkaline | | MASP3 | | IgG4, | RCAN3 | | 0.676 |
| | | phosphatase, | | | | Kappa | | | |
| | | placental | | | | | | | |
| DCC | | Alkaline | SCF | | | IgG4, | RCAN3 | | 0.676 |
| | | phosphatase, | | | | Kappa | | | |
| | | placental | | | | | | | |
| DCC | | Alkaline | | MASP3 | | IgG4, | | | 0.675 |
| | | phosphatase, | | | | Kappa | | | |
| | | placental | | | | | | | |
| DCC | | Alkaline | | MASP3 | DUS10 | | | CD248 | 0.675 |
| | | phosphatase, | | | | | | | |
| | | placental | | | | | | | |
| DCC | | Alkaline | | MASP3 | DUS10 | | RCAN3 | CD248 | 0.675 |
| | | phosphatase, | | | | | | | |
| | | placental | | | | | | | |
| DCC | EPHA6 | Alkaline | | MASP3 | DUS10 | | | CD248 | 0.674 |
| | | phosphatase, | | | | | | | |
| | | placental | | | | | | | |
| DCC | EPHA6 | Alkaline | | MASP3 | DUS10 | | RCAN3 | CD248 | 0.674 |
| | | phosphatase, | | | | | | | |
| | | placental | | | | | | | |
| DCC | EPHA6 | Alkaline | SCF | | DUS10 | | RCAN3 | CD248 | 0.674 |
| | | phosphatase, | | | | | | | |
| | | placental | | | | | | | |
| DCC | | Alkaline | SCF | | | IgG4, | | | 0.673 |
| | | phosphatase, | | | | Kappa | | | |
| | | placental | | | | | | | |
| DCC | EPHA6 | Alkaline | | MASP3 | DUS10 | | | | 0.673 |
| | | phosphatase, | | | | | | | |
| | | placental | | | | | | | |
| DCC | EPHA6 | Alkaline | | MASP3 | DUS10 | | RCAN3 | | 0.673 |
| | | phosphatase, | | | | | | | |
| | | placental | | | | | | | |
| DCC | | Alkaline | | MASP3 | DUS10 | | | | 0.672 |
| | | phosphatase, | | | | | | | |
| | | placental | | | | | | | |
| DCC | | Alkaline | | MASP3 | DUS10 | | RCAN3 | | 0.672 |
| | | phosphatase, | | | | | | | |
| | | placental | | | | | | | |
| DCC | | Alkaline | SCF | | DUS10 | | RCAN3 | CD248 | 0.672 |
| | | phosphatase, | | | | | | | |
| | | placental | | | | | | | |
| DCC | EPHA6 | Alkaline | SCF | | DUS10 | | | CD248 | 0.672 |
| | | phosphatase, | | | | | | | |
| | | placental | | | | | | | |
| DCC | EPHA6 | Alkaline | SCF | | DUS10 | | RCAN3 | | 0.672 |
| | | phosphatase, | | | | | | | |
| | | placental | | | | | | | |
| DCC | | Alkaline | SCF | | DUS10 | | | CD248 | 0.67 |
| | | phosphatase, | | | | | | | |
| | | placental | | | | | | | |
| DCC | EPHA6 | Alkaline | SCF | | DUS10 | | | | 0.669 |
| | | phosphatase, | | | | | | | |
| | | placental | | | | | | | |
| DCC | | Alkaline | | MASP3 | | | | CD248 | 0.668 |
| | | phosphatase, | | | | | | | |
| | | placental | | | | | | | |
| DCC | | Alkaline | | MASP3 | | | RCAN3 | CD248 | 0.668 |
| | | phosphatase, | | | | | | | |
| | | placental | | | | | | | |
| DCC | EPHA6 | Alkaline | | MASP3 | | | | CD248 | 0.668 |
| | | phosphatase, | | | | | | | |
| | | placental | | | | | | | |
| DCC | EPHA6 | Alkaline | | MASP3 | | | RCAN3 | CD248 | 0.668 |
| | | phosphatase, | | | | | | | |
| | | placental | | | | | | | |
| DCC | | Alkaline | | | DUS10 | IgG4, | | CD248 | 0.667 |
| | | phosphatase, | | | | Kappa | | | |
| | | placental | | | | | | | |
| DCC | | Alkaline | | | DUS10 | IgG4, | RCAN3 | CD248 | 0.667 |
| | | phosphatase, | | | | Kappa | | | |
| | | placental | | | | | | | |
| DCC | | Alkaline | SCF | | DUS10 | | RCAN3 | | 0.667 |
| | | phosphatase, | | | | | | | |
| | | placental | | | | | | | |
| DCC | EPHA6 | Alkaline | | | DUS10 | IgG4, | | CD248 | 0.667 |
| | | phosphatase, | | | | Kappa | | | |
| | | placental | | | | | | | |
| DCC | EPHA6 | Alkaline | | | DUS10 | IgG4, | RCAN3 | CD248 | 0.667 |
| | | phosphatase, | | | | Kappa | | | |
| | | placental | | | | | | | |
| DCC | EPHA6 | Alkaline | SCF | | | | RCAN3 | CD248 | 0.667 |
| | | phosphatase, | | | | | | | |
| | | placental | | | | | | | |
| DCC | | Alkaline | | MASP3 | | | | | 0.666 |
| | | phosphatase, | | | | | | | |
| | | placental | | | | | | | |
| DCC | | Alkaline | | MASP3 | | | RCAN3 | | 0.666 |
| | | phosphatase, | | | | | | | |
| | | placental | | | | | | | |
| DCC | EPHA6 | Alkaline | | | DUS10 | IgG4, | | | 0.666 |
| | | phosphatase, | | | | Kappa | | | |
| | | placental | | | | | | | |
| DCC | EPHA6 | Alkaline | | | DUS10 | IgG4, | RCAN3 | | 0.666 |
| | | phosphatase, | | | | Kappa | | | |
| | | placental | | | | | | | |
| DCC | EPHA6 | Alkaline | | MASP3 | | | | | 0.666 |
| | | phosphatase, | | | | | | | |
| | | placental | | | | | | | |
| DCC | EPHA6 | Alkaline | | MASP3 | | | RCAN3 | | 0.666 |
| | | phosphatase, | | | | | | | |
| | | placental | | | | | | | |
| DCC | | Alkaline | | | DUS10 | IgG4, | | | 0.665 |
| | | phosphatase, | | | | Kappa | | | |
| | | placental | | | | | | | |
| DCC | | Alkaline | | | DUS10 | IgG4, | RCAN3 | | 0.665 |
| | | phosphatase, | | | | Kappa | | | |
| | | placental | | | | | | | |
| DCC | | Alkaline | SCF | | | | RCAN3 | CD248 | 0.665 |
| | | phosphatase, | | | | | | | |
| | | placental | | | | | | | |
| DCC | EPHA6 | Alkaline | SCF | | | | RCAN3 | | 0.665 |
| | | phosphatase, | | | | | | | |
| | | placental | | | | | | | |
| DCC | | Alkaline | SCF | | DUS10 | | | | 0.664 |
| | | phosphatase, | | | | | | | |
| | | placental | | | | | | | |
| DCC | EPHA6 | Alkaline | SCF | | | | | CD248 | 0.664 |
| | | phosphatase, | | | | | | | |
| | | placental | | | | | | | |
| DCC | | Alkaline | SCF | | | | | CD248 | 0.662 |
| | | phosphatase, | | | | | | | |
| | | placental | | | | | | | |
| DCC | EPHA6 | Alkaline | SCF | | | | | | 0.662 |
| | | phosphatase, | | | | | | | |
| | | placental | | | | | | | |
| DCC | EPHA6 | Alkaline | | | | IgG4, | | CD248 | 0.661 |
| | | phosphatase, | | | | Kappa | | | |
| | | placental | | | | | | | |
| DCC | EPHA6 | Alkaline | | | | IgG4, | RCAN3 | CD248 | 0.661 |
| | | phosphatase, | | | | Kappa | | | |
| | | placental | | | | | | | |
| DCC | | Alkaline | | | | IgG4, | | CD248 | 0.66 |
| | | phosphatase, | | | | Kappa | | | |
| | | placental | | | | | | | |
| DCC | | Alkaline | | | | IgG4, | RCAN3 | CD248 | 0.66 |
| | | phosphatase, | | | | Kappa | | | |
| | | placental | | | | | | | |
| DCC | | Alkaline | SCF | | | | RCAN3 | | 0.66 |
| | | phosphatase, | | | | | | | |
| | | placental | | | | | | | |
| DCC | EPHA6 | Alkaline | | | | IgG4, | | | 0.659 |
| | | phosphatase, | | | | Kappa | | | |
| | | placental | | | | | | | |
| DCC | EPHA6 | Alkaline | | | | IgG4, | RCAN3 | | 0.659 |
| | | phosphatase, | | | | Kappa | | | |
| | | placental | | | | | | | |
| DCC | | Alkaline | | | | IgG4, | | | 0.657 |
| | | phosphatase, | | | | Kappa | | | |
| | | placental | | | | | | | |
| DCC | | Alkaline | | | | IgG4, | RCAN3 | | 0.657 |
| | | phosphatase, | | | | Kappa | | | |
| | | placental | | | | | | | |
| DCC | EPHA6 | Alkaline | | | DUS10 | | | CD248 | 0.656 |
| | | phosphatase, | | | | | | | |
| | | placental | | | | | | | |
| DCC | EPHA6 | Alkaline | | | DUS10 | | RCAN3 | CD248 | 0.656 |
| | | phosphatase, | | | | | | | |
| | | placental | | | | | | | |
| DCC | | Alkaline | | | DUS10 | | | CD248 | 0.655 |
| | | phosphatase, | | | | | | | |
| | | placental | | | | | | | |
| DCC | | Alkaline | | | DUS10 | | RCAN3 | CD248 | 0.655 |
| | | phosphatase, | | | | | | | |
| | | placental | | | | | | | |
| DCC | | Alkaline | SCF | | | | | | 0.655 |
| | | phosphatase, | | | | | | | |
| | | placental | | | | | | | |
| DCC | EPHA6 | Alkaline | | | DUS10 | | | | 0.655 |
| | | phosphatase, | | | | | | | |
| | | placental | | | | | | | |
| DCC | EPHA6 | Alkaline | | | DUS10 | | RCAN3 | | 0.655 |
| | | phosphatase, | | | | | | | |
| | | placental | | | | | | | |
| DCC | | Alkaline | | | DUS10 | | RCAN3 | | 0.653 |
| | | phosphatase, | | | | | | | |
| | | placental | | | | | | | |
| DCC | | Alkaline | | | DUS10 | | | | 0.652 |
| | | phosphatase, | | | | | | | |
| | | placental | | | | | | | |
| DCC | EPHA6 | Alkaline | | | | | RCAN3 | CD248 | 0.648 |
| | | phosphatase, | | | | | | | |
| | | placental | | | | | | | |
| DCC | | Alkaline | | | | | RCAN3 | CD248 | 0.647 |
| | | phosphatase, | | | | | | | |
| | | placental | | | | | | | |
| DCC | EPHA6 | Alkaline | | | | | | CD248 | 0.647 |
| | | phosphatase, | | | | | | | |
| | | placental | | | | | | | |
| DCC | EPHA6 | Alkaline | | | | | RCAN3 | | 0.647 |
| | | phosphatase, | | | | | | | |
| | | placental | | | | | | | |
| DCC | EPHA6 | Alkaline | | | | | | | 0.646 |
| | | phosphatase, | | | | | | | |
| | | placental | | | | | | | |
| DCC | | Alkaline | | | | | | CD248 | 0.645 |
| | | phosphatase, | | | | | | | |
| | | placental | | | | | | | |
| DCC | | Alkaline | | | | | RCAN3 | | 0.644 |
| | | phosphatase, | | | | | | | |
| | | placental | | | | | | | |
| DCC | | Alkaline | | | | | | | 0.643 |
| | | phosphatase, | | | | | | | |
| | | placental | | | | | | | |
| DCC | EPHA6 | | SCF | MASP3 | | IgG4, | | CD248 | 0.635 |
| | | | | | | Kappa | | | |
| DCC | EPHA6 | | SCF | MASP3 | | IgG4, | RCAN3 | CD248 | 0.635 |
| | | | | | | Kappa | | | |
| DCC | EPHA6 | | SCF | MASP3 | DUS10 | IgG4, | | | 0.634 |
| | | | | | | Kappa | | | |
| DCC | EPHA6 | | SCF | MASP3 | DUS10 | IgG4, | | CD248 | 0.634 |
| | | | | | | Kappa | | | |
| DCC | EPHA6 | | SCF | MASP3 | DUS10 | IgG4, | RCAN3 | | 0.634 |
| | | | | | | Kappa | | | |
| DCC | EPHA6 | | SCF | MASP3 | DUS10 | IgG4, | RCAN3 | CD248 | 0.634 |
| | | | | | | Kappa | | | |
| DCC | EPHA6 | | SCF | MASP3 | | IgG4, | | | 0.633 |
| | | | | | | Kappa | | | |
| DCC | EPHA6 | | SCF | MASP3 | | IgG4, | RCAN3 | | 0.633 |
| | | | | | | Kappa | | | |
| DCC | | | SCF | MASP3 | | IgG4, | | CD248 | 0.631 |
| | | | | | | Kappa | | | |
| DCC | | | SCF | MASP3 | | IgG4, | RCAN3 | CD248 | 0.631 |
| | | | | | | Kappa | | | |
| DCC | | | SCF | MASP3 | DUS10 | IgG4, | | CD248 | 0.631 |
| | | | | | | Kappa | | | |
| DCC | | | SCF | MASP3 | DUS10 | IgG4, | RCAN3 | CD248 | 0.63 |
| | | | | | | Kappa | | | |
| DCC | | | SCF | MASP3 | | IgG4, | | | 0.629 |
| | | | | | | Kappa | | | |
| DCC | | | SCF | MASP3 | | IgG4, | RCAN3 | | 0.629 |
| | | | | | | Kappa | | | |
| DCC | | | SCF | MASP3 | DUS10 | IgG4, | | | 0.629 |
| | | | | | | Kappa | | | |
| DCC | | | SCF | MASP3 | DUS10 | IgG4, | RCAN3 | | 0.629 |
| | | | | | | Kappa | | | |
| DCC | EPHA6 | | SCF | | | IgG4, | | CD248 | 0.629 |
| | | | | | | Kappa | | | |
| DCC | EPHA6 | | SCF | | | IgG4, | RCAN3 | | 0.629 |
| | | | | | | Kappa | | | |
| DCC | EPHA6 | | SCF | | | IgG4, | RCAN3 | CD248 | 0.629 |
| | | | | | | Kappa | | | |
| DCC | EPHA6 | | SCF | | DUS10 | IgG4, | | | 0.629 |
| | | | | | | Kappa | | | |
| DCC | EPHA6 | | SCF | | DUS10 | IgG4, | | CD248 | 0.629 |
| | | | | | | Kappa | | | |
| DCC | EPHA6 | | SCF | | DUS10 | IgG4, | RCAN3 | | 0.629 |
| | | | | | | Kappa | | | |
| DCC | EPHA6 | | SCF | | DUS10 | IgG4, | RCAN3 | CD248 | 0.629 |
| | | | | | | Kappa | | | |
| DCC | EPHA6 | | SCF | | | IgG4, | | | 0.628 |
| | | | | | | Kappa | | | |
| DCC | | | SCF | | DUS10 | IgG4, | | CD248 | 0.627 |
| | | | | | | Kappa | | | |
| DCC | | | SCF | | DUS10 | IgG4, | RCAN3 | CD248 | 0.627 |
| | | | | | | Kappa | | | |
| DCC | | | SCF | | | IgG4, | | CD248 | 0.626 |
| | | | | | | Kappa | | | |
| DCC | | | SCF | | | IgG4, | RCAN3 | CD248 | 0.626 |
| | | | | | | Kappa | | | |
| DCC | | | SCF | | DUS10 | IgG4, | | | 0.623 |
| | | | | | | Kappa | | | |
| DCC | | | SCF | | DUS10 | IgG4, | RCAN3 | | 0.623 |
| | | | | | | Kappa | | | |
| DCC | | | SCF | | | IgG4, | RCAN3 | | 0.622 |
| | | | | | | Kappa | | | |
| DCC | | | SCF | | | IgG4, | | | 0.621 |
| | | | | | | Kappa | | | |
| DCC | EPHA6 | | SCF | MASP3 | | | | CD248 | 0.615 |
| DCC | EPHA6 | | SCF | MASP3 | | | RCAN3 | CD248 | 0.615 |
| DCC | EPHA6 | | SCF | MASP3 | DUS10 | | | | 0.615 |
| DCC | EPHA6 | | SCF | MASP3 | DUS10 | | | CD248 | 0.615 |
| DCC | EPHA6 | | SCF | MASP3 | DUS10 | | RCAN3 | | 0.615 |
| DCC | EPHA6 | | SCF | MASP3 | DUS10 | | RCAN3 | CD248 | 0.615 |
| DCC | EPHA6 | | SCF | MASP3 | | | | | 0.614 |
| DCC | EPHA6 | | SCF | MASP3 | | | RCAN3 | | 0.614 |
| DCC | EPHA6 | | SCF | | DUS10 | | RCAN3 | CD248 | 0.613 |
| DCC | EPHA6 | | SCF | | DUS10 | | | | 0.612 |
| DCC | EPHA6 | | SCF | | DUS10 | | | CD248 | 0.612 |
| DCC | EPHA6 | | SCF | | DUS10 | | RCAN3 | | 0.612 |
| DCC | | | SCF | MASP3 | DUS10 | | | CD248 | 0.611 |
| DCC | | | SCF | MASP3 | DUS10 | | RCAN3 | CD248 | 0.611 |
| DCC | EPHA6 | | SCF | | | | RCAN3 | CD248 | 0.611 |
| DCC | | | SCF | MASP3 | | | | CD248 | 0.61 |
| DCC | | | SCF | MASP3 | | | RCAN3 | CD248 | 0.61 |
| DCC | EPHA6 | | SCF | | | | | CD248 | 0.61 |
| DCC | EPHA6 | | SCF | | | | | | 0.609 |
| DCC | EPHA6 | | SCF | | | | RCAN3 | | 0.609 |
| DCC | | | SCF | MASP3 | DUS10 | | | | 0.607 |
| DCC | | | SCF | MASP3 | DUS10 | | RCAN3 | | 0.607 |
| DCC | | | SCF | MASP3 | | | | | 0.606 |
| DCC | | | SCF | MASP3 | | | RCAN3 | | 0.606 |
| DCC | | | SCF | | DUS10 | | | CD248 | 0.605 |
| DCC | | | SCF | | DUS10 | | RCAN3 | CD248 | 0.605 |
| DCC | | | SCF | | | | RCAN3 | CD248 | 0.603 |
| DCC | | | SCF | | | | | CD248 | 0.602 |
| DCC | | | SCF | | DUS10 | | | | 0.602 |
| DCC | | | SCF | | DUS10 | | RCAN3 | | 0.602 |
| DCC | EPHA6 | | | MASP3 | DUS10 | IgG4, | | | 0.602 |
| | | | | | | Kappa | | | |
| DCC | EPHA6 | | | MASP3 | DUS10 | IgG4, | | CD248 | 0.602 |
| | | | | | | Kappa | | | |
| DCC | EPHA6 | | | MASP3 | DUS10 | IgG4, | RCAN3 | CD248 | 0.602 |
| | | | | | | Kappa | | | |
| DCC | | | | MASP3 | DUS10 | IgG4, | | | 0.601 |
| | | | | | | Kappa | | | |
| DCC | | | | MASP3 | DUS10 | IgG4, | | CD248 | 0.601 |
| | | | | | | Kappa | | | |
| DCC | | | | MASP3 | DUS10 | IgG4, | RCAN3 | CD248 | 0.601 |
| | | | | | | Kappa | | | |
| DCC | EPHA6 | | | MASP3 | DUS10 | IgG4, | RCAN3 | | 0.601 |
| | | | | | | Kappa | | | |
| DCC | | | | MASP3 | DUS10 | IgG4, | RCAN3 | | 0.6 |
| | | | | | | Kappa | | | |
| DCC | EPHA6 | | | MASP3 | | IgG4, | | CD248 | 0.6 |
| | | | | | | Kappa | | | |
| DCC | EPHA6 | | | MASP3 | | IgG4, | RCAN3 | CD248 | 0.6 |
| | | | | | | Kappa | | | |

TABLE 22
RCAN3 Biomarker Panels

| | | Alkaline | | | | | | | |
|---|---|---|---|---|---|---|---|---|---|
| | | phosphatase, | | | | IgG4, | | | |
| RCAN3 | EPHA6 | placental | SCF | MASP3 | DUS10 | Kappa | DCC | CD248 | AUC |
| RCAN3 | EPHA6 | Alkaline | SCF | MASP3 | DUS10 | IgG4, | DCC | CD248 | 0.698 |
| | | phosphatase, | | | | Kappa | | | |
| | | placental | | | | | | | |
| RCAN3 | | Alkaline | SCF | MASP3 | DUS10 | IgG4, | DCC | CD248 | 0.697 |
| | | phosphatase, | | | | Kappa | | | |
| | | placental | | | | | | | |
| RCAN3 | | Alkaline | SCF | MASP3 | DUS10 | IgG4, | | CD248 | 0.696 |
| | | phosphatase, | | | | Kappa | | | |
| | | placental | | | | | | | |
| RCAN3 | EPHA6 | Alkaline | SCF | MASP3 | DUS10 | IgG4, | | CD248 | 0.696 |
| | | phosphatase, | | | | Kappa | | | |
| | | placental | | | | | | | |
| RCAN3 | EPHA6 | Alkaline | SCF | MASP3 | DUS10 | IgG4, | DCC | | 0.696 |
| | | phosphatase, | | | | Kappa | | | |
| | | placental | | | | | | | |
| RCAN3 | EPHA6 | Alkaline | SCF | MASP3 | DUS10 | IgG4, | | | 0.695 |
| | | phosphatase, | | | | Kappa | | | |
| | | placental | | | | | | | |
| RCAN3 | | Alkaline | SCF | MASP3 | | IgG4, | DCC | CD248 | 0.694 |
| | | phosphatase, | | | | Kappa | | | |
| | | placental | | | | | | | |
| RCAN3 | | Alkaline | SCF | MASP3 | DUS10 | IgG4, | | | 0.694 |
| | | phosphatase, | | | | Kappa | | | |
| | | placental | | | | | | | |
| RCAN3 | | Alkaline | SCF | MASP3 | DUS10 | IgG4, | DCC | | 0.694 |
| | | phosphatase, | | | | Kappa | | | |
| | | placental | | | | | | | |
| RCAN3 | EPHA6 | Alkaline | SCF | MASP3 | | IgG4, | DCC | CD248 | 0.694 |
| | | phosphatase, | | | | Kappa | | | |
| | | placental | | | | | | | |
| RCAN3 | | Alkaline | SCF | MASP3 | | IgG4, | | CD248 | 0.693 |
| | | phosphatase, | | | | Kappa | | | |
| | | placental | | | | | | | |
| RCAN3 | EPHA6 | Alkaline | SCF | MASP3 | | IgG4, | | CD248 | 0.693 |
| | | phosphatase, | | | | Kappa | | | |
| | | placental | | | | | | | |
| RCAN3 | EPHA6 | Alkaline | SCF | MASP3 | | IgG4, | | | 0.692 |
| | | phosphatase, | | | | Kappa | | | |
| | | placental | | | | | | | |
| RCAN3 | EPHA6 | Alkaline | SCF | MASP3 | | IgG4, | DCC | | 0.692 |
| | | phosphatase, | | | | Kappa | | | |
| | | placental | | | | | | | |
| RCAN3 | | Alkaline | SCF | MASP3 | | IgG4, | | | 0.691 |
| | | phosphatase, | | | | Kappa | | | |
| | | placental | | | | | | | |
| RCAN3 | | Alkaline | SCF | MASP3 | | IgG4, | DCC | | 0.691 |
| | | phosphatase, | | | | Kappa | | | |
| | | placental | | | | | | | |
| RCAN3 | EPHA6 | Alkaline | SCF | MASP3 | DUS10 | | DCC | CD248 | 0.689 |
| | | phosphatase, | | | | | | | |
| | | placental | | | | | | | |
| RCAN3 | | Alkaline | SCF | | DUS10 | IgG4, | DCC | CD248 | 0.687 |
| | | phosphatase, | | | | Kappa | | | |
| | | placental | | | | | | | |
| RCAN3 | | Alkaline | SCF | MASP3 | DUS10 | | DCC | CD248 | 0.687 |
| | | phosphatase, | | | | | | | |
| | | placental | | | | | | | |
| RCAN3 | EPHA6 | Alkaline | SCF | | DUS10 | IgG4, | DCC | CD248 | 0.687 |
| | | phosphatase, | | | | Kappa | | | |
| | | placental | | | | | | | |
| RCAN3 | EPHA6 | Alkaline | SCF | MASP3 | DUS10 | | DCC | | 0.686 |
| | | phosphatase, | | | | | | | |
| | | placental | | | | | | | |
| RCAN3 | | Alkaline | SCF | MASP3 | DUS10 | | | CD248 | 0.685 |
| | | phosphatase, | | | | | | | |
| | | placental | | | | | | | |
| RCAN3 | EPHA6 | Alkaline | SCF | MASP3 | DUS10 | | | CD248 | 0.685 |
| | | phosphatase, | | | | | | | |
| | | placental | | | | | | | |
| RCAN3 | EPHA6 | Alkaline | SCF | | DUS10 | IgG4, | DCC | | 0.684 |
| | | phosphatase, | | | | Kappa | | | |
| | | placental | | | | | | | |
| RCAN3 | EPHA6 | Alkaline | SCF | MASP3 | | | DCC | CD248 | 0.684 |
| | | phosphatase, | | | | | | | |
| | | placental | | | | | | | |
| RCAN3 | | Alkaline | SCF | MASP3 | | | DCC | CD248 | 0.683 |
| | | phosphatase, | | | | | | | |
| | | placental | | | | | | | |
| RCAN3 | | Alkaline | SCF | MASP3 | DUS10 | | | | 0.683 |
| | | phosphatase, | | | | | | | |
| | | placental | | | | | | | |
| RCAN3 | | Alkaline | SCF | MASP3 | DUS10 | | DCC | | 0.683 |
| | | phosphatase, | | | | | | | |
| | | placental | | | | | | | |
| RCAN3 | EPHA6 | Alkaline | SCF | MASP3 | DUS10 | | | | 0.683 |
| | | phosphatase, | | | | | | | |
| | | placental | | | | | | | |
| RCAN3 | | Alkaline | | MASP3 | DUS10 | IgG4, | DCC | CD248 | 0.682 |
| | | phosphatase, | | | | Kappa | | | |
| | | placental | | | | | | | |
| RCAN3 | EPHA6 | Alkaline | | MASP3 | DUS10 | IgG4, | DCC | CD248 | 0.682 |
| | | phosphatase, | | | | Kappa | | | |
| | | placental | | | | | | | |
| RCAN3 | EPHA6 | Alkaline | SCF | | | IgG4, | DCC | CD248 | 0.682 |
| | | phosphatase, | | | | Kappa | | | |
| | | placental | | | | | | | |
| RCAN3 | | Alkaline | SCF | | | IgG4, | DCC | CD248 | 0.681 |
| | | phosphatase, | | | | Kappa | | | |
| | | placental | | | | | | | |
| RCAN3 | | Alkaline | SCF | | DUS10 | IgG4, | | CD248 | 0.681 |
| | | phosphatase, | | | | Kappa | | | |
| | | placental | | | | | | | |
| RCAN3 | | Alkaline | SCF | | DUS10 | IgG4, | DCC | | 0.681 |
| | | phosphatase, | | | | Kappa | | | |
| | | placental | | | | | | | |
| RCAN3 | | Alkaline | SCF | MASP3 | | | | CD248 | 0.681 |
| | | phosphatase, | | | | | | | |
| | | placental | | | | | | | |
| RCAN3 | EPHA6 | Alkaline | | MASP3 | DUS10 | IgG4, | | CD248 | 0.681 |
| | | phosphatase, | | | | Kappa | | | |
| | | placental | | | | | | | |
| RCAN3 | EPHA6 | Alkaline | SCF | | DUS10 | IgG4, | | CD248 | 0.681 |
| | | phosphatase, | | | | Kappa | | | |
| | | placental | | | | | | | |
| RCAN3 | EPHA6 | Alkaline | SCF | MASP3 | | | | CD248 | 0.681 |
| | | phosphatase, | | | | | | | |
| | | placental | | | | | | | |
| RCAN3 | EPHA6 | Alkaline | SCF | MASP3 | | | DCC | | 0.681 |
| | | phosphatase, | | | | | | | |
| | | placental | | | | | | | |
| RCAN3 | | Alkaline | | MASP3 | DUS10 | IgG4, | | | 0.68 |
| | | phosphatase, | | | | Kappa | | | |
| | | placental | | | | | | | |
| RCAN3 | | Alkaline | | MASP3 | DUS10 | IgG4, | | CD248 | 0.68 |
| | | phosphatase, | | | | Kappa | | | |
| | | placental | | | | | | | |
| RCAN3 | | Alkaline | | MASP3 | DUS10 | IgG4, | DCC | | 0.68 |
| | | phosphatase, | | | | Kappa | | | |
| | | placental | | | | | | | |
| RCAN3 | EPHA6 | Alkaline | | MASP3 | DUS10 | IgG4, | | | 0.68 |
| | | phosphatase, | | | | Kappa | | | |
| | | placental | | | | | | | |
| RCAN3 | EPHA6 | Alkaline | | MASP3 | DUS10 | IgG4, | DCC | | 0.68 |
| | | phosphatase, | | | | Kappa | | | |
| | | placental | | | | | | | |
| RCAN3 | EPHA6 | Alkaline | SCF | | | IgG4, | DCC | | 0.68 |
| | | phosphatase, | | | | Kappa | | | |
| | | placental | | | | | | | |
| RCAN3 | EPHA6 | Alkaline | SCF | | DUS10 | IgG4, | | | 0.68 |
| | | phosphatase, | | | | Kappa | | | |
| | | placental | | | | | | | |
| RCAN3 | EPHA6 | Alkaline | SCF | MASP3 | | | | | 0.68 |
| | | phosphatase, | | | | | | | |
| | | placental | | | | | | | |
| RCAN3 | | Alkaline | SCF | | DUS10 | IgG4, | | | 0.679 |
| | | phosphatase, | | | | Kappa | | | |
| | | placental | | | | | | | |
| RCAN3 | | Alkaline | SCF | MASP3 | | | | | 0.679 |
| | | phosphatase, | | | | | | | |
| | | placental | | | | | | | |
| RCAN3 | | Alkaline | | MASP3 | | IgG4, | DCC | CD248 | 0.678 |
| | | phosphatase, | | | | Kappa | | | |
| | | placental | | | | | | | |
| RCAN3 | | Alkaline | SCF | MASP3 | | | DCC | | 0.678 |
| | | phosphatase, | | | | | | | |
| | | placental | | | | | | | |
| RCAN3 | EPHA6 | Alkaline | | MASP3 | | IgG4, | DCC | CD248 | 0.678 |
| | | phosphatase, | | | | Kappa | | | |
| | | placental | | | | | | | |
| RCAN3 | EPHA6 | Alkaline | | MASP3 | | IgG4, | DCC | | 0.677 |
| | | phosphatase, | | | | Kappa | | | |
| | | placental | | | | | | | |
| RCAN3 | | Alkaline | | MASP3 | | IgG4, | | | 0.676 |
| | | phosphatase, | | | | Kappa | | | |
| | | placental | | | | | | | |
| RCAN3 | | Alkaline | | MASP3 | | IgG4, | DCC | | 0.676 |
| | | phosphatase, | | | | Kappa | | | |
| | | placental | | | | | | | |
| RCAN3 | | Alkaline | SCF | | | IgG4, | | CD248 | 0.676 |
| | | phosphatase, | | | | Kappa | | | |
| | | placental | | | | | | | |
| RCAN3 | | Alkaline | SCF | | | IgG4, | DCC | | 0.676 |
| | | phosphatase, | | | | Kappa | | | |
| | | placental | | | | | | | |
| RCAN3 | EPHA6 | Alkaline | SCF | | | IgG4, | | CD248 | 0.676 |
| | | phosphatase, | | | | Kappa | | | |
| | | placental | | | | | | | |
| RCAN3 | | Alkaline | | MASP3 | | IgG4, | | CD248 | 0.675 |
| | | phosphatase, | | | | Kappa | | | |
| | | placental | | | | | | | |
| RCAN3 | | Alkaline | | MASP3 | DUS10 | | DCC | CD248 | 0.675 |
| | | phosphatase, | | | | | | | |
| | | placental | | | | | | | |
| RCAN3 | EPHA6 | Alkaline | | MASP3 | | IgG4, | | | 0.675 |
| | | phosphatase, | | | | Kappa | | | |
| | | placental | | | | | | | |
| RCAN3 | EPHA6 | Alkaline | | MASP3 | | IgG4, | | CD248 | 0.675 |
| | | phosphatase, | | | | Kappa | | | |
| | | placental | | | | | | | |
| RCAN3 | | Alkaline | SCF | | | IgG4, | | | 0.674 |
| | | phosphatase, | | | | Kappa | | | |
| | | placental | | | | | | | |
| RCAN3 | EPHA6 | Alkaline | | MASP3 | DUS10 | | DCC | CD248 | 0.674 |
| | | phosphatase, | | | | | | | |
| | | placental | | | | | | | |
| RCAN3 | EPHA6 | Alkaline | SCF | | | IgG4, | | | 0.674 |
| | | phosphatase, | | | | Kappa | | | |
| | | placental | | | | | | | |
| RCAN3 | EPHA6 | Alkaline | SCF | | DUS10 | | DCC | CD248 | 0.674 |
| | | phosphatase, | | | | | | | |
| | | placental | | | | | | | |
| RCAN3 | EPHA6 | Alkaline | | MASP3 | DUS10 | | DCC | | 0.673 |
| | | phosphatase, | | | | | | | |
| | | placental | | | | | | | |
| RCAN3 | | Alkaline | | MASP3 | DUS10 | | DCC | | 0.672 |
| | | phosphatase, | | | | | | | |
| | | placental | | | | | | | |
| RCAN3 | | Alkaline | SCF | | DUS10 | | DCC | CD248 | 0.672 |
| | | phosphatase, | | | | | | | |
| | | placental | | | | | | | |
| RCAN3 | EPHA6 | Alkaline | SCF | | DUS10 | | DCC | | 0.672 |
| | | phosphatase, | | | | | | | |
| | | placental | | | | | | | |
| RCAN3 | | Alkaline | | MASP3 | DUS10 | | | | 0.671 |
| | | phosphatase, | | | | | | | |
| | | placental | | | | | | | |
| RCAN3 | | Alkaline | | MASP3 | DUS10 | | | CD248 | 0.671 |
| | | phosphatase, | | | | | | | |
| | | placental | | | | | | | |
| RCAN3 | EPHA6 | Alkaline | | MASP3 | DUS10 | | | CD248 | 0.671 |
| | | phosphatase, | | | | | | | |
| | | placental | | | | | | | |
| RCAN3 | EPHA6 | Alkaline | | MASP3 | DUS10 | | | | 0.67 |
| | | phosphatase, | | | | | | | |
| | | placental | | | | | | | |
| RCAN3 | | Alkaline | | MASP3 | | | DCC | CD248 | 0.668 |
| | | phosphatase, | | | | | | | |
| | | placental | | | | | | | |
| RCAN3 | EPHA6 | Alkaline | | MASP3 | | | DCC | CD248 | 0.668 |
| | | phosphatase, | | | | | | | |
| | | placental | | | | | | | |
| RCAN3 | | Alkaline | | | DUS10 | IgG4, | DCC | CD248 | 0.667 |
| | | phosphatase, | | | | Kappa | | | |
| | | placental | | | | | | | |
| RCAN3 | | Alkaline | SCF | | DUS10 | | DCC | | 0.667 |
| | | phosphatase, | | | | | | | |
| | | placental | | | | | | | |
| RCAN3 | EPHA6 | Alkaline | | | DUS10 | IgG4, | DCC | CD248 | 0.667 |
| | | phosphatase, | | | | Kappa | | | |
| | | placental | | | | | | | |
| RCAN3 | EPHA6 | Alkaline | SCF | | | | DCC | CD248 | 0.667 |
| | | phosphatase, | | | | | | | |
| | | placental | | | | | | | |
| RCAN3 | | Alkaline | | MASP3 | | | DCC | | 0.666 |
| | | phosphatase, | | | | | | | |
| | | placental | | | | | | | |
| RCAN3 | EPHA6 | Alkaline | | | DUS10 | IgG4, | DCC | | 0.666 |
| | | phosphatase, | | | | Kappa | | | |
| | | placental | | | | | | | |
| RCAN3 | EPHA6 | Alkaline | | MASP3 | | | DCC | | 0.666 |
| | | phosphatase, | | | | | | | |
| | | placental | | | | | | | |
| RCAN3 | | Alkaline | | | DUS10 | IgG4, | DCC | | 0.665 |
| | | phosphatase, | | | | Kappa | | | |
| | | placental | | | | | | | |
| RCAN3 | | Alkaline | SCF | | | | DCC | CD248 | 0.665 |
| | | phosphatase, | | | | | | | |
| | | placental | | | | | | | |
| RCAN3 | | Alkaline | SCF | | DUS10 | | | CD248 | 0.665 |
| | | phosphatase, | | | | | | | |
| | | placental | | | | | | | |
| RCAN3 | EPHA6 | Alkaline | SCF | | | | DCC | | 0.665 |
| | | phosphatase, | | | | | | | |
| | | placental | | | | | | | |
| RCAN3 | EPHA6 | Alkaline | SCF | | DUS10 | | | CD248 | 0.665 |
| | | phosphatase, | | | | | | | |
| | | placental | | | | | | | |
| RCAN3 | | Alkaline | | MASP3 | | | | | 0.664 |
| | | phosphatase, | | | | | | | |
| | | placental | | | | | | | |
| RCAN3 | | Alkaline | | MASP3 | | | | CD248 | 0.664 |
| | | phosphatase, | | | | | | | |
| | | placental | | | | | | | |
| RCAN3 | EPHA6 | Alkaline | | MASP3 | | | | | 0.664 |
| | | phosphatase, | | | | | | | |
| | | placental | | | | | | | |
| RCAN3 | EPHA6 | Alkaline | | MASP3 | | | | CD248 | 0.664 |
| | | phosphatase, | | | | | | | |
| | | placental | | | | | | | |
| RCAN3 | EPHA6 | Alkaline | SCF | | DUS10 | | | | 0.664 |
| | | phosphatase, | | | | | | | |
| | | placental | | | | | | | |
| RCAN3 | | Alkaline | SCF | | DUS10 | | | | 0.663 |
| | | phosphatase, | | | | | | | |
| | | placental | | | | | | | |
| RCAN3 | | Alkaline | | | DUS10 | IgG4, | | | 0.661 |
| | | phosphatase, | | | | Kappa | | | |
| | | placental | | | | | | | |
| RCAN3 | EPHA6 | Alkaline | | | | IgG4, | DCC | CD248 | 0.661 |
| | | phosphatase, | | | | Kappa | | | |
| | | placental | | | | | | | |
| RCAN3 | | Alkaline | | | | IgG4, | DCC | CD248 | 0.66 |
| | | phosphatase, | | | | Kappa | | | |
| | | placental | | | | | | | |
| RCAN3 | | Alkaline | | | DUS10 | IgG4, | | CD248 | 0.66 |
| | | phosphatase, | | | | Kappa | | | |
| | | placental | | | | | | | |
| RCAN3 | | Alkaline | SCF | | | | DCC | | 0.66 |
| | | phosphatase, | | | | | | | |
| | | placental | | | | | | | |
| RCAN3 | EPHA6 | Alkaline | | | DUS10 | IgG4, | | | 0.66 |
| | | phosphatase, | | | | Kappa | | | |
| | | placental | | | | | | | |
| RCAN3 | EPHA6 | Alkaline | | | DUS10 | IgG4, | | CD248 | 0.66 |
| | | phosphatase, | | | | Kappa | | | |
| | | placental | | | | | | | |
| RCAN3 | EPHA6 | Alkaline | | | | IgG4, | DCC | | 0.659 |
| | | phosphatase, | | | | Kappa | | | |
| | | placental | | | | | | | |
| RCAN3 | | Alkaline | SCF | | | | | CD248 | 0.658 |
| | | phosphatase, | | | | | | | |
| | | placental | | | | | | | |
| RCAN3 | EPHA6 | Alkaline | SCF | | | | | | 0.658 |
| | | phosphatase, | | | | | | | |
| | | placental | | | | | | | |
| RCAN3 | EPHA6 | Alkaline | SCF | | | | | CD248 | 0.658 |
| | | phosphatase, | | | | | | | |
| | | placental | | | | | | | |
| RCAN3 | | Alkaline | | | | IgG4, | DCC | | 0.657 |
| | | phosphatase, | | | | Kappa | | | |
| | | placental | | | | | | | |
| RCAN3 | | Alkaline | SCF | | | | | | 0.656 |
| | | phosphatase, | | | | | | | |
| | | placental | | | | | | | |
| RCAN3 | EPHA6 | Alkaline | | | DUS10 | | DCC | CD248 | 0.656 |
| | | phosphatase, | | | | | | | |
| | | placental | | | | | | | |
| RCAN3 | | Alkaline | | | DUS10 | | DCC | CD248 | 0.655 |
| | | phosphatase, | | | | | | | |
| | | placental | | | | | | | |
| RCAN3 | EPHA6 | Alkaline | | | DUS10 | | DCC | | 0.655 |
| | | phosphatase, | | | | | | | |
| | | placental | | | | | | | |
| RCAN3 | | Alkaline | | | | IgG4, | | | 0.654 |
| | | phosphatase, | | | | Kappa | | | |
| | | placental | | | | | | | |
| RCAN3 | | Alkaline | | | DUS10 | | DCC | | 0.653 |
| | | phosphatase, | | | | | | | |
| | | placental | | | | | | | |
| RCAN3 | EPHA6 | Alkaline | | | | IgG4, | | | 0.652 |
| | | phosphatase, | | | | Kappa | | | |
| | | placental | | | | | | | |
| RCAN3 | EPHA6 | Alkaline | | | | IgG4, | | CD248 | 0.652 |
| | | phosphatase, | | | | Kappa | | | |
| | | placental | | | | | | | |
| RCAN3 | | Alkaline | | | | IgG4, | | CD248 | 0.651 |
| | | phosphatase, | | | | Kappa | | | |
| | | placental | | | | | | | |
| RCAN3 | | Alkaline | | | DUS10 | | | | 0.648 |
| | | phosphatase, | | | | | | | |
| | | placental | | | | | | | |
| RCAN3 | EPHA6 | Alkaline | | | | | DCC | CD248 | 0.648 |
| | | phosphatase, | | | | | | | |
| | | placental | | | | | | | |
| RCAN3 | | Alkaline | | | | | DCC | CD248 | 0.647 |
| | | phosphatase, | | | | | | | |
| | | placental | | | | | | | |
| RCAN3 | EPHA6 | Alkaline | | | | | DCC | | 0.647 |
| | | phosphatase, | | | | | | | |
| | | placental | | | | | | | |
| RCAN3 | | Alkaline | | | DUS10 | | | CD248 | 0.646 |
| | | phosphatase, | | | | | | | |
| | | placental | | | | | | | |
| RCAN3 | EPHA6 | Alkaline | | | DUS10 | | | | 0.646 |
| | | phosphatase, | | | | | | | |
| | | placental | | | | | | | |
| RCAN3 | EPHA6 | Alkaline | | | DUS10 | | | CD248 | 0.645 |
| | | phosphatase, | | | | | | | |
| | | placental | | | | | | | |
| RCAN3 | | Alkaline | | | | | DCC | | 0.644 |
| | | phosphatase, | | | | | | | |
| | | placental | | | | | | | |
| RCAN3 | EPHA6 | | SCF | MASP3 | | IgG4, | DCC | CD248 | 0.635 |
| | | | | | | Kappa | | | |
| RCAN3 | EPHA6 | | SCF | MASP3 | DUS10 | IgG4, | DCC | | 0.634 |
| | | | | | | Kappa | | | |
| RCAN3 | EPHA6 | | SCF | MASP3 | DUS10 | IgG4, | DCC | CD248 | 0.634 |
| | | | | | | Kappa | | | |
| RCAN3 | | Alkaline | | | | | | | 0.633 |
| | | phosphatase, | | | | | | | |
| | | placental | | | | | | | |
| RCAN3 | EPHA6 | | SCF | MASP3 | | IgG4, | DCC | | 0.633 |
| | | | | | | Kappa | | | |
| RCAN3 | | | SCF | MASP3 | | IgG4, | DCC | CD248 | 0.631 |
| | | | | | | Kappa | | | |
| RCAN3 | | Alkaline | | | | | | CD248 | 0.631 |
| | | phosphatase, | | | | | | | |
| | | placental | | | | | | | |
| RCAN3 | EPHA6 | Alkaline | | | | | | | 0.631 |
| | | phosphatase, | | | | | | | |
| | | placental | | | | | | | |
| RCAN3 | | | SCF | MASP3 | DUS10 | IgG4, | DCC | CD248 | 0.63 |
| | | | | | | Kappa | | | |
| RCAN3 | EPHA6 | Alkaline | | | | | | CD248 | 0.63 |
| | | phosphatase, | | | | | | | |
| | | placental | | | | | | | |
| RCAN3 | | | SCF | MASP3 | | IgG4, | DCC | | 0.629 |
| | | | | | | Kappa | | | |
| RCAN3 | | | SCF | MASP3 | DUS10 | IgG4, | DCC | | 0.629 |
| | | | | | | Kappa | | | |
| RCAN3 | EPHA6 | | SCF | | | IgG4, | DCC | | 0.629 |
| | | | | | | Kappa | | | |
| RCAN3 | EPHA6 | | SCF | | | lgG4, | DCC | CD248 | 0.629 |
| | | | | | | Kappa | | | |
| RCAN3 | EPHA6 | | SCF | | DUS10 | IgG4, | DCC | | 0.629 |
| | | | | | | Kappa | | | |
| RCAN3 | EPHA6 | | SCF | | DUS10 | IgG4, | DCC | CD248 | 0.629 |
| | | | | | | Kappa | | | |
| RCAN3 | | | SCF | | DUS10 | IgG4, | DCC | CD248 | 0.627 |
| | | | | | | Kappa | | | |
| RCAN3 | | | SCF | MASP3 | DUS10 | IgG4, | | CD248 | 0.627 |
| | | | | | | Kappa | | | |
| RCAN3 | EPHA6 | | SCF | MASP3 | DUS10 | IgG4, | | | 0.627 |
| | | | | | | Kappa | | | |
| RCAN3 | EPHA6 | | SCF | MASP3 | DUS10 | IgG4, | | CD248 | 0.627 |
| | | | | | | Kappa | | | |
| RCAN3 | | | SCF | | | IgG4, | DCC | CD248 | 0.626 |
| | | | | | | Kappa | | | |
| RCAN3 | | | SCF | MASP3 | | IgG4, | | CD248 | 0.626 |
| | | | | | | Kappa | | | |
| RCAN3 | | | SCF | MASP3 | DUS10 | IgG4, | | | 0.626 |
| | | | | | | Kappa | | | |
| RCAN3 | EPHA6 | | SCF | MASP3 | | IgG4, | | CD248 | 0.626 |
| | | | | | | Kappa | | | |
| RCAN3 | | | SCF | MASP3 | | IgG4, | | | 0.625 |
| | | | | | | Kappa | | | |
| RCAN3 | EPHA6 | | SCF | MASP3 | | IgG4, | | | 0.625 |
| | | | | | | Kappa | | | |
| RCAN3 | | | SCF | | DUS10 | IgG4, | DCC | | 0.623 |
| | | | | | | Kappa | | | |
| RCAN3 | | | SCF | | | IgG4, | DCC | | 0.622 |
| | | | | | | Kappa | | | |
| RCAN3 | | | SCF | | DUS10 | IgG4, | | CD248 | 0.618 |
| | | | | | | Kappa | | | |
| RCAN3 | EPHA6 | | SCF | | DUS10 | IgG4, | | | 0.618 |
| | | | | | | Kappa | | | |
| RCAN3 | EPHA6 | | SCF | | DUS10 | IgG4, | | CD248 | 0.618 |
| | | | | | | Kappa | | | |
| RCAN3 | | | SCF | | | IgG4, | | CD248 | 0.616 |
| | | | | | | Kappa | | | |
| RCAN3 | | | SCF | | DUS10 | IgG4, | | | 0.616 |
| | | | | | | Kappa | | | |
| RCAN3 | EPHA6 | | SCF | | | IgG4, | | | 0.616 |
| | | | | | | Kappa | | | |
| RCAN3 | EPHA6 | | SCF | | | IgG4, | | CD248 | 0.616 |
| | | | | | | Kappa | | | |
| RCAN3 | | | SCF | | | IgG4, | | | 0.615 |
| | | | | | | Kappa | | | |
| RCAN3 | EPHA6 | | SCF | MASP3 | | | DCC | CD248 | 0.615 |
| RCAN3 | EPHA6 | | SCF | MASP3 | DUS10 | | DCC | | 0.615 |
| RCAN3 | EPHA6 | | SCF | MASP3 | DUS10 | | DCC | CD248 | 0.615 |
| RCAN3 | EPHA6 | | SCF | MASP3 | | | DCC | | 0.614 |
| RCAN3 | EPHA6 | | SCF | | DUS10 | | DCC | CD248 | 0.613 |
| RCAN3 | EPHA6 | | SCF | | DUS10 | | DCC | | 0.612 |
| RCAN3 | | | SCF | MASP3 | DUS10 | | DCC | CD248 | 0.611 |
| RCAN3 | EPHA6 | | SCF | | | | DCC | CD248 | 0.611 |
| RCAN3 | | | SCF | MASP3 | | | DCC | CD248 | 0.61 |
| RCAN3 | EPHA6 | | SCF | | | | DCC | | 0.609 |
| RCAN3 | | | SCF | MASP3 | DUS10 | | DCC | | 0.607 |
| RCAN3 | | | SCF | MASP3 | | | DCC | | 0.606 |
| RCAN3 | | | SCF | | DUS10 | | DCC | CD248 | 0.605 |
| RCAN3 | EPHA6 | | SCF | MASP3 | DUS10 | | | | 0.605 |
| RCAN3 | EPHA6 | | SCF | MASP3 | DUS10 | | | CD248 | 0.605 |
| RCAN3 | | | SCF | MASP3 | DUS10 | | | CD248 | 0.604 |
| RCAN3 | EPHA6 | | SCF | MASP3 | | | | | 0.604 |
| RCAN3 | EPHA6 | | SCF | MASP3 | | | | CD248 | 0.604 |
| RCAN3 | | | SCF | | | | DCC | CD248 | 0.603 |
| RCAN3 | | | SCF | MASP3 | | | | CD248 | 0.603 |
| RCAN3 | | | SCF | MASP3 | DUS10 | | | | 0.603 |
| RCAN3 | | | SCF | | DUS10 | | DCC | | 0.602 |
| RCAN3 | | | SCF | MASP3 | | | | | 0.602 |
| RCAN3 | EPHA6 | | | MASP3 | DUS10 | IgG4, | DCC | CD248 | 0.602 |
| | | | | | | Kappa | | | |
| RCAN3 | | | | MASP3 | DUS10 | IgG4, | | | 0.601 |
| | | | | | | Kappa | | | |
| RCAN3 | | | | MASP3 | DUS10 | IgG4, | DCC | CD248 | 0.601 |
| | | | | | | Kappa | | | |
| RCAN3 | EPHA6 | | | MASP3 | DUS10 | IgG4, | | | 0.601 |
| | | | | | | Kappa | | | |
| RCAN3 | EPHA6 | | | MASP3 | DUS10 | IgG4, | | CD248 | 0.601 |
| | | | | | | Kappa | | | |
| RCAN3 | EPHA6 | | | MASP3 | DUS10 | IgG4, | DCC | | 0.601 |
| | | | | | | Kappa | | | |
| RCAN3 | | | | MASP3 | DUS10 | IgG4, | | CD248 | 0.6 |
| | | | | | | Kappa | | | |
| RCAN3 | | | | MASP3 | DUS10 | IgG4, | DCC | | 0.6 |
| | | | | | | Kappa | | | |
| RCAN3 | EPHA6 | | | MASP3 | | IgG4, | DCC | CD248 | 0.6 |
| | | | | | | Kappa | | | |

TABLE 23
CD248 Biomarker Panels

| | | Alkaline | | | | | | | |
|---|---|---|---|---|---|---|---|---|---|
| | | phosphatase, | | | | IgG4, | | | |
| CD248 | EPHA6 | placental | SCF | MASP3 | DUS10 | Kappa | DCC | RCAN3 | AUC |
| CD248 | EPHA6 | Alkaline | SCF | MASP3 | DUS10 | IgG4, | DCC | RCAN3 | 0.698 |
| | | phosphatase, | | | | Kappa | | | |
| | | placental | | | | | | | |
| CD248 | | Alkaline | SCF | MASP3 | DUS10 | IgG4, | DCC | RCAN3 | 0.697 |
| | | phosphatase, | | | | Kappa | | | |
| | | placental | | | | | | | |
| CD248 | EPHA6 | Alkaline | SCF | MASP3 | DUS10 | IgG4, | DCC | | 0.697 |
| | | phosphatase, | | | | Kappa | | | |
| | | placental | | | | | | | |
| CD248 | | Alkaline | SCF | MASP3 | DUS10 | IgG4, | | | 0.696 |
| | | phosphatase, | | | | Kappa | | | |
| | | placental | | | | | | | |
| CD248 | | Alkaline | SCF | MASP3 | DUS10 | IgG4, | | RCAN3 | 0.696 |
| | | phosphatase, | | | | Kappa | | | |
| | | placental | | | | | | | |
| CD248 | | Alkaline | SCF | MASP3 | DUS10 | IgG4, | DCC | | 0.696 |
| | | phosphatase, | | | | Kappa | | | |
| | | placental | | | | | | | |
| CD248 | EPHA6 | Alkaline | SCF | MASP3 | DUS10 | IgG4, | | | 0.696 |
| | | phosphatase, | | | | Kappa | | | |
| | | placental | | | | | | | |
| CD248 | EPHA6 | Alkaline | SCF | MASP3 | DUS10 | IgG4, | | RCAN3 | 0.696 |
| | | phosphatase, | | | | Kappa | | | |
| | | placental | | | | | | | |
| CD248 | | Alkaline | SCF | MASP3 | | IgG4, | DCC | RCAN3 | 0.694 |
| | | phosphatase, | | | | Kappa | | | |
| | | placental | | | | | | | |
| CD248 | EPHA6 | Alkaline | SCF | MASP3 | | IgG4, | DCC | RCAN3 | 0.694 |
| | | phosphatase, | | | | Kappa | | | |
| | | placental | | | | | | | |
| CD248 | | Alkaline | SCF | MASP3 | | IgG4, | | | 0.693 |
| | | phosphatase, | | | | Kappa | | | |
| | | placental | | | | | | | |
| CD248 | | Alkaline | SCF | MASP3 | | IgG4, | | RCAN3 | 0.693 |
| | | phosphatase, | | | | Kappa | | | |
| | | placental | | | | | | | |
| CD248 | | Alkaline | SCF | MASP3 | | IgG4, | DCC | | 0.693 |
| | | phosphatase, | | | | Kappa | | | |
| | | placental | | | | | | | |
| CD248 | EPHA6 | Alkaline | SCF | MASP3 | | IgG4, | | RCAN3 | 0.693 |
| | | phosphatase, | | | | Kappa | | | |
| | | placental | | | | | | | |
| CD248 | EPHA6 | Alkaline | SCF | MASP3 | | IgG4, | DCC | | 0.693 |
| | | phosphatase, | | | | Kappa | | | |
| | | placental | | | | | | | |
| CD248 | EPHA6 | Alkaline | SCF | MASP3 | | IgG4, | | | 0.692 |
| | | phosphatase, | | | | Kappa | | | |
| | | placental | | | | | | | |
| CD248 | EPHA6 | Alkaline | SCF | MASP3 | DUS10 | | DCC | RCAN3 | 0.689 |
| | | phosphatase, | | | | | | | |
| | | placental | | | | | | | |
| CD248 | | Alkaline | SCF | | DUS10 | IgG4, | DCC | RCAN3 | 0.687 |
| | | phosphatase, | | | | Kappa | | | |
| | | placental | | | | | | | |
| CD248 | | Alkaline | SCF | MASP3 | DUS10 | | DCC | RCAN3 | 0.687 |
| | | phosphatase, | | | | | | | |
| | | placental | | | | | | | |
| CD248 | EPHA6 | Alkaline | SCF | | DUS10 | IgG4, | DCC | RCAN3 | 0.687 |
| | | phosphatase, | | | | Kappa | | | |
| | | placental | | | | | | | |
| CD248 | EPHA6 | Alkaline | SCF | MASP3 | DUS10 | | DCC | | 0.687 |
| | | phosphatase, | | | | | | | |
| | | placental | | | | | | | |
| CD248 | | Alkaline | SCF | MASP3 | DUS10 | | DCC | | 0.686 |
| | | phosphatase, | | | | | | | |
| | | placental | | | | | | | |
| CD248 | EPHA6 | Alkaline | SCF | | DUS10 | IgG4, | DCC | | 0.686 |
| | | phosphatase, | | | | Kappa | | | |
| | | placental | | | | | | | |
| CD248 | | Alkaline | SCF | | DUS10 | IgG4, | DCC | | 0.685 |
| | | phosphatase, | | | | Kappa | | | |
| | | placental | | | | | | | |
| CD248 | | Alkaline | SCF | MASP3 | DUS10 | | | RCAN3 | 0.685 |
| | | phosphatase, | | | | | | | |
| | | placental | | | | | | | |
| CD248 | EPHA6 | Alkaline | SCF | MASP3 | DUS10 | | | RCAN3 | 0.685 |
| | | phosphatase, | | | | | | | |
| | | placental | | | | | | | |
| CD248 | | Alkaline | SCF | MASP3 | DUS10 | | | | 0.684 |
| | | phosphatase, | | | | | | | |
| | | placental | | | | | | | |
| CD248 | EPHA6 | Alkaline | SCF | MASP3 | | | DCC | RCAN3 | 0.684 |
| | | phosphatase, | | | | | | | |
| | | placental | | | | | | | |
| CD248 | EPHA6 | Alkaline | SCF | MASP3 | DUS10 | | | | 0.684 |
| | | phosphatase, | | | | | | | |
| | | placental | | | | | | | |
| CD248 | | Alkaline | | MASP3 | DUS10 | IgG4, | DCC | | 0.683 |
| | | phosphatase, | | | | Kappa | | | |
| | | placental | | | | | | | |
| CD248 | | Alkaline | SCF | MASP3 | | | DCC | RCAN3 | 0.683 |
| | | phosphatase, | | | | | | | |
| | | placental | | | | | | | |
| CD248 | EPHA6 | Alkaline | | MASP3 | DUS10 | IgG4, | DCC | | 0.683 |
| | | phosphatase, | | | | Kappa | | | |
| | | placental | | | | | | | |
| CD248 | | Alkaline | | MASP3 | DUS10 | IgG4, | DCC | RCAN3 | 0.682 |
| | | phosphatase, | | | | Kappa | | | |
| | | placental | | | | | | | |
| CD248 | EPHA6 | Alkaline | | MASP3 | DUS10 | IgG4, | DCC | RCAN3 | 0.682 |
| | | phosphatase, | | | | Kappa | | | |
| | | placental | | | | | | | |
| CD248 | EPHA6 | Alkaline | SCF | | | IgG4, | DCC | RCAN3 | 0.682 |
| | | phosphatase, | | | | Kappa | | | |
| | | placental | | | | | | | |
| CD248 | EPHA6 | Alkaline | SCF | MASP3 | | | DCC | | 0.682 |
| | | phosphatase, | | | | | | | |
| | | placental | | | | | | | |
| CD248 | | Alkaline | SCF | | | IgG4, | DCC | RCAN3 | 0.681 |
| | | phosphatase, | | | | Kappa | | | |
| | | placental | | | | | | | |
| CD248 | | Alkaline | SCF | | DUS10 | IgG4, | | RCAN3 | 0.681 |
| | | phosphatase, | | | | Kappa | | | |
| | | placental | | | | | | | |
| CD248 | | Alkaline | SCF | MASP3 | | | | RCAN3 | 0.681 |
| | | phosphatase, | | | | | | | |
| | | placental | | | | | | | |
| CD248 | | Alkaline | SCF | MASP3 | | | DCC | | 0.681 |
| | | phosphatase, | | | | | | | |
| | | placental | | | | | | | |
| CD248 | EPHA6 | Alkaline | | MASP3 | DUS10 | IgG4, | | | 0.681 |
| | | phosphatase, | | | | Kappa | | | |
| | | placental | | | | | | | |
| CD248 | EPHA6 | Alkaline | | MASP3 | DUS10 | IgG4, | | RCAN3 | 0.681 |
| | | phosphatase, | | | | Kappa | | | |
| | | placental | | | | | | | |
| CD248 | EPHA6 | Alkaline | SCF | | DUS10 | IgG4, | | RCAN3 | 0.681 |
| | | phosphatase, | | | | Kappa | | | |
| | | placental | | | | | | | |
| CD248 | EPHA6 | Alkaline | SCF | MASP3 | | | | RCAN3 | 0.681 |
| | | phosphatase, | | | | | | | |
| | | placental | | | | | | | |
| CD248 | | Alkaline | | MASP3 | DUS10 | IgG4, | | | 0.68 |
| | | phosphatase, | | | | Kappa | | | |
| | | placental | | | | | | | |
| CD248 | | Alkaline | | MASP3 | DUS10 | IgG4, | | RCAN3 | 0.68 |
| | | phosphatase, | | | | Kappa | | | |
| | | placental | | | | | | | |
| CD248 | | Alkaline | SCF | | DUS10 | IgG4, | | | 0.68 |
| | | phosphatase, | | | | Kappa | | | |
| | | placental | | | | | | | |
| CD248 | | Alkaline | SCF | MASP3 | | | | | 0.68 |
| | | phosphatase, | | | | | | | |
| | | placental | | | | | | | |
| CD248 | EPHA6 | Alkaline | SCF | | | IgG4, | DCC | | 0.68 |
| | | phosphatase, | | | | Kappa | | | |
| | | placental | | | | | | | |
| CD248 | EPHA6 | Alkaline | SCF | | DUS10 | IgG4, | | | 0.68 |
| | | phosphatase, | | | | Kappa | | | |
| | | placental | | | | | | | |
| CD248 | EPHA6 | Alkaline | SCF | MASP3 | | | | | 0.68 |
| | | phosphatase, | | | | | | | |
| | | placental | | | | | | | |
| CD248 | | Alkaline | SCF | | | IgG4, | DCC | | 0.679 |
| | | phosphatase, | | | | Kappa | | | |
| | | placental | | | | | | | |
| CD248 | | Alkaline | | MASP3 | | IgG4, | DCC | | 0.678 |
| | | phosphatase, | | | | Kappa | | | |
| | | placental | | | | | | | |
| CD248 | | Alkaline | | MASP3 | | IgG4, | DCC | RCAN3 | 0.678 |
| | | phosphatase, | | | | Kappa | | | |
| | | placental | | | | | | | |
| CD248 | EPHA6 | Alkaline | | MASP3 | | IgG4, | DCC | | 0.678 |
| | | phosphatase, | | | | Kappa | | | |
| | | placental | | | | | | | |
| CD248 | EPHA6 | Alkaline | | MASP3 | | IgG4, | DCC | RCAN3 | 0.678 |
| | | phosphatase, | | | | Kappa | | | |
| | | placental | | | | | | | |
| CD248 | | Alkaline | SCF | | | IgG4, | | RCAN3 | 0.676 |
| | | phosphatase, | | | | Kappa | | | |
| | | placental | | | | | | | |
| CD248 | EPHA6 | Alkaline | | MASP3 | | IgG4, | | | 0.676 |
| | | phosphatase, | | | | Kappa | | | |
| | | placental | | | | | | | |
| CD248 | EPHA6 | Alkaline | SCF | | | IgG4, | | RCAN3 | 0.676 |
| | | phosphatase, | | | | Kappa | | | |
| | | placental | | | | | | | |
| CD248 | | Alkaline | | MASP3 | | IgG4, | | | 0.675 |
| | | phosphatase, | | | | Kappa | | | |
| | | placental | | | | | | | |
| CD248 | | Alkaline | | MASP3 | | IgG4, | | RCAN3 | 0.675 |
| | | phosphatase, | | | | Kappa | | | |
| | | placental | | | | | | | |
| CD248 | | Alkaline | | MASP3 | DUS10 | | DCC | | 0.675 |
| | | phosphatase, | | | | | | | |
| | | placental | | | | | | | |
| CD248 | | Alkaline | | MASP3 | DUS10 | | DCC | RCAN3 | 0.675 |
| | | phosphatase, | | | | | | | |
| | | placental | | | | | | | |
| CD248 | | Alkaline | SCF | | | IgG4, | | | 0.675 |
| | | phosphatase, | | | | Kappa | | | |
| | | placental | | | | | | | |
| CD248 | EPHA6 | Alkaline | | MASP3 | | IgG4, | | RCAN3 | 0.675 |
| | | phosphatase, | | | | Kappa | | | |
| | | placental | | | | | | | |
| CD248 | EPHA6 | Alkaline | | MASP3 | DUS10 | | DCC | | 0.674 |
| | | phosphatase, | | | | | | | |
| | | placental | | | | | | | |
| CD248 | EPHA6 | Alkaline | | MASP3 | DUS10 | | DCC | RCAN3 | 0.674 |
| | | phosphatase, | | | | | | | |
| | | placental | | | | | | | |
| CD248 | EPHA6 | Alkaline | SCF | | | IgG4, | | | 0.674 |
| | | phosphatase, | | | | Kappa | | | |
| | | placental | | | | | | | |
| CD248 | EPHA6 | Alkaline | SCF | | DUS10 | | DCC | RCAN3 | 0.674 |
| | | phosphatase, | | | | | | | |
| | | placental | | | | | | | |
| CD248 | | Alkaline | SCF | | DUS10 | | DCC | RCAN3 | 0.672 |
| | | phosphatase, | | | | | | | |
| | | placental | | | | | | | |
| CD248 | EPHA6 | Alkaline | SCF | | DUS10 | | DCC | | 0.672 |
| | | phosphatase, | | | | | | | |
| | | placental | | | | | | | |
| CD248 | | Alkaline | | MASP3 | DUS10 | | | | 0.671 |
| | | phosphatase, | | | | | | | |
| | | placental | | | | | | | |
| CD248 | | Alkaline | | MASP3 | DUS10 | | | RCAN3 | 0.671 |
| | | phosphatase, | | | | | | | |
| | | placental | | | | | | | |
| CD248 | EPHA6 | Alkaline | | MASP3 | DUS10 | | | RCAN3 | 0.671 |
| | | phosphatase, | | | | | | | |
| | | placental | | | | | | | |
| CD248 | | Alkaline | SCF | | DUS10 | | DCC | | 0.67 |
| | | phosphatase, | | | | | | | |
| | | placental | | | | | | | |
| CD248 | EPHA6 | Alkaline | | MASP3 | DUS10 | | | | 0.67 |
| | | phosphatase, | | | | | | | |
| | | placental | | | | | | | |
| CD248 | | Alkaline | | MASP3 | | | DCC | | 0.668 |
| | | phosphatase, | | | | | | | |
| | | placental | | | | | | | |
| CD248 | | Alkaline | | MASP3 | | | DCC | RCAN3 | 0.668 |
| | | phosphatase, | | | | | | | |
| | | placental | | | | | | | |
| CD248 | EPHA6 | Alkaline | | MASP3 | | | DCC | | 0.668 |
| | | phosphatase, | | | | | | | |
| | | placental | | | | | | | |
| CD248 | EPHA6 | Alkaline | | MASP3 | | | DCC | RCAN3 | 0.668 |
| | | phosphatase, | | | | | | | |
| | | placental | | | | | | | |
| CD248 | | Alkaline | | | DUS10 | IgG4, | DCC | | 0.667 |
| | | phosphatase, | | | | Kappa | | | |
| | | placental | | | | | | | |
| CD248 | | Alkaline | | | DUS10 | IgG4, | DCC | RCAN3 | 0.667 |
| | | phosphatase, | | | | Kappa | | | |
| | | placental | | | | | | | |
| CD248 | EPHA6 | Alkaline | | | DUS10 | IgG4, | DCC | | 0.667 |
| | | phosphatase, | | | | Kappa | | | |
| | | placental | | | | | | | |
| CD248 | EPHA6 | Alkaline | | | DUS10 | IgG4, | DCC | RCAN3 | 0.667 |
| | | phosphatase, | | | | Kappa | | | |
| | | placental | | | | | | | |
| CD248 | EPHA6 | Alkaline | SCF | | | | DCC | RCAN3 | 0.667 |
| | | phosphatase, | | | | | | | |
| | | placental | | | | | | | |
| CD248 | | Alkaline | SCF | | | | DCC | RCAN3 | 0.665 |
| | | phosphatase, | | | | | | | |
| | | placental | | | | | | | |
| CD248 | | Alkaline | SCF | | DUS10 | | | RCAN3 | 0.665 |
| | | phosphatase, | | | | | | | |
| | | placental | | | | | | | |
| CD248 | EPHA6 | Alkaline | SCF | | DUS10 | | | RCAN3 | 0.665 |
| | | phosphatase, | | | | | | | |
| | | placental | | | | | | | |
| CD248 | | Alkaline | | MASP3 | | | | | 0.664 |
| | | phosphatase, | | | | | | | |
| | | placental | | | | | | | |
| CD248 | | Alkaline | | MASP3 | | | | RCAN3 | 0.664 |
| | | phosphatase, | | | | | | | |
| | | placental | | | | | | | |
| CD248 | EPHA6 | Alkaline | | MASP3 | | | | | 0.664 |
| | | phosphatase, | | | | | | | |
| | | placental | | | | | | | |
| CD248 | EPHA6 | Alkaline | | MASP3 | | | | RCAN3 | 0.664 |
| | | phosphatase, | | | | | | | |
| | | placental | | | | | | | |
| CD248 | EPHA6 | Alkaline | SCF | | | | DCC | | 0.664 |
| | | phosphatase, | | | | | | | |
| | | placental | | | | | | | |
| CD248 | | Alkaline | SCF | | | | DCC | | 0.662 |
| | | phosphatase, | | | | | | | |
| | | placental | | | | | | | |
| CD248 | | Alkaline | SCF | | DUS10 | | | | 0.662 |
| | | phosphatase, | | | | | | | |
| | | placental | | | | | | | |
| CD248 | EPHA6 | Alkaline | SCF | | DUS10 | | | | 0.662 |
| | | phosphatase, | | | | | | | |
| | | placental | | | | | | | |
| CD248 | EPHA6 | Alkaline | | | | IgG4, | DCC | | 0.661 |
| | | phosphatase, | | | | Kappa | | | |
| | | placental | | | | | | | |
| CD248 | EPHA6 | Alkaline | | | | IgG4, | DCC | RCAN3 | 0.661 |
| | | phosphatase, | | | | Kappa | | | |
| | | placental | | | | | | | |
| CD248 | | Alkaline | | | | IgG4, | DCC | | 0.66 |
| | | phosphatase, | | | | Kappa | | | |
| | | placental | | | | | | | |
| CD248 | | Alkaline | | | | IgG4, | DCC | RCAN3 | 0.66 |
| | | phosphatase, | | | | Kappa | | | |
| | | placental | | | | | | | |
| CD248 | | Alkaline | | | DUS10 | IgG4, | | | 0.66 |
| | | phosphatase, | | | | Kappa | | | |
| | | placental | | | | | | | |
| CD248 | | Alkaline | | | DUS10 | IgG4, | | RCAN3 | 0.66 |
| | | phosphatase, | | | | Kappa | | | |
| | | placental | | | | | | | |
| CD248 | EPHA6 | Alkaline | | | DUS10 | IgG4, | | | 0.66 |
| | | phosphatase, | | | | Kappa | | | |
| | | placental | | | | | | | |
| CD248 | EPHA6 | Alkaline | | | DUS10 | IgG4, | | RCAN3 | 0.66 |
| | | phosphatase, | | | | Kappa | | | |
| | | placental | | | | | | | |
| CD248 | | Alkaline | SCF | | | | | RCAN3 | 0.658 |
| | | phosphatase, | | | | | | | |
| | | placental | | | | | | | |
| CD248 | EPHA6 | Alkaline | SCF | | | | | RCAN3 | 0.658 |
| | | phosphatase, | | | | | | | |
| | | placental | | | | | | | |
| CD248 | EPHA6 | Alkaline | | | DUS10 | | DCC | | 0.656 |
| | | phosphatase, | | | | | | | |
| | | placental | | | | | | | |
| CD248 | EPHA6 | Alkaline | | | DUS10 | | DCC | RCAN3 | 0.656 |
| | | phosphatase, | | | | | | | |
| | | placental | | | | | | | |
| CD248 | | Alkaline | | | DUS10 | | DCC | | 0.655 |
| | | phosphatase, | | | | | | | |
| | | placental | | | | | | | |
| CD248 | | Alkaline | | | DUS10 | | DCC | RCAN3 | 0.655 |
| | | phosphatase, | | | | | | | |
| | | placental | | | | | | | |
| CD248 | | Alkaline | SCF | | | | | | 0.655 |
| | | phosphatase, | | | | | | | |
| | | placental | | | | | | | |
| CD248 | EPHA6 | Alkaline | SCF | | | | | | 0.654 |
| | | phosphatase, | | | | | | | |
| | | placental | | | | | | | |
| CD248 | EPHA6 | Alkaline | | | | IgG4, | | RCAN3 | 0.652 |
| | | phosphatase, | | | | Kappa | | | |
| | | placental | | | | | | | |
| CD248 | | Alkaline | | | | IgG4, | | | 0.651 |
| | | phosphatase, | | | | Kappa | | | |
| | | placental | | | | | | | |
| CD248 | | Alkaline | | | | IgG4, | | RCAN3 | 0.651 |
| | | phosphatase, | | | | Kappa | | | |
| | | placental | | | | | | | |
| CD248 | EPHA6 | Alkaline | | | | IgG4, | | | 0.651 |
| | | phosphatase, | | | | Kappa | | | |
| | | placental | | | | | | | |
| CD248 | EPHA6 | Alkaline | | | | | DCC | RCAN3 | 0.648 |
| | | phosphatase, | | | | | | | |
| | | placental | | | | | | | |
| CD248 | | Alkaline | | | | | DCC | RCAN3 | 0.647 |
| | | phosphatase, | | | | | | | |
| | | placental | | | | | | | |
| CD248 | EPHA6 | Alkaline | | | | | DCC | | 0.647 |
| | | phosphatase, | | | | | | | |
| | | placental | | | | | | | |
| CD248 | | Alkaline | | | DUS10 | | | RCAN3 | 0.646 |
| | | phosphatase, | | | | | | | |
| | | placental | | | | | | | |
| CD248 | | Alkaline | | | | | DCC | | 0.645 |
| | | phosphatase, | | | | | | | |
| | | placental | | | | | | | |
| CD248 | | Alkaline | | | DUS10 | | | | 0.645 |
| | | phosphatase, | | | | | | | |
| | | placental | | | | | | | |
| CD248 | EPHA6 | Alkaline | | | DUS10 | | | RCAN3 | 0.645 |
| | | phosphatase, | | | | | | | |
| | | placental | | | | | | | |
| CD248 | EPHA6 | Alkaline | | | DUS10 | | | | 0.644 |
| | | phosphatase, | | | | | | | |
| | | placental | | | | | | | |
| CD248 | EPHA6 | | SCF | MASP3 | | IgG4, | DCC | | 0.635 |
| | | | | | | Kappa | | | |
| CD248 | EPHA6 | | SCF | MASP3 | | IgG4, | DCC | RCAN3 | 0.635 |
| | | | | | | Kappa | | | |
| CD248 | EPHA6 | | SCF | MASP3 | DUS10 | IgG4, | DCC | | 0.634 |
| | | | | | | Kappa | | | |
| CD248 | EPHA6 | | SCF | MASP3 | DUS10 | IgG4, | DCC | RCAN3 | 0.634 |
| | | | | | | Kappa | | | |
| CD248 | | | SCF | MASP3 | | IgG4, | DCC | | 0.631 |
| | | | | | | Kappa | | | |
| CD248 | | | SCF | MASP3 | | IgG4, | DCC | RCAN3 | 0.631 |
| | | | | | | Kappa | | | |
| CD248 | | | SCF | MASP3 | DUS10 | IgG4, | DCC | | 0.631 |
| | | | | | | Kappa | | | |
| CD248 | | Alkaline | | | | | | RCAN3 | 0.631 |
| | | phosphatase, | | | | | | | |
| | | placental | | | | | | | |
| CD248 | | | SCF | MASP3 | DUS10 | IgG4, | DCC | RCAN3 | 0.63 |
| | | | | | | Kappa | | | |
| CD248 | | Alkaline | | | | | | | 0.63 |
| | | phosphatase, | | | | | | | |
| | | placental | | | | | | | |
| CD248 | EPHA6 | Alkaline | | | | | | RCAN3 | 0.63 |
| | | phosphatase, | | | | | | | |
| | | placental | | | | | | | |
| CD248 | EPHA6 | | SCF | | | IgG4, | DCC | | 0.629 |
| | | | | | | Kappa | | | |
| CD248 | EPHA6 | | SCF | | | IgG4, | DCC | RCAN3 | 0.629 |
| | | | | | | Kappa | | | |
| CD248 | EPHA6 | | SCF | | DUS10 | IgG4, | DCC | | 0.629 |
| | | | | | | Kappa | | | |
| CD248 | EPHA6 | | SCF | | DUS10 | IgG4, | DCC | RCAN3 | 0.629 |
| | | | | | | Kappa | | | |
| CD248 | EPHA6 | Alkaline | | | | | | | 0.629 |
| | | phosphatase, | | | | | | | |
| | | placental | | | | | | | |
| CD248 | | | SCF | | DUS10 | IgG4, | DCC | | 0.627 |
| | | | | | | Kappa | | | |
| CD248 | | | SCF | | DUS10 | IgG4, | DCC | RCAN3 | 0.627 |
| | | | | | | Kappa | | | |
| CD248 | | | SCF | MASP3 | DUS10 | IgG4, | | | 0.627 |
| | | | | | | Kappa | | | |
| CD248 | | | SCF | MASP3 | DUS10 | IgG4, | | RCAN3 | 0.627 |
| | | | | | | Kappa | | | |
| CD248 | EPHA6 | | SCF | MASP3 | DUS10 | IgG4, | | | 0.627 |
| | | | | | | Kappa | | | |
| CD248 | EPHA6 | | SCF | MASP3 | DUS10 | IgG4, | | RCAN3 | 0.627 |
| | | | | | | Kappa | | | |
| CD248 | | | SCF | | | IgG4, | DCC | | 0.626 |
| | | | | | | Kappa | | | |
| CD248 | | | SCF | | | IgG4, | DCC | RCAN3 | 0.626 |
| | | | | | | Kappa | | | |
| CD248 | | | SCF | MASP3 | | IgG4, | | | 0.626 |
| | | | | | | Kappa | | | |
| CD248 | | | SCF | MASP3 | | IgG4, | | RCAN3 | 0.626 |
| | | | | | | Kappa | | | |
| CD248 | EPHA6 | | SCF | MASP3 | | IgG4, | | | 0.626 |
| | | | | | | Kappa | | | |
| CD248 | EPHA6 | | SCF | MASP3 | | IgG4, | | RCAN3 | 0.626 |
| | | | | | | Kappa | | | |
| CD248 | | | SCF | | DUS10 | IgG4, | | | 0.618 |
| | | | | | | Kappa | | | |
| CD248 | | | SCF | | DUS10 | IgG4, | | RCAN3 | 0.618 |
| | | | | | | Kappa | | | |
| CD248 | EPHA6 | | SCF | | DUS10 | IgG4, | | | 0.618 |
| | | | | | | Kappa | | | |
| CD248 | EPHA6 | | SCF | | DUS10 | IgG4, | | RCAN3 | 0.618 |
| | | | | | | Kappa | | | |
| CD248 | | | SCF | | | IgG4, | | | 0.616 |
| | | | | | | Kappa | | | |
| CD248 | | | SCF | | | IgG4, | | RCAN3 | 0.616 |
| | | | | | | Kappa | | | |
| CD248 | EPHA6 | | SCF | | | IgG4, | | RCAN3 | 0.616 |
| | | | | | | Kappa | | | |
| CD248 | EPHA6 | | SCF | | | IgG4, | | | 0.615 |
| | | | | | | Kappa | | | |
| CD248 | EPHA6 | | SCF | MASP3 | | | DCC | | 0.615 |
| CD248 | EPHA6 | | SCF | MASP3 | | | DCC | RCAN3 | 0.615 |
| CD248 | EPHA6 | | SCF | MASP3 | DUS10 | | DCC | | 0.615 |
| CD248 | EPHA6 | | SCF | MASP3 | DUS10 | | DCC | RCAN3 | 0.615 |
| CD248 | EPHA6 | | SCF | | DUS10 | | DCC | RCAN3 | 0.613 |
| CD248 | EPHA6 | | SCF | | DUS10 | | DCC | | 0.612 |
| CD248 | | | SCF | MASP3 | DUS10 | | DCC | | 0.611 |
| CD248 | | | SCF | MASP3 | DUS10 | | DCC | RCAN3 | 0.611 |
| CD248 | EPHA6 | | SCF | | | | DCC | RCAN3 | 0.611 |
| CD248 | | | SCF | MASP3 | | | DCC | | 0.61 |
| CD248 | | | SCF | MASP3 | | | DCC | RCAN3 | 0.61 |
| CD248 | EPHA6 | | SCF | | | | DCC | | 0.61 |
| CD248 | | | SCF | | DUS10 | | DCC | | 0.605 |
| CD248 | | | SCF | | DUS10 | | DCC | RCAN3 | 0.605 |
| CD248 | EPHA6 | | SCF | MASP3 | DUS10 | | | | 0.605 |
| CD248 | EPHA6 | | SCF | MASP3 | DUS10 | | | RCAN3 | 0.605 |
| CD248 | | | SCF | MASP3 | DUS10 | | | | 0.604 |
| CD248 | | | SCF | MASP3 | DUS10 | | | RCAN3 | 0.604 |
| CD248 | EPHA6 | | SCF | MASP3 | | | | | 0.604 |
| CD248 | EPHA6 | | SCF | MASP3 | | | | RCAN3 | 0.604 |
| CD248 | | | SCF | | | | DCC | RCAN3 | 0.603 |
| CD248 | | | SCF | MASP3 | | | | | 0.603 |
| CD248 | | | SCF | MASP3 | | | | RCAN3 | 0.603 |
| CD248 | | | SCF | | | | DCC | | 0.602 |
| CD248 | EPHA6 | | | MASP3 | DUS10 | IgG4, | DCC | | 0.602 |
| | | | | | | Kappa | | | |
| CD248 | EPHA6 | | | MASP3 | DUS10 | IgG4, | DCC | RCAN3 | 0.602 |
| | | | | | | Kappa | | | |
| CD248 | | | | MASP3 | DUS10 | IgG4, | DCC | | 0.601 |
| | | | | | | Kappa | | | |
| CD248 | | | | MASP3 | DUS10 | IgG4, | DCC | RCAN3 | 0.601 |
| | | | | | | Kappa | | | |
| CD248 | EPHA6 | | | MASP3 | DUS10 | IgG4, | | RCAN3 | 0.601 |
| | | | | | | Kappa | | | |
| CD248 | | | | MASP3 | DUS10 | IgG4, | | RCAN3 | 0.6 |
| | | | | | | Kappa | | | |
| CD248 | EPHA6 | | | MASP3 | | IgG4, | DCC | | 0.6 |
| | | | | | | Kappa | | | |
| CD248 | EPHA6 | | | MASP3 | | IgG4, | DCC | RCAN3 | 0.6 |
| | | | | | | Kappa | | | |

Biomarker Panels, Includes * EPHA6, Alkaline phosphatase-placental, SCF,
MASP3:Heavy, DUS10, IgG4-Kappa, DCC, RCAN3, CD248 plus additional markers

| AUC | * | EDIL3 | REN | MSMB | PIGR | CCDC80 | AGRN | SCARF2 | SCGB3A1 | SOX2 | IGFALS | SLPI | REG4 | WFDC1 |
|---|---|---|---|---|---|---|---|---|---|---|---|---|---|---|
| 0.721334 | * | EDIL3 | | | | | | | | | | | | |
| 0.726299 | * | | REN | | | | | | | | | | | |
| 0.721408 | * | | | MSMB | | | | | | | | | | |
| 0.700731 | * | | | | PIGR | | | | | | | | | |
| 0.709309 | * | | | | | CCDC80 | | | | | | | | |
| 0.702793 | * | | | | | | AGRN | | | | | | | |
| 0.702331 | * | | | | | | | SCARF2 | | | | | | |
| 0.707717 | * | | | | | | | | SCGB3A1 | | | | | |
| 0.699452 | * | | | | | | | | | SOX2 | | | | |
| 0.704038 | * | | | | | | | | | | IGFALS | | | |
| 0.722142 | * | | | | | | | | | | | SLPI | | |
| 0.722976 | * | | | | | | | | | | | | REG4 | |
| 0.716748 | * | | | | | | | | | | | | | WFDC1 |

| AUC | * | BPIFB1 | MMP8 | NTN4 | REG1A | DLD | TCP10L3 | MCAM | TAGLN | TIMP4 | FAM3B | FBLN1 | CRTAC1 | ALPG |
|---|---|---|---|---|---|---|---|---|---|---|---|---|---|---|
| 0.705325 | * | BPIFB1 | | | | | | | | | | | | |
| 0.699304 | * | | MMP8 | | | | | | | | | | | |
| 0.710183 | * | | | NTN4 | | | | | | | | | | |
| 0.715915 | * | | | | REG1A | | | | | | | | | |
| 0.706183 | * | | | | | DLD | | | | | | | | |
| 0.699122 | * | | | | | | TCP10L3 | | | | | | | |
| 0.702694 | * | | | | | | | MCAM | | | | | | |
| 0.706496 | * | | | | | | | | TAGLN | | | | | |
| 0.703271 | * | | | | | | | | | TIMP4 | | | | |
| 0.718728 | * | | | | | | | | | | FAM3B | | | |
| 0.697687 | * | | | | | | | | | | | FBLN1 | | |
| 0.69777 | * | | | | | | | | | | | | CRTAC1 | |
| 0.698537 | * | | | | | | | | | | | | | ALPG |

| AUC | * | CCL21 | CTSV | WFDC2 | PPP2R3A | DCTPP1 | MXRA8 | TCP11L1 |
|---|---|---|---|---|---|---|---|---|
| 0.704129 | * | CCL21 | | | | | | |
| 0.698017 | * | | CTSV | | | | | |
| 0.732378 | * | | | WFDC2 | | | | |
| 0.700533 | * | | | | PPP2R3A | | | |
| 0.696994 | * | | | | | DCTPP1 | | |
| 0.698331 | * | | | | | | MXRA8 | |
| 0.697151 | * | | | | | | | TCP11L1 |

TABLE 25
Univariate performance for each marker from Table 4 alone

| Target_Names | AUC |
|---|---|
| ALPP | 0.632083 |
| WFDC2 | 0.623315 |
| SLPI | 0.620169 |
| REN | 0.618094 |
| ALPG | 0.614779 |
| MSMB | 0.608514 |
| REG1A | 0.600815 |
| REG4 | 0.600106 |
| EDIL3 | 0.599219 |
| WFDC1 | 0.593763 |
| PIGR | 0.591565 |
| FAM3B | 0.588006 |
| KITLG | 0.587243 |
| IGFALS | 0.581102 |
| SCGB3A1 | 0.579329 |
| BPIFB1 | 0.57756 |
| TCP11L1 | 0.572978 |
| CCDC80 | 0.570788 |
| DLD | 0.570508 |
| IGHG4 | 0.568301 |
| MASP1 | 0.564095 |
| TIMP4 | 0.563979 |
| AGRN | 0.561633 |
| NTN4 | 0.55682 |
| CCL21 | 0.555113 |
| DCC | 0.553719 |
| SOX2 | 0.550185 |
| TAGLN | 0.547389 |
| DUSP10 | 0.544052 |
| CTSV | 0.544011 |
| PPP2R3A | 0.534378 |
| SCARF2 | 0.524035 |
| TCP10L3 | 0.522657 |
| FBLN1 | 0.522554 |
| RCAN3 | 0.518764 |
| MMP8 | 0.514178 |
| EPHA6 | 0.514038 |
| MCAM | 0.512273 |
| CD248 | 0.510112 |
| MXRA8 | 0.508269 |
| DCTPP1 | 0.506594 |
| CRTAC1 | 0.502314 |

What is claimed is:

1. A method of predicting a probability of a subject ever having been a tobacco user, comprising detecting a level of EPHA6 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

2. A method of detecting levels of N biomarker proteins in a sample, comprising obtaining the sample from a subject, and detecting the level of each of the N biomarker proteins in the sample from the subject, wherein Nis at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

3. A method of predicting a probability of a subject ever having been a tobacco user, comprising detecting a level of Alkaline phosphatase-placental biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

4. A method of predicting a probability of a subject ever having been a tobacco user, comprising detecting a level of SCF biomarker protein and a level of each of N biomarker proteins in a sample from the subject, wherein Nis at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

5. A method of predicting a probability of a subject ever having been a tobacco user, comprising detecting a level of MASP3: Heavy biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

6. A method of predicting a probability of a subject ever having been a tobacco user, comprising detecting a level of DUS10 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

7. A method of predicting a probability of a subject ever having been a tobacco user, comprising detecting a level of IgG4-Kappa biomarker protein and a level of each of N biomarker proteins in a sample from the subject, wherein Nis at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, DUS10, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

8. A method of predicting a probability of a subject ever having been a tobacco user, comprising detecting a level of DCC biomarker protein and a level of each of N biomarker proteins in a sample from the subject, wherein Nis at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, DUS10, IgG4-Kappa, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

9. A method of predicting a probability of a subject ever having been a tobacco user, comprising detecting a level of RCAN3 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, DUS10, IgG4-Kappa, DCC, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

10. A method of predicting a probability of a subject ever having been a tobacco user, comprising detecting a level of CD248 biomarker protein and a level of each of N biomarker proteins in a sample from the subject, wherein Nis at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker proteins is selected from EPHA6, Alkaline phosphatase-placental, SCF, MASP3: Heavy, DUS10, IgG4-Kappa, DCC, RCAN3, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

11. The method of any one of the preceding claims, wherein N is 2 to 39, or N is 3 to 39, or N is 4 to 39, or N is 5 to 39, or N is 6 to 39, or N is 7 to 39, or N is 8 to 39, or N is 9 to 39, or N is 10 to 39, or N is 11 to 39, or N is 12 to 39, or N is 13 to 39, or N is 14 to 39, Nis 15 to 39, N is 16 to 39, N is 17 to 39, N is 18 to 39, N is 19 to 39, N is 20 to 39, N is 21 to 39, N is 22 to 39, N is 23 to 39, N is 24 to 39, N is 25 to 39, N is 26 to 39, N is 27 to 39, N is 28 to 39, N is 29 to 39, N is 30 to 39, N is 31 to 39, N is 32 to 39, N is 33 to 39, N is 34 to 39, N is 35 to 39, N is 36 to 39, N is 37 to 39, or N is 38 to 39.

12. The method of any one of the preceding claims, wherein N is 2, N is 3, or N is 4, or Nis 5, or N is 6, or N is 7, or N is 8, or N is 9, or N is 10, or N is 11, or N is 12, or N is 13, or N is 14, or N is 15, or N is 16, or N is 17, or N is 18, or N is 19, or N is 20, or N is 21, or Nis 22, or N is 23, or N is 24, N is 25, N is 26, N is 27, N is 28, N is 29, N is 30, N is 31, N is 32, N is 33, N is 34, N is 35, N is 36, N is 37, N is 38, or N is 39.

13. The method of any one of the preceding claims, wherein each of the N biomarker proteins is selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

14. The method of any one of the preceding claims, wherein at least one of the N biomarker proteins is selected from EPHA6, Alkaline phosphatase-placental, SCF, MASP3: Heavy, DUS10, IgG4-Kappa, DCC, RCAN3, and CD248.

15. The method of any one of the preceding claims, wherein at least 2, at least 3, at least 4, at least 5, at least 6, at least 7, at least 8, or at least 9 of the N protein biomarkers are selected from EPHA6, Alkaline phosphatase-placental, SCF, MASP3: Heavy, DUS10, IgG4-Kappa, DCC, RCAN3, and CD248.

16. The method of any one of the preceding claims, wherein two of the N biomarker proteins are EPHA6 and Alkaline phosphatase-placental, or two of the N biomarker proteins are EPHA6 and SCF, or two of the N biomarker proteins are EPHA6 and MASP3: Heavy, or two of the N biomarker proteins are EPHA6 and DUS10, or two of the N biomarker proteins are EPHA6 and IgG4-Kappa, or two of the N biomarker proteins are EPHA6 and DCC, or two of the N biomarker proteins are EPHA6 and RCAN3, or two of the N biomarker proteins are EPHA6 and CD248.

17. The method of any one of the preceding claims, wherein two of the N biomarker proteins are Alkaline phosphatase-placental and SCF, or two of the N biomarker proteins are Alkaline phosphatase-placental and MASP3: Heavy, or two of the N biomarker proteins are Alkaline phosphatase-placental and DUS10, or two of the N biomarker proteins are Alkaline phosphatase-placental and IgG4-Kappa, or two of the N biomarker proteins are Alkaline phosphatase-placental and DCC, or two of the N biomarker proteins are Alkaline phosphatase-placental and RCAN3, or two of the N biomarker proteins are Alkaline phosphatase-placental and CD248.

18. The method of any one of the preceding claims, wherein two of the N biomarker proteins are SCF and MASP3: Heavy, or two of the N biomarker proteins are SCF and DUS10, or two of the N biomarker proteins are SCF and IgG4-Kappa, or two of the N biomarker proteins are SCF and DCC, or two of the N biomarker proteins are SCF and RCAN3, or two of the N biomarker proteins are SCF and CD248.

19. The method of any one of the preceding claims, wherein two of the N biomarker proteins are MASP3: Heavy and DUS10, or two of the N biomarker proteins are MASP3: Heavy and IgG4-Kappa, or two of the N biomarker proteins are MASP3: Heavy and DCC, or two of the N biomarker proteins are MASP3: Heavy and RCAN3, or two of the N biomarker proteins are MASP3: Heavy and CD248.

20. The method of any one of the preceding claims, wherein two of the N biomarker proteins are DUS10 and IgG4-Kappa, or two of the N biomarker proteins are DUS10 and DCC, or two of the N biomarker proteins are DUS10 and RCAN3, or two of the N biomarker proteins are DUS10 and CD248.

21. The method of any one of the preceding claims, wherein two of the N biomarker proteins are IgG4-Kappa and DCC, or two of the N biomarker proteins are IgG4-Kappa and RCAN3, or two of the N biomarker proteins are IgG4-Kappa and CD248.

22. The method of any one of the preceding claims, wherein two of the N biomarker proteins are DCC and RCAN3, or two of the N biomarker proteins are DCC and CD248.

23. The method of any one of the preceding claims, wherein two of the N biomarker proteins are RCAN3 and CD248.

24. A method of predicting a probability of a subject ever having been a tobacco user, comprising detecting a level of T11L1 biomarker protein.

25. A method of predicting a probability of a subject ever having been a tobacco user, comprising detecting a level of MXRA8 biomarker protein.

26. A method of predicting a probability of a subject ever having been a tobacco user, comprising detecting a level of XTP3A biomarker protein.

27. A method of predicting a probability of a subject ever having been a tobacco user, comprising detecting a level of CRAC1 biomarker protein.

28. The method of any one of claims 24-27, further comprising detecting at least one biomarker protein selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit.

29. The method of any one of the preceding claims, wherein the sample is a blood sample, a plasma sample, or a serum sample.

30. The method of any one of the preceding claims, wherein detecting is performed using mass spectrometry, an aptamer based assay and/or an antibody based assay.

31. The method of any one of the preceding claims, wherein the method comprises contacting biomarker proteins of the sample or samples with a set of biomarker capture reagents, wherein each biomarker capture reagent of the set of biomarker capture reagents specifically binds to a different biomarker protein being detected.

32. The method of claim 31, wherein each biomarker capture reagent is an antibody or an aptamer.

33. The method of claim 32, wherein each biomarker capture reagent is an aptamer.

34. The method of claim 33, wherein at least one aptamer is a slow off-rate aptamer.

35. The method of claim 34, wherein at least one slow off-rate aptamer comprises at least one, at least two, at least three, at least four, at least five, at least six, at least seven, at least eight, at least nine, or at least 10 nucleotides with modifications.

36. The method of claim 34 or claim 35, wherein each slow off-rate aptamer binds to its target protein with an off rate (t½) of ≥20 minutes, ≥30 minutes, ≥60 minutes, ≥90 minutes, ≥120 minutes, ≥150 minutes, ≥180 minutes, ≥210 minutes, or ≥240 minutes.

37. The method of any one of claims 30-36, wherein the level of each biomarker protein measured is determined from a relative florescence unit (RFU) or a protein concentration.

38. The method of any one of the preceding claims, wherein predicting the probability of the subject ever having been a tobacco user is based on input of the levels of the N biomarker proteins measured in a statistical model.

39. The method of claim 38, wherein the determining comprises analyzing the levels of the N biomarker protein using an elastic net logistic regression model.

40. The method of claim 38 or 39, wherein the model has an area under the curve (AUC) selected from at least 0.65, at least 0.66, at least 0.67, at least 0.68, at least 0.69, at least 0.7, at least 0.75, at least 0.8, at least 0.85, at least 0.9, or at least 0.95.

41. The method of any one of claims 33-35, wherein the model provides an absolute risk probability of having ever been a tobacco user.

42. The method of any one of claims 38-41, wherein the model provides a value between 0 and 1, wherein a value>0.460 is predictive of an ever tobacco user.

43. The method of any one of claims 38-42, wherein the model provides the absolute probability of having been an ever tobacco user based on the level of each of the biomarker proteins selected from EPHA6, Alkaline phosphatase-placental, SCF, MASP3: Heavy, DUS10, IgG4-Kappa, DCC, RCAN3, and CD248.

44. The method of any one of claims 38-43, wherein the model provides the absolute probability of having been an ever tobacco user based on the level of each of the biomarker proteins selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

45. The method of any one of the preceding claims, wherein the method comprises predicting a probability of a subject ever having been a tobacco user for the purpose of determining a medical insurance premium or life insurance premium.

46. The method of claim 45, wherein the method further comprises determining coverage or premium for medical insurance or life insurance.

47. The method of any one of claims 1-46, wherein the method further comprises using information resulting from the method to predict and/or manage the utilization of medical resources.

48. The method of any one of claims 1-47, wherein the method further comprises using information resulting from the method to enable a decision to acquire or purchase a medical practice, hospital, or company.

49. The method of any one of claims 1-40, wherein the tobacco user is a smoker.

50. A kit comprising N biomarker protein capture reagents, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39, and wherein at least one of the N biomarker protein capture reagents specifically binds to a biomarker protein selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

51. The kit of claim 50, wherein Nis at least two and at least one of the two N biomarker protein capture reagents specifically binds to the biomarker protein selected from EPHA6, Alkaline phosphatase-placental, SCF, MASP3: Heavy, DUS10, IgG4-Kappa, DCC, RCAN3, and CD248 and the other of the two N biomarker proteins is selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

52. The kit of claim 50 or 51, wherein N is 2 to 39, or N is 3 to 39, or N is 4 to 39, or N is 5 to 39, or N is 6 to 39, or N is 7 to 39, or N is 8 to 39, or N is 9 to 39, or N is 10 to 39, or Nis 11 to 39, or N is 12 to 39, or N is 13 to 39, or N is 14 to 39, N is 15 to 39, N is 16 to 39, Nis 17 to 39, N is 18 to 39, N is 19 to 39, N is 20 to 39, N is 21 to 39, N is 22 to 39, N is 23 to 39, N is 24 to 39, N is 25 to 39, N is 26 to 39, N is 27 to 39, N is 28 to 39, N is 29 to 39, N is 30 to 39, N is 31 to 39, N is 32 to 39, N is 33 to 39, N is 34 to 39, N is 35 to 39, N is 36 to 39, N is 37 to 39, or N is 38 to 39.

53. The kit of any one of claims 50-52, wherein N is 2, N is 3, or N is 4, or N is 5, or N is 6, or N is 7, or N is 8, or N is 9, or N is 10, or N is 11, or N is 12, or N is 13, or N is 14, or Nis 15, or N is 16, or N is 17, or N is 18, or N is 19, or N is 20, or N is 21, or N is 22, or N is 23, or N is 24, N is 25, N is 26, N is 27, N is 28, N is 29, N is 30, N is 31, N is 32, Nis 33, N is 34, N is 35, N is 36, N is 37, N is 38, or N is 39.

54. The kit of any one of claims 50-53, wherein each of the N biomarker protein capture reagents specifically binds to a different biomarker protein.

55. The kit of any one of claims 50-54, wherein each of the N biomarker protein capture reagents specifically binds to a biomarker protein selected from EPHA6, Alkaline phosphatase-placental, SCF, MASP3: Heavy, DUS10, IgG4-Kappa, DCC, RCAN3, CD248, EDIL3, Renin, PSP-94, PIGR, URB, Agrin, Secretoglobin family 3A member 1, SOX2, SREC-II, IGFALS, SLPI, WFDC1, REG4, MMP-8, LPLC1, NET4, PSP, DLDH, TCP10, MUC18, TAGL, TIMP-4, FAM3B, fibulin 1, CRAC1, PPBN, 6Ckine, Cathepsin V, HE4, and PP2A-subunit B.

56. The kit of any one of claims 50-55, wherein at least 1 of the N biomarker protein capture reagents specifically binds to a biomarker protein selected from EPHA6, Alkaline phosphatase-placental, SCF, MASP3: Heavy, DUS10, IgG4-Kappa, DCC, RCAN3, and CD248.

57. The kit of any one of claims 50-56, wherein at least 2, at least 3, at least 4, at least 5, at least 6, at least 7, at least 8, or at least 9 of the N biomarker protein capture reagents specifically binds to a biomarker protein selected from EPHA6, Alkaline phosphatase-placental, SCF, MASP3: Heavy, DUS10, IgG4-Kappa, DCC, RCAN3, and CD248.

58. The kit of any one of claims 50-57, wherein two of the N biomarker protein capture reagents specifically bind EPHA6 and Alkaline phosphatase-placental, or two of the N biomarker protein capture reagents specifically bind EPHA6 and SCF, or two of the N biomarker protein capture reagents specifically bind EPHA6 and MASP3: Heavy, or two of the N biomarker protein capture reagents specifically bind EPHA6 and DUS10, or two of the N biomarker protein capture reagents specifically bind EPHA6 and IgG4-Kappa, or two of the N biomarker protein capture reagents specifically bind EPHA6 and DCC, or two of the N biomarker protein capture reagents specifically bind EPHA6 and RCAN3, or two of the N biomarker protein capture reagents specifically bind EPHA6 and CD248.

59. The kit of any one of claims 50-57, wherein two of the N biomarker protein capture reagents specifically bind Alkaline phosphatase-placental and SCF, or two of the N biomarker protein capture reagents specifically bind Alkaline phosphatase-placental and MASP3: Heavy, or two of the N biomarker protein capture reagents specifically bind Alkaline phosphatase-placental and DUS10, or two of the N biomarker protein capture reagents specifically bind Alkaline phosphatase-placental and IgG4-Kappa, or two of the N biomarker protein capture reagents specifically bind Alkaline phosphatase-placental and DCC, or two of the N biomarker protein capture reagents specifically bind Alkaline phosphatase-placental and RCAN3, or two of the N biomarker protein capture reagents specifically bind Alkaline phosphatase-placental and CD248.

60. The kit of any one of claims 50-57, wherein two of the N biomarker protein capture reagents specifically bind SCF and MASP3: Heavy, or two of the N biomarker protein capture reagents specifically bind SCF and DUS10, or two of the N biomarker protein capture reagents specifically bind SCF and IgG4-Kappa, or two of the N biomarker protein capture reagents specifically bind SCF and DCC, or two of the N biomarker protein capture reagents specifically bind SCF and RCAN3, or two of the N biomarker protein capture reagents specifically bind SCF and CD248.

61. The method of any one of claims 50-57, wherein two of the N biomarker protein capture reagents specifically bind MASP3: Heavy and DUS10, or two of the N biomarker protein capture reagents specifically bind MASP3: Heavy and IgG4-Kappa, or two of the N biomarker protein capture reagents specifically bind MASP3: Heavy and DCC, or two of the N biomarker protein capture reagents specifically bind MASP3: Heavy and RCAN3, or two of the N biomarker protein capture reagents specifically bind MASP3: Heavy and CD248.

62. The kit of any one of claims 50-57, wherein two of the N biomarker protein capture reagents specifically bind DUS10 and IgG4-Kappa, or two of the N biomarker protein capture reagents specifically bind DUS10 and DCC, or two of the N biomarker protein capture reagents specifically bind DUS10 and RCAN3, or two of the N biomarker protein capture reagents specifically bind DUS10 and CD248.

63. The kit of any one of claims 50-57, wherein two of the N biomarker protein capture reagents specifically bind IgG4-Kappa and DCC, or two of the N biomarker protein capture reagents specifically bind IgG4-Kappa and RCAN3, or two of the N biomarker protein capture reagents specifically bind IgG4-Kappa and CD248.

64. The kit of any one of claims 50-57, wherein two of the N biomarker protein capture reagents specifically bind DCC and RCAN3, or two of the N biomarker protein capture reagents specifically bind DCC and CD248.

65. The kit of any one of claims 50-57, wherein two of the N biomarker protein capture reagents specifically bind RCAN3 and CD248.

66. A kit comprising N biomarker protein capture reagents, wherein the kit comprises biomarker protein capture reagents for carrying out the method of any one of claims 1-49.

67. The kit of any one of claims 50-66, wherein each of the N biomarker protein capture reagents is an antibody or an aptamer.

68. The kit of claim 67, wherein each biomarker protein capture reagent is an aptamer.

69. The kit of claim 68, wherein at least one aptamer is a slow off-rate aptamer.

70. The kit of claim 69, wherein at least one slow off-rate aptamer comprises at least one, at least two, at least three, at least four, at least five, at least six, at least seven, at least eight, at least nine, or at least 10 nucleotides with modifications.

71. The kit of claim 69 or claim 70, wherein each slow off-rate aptamer binds to its target protein with an off rate (t½) of ≥20 minutes, ≥30 minutes, ≥60 minutes, ≥90 minutes, ≥120 minutes, ≥150 minutes, ≥180 minutes, ≥210 minutes, or ≥240 minutes.

72. The kit of any one of claims 50-71, for use in detecting the N biomarker proteins in a sample from a subject.

73. The kit of claim 72, for use in predicting a probability of a subject ever having been a tobacco user.

74. The kit of claim 73, wherein the tobacco user is a smoker.
